(19) United States
(12) Patent Application Publication
(10) Pub. No.: US 2006/0160869 A1
(43) Pub. Date: Jul. 20, 2006

(54) Ubiquitin ligase inhibitors

(75) Inventors: Rajinder Singh, Belmont, CA (US); Usha Ramesh, Cupertino, CA (US); Sarkiz D. Issakani, San Jose, CA (US); Jianing Huang, Foster City, CA (US); Taisei Kinoshita, San Mateo, CA (US); Tarikere Gururaja, Santa Clara, CA (US)

Correspondence Address: Michael S. Greenfield;McDonnell Boehnen Hulbert & Berghoff LLP, 31st Floor, 300 S. Wacker Drive, Chicago, IL, 60606, US

(73) Assignee: Rigel Pharmaceuticals, Inc., South San Francisco, CA (US)

(21) Appl. No.: 11/325,845

(22) Filed: Jan. 5, 2006

Related U.S. Application Data
(60) Provisional application No. 60/641,495, filed on Jan. 5, 2005.

(51) Int. Cl. A61K 31/427; A61K 31/426; C07D 277/38; C07D 417/02

(52) U.S. Cl. 514/370; 548/190

(57) ABSTRACT

This invention describes compounds and pharmaceutical compositions useful as ubiquitin agent inhibitors, particularly ubiquitin ligase inhibitors. The compounds and pharmaceutical compositions of the invention are useful as inhibitors of the biochemical pathways of organisms in which ubiquitination is involved, such as signal transduction pathways. The invention also comprises the use of the compounds and pharmaceutical compositions of the invention for the treatment of conditions that require inhibition of ubiquitination. Furthermore, the invention comprises methods of inhibiting ubiquitination in a cell comprising contacting a cell in which inhibition of ubiquitination is desired with a compound or pharmaceutical composition according to the invention. Particularly, the compounds and pharmaceutical compositions are useful to inhibit the ubiquitin ligase activity of MDM2.

This application claims the benefit of U.S. provisional application 60/641,495, filed Jan. 5, 2005.

### BACKGROUND OF THE INVENTION

1. Field of the Invention

This invention relates to the inhibition of ubiquitination. More particularly, the invention relates to compounds and methods for inhibiting ubiquitin ligase activity.

2. Summary of the Related Art

Ubiquitin is a 76 amino acid protein present throughout the eukaryotic kingdom. It is a highly conserved protein and is essentially the identical protein in diverse organisms ranging from humans to yeasts to fruit flies. In eukaryotes, ubiquitin is the key component of the ATP-dependent pathway for protein degradation. Proteins slated for degradation are covalently linked to ubiquitin via an ATP-dependent process catalyzed by three separate enzymes.

Ubiquitin has also been implicated as key components in other biochemical processes. Ubiquitination of the Gag structural protein of Rous Sarcoma virus has been linked to the targeting of Gag to the cell membrane of the host cell where it can assemble into spherical particles and bud from the cell surface. Production of HIV particles has also been associated with ubiquitination and may constitute an important cellular pathway for producing infectious particles. Thus, the ubiquitin pathway may be an important target for treatment of HIV positive patients.

There is a need for inhibitors of ubiquitin ligases that can alter the ATP-dependent ubiquitination of proteins. Inhibition of ubiquitination can regulate the degradation of proteins in ways that assist in treating various disorders. Inhibitors of ubiquitin ligases may also help in treating infectious diseases such as bacterial and viral infections that depend on the cellular biochemical machinery.

The ubiquitination of these target proteins is known to be mediated by the enzymatic activity of three ubiquitin agents. Ubiquitin is first activated in an ATP-dependent manner by a ubiquitin activating agent, for example, an E1. The C-terminus of a ubiquitin forms a high energy thiolester bond with the ubiquitin activating agent. The ubiquitin is then transferred to a ubiquitin conjugating agent, for example, an E2 (also called ubiquitin moiety carrier protein), also linked to this second ubiquitin agent via a thiolester bond. The ubiquitin is finally linked to its target protein (e.g. substrate) to form a terminal isopeptide bond under the guidance of a ubiquitin ligating agent, for example, an E3. In this process, monomers or oligomers of ubiquitin are attached to the target protein. On the target protein, each ubiquitin is covalently ligated to the next ubiquitin through the activity of a ubiquitin ligating agent to form polymers of ubiquitin.

The enzymatic components of the ubiquitination pathway have received considerable attention (for a review, see Weissman, Nature Reviews 2:169-178 (2001)). The members of the E1 ubiquitin activating agents and E2 ubiquitin conjugating agents are structurally related and well characterized enzymes. There are numerous species of E2 ubiquitin conjugating agents, some of which act in preferred pairs with specific E3 ubiquitin ligating agents to confer specificity for different target proteins. While the nomenclature for the E2 ubiquitin conjugating agents is not standardized across species, investigators in the field have addressed this issue and the skilled artisan can readily identify various E2 ubiquitin conjugating agents, as well as species homologues (See Haas and Siepmann, FASEB J. 11:1257-1268 (1997)).

Generally, ubiquitin ligating agents contain two separate activities: a ubiquitin ligase activity to attach, via an isopeptide bond, monomers or oligomers of ubiquitin to a target protein, and a targeting activity to physically bring the ligase and substrate together. The substrate specificity of different ubiquitin ligating agents is a major determinant in the selectivity of the ubiquitin-mediated protein degradation process.

In eukaryotes, some ubiquitin ligating agents contain multiple subunits that form a complex called the SCF having ubiquitin ligating activity. SCFs play an important role in regulating G1 progression, and consists of at least three subunits, SKP1, Cullins (having at least seven family members) and an F-box protein (of which hundreds of species are known) which bind directly to and recruit the substrate to the complex. The combinatorial interactions between the SCF's and a recently discovered family of RING finger proteins, the ROC/APC11 proteins, have been shown to be the key elements conferring ligase activity to ubiquitin ligating agents. Particular ROC/Cullin combinations can regulate specific cellular pathways, as exemplified by the function of APC11-APC2, involved in the proteolytic control of sister chromatid separation and exit from telophase into G1 in mitosis (see King et al., supra; Koepp et al., Cell 97:431-34 (1999)), and ROC1-Cullin 1, involved in the proteolytic degradation of IκB in NF-κB/IκB mediated transcription regulation (Tan et al., Mol. Cell 3(4):527-533 (1999); Laney et al., Cell 97:427-30 (1999)).

The best characterized ubiquitin ligating agent is the APC (anaphase promoting complex), which is multi-component complex that is required for both entry into anaphase as well as exit from mitosis (see King et al., Science 274:1652-59 (1996) for review). The APC plays a crucial role in regulating the passage of cells through anaphase by promoting ubiquitin-mediated proteolysis of many proteins. In addition to degrading the mitotic B-type cyclin for inactivation of CDC2 kinase activity, the APC is also required for degradation of other proteins for sister chromatid separation and spindle disassembly. Most proteins known to be degraded by the APC contain a conserved nine amino acid motif known as the “destruction box” that targets them for ubiquitin ubiquitination and subsequent degradation. However, proteins that are degraded during G1, including G1 cyclins, CDK inhibitors, transcription factors and signaling intermediates, do not contain this conserved amino acid motif. Instead, substrate phosphorylation appears to play an important role in targeting their interaction with a ubiquitin ligating agent for ubiquitin ubiquitination (see Hershko et al., Ann. Rev. Biochem. 67:429-75 (1998)).

Two major classes of E3 ubiquitin ligating agents are known: the HECT (homologous to E6-AP carboxy terminus) domain E3 ligating agents; and the RING finger domain E3 ligating agents. E6AP is the prototype for the HECT domain subclass of E3 ligating agents and is a multi-subunit complex that functions as a ubiquitin ligating agent for the tumor suppressor p53 which is activated by papillomavirus in cervical cancer (Huang et al. (1999) Science 286:1321-1326). Members of this class are homologous to the carboxyl terminus of E6AP and utilize a Cys active site to form a thiolester bond with ubiquitin, analogous to the E1 activating agents and E2 conjugating agents. However, in contrast, the members of the RING finger domain class of E3 ligating agents are thought to interact with an ubiquitin-conjugated-E2 intermediate to activate the complex for the transfer of ubiquitin to an acceptor. Examples of the RING domain class of E3 ligating agents are TRAF6, involved in IKK activation; Cbl, which targets insulin and EGF; Sina/Siah, which targets DCC; Itchy, which is involved in haematopoesis (B, T and mast cells); IAP, involved with inhibitors of apoptosis; and MDM2 which is involved in the regulation of p53.

Recently, an E2-E3 pair has been identified to consist of the UBC13-TRAF6 in conjuction with the Ubc-like protein Uev1A. This newly identified E2-E3 pair participates in many signal transduction pathways. TRAF6 (tumor necrosis factor (TNF) receptor associated factors) was first identified as intracellular proteins associated with TNF-R2 (reviewed by Wu et al., BioEssays 25, 1096-1105). Deng et al. (Cell 103, 351-361 (2000)) demonstrated that the RING domain of TRAF6, in conjunction with the ubiquitin-conjugating enzyme UBC13 and the UBC-like protein Uev1A, exhibited ubiquitin ligase activity and catalyzed polyubiquitin involving Lys-63, instead of Lys-48 of ubiquitin. It was later shown that the ubiquitin ligase activity of TRAF6 catalyzed the polyubiquitination of TAK1 which in turn activates a number of important kinases such as IkB kinase and MAP kinases (reviewed by Wu et al., BioEssays 25, 1096-1105).

The ubiquitination of TAK1 by TRAF6 does not lead to degradation of TAK1, but rather to the activation of TAK1 which then activates IkB kinase. IkB kinase in turn activates the NF-kB pathway as well as phosphorylates MKK6 in the JNK-p38 kinase pathway. The NF-kB pathway includes many important processes such as inflammation, LPS-induces septic shock, viral infection such as HIV, and cell survival among others. Thus, the ubiquitin ligase activity of TRAF6 plays important regulatory roles in many cellular processes.

The RING finger domain subclass of E3 ligating agents can be further grouped into two subclasses. In one subclass, the RING finger domain and the substrate recognition domain are contained on different subunits of a complex forming the ubiquitin ligating agent (e.g., the RB×1 and the F-box subunit of the SCF complex). In the second subclass of ubiquitin ligating agents, the ligating agents have the RING finger domain and substrate recognition domain on a single subunit. (e.g., MDM2 and cbl) (Tyers et al. (1999) Science 284:601, 603-604; Joazeiro et al. (2000) 102:549-552). A further class of ligating agents are those having a “PHD” domain and are homologs of the RING finger domain ligating agents (Coscoy et al. (2001) J. Cell Biol. 155(7):1265-1273), e.g., MEKK1. The PHD domain ligating agents are a novel class of membrane-bound E3 ligating agents.

In addition, a new class of ubiquitin ligases has been characterized. These are the U-box-containing proteins. (Patterson, Sci STKE 2002(116:PE4 (220)). This class, for the present, represents a small number of ligases which have yet to be extensively characterized.

MDM2 belongs to the second subclass of single subunit E3 ligating agents and is involved in regulating the function and stability of p53, an important tumor suppressor. In cells, p53 functions as a DNA-binding transcription factor which induces the expression of genes involved in DNA repair, apoptosis, and the arrest of cell growth. In approximately 50% of all human cancer p53 is inactivate by deletion or mutation. The level of p53 in the cell is maintained at low steady-state levels, and is induced and activated post-translationally by various signal pathways responsive to cellular stress (Lakin et al. (1999) Oncogene 18:7644-7655; Oren, M. (1999) J. Biol. Chem 274:36031-36,034). Stimuli that trigger the stress response and activate p53 include oxygen stress, inappropriate activation of oncogenes and agents that cause damage to DNA (e.g., ionizing radiation, chemicals, and ultra violet light).

The carboxyl terminus of MDM2 contains a variant of the RING finger domain (Saurin et al. (1996) Trends Biochem. Sci. 21:208-214) that is critical for the activity of this E3 ligating agent. Recent studies have shown that MDM2 mediates the ubiquitination of itself resulting in the formation of poly-ubiquitin chains on the protein (Zhihong et al. (2001) J. B. C. 276:31,357-31,367; Honda et al. (2000) Oncogene 19:1473-1476; Shengyun et al. (2000) 275:8945-8951). Further, the ubiquitin ligating activity of MDM2 is dependent on its RING finger domain.

Typically, the ubiquitination of target proteins by E3 in cells results in the formation of poly-ubiquitin chains. An isopeptide bond is formed between the carboxyl terminus of the ubiquitin and the ε-amino group of Lys in the target protein. The extension or formation of ubiquitin chains results from the formation of additional isopeptide bonds with the Lys⁴⁸ (and sometimes Lys⁶³) of a previously conjugated ubiquitin and the carboxyl-terminal Gly of an additional ubiquitin. The efficient recognition of a ubiquitinated target protein by a proteosome requires at least four ubiquitins linked in this configuration. However, in the case of MDM2-mediated ubiquitination of p53, neither Lys⁴⁸ or Lys⁶³ is involved in the formation of poly-ubiquitin chains. Recent studies show that human MDM2 mediates multiple mono-ubiquitination of p53 by a mechanism requiring enzyme isomerization (Zhihong et al. (2001) J. Biol. Chem. 276:31,357-31,367). Further, in vitro, the transfer of ubiquitin to p53 can occur independent of E1 when using an E2 pre-conjugated with ubiquitin. These results suggest that the pre-conjugated E2 can bind to MDM2 and thereafter transfer the ubiquitin to the MDM2 in the absence of an E1.

Thus, ubiquitin agents, such as the ubiquitin activating agents, ubiquitin conjugating agents, and ubiquitin ligating agents, are key determinants of the ubiquitin-mediated proteolytic pathway that results in the degradation of targeted proteins and regulation of cellular processes. Consequently, agents that modulate the activity of such ubiquitin agents may be used to up-regulate or down-regulate specific molecules involved in cellular signal transduction. Disease processes can be treated by such up- or down regulation of signal transducers to enhance or dampen specific cellular responses. This principle has been used in the design of a number of therapeutics, including phosphodiesterase inhibitors for airway disease and vascular insufficiency, kinase inhibitors for malignant transformation and Proteasome inhibitors for inflammatory conditions such as arthritis.

Due to the importance of ubiquitin-mediated proteolysis in cellular process, for example cell cycle regulation, there is a need for a fast and simple means for identifying the physiological role of ubiquitin agents that are catalytic components of this enzymatic pathway, and for identifying which ubiquitin agents are involved in various regulatory pathways. Thus, an object of the present invention is to provide methods of assaying for the physiological role of ubiquitin agents, and for providing methods for determining which ubiquitin agents are involved together in a variety of different physiological pathways.

### BRIEF SUMMARY OF THE INVENTION

The invention comprises compounds and pharmaceutical compositions of the compounds for inhibiting ubiquitin agents. The pharmaceutical compositions can be used in treating various conditions where ubiquitination is involved. They can also be used as research tools to study the role of ubiquitin in various natural and pathological processes. These findings suggest that inhibition of ubiquitin ligase activity represents a novel approach for intervening in cell cycle regulation and ubiquitin ligase inhibitors have great therapeutic potential in the treatment of cell proliferative diseases or conditions.

In a first aspect, the invention comprises compounds that inhibit ubiquitination of target proteins.

In a second aspect, the invention comprises a pharmaceutical composition comprising an inhibitor of ubiquitination according to the invention and a pharmaceutically acceptable carrier, excipient, or diluent.

In a third aspect, the invention comprises methods of inhibiting ubiquitination in a cell, comprising contacting a cell in which inhibition of ubiquitination is desired with a pharmaceutical composition comprising a ubiquitin agent inhibitor according to the invention.

In a fourth aspect, the invention provides methods for treating cell proliferative diseases or conditions, comprising administering to a patient in need thereof a pharmaceutical composition comprising an effective amount of a ubiquitin agent inhibitor according to the invention.

In a fifth aspect, the invention provides methods for inhibiting MDM2 activity in a cell, comprising administering to the cell a compound of the invention or a pharmaceutical composition comprising an effective amount of a compound according to the invention.

The foregoing only summarizes certain aspects of the invention and is not intended to be limiting in nature. These aspects and other aspects and embodiments are described more fully below. All patent applications and publications of any sort referred to in this specification are hereby incorporated by reference in their entirety. In the event of a discrepancy between the express disclosure of this specification and a patent application or publication incorporated by reference, the express disclosure of this specification shall control.

### DETAILED DESCRIPTION OF THE PREFERRED EMBODIMENTS

The invention provides compounds and methods for inhibiting ubiquitin ligase activity. The invention also provides compositions and methods for treating cell proliferative diseases and conditions.

In the first aspect, the invention provides compounds of Formula I 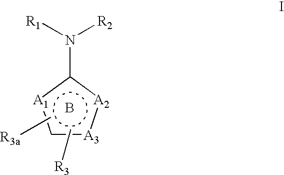
or pharmaceutically acceptable salts thereof, wherein
A₁, A₂ and A₃ are independently —C(R₆)(R₆ₐ)—, —C(R₆)═, —N═, —N(R₆)—, —O—, or —S—;
the dashed circle in the B ring indicates that the bonds of the ring are single or double bonds so that the B ring is a saturated, partially unsaturated, aromatic or nonaromatic ring;
R₁ is —H, natural or non-natural amino acids, C₁-C₆ alkyl, C₃-C₆ cycloalkyl, —C₁-C₆ alkylaryl, —C₁-C₆ alkylheteroaryl, —C₁-C₆ alkylheterocyclyl, —C₀-C₆ alkylaryl-C(O)N(R₅)(R₅ₐ), —C₀-C₆ alkylheteroaryl-C(O)—N(R₅)(R₅ₐ), —C₀-C₆ alkylaryl-NR₅—C(O)—R₅, —C₀-C₆ alkylheteroaryl-NR₅—C(O)—R₅, —C₀-C₆ alkylaryl-C(O)-heterocyclyl, —C₀-C₆ alkylheteroaryl-C(O)-heterocyclyl, —C₀-C₆ alkylaryl-C(O)-heteroaryl, —C₀-C₆ alkylheteroaryl-C(O)-heteroaryl, aryl, heteroaryl, or heterocyclyl, wherein each of the alkyl, aryl, heterocyclyl, and heteroaryl is optionally substituted with 1 to 4 groups independently selected from R₇;
R₂ is —H, C₁-C₆ alkyl, C₂-C₆ alkenyl, C₂-C₆ alkynyl, —C₁-C₆ alkyl-N(R₄)(R₄ₐ), —C₁-C₆ alkylaryl, —C₁-C₆ alkylheteroaryl, —C₁-C₆ alkylheterocyclyl, —C₁-C₆ alkyl-O-aryl, —C₁-C₆ alkyl-O-heteroaryl, aryl, heteroaryl, or heterocyclyl, wherein each of the alkyl, alkenyl, alkynyl, aryl, heterocyclyl, and heteroaryl is optionally substituted with 1 to 4 groups selected from oxo, —OH, —SH, C₁-C₆ alkyl, C₁-C₆ alkoxy, mono- to per-halogenated C₁-C₆ alkyl, mono- to per-halogenated C₁-C₆ alkoxy, —N(R₈)(R₈ₐ), —NO₂, halo, or —CN;
R₄ and R₄ₐ are independently —H, —OH, C₁-C₆ alkyl, aryl, —SH, C₂-C₆ alkenyl, C₂-C₆ alkynyl, —N(R₈)(R₈ₐ) or C₁-C₆ alkoxy;
R₅ and R₅ₐ are independently —H, —OH, C₁-C₆ alkyl, —C₀-C₆ alkyl-C(O)—OH, C₁-C₆ alkoxy, —C₁-C₆ alkyl-C₃-C₆ cycloalkyl, —C₀-C₆ alkylaryl, or —C₀-C₆ alkylheteroaryl, wherein each of the alkyl, aryl, and heteroaryl is optionally substituted with 1 to 4 groups selected from oxo, —OH, —SH, —C(O)—OH, C₁-C₆ alkyl, C₁-C₆ alkoxy, —C(O)—OH, mono- to per-halogenated C₁-C₆ alkyl, mono- to per-halogenated C₁-C₆ alkoxy, —C₁-C₆ alkyl-N(R₄)(R₄ₐ), —N(R₈)(R₈ₐ), —NO₂, halo, or —CN;
R₈ and R₈ₐ are independently —H or —C₁-C₆ alkyl;
R₆ and R₆ₐ are independently —H, —OH, C₁-C₆ alkyl, —SH, C₂-C₆ alkenyl, C₂-C₆ alkynyl, aryl, heteroaryl, heterocyclyl, —C(O)-aryl, —C(O)-heteroaryl, —C(O)-heterocyclyl, —C(O)—R₄, —N(R₈)(R₈ₐ) or C₁-C₆ alkoxy, wherein each of the alkyl, aryl, heterocyclyl, and heteroaryl is optionally substituted with 1 to 4 groups selected from oxo, —OH, —SH, C₁-C₆ alkyl, C₁-C₆ alkoxy, mono- to per-halogenated C₁-C₆ alkyl, mono- to per-halogenated C₁-C₆ alkoxy, aryl, heteroaryl, heterocyclyl, C₁-C₆ alkyl-N(R₄)(R₄ₐ), —N(R₈)(R₈ₐ), —NO₂, halo, or —CN;
R₇ is oxo, —OH, —SH, C₁-C₆ alkyl, C₁-C₆ alkoxy, —C(O)—OH, mono- to per-halogenated C₁-C₆ alkyl, mono- to per-halogenated C₁-C₆ alkoxy, —N(R₈)(R₈ₐ), —NO₂, halo, or —CN;
R₃ and R₃ₐ are independently —H, C₁-C₆ alkyl, C₁-C₆ alkoxy, mono- to per-halogenated C₁-C₆ alkyl, C₁-C₆ alkylaryl, —C₁-C₆ alkylheteroaryl, —C₁-C₆ alkylheterocyclyl, aryl, heteroaryl, or heterocyclyl, wherein each of the alkyl, aryl, heterocyclyl, and heteroaryl is optionally substituted with 1 to 4 groups selected from oxo, —OH, —SH, C₁-C₆ alkyl, C₁-C₆ alkoxy, mono- to per-halogenated C₁-C₆ alkyl, mono- to per-halogenated C₁-C₆ alkoxy, aryl, heteroaryl, heterocyclyl, C₁-C₆ alkyl-N(R₄)(R₄ₐ), —N(R₈)(R₈ₐ), —NO₂, halo, or —CN; or
R₃ or R₃ₐ together with a substituent attached to A₃ forms an aryl, heteroaryl, heterocyclyl or cycloalkyl group, wherein each of the aryl, heterocyclyl, cycloalkyl, and heteroaryl is optionally substituted with 1 to 4 groups selected from oxo, —OH, —SH, C₁-C₆ alkyl, C₁-C₆ alkoxy, mono- to per-halogenated C₁-C₆ alkyl, mono- to per-halogenated C₁-C₆ alkoxy, aryl, heteroaryl, heterocyclyl, —C₁-C₆ alkyl-N(R₄)(R₄ₐ), —N(R₈)(R₈ₐ), —NO₂, halo, or —CN; or
R₃ or R₃ₐ is aryl optionally substituted in the meta and para position with 1 to 4 groups selected from oxo, —OH, —SH, C₁-C₆ alkyl, C₁-C₆ alkoxy, mono- to per-halogenated C₁-C₆ alkyl, mono- to per-halogenated C₁-C₆ alkoxy, aryl, heteroaryl, heterocyclyl, C₁-C₆ alkyl-N(R₄)(R₄ₐ), —N(R₈)(R₈ₐ), —NO₂, halo, or —CN; and
provided that when R₃ together with a substituent attached to A₃ form an aryl, heteroaryl, heterocyclyl or cycloalkyl group, R₁ is —C₁-C₆ alkylaryl-C(O)—N(R₅)(R₅ₐ) or —C₁-C₆ alkylaryl-C(O)-heterocyclyl, and R₂ is C₁-C₆ alkyl, C₂-C₆ alkenyl, —C₁-C₆ alkyl-N(R₄)(R₄ₐ), —C₁-C₆ alkylaryl, —C₁-C₆ alkylheteroaryl, —C₁-C₆ alkyl-O-aryl or aryl.

Embodiment A according to formula I comprises compounds wherein R₁ is —C₀-C₆ alkylaryl-C(O)—N(R₅)(R₅ₐ), or —C₀-C₆ alkylaryl-C(O)-heterocyclyl; R₅ is —H or C₁-C₆ alkyl optionally substituted with —C(O)—OH, —C₀-C₆ alkyl-C(O)—OH, —C₁-C₆ alkyl-C₃-C₆ cycloalkyl, —C₀-C₆ alkylaryl, C₁-C₆ alkoxy, —C(O)—OH, —NH₂, or —C₁-C₆ alkyl-N(R₄)(R₄ₐ); and R₄ is —H.

Embodiment A′ according to formula I comprises compound wherein R₁ is —C₀-C₆ alkylaryl-C(O)-heterocyclyl. Preferably, R₁ is one of the following structure: 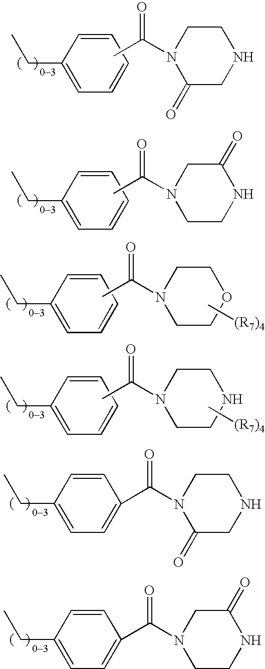
wherein each of the R₇ is the same or different.

Embodiment A″ according to formula I comprises compound wherein R₁ is —C₀-C₆ alkylheteroaryl-C(O)-heterocyclyl. Preferably, R₁ is one of the following structure:

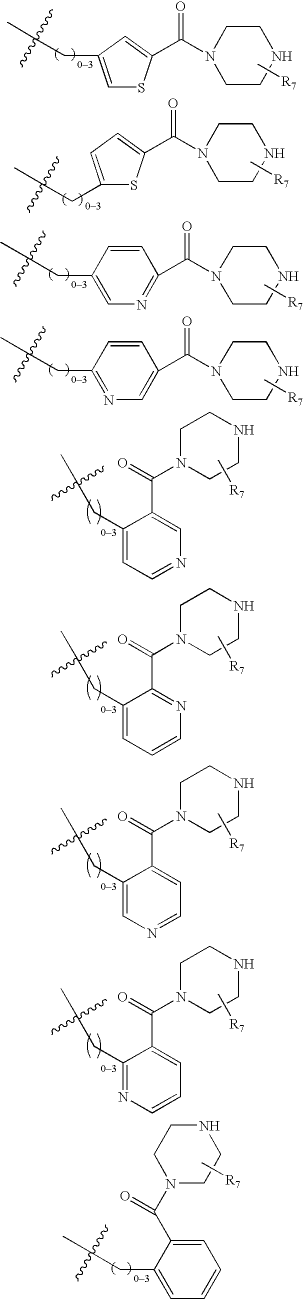

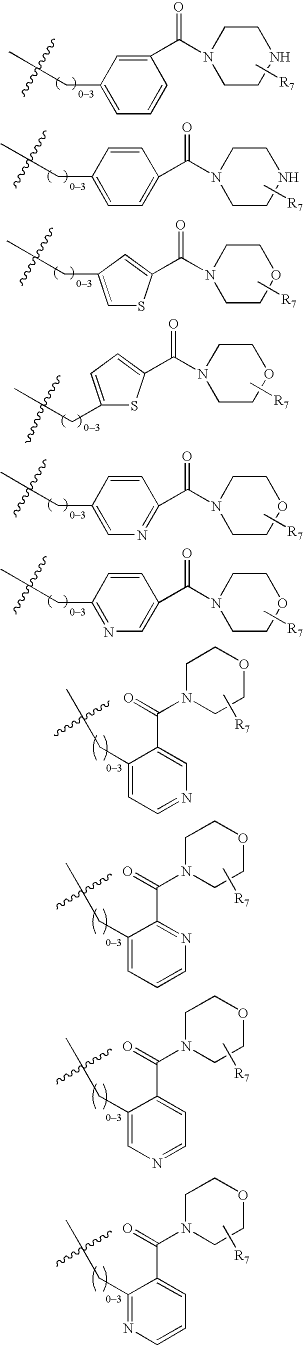

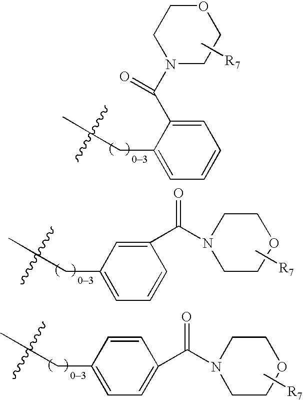

Embodiment A′″ according to formula I comprises compound wherein A₁, A₂ and A₃ is one of the following combination:

| A₁ | A₂ | A₃ |
|---|---|---|
| —N═ | —O— | —C(H)═ |
| —N═ | —S— | —C(H)═ |
| —C(H)═ | —O— | —N═ |
| —N═ | —S— | —N═ |

Embodiment B according to formula I comprises compounds wherein R₂ is C₁-C₆ alkyl, C₂-C₆ alkenyl, —C₁-C₆ alkyl-N(R₄)(R₄ₐ), —C₁-C₆ alkylaryl, —C₁-C₆ alkylheteroaryl, —C₁-C₆ alkyl-O-aryl, or aryl, wherein each of the alkyl, alkenyl, aryl, and heteroaryl is optionally substituted with 1 to 4 groups selected from mono- to per-halogenated C₁-C₆ alkoxy, or halo; and R₄ is C₁-C₆ alkyl.

Embodiment C according to formula I comprises compounds wherein R₃ is C₁-C₆ alkyl, mono- to per-halogenated C₁-C₆ alkyl, or aryl wherein each of the alkyl or aryl is optionally substituted with 1 to 4 groups selected from C₁-C₆ alkyl, C₁-C₆ alkoxy, aryl, —NO₂, or halo. Preferably, R₃ is phenyl optionally substituted in the meta and para position with 1 or 2 groups selected from halo, C₁-C₆ alkyl, C₁-C₆ alkoxy, aryl, or —NO₂. More preferably, R₃ is phenyl substituted in the meta and para position with 1 or 2 groups selected from —NO₂; methoxy, chloro, fluoro, bromo, methyl, or phenyl. Also preferred are compounds wherein R₃ is thiofuranyl, pyridinyl, pyrazinyl, pyrimidinyl or triazinyl.

Embodiment D according to formula I comprises compounds of the formula 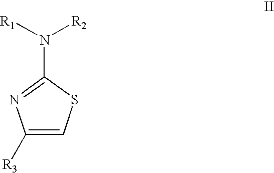
or pharmaceutically acceptable salts thereof, wherein
R₁ is —C₁-C₆ alkylaryl, —C₁-C₆ alkylheteroaryl, —C₁-C₆ alkylheterocyclyl, —C₀-C₆ alkylaryl-C(O)—N(R₅)(R₅ₐ), —C₀-C₆ alkylheteroaryl-C(O)—N(R₅)(R₅ₐ), —C₀-C₆ alkylaryl-NR₅—C(O)—R₅, —C₀-C₆ alkylheteroaryl-NR₅—C(O)—R₅, —C₀-C₆ alkylaryl-C(O)-heterocyclyl, —C₀-C₆ alkylheteroaryl-C(O)-heterocyclyl, —C₀-C₆ alkylaryl-C(O)-heteroaryl, —C₀-C₆ alkylheteroaryl-C(O)-heteroaryl; aryl, heteroaryl, or heterocyclyl, wherein each of the alkyl, aryl; heterocyclyl, and heteroaryl is optionally substituted with 1 to 4 groups independently selected from R₇;
R₂ is —H, C₁-C₆ alkyl, C₂-C₆ alkenyl, C₂-C₆ alkynyl, —C₁-C₆ alkyl-N(R₅)(R₅ₐ), —C₁-C₆ alkylaryl, —C₁-C₆ alkylheteroaryl, —C₁-C₆ alkylheterocyclyl, —C₁-C₆ alkyl-aryl, aryl, heteroaryl, or heterocyclyl, wherein each of the alkyl, alkenyl, alkynyl, aryl, heterocyclyl, and heteroaryl is optionally substituted with 1 to 4 groups selected from oxo, —OH, —SH, C₁-C₆ alkyl, C₁-C₆ alkoxy, mono- to per-halogenated C₁-C₆ alkyl, mono- to per-halogenated C₁-C₆ alkoxy, —N(R₈)(R₈ₐ), —NO₂, halo, or —CN;
R₄ and R₄ₐ are independently —H, —OH, C₁-C₆ alkyl, C₁-C₆ alkoxy, —SH, C₂-C₆ alkenyl, C₂-C₆ alkynyl, —N(R₈)(R₈ₐ) or C₁-C₆ alkoxy;
R₅ and R₅ₐ are independently —H, —OH, C₁-C₆ alkyl, —C₀-C₆ alkyl-C(O)—OH, C₁-C₆ alkoxy, —C₁-C₆ alkyl-C₃-C₆ cycloalkyl, —C₀-C₆ alkylaryl, or —C₁-C₆ alkylheteroaryl, wherein each of the alkyl, aryl, and heteroaryl are optionally substituted with 1 to 4 groups selected from oxo, —C(O)—OH, —OH, —SH, C₁-C₆ alkyl, C₁-C₆ alkoxy, mono- to per-halogenated C₁-C₆ alkyl, mono- to per-halogenated C₁-C₆ alkoxy, —C₁-C₆ alkyl-N(R₄)(R₄ₐ), —N(R₈)(R₈ₐ), —NO₂, halo, or —CN;
R₈ and R₈ₐ are independently —H or —C₁-C₆ alkyl;
R₇ is oxo, —OH, —SH, C₁-C₆ alkyl, C₁-C₆ alkoxy, —C(O)—OH, mono- to per-halogenated C₁-C₆ alkyl, mono- to per-halogenated C₁-C₆ alkoxy, —N(R₈)(R₈ₐ), —NO₂, halo, or —CN; and
R₃ is C₁-C₆ alkyl, mono- to per-halogenated C₁-C₆ alkyl, —C₁-C₆ alkylaryl, —C₁-C₆ alkylheteroaryl, —C₁-C₆ alkylheterocyclyl, aryl, heteroaryl, or heterocyclyl, wherein each of the alkyl, aryl, heterocyclyl, and heteroaryl is optionally substituted with 1 to 4 groups selected from oxo, —OH, —SH, C₁-C₆ alkyl, C₁-C₆ alkoxy, mono- to per-halogenated C₁-C₆ alkyl, mono- to per-halogenated C₁-C₆ alkoxy, aryl, heteroaryl, heterocyclyl, —C₁-C₆ alkyl-N(R₄)(R₄ₐ), —N(R₈)(R₈ₐ), —NO₂, halo, or —CN; or
R₃ is aryl optionally substituted in the meta and para position with 1 to 4 groups selected from oxo, —OH, —SH, C₁-C₆ alkyl, C₁-C₆ alkoxy, mono- to per-halogenated C₁-C₆ alkyl, mono- to per-halogenated C₁-C₆ alkoxy, aryl, heteroaryl, heterocyclyl, —C₁-C₆ alkyl-N(R₄)(R₄ₐ), —N(R₈)(R₈ₐ), —NO₂, halo, or —CN.

Embodiment E according to formula II comprises compounds wherein
R₁ is —C₀-C₆ alkylaryl-C(O)—N(R₅)(R₅ₐ) or —C₀-C₆ alkylaryl-C(O)-heterocyclyl;
R₂ is C₁-C₆ alkyl, C₂-C₆ alkenyl, —C₁-C₆ alkyl-N(R₄)(R₄ₐ), —C₁-C₆ alkylaryl, —C₁-C₆ alkylheteroaryl, —C₁-C₆ alkyl-O-aryl, or aryl, wherein each of the alkyl, alkenyl, aryl, and heteroaryl is optionally substituted with 1 to 4 groups selected from mono- to per-halogenated C₁-C₆ alkoxy, C₁-C₆ alkoxy or halo;
R₃ is C₁-C₆ alkyl, mono- to per-halogenated C₁-C₆ alkyl, or aryl wherein each of the alkyl or aryl is optionally substituted with 1 to 4 groups selected from C₁-C₆ alkyl, C₁-C₆ alkoxy, aryl, —NO₂, or halo; or
R₃ is aryl optionally substituted in the meta and para position with 1 to 4 groups selected from C₁-C₆ alkyl, C₁-C₆ alkoxy, aryl, halo, or —NO₂;
R₄ and R₄ₐ are independently —H or C₁-C₆ alkyl; and
R₅ and R₅ₐ are independently —H, C₁-C₆ alkyl, —C₀-C₆ alkyl-C(O)—OH —C₁-C₆ alkyl-C₃-C₆ cycloalkyl, or —C₀-C₆ alkylaryl, wherein each of the alkyl and aryl is optionally substituted with 1 to 4 groups selected from C₁-C₆ alkoxy, —C(O)—OH, —NH₂, or —C₁-C₆ alkyl-N(R₄)(R₄ₐ).

Embodiment F comprises compounds according to Embodiment E wherein
R₁ is —C₀-C₆ alkylaryl-C(O)—N(R₅)(R₅ₐ);
R₂ is C₁-C₆ alkyl, C₂-C₆ alkenyl, —C₁-C₆ alkylaryl, or aryl, wherein the aryl is optionally substituted with 1 to 4 groups selected from halo;
R₃ is aryl optionally substituted with 1 to 4 groups selected from C₁-C₆ alkoxy, —NO₂, aryl, or halo; or
R₃ is aryl optionally substituted in the meta and para position with 1 to 4 groups selected from C₁-C₆ alkoxy, aryl, —NO₂, or halo;
R₄ and R₄ₐ are —H; and
R₅ and R₅ₐ are independently —H, C₁-C₆ alkyl, —C₀-C₆ alkyl-C(O)—OH or C₀-C₆ alkylaryl, wherein each of the alkyl and aryl is optionally substituted with 1 to 4 groups selected from —C(O)—OH or —C₁-C₆ alkyl-N(R₄)(R₄ₐ).

Embodiment G comprises compounds according to Embodiment F wherein R₁ is —C₁-C₃ alkylaryl-C(O)—N(R₅)(R₅ₐ). Preferably, R₁ is —CH₂-aryl-C(O)—N(R₅)(R₅ₐ). More preferably, the aryl is phenyl,
R₅ is —H and R₅ₐ is C₁-C₃ alkyl-C(O)—OH. More preferably, R₅ₐ is —CH₂—C(O)—OH.

Embodiment H comprises compounds according to Embodiment F wherein R₅ is —H and R₅ₐ is C₁-C₃ alkyl substituted with —C(O)—OH. Preferably, R₅ₐ is —CH(C(O)—OH)—CH₃.

Embodiment I comprises compounds according to Embodiment F wherein R₅ is —C₁-C₃ alkylaryl, wherein each of the alkyl and aryl is optionally substituted with a group selected from —C(O)—OH or —C₁-C₃ alkyl-NH₂. Preferably, R₅ is —CH₂-aryl, wherein the aryl is substituted with —CH₂—NH₂. More preferably, the aryl is phenyl. Also preferred are compounds wherein R₅ is —CH₂-aryl, wherein the methyl is substituted with —C(O)—OH. Preferably, the aryl is phenyl.

Embodiment J comprises compounds according to Embodiment F wherein R₂ is C₁-C₄ alkyl. Preferably, R₂ is selected from the group consisting of methyl, ethyl, propyl and butyl.

Embodiment K comprises compounds according to Embodiment F wherein R₂ is C₂-C₃ alkenyl. Preferably, R₂ is propenyl.

Embodiment L comprises compounds according to Embodiment F wherein R₂ is —C₁-C₃ alkylaryl. Preferably, R₂ is —CH₂-aryl or —C₂H₄-aryl. More preferably, the aryl is phenyl.

Embodiment M comprises compounds according to Embodiment F wherein R₂ is aryl optionally substituted with 1 to 2 groups selected from halo. Preferably, the halo is fluoro, chloro or bromo. More preferably, the aryl is phenyl.

Embodiment N comprises compounds according to Embodiment F wherein R₃ is aryl optionally substituted with 1 to 2 groups selected from C₁-C₃ alkoxy, phenyl, —NO₂, or halo. Preferably, R₃ is phenyl substituted with 1 to 2 groups selected from methoxy, phenyl, choro, fluoro or bromo. Also preferred are compounds wherein R₃ is phenyl or naphthyl. Preferably, R₃ is phenyl optionally substituted in the meta and para position with 1 or 2 groups selected from halo, C₁-C₆ alkyl, C₁-C₆ alkoxy, aryl, or —NO₂. More preferably, R₃ is phenyl substituted in the meta and para position with 1 or 2 groups selected from —NO₂, methoxy, chloro, fluoro, bromo, methyl, or phenyl.

Embodiment O comprises compounds according to Embodiment E wherein
R₁ is —C₀-C₆ alkylaryl-C(O)-heterocyclyl;
R₂ is —C₁-C₆ alkyl-N(R₄)(R₄ₐ), —C₁-C₆ alkylaryl, —C₁-C₆ alkyl-O-aryl, or aryl, wherein each of the alkyl and aryl is optionally substituted with 1 to 4 groups selected from mono- to per-halogenated C₁-C₆ alkoxy, or C₁-C₆ alkoxy;
R₃ is aryl optionally substituted with 1 to 4 groups selected from C₁-C₆ alkoxy, aryl, —NO₂, or halo; or
R₃ is aryl optionally substituted in the meta and para position with 1 to 4 groups selected from C₁-C₆ alkoxy, aryl, —NO₂, or halo; and
R₄ and R₄ₐ are C₁-C₆ alkyl.

Embodiment P comprises compounds according to Embodiment O wherein R₁ is —C₁-C₃ alkylaryl-C(O)-heterocyclyl. Preferably, the aryl is phenyl. More preferably, R₁ is —CH₂-phenyl-C(O)-heterocyclyl, the heterocyclyl is selected from the group consisting of piperazinyl and morpholinyl.

Embodiment Q comprises compounds according to Embodiment O wherein R₂ is —C₁-C₆ alkylaryl wherein the aryl is optionally substituted with 1 to 2 groups selected from mono- to per-halogenated C₁-C₆ alkoxy, or C₁-C₆ alkoxy. Preferably, R₂ is —C₁-C₃ alkylaryl substituted with 1 to 2 groups selected from mono- to per-halogenated C₁-C₆ alkoxy or C₁-C₆ alkoxy. More preferably, R₂ is —CH₂-phenyl substituted with 1 to 2 groups selected from trifluoromethoxy or methoxy.

Embodiment R comprises compounds according to Embodiment O wherein R₂ is —C₁-C₃ alkyl-O-aryl. Preferably, R₂ is —C₂H₄—O-phenyl.

Embodiment S comprises compounds according to Embodiment O wherein R₂ is —C₁-C₃ alkyl-N(R₄)(R₄ₐ) and R₄ is C₁-C₃ alkyl. Preferably, R₂ is C₁-C₃ alkyl-N(isopropyl)₂. More preferably, R₂ is —C₂H₄—N(isopropyl)₂.

Embodiment T comprises compounds according to Embodiment O wherein R₂ is aryl. Preferably, the aryl is naphthyl.

Embodiment U comprises compounds according to Embodiment O wherein R₃ is aryl optionally substituted with 1 to 2 groups selected from C₁-C₃ alkoxy, phenyl, —NO₂, or halo. Preferably, R₃ is phenyl. More preferably, R₃ is phenyl substituted with 1 to 2 groups selected from methoxy, phenyl, —NO₂, or halo. Preferably, the halo is selected from the group consisting of chloro, fluoro, and bromo. Preferably, R₃ is phenyl optionally substituted in the meta and para position with 1 or 2 groups selected from halo, C₁-C₆ alkyl, C₁-C₆ alkoxy, aryl, or —NO₂. More preferably, R₃ is phenyl substituted in the meta and para position with 1 or 2 groups selected from —NO₂, methoxy, chloro, fluoro, bromo, methyl, or phenyl.

Embodiment V comprises compounds according to formula I of the formula 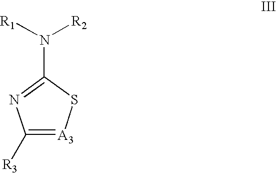
or pharmaceutically acceptable salts thereof, wherein
A₃ is —C(R₆)═;
R₁ is —C₁-C₆ alkylaryl, —C₁-C₆ alkylheteroaryl, —C₁-C₆ alkylheterocyclyl, —C₀-C₆ alkylaryl-C(O)—N(R₅)(R₅ₐ), —C₀-C₆ alkylheteroaryl-C(O)—N(R₅)(R₅ₐ), —C₀-C₆ alkylaryl-NR₅—C(O)—R₅, —C₀-C₆ alkylheteroaryl-NR₅—C(O)—R₅, —C₀-C₆ alkylaryl-C(O)-heterocyclyl, —C₀-C₆ alkylheteroaryl-C(O)—heterocyclyl, —C₀-C₆ alkylaryl-C(O)-heteroaryl, —C₀-C₆ alkylheteroaryl-C(O)-heteroaryl, aryl, heteroaryl, heterocyclyl, wherein each of the alkyl, aryl, heterocyclyl, and heteroaryl is optionally substituted with 1 to 4 groups independently selected from R₇;
R₂ is —H, C₁-C₆ alkyl, C₂-C₆ alkenyl, C₂-C₆ alkynyl, —C₁-C₆ alkyl-N(R₄)(R₄ₐ), —C₁-C₆ alkylaryl, —C₁-C₆ alkylheteroaryl, —C₁-C₆ alkylheterocyclyl, —C₁-C₆ alkyl-O-aryl, aryl, heteroaryl, heterocyclyl, wherein each of the alkyl, alkenyl, alkynyl, aryl, heterocyclyl, and heteroaryl is optionally substituted with 1 to 4 groups selected from oxo, —OH, —SH, C₁-C₆ alkyl, C₁-C₆ alkoxy, mono- to per-halogenated C₁-C₆ alkyl, mono- to per-halogenated C₁-C₆ alkoxy, —N(R₈)(R₈ₐ), —NO₂, halo, or —CN;
R₄ and R₄ₐ are independently —H, —OH, C₁-C₆ alkyl, aryl, —SH, C₂-C₆ alkenyl, C₂-C₆ alkynyl, —N(R₈)(R₈ₐ) or C₁-C₆ alkoxy;
R₅ and R₅ₐ are independently —H, —OH, C₁-C₆ alkyl, —C₀-C₆ alkyl-C(O)—OH, C₁-C₆ alkoxy, —C₁-C₆ alkyl-C₃-C₆ cycloalkyl, —C₀-C₆ alkylaryl, —C₀-C₆ alkylheteroaryl, wherein each of the alkyl, aryl, and heteroaryl are optionally substituted with 1 to 4 groups selected from oxo, —C(O)—OH, —OH, —SH, C₁-C₆ alkyl, C₁-C₆ alkoxy, mono- to per-halogenated C₁-C₆ alkyl, mono- to per-halogenated C₁-C₆ alkoxy, —C₁-C₆ alkyl-N(R₄)₂, —N(R₈)(R₈ₐ), —NO₂, halo, or —CN;
R₈ and R₈ₐ are independently —H or —C₁-C₆ alkyl;
R₆ is —H, —OH, C₁-C₆ alkyl, —SH, C₂-C₆ alkenyl, C₂-C₆ alkynyl, aryl, heteroaryl, heterocyclyl, —C(O)-aryl, —C(O)-heteroaryl, —C(O)-heterocyclyl, —C(O)—R₄, —N(R₈)(R₈ₐ) or C₁-C₆ alkoxy, wherein each of the alkyl, aryl, heterocyclyl, and heteroaryl is optionally substituted with 1 to 4 groups selected from oxo, —OH, —SH, C₁-C₆ alkyl, C₁-C₆ alkoxy, mono- to per-halogenated C₁-C₆ alkyl, mono- to per-halogenated C₁-C₆ alkoxy, aryl, heteroaryl, heterocyclyl, C₁-C₆ alkyl-N(R₄)(R₄ₐ), —N(R₈)(R₈ₐ), —NO₂, halo, or —CN;
R₇ is oxo, —OH, —SH, C₁-C₆ alkyl, C₁-C₆ alkoxy, —C(O)—OH, mono- to per-halogenated C₁-C₆ alkyl, mono- to per-halogenated C₁-C₆ alkoxy, —N(R₈)(R₈ₐ), —NO₂, halo, or —CN; and
R₃ is C₁-C₆ alkyl, mono- to per-halogenated C₁-C₆ alkyl, —C₁-C₆ alkylaryl, —C₁-C₆ alkylheteroaryl, —C₁-C₆ alkylheterocyclyl, aryl, heteroaryl, heterocyclyl, wherein each of the alkyl, aryl, heterocyclyl, and heteroaryl is optionally substituted with 1 to 4 groups selected from oxo, —OH, —SH, C₁-C₆ alkyl, C₁-C₆ alkoxy, mono- to per-halogenated C₁-C₆ alkyl, mono- to per-halogenated C₁-C₆ alkoxy, aryl, heteroaryl, heterocyclyl, —C₁-C₆ alkyl-N(R₄)(R₄ₐ), —N(R₈)(R₈ₐ), —NO₂, halo, or —CN; or
R₃ is aryl optionally substituted in the meta and para position with 1 to 4 groups selected from oxo, —OH, —SH, C₁-C₆ alkyl, C₁-C₆ alkoxy, mono- to per-halogenated C₁-C₆ alkyl, mono- to per-halogenated C₁-C₆ alkoxy, aryl, heteroaryl, heterocyclyl, —C₁-C₆ alkyl-N(R₄)(R₄ₐ), —N(R₈)(R₈ₐ), —NO₂, halo, or —CN.

Embodiment W comprises compounds according to Embodiment V wherein
R₁ is —C₀-C₆ alkylaryl-C(O)—N(R₅)(R₅ₐ) or —C₀-C₆ alkylaryl-C(O)-heterocyclyl;
R₂ is C₁-C₆ alkyl, C₂-C₆ alkenyl, —C₁-C₆ alkyl-N(R₄)(R₄ₐ), —C₁-C₆ alkylaryl, —C₁-C₆ alkylheteroaryl, —C₁-C₆ alkyl-O-aryl, or aryl, wherein each of the alkyl, alkenyl, aryl, and heteroaryl is optionally substituted with 1 to 4 groups selected from mono- to per-halogenated C₁-C₆ alkoxy, C₁-C₆ alkoxy or halo;
R₃ is C₁-C₆ alkyl, mono- to per-halogenated C₁-C₆ alkyl, or aryl wherein each of the alkyl or aryl is optionally substituted with 1 to 4 groups selected from C₁-C₆ alkyl, C₁-C₆ alkoxy, aryl, —NO₂, or halo; or
R₃ is aryl optionally substituted in the meta and para position with 1 to 4 groups selected from C₁-C₆ alkyl, C₁-C₆ alkoxy, aryl, —NO₂, or halo;
R₄ is —H or C₁-C₆ alkyl;
R₆ is C₁-C₆ alkyl; and
R₅ is —H, C₁-C₆ alkyl, —C₀-C₆ alkyl-C(O)—OH, —C₁-C₆ alkyl-C₃-C₆ cycloalkyl, —C₀-C₆ alkylaryl, wherein each of the alkyl and aryl is optionally substituted with 1 to 4 groups selected from C₁-C₆ alkoxy, —C(O)—OH, —NH₂, or —C₁-C₆ alkyl-N(R₄)(R₄ₐ).

Embodiment X comprises compounds according to Embodiment W wherein
R₁ is —C₀-C₆ alkylaryl-C(O)—N(R₅)(R₅ₐ);
R₂ is C₁-C₆ alkyl, C₂-C₆ alkenyl, C₁-C₆ alkylaryl, or aryl, wherein the aryl is optionally substituted with 1 to 4 groups selected from halo;
R₃ is aryl optionally substituted with 1 to 4 groups selected from C₁-C₆ alkoxy, —NO₂, aryl, or halo; or
R₃ is aryl optionally substituted in the meta and para position with 1 to 4 groups selected from C₁-C₆ alkoxy, —NO₂, aryl, or halo;
R₄ is —H;
R₆ is C₁-C₃ alkyl; and
R₅ is —H, C₁-C₆ alkyl, —C₀-C₆ alkyl-C(O)—OH or —C₀-C₆ alkylaryl, wherein each of the alkyl and aryl is optionally substituted with 1 to 4 groups selected from —C(O)—OH or —C₁-C₆ alkyl-N(R₄)(R₄ₐ).

Embodiment Y comprises compounds according to Embodiment X wherein R₁ is —C₁-C₃ alkylaryl-C(O)—N(R₅)(R₅ₐ). Preferably, R₁ is —CH₂-aryl-C(O)—N(R₅)(R₅ₐ). More preferably, the aryl is phenyl. Preferably, R₅ is —H and R₅ₐ is —C₁-C₃ alkylaryl, wherein the aryl is optionally substituted with —C₁-C₃ alkyl-NH₂. More preferably, R₅ₐ is —CH₂-aryl, wherein the aryl is substituted with —CH₂—NH₂. Preferably, the aryl is phenyl.

Embodiment Z comprises compounds according to Embodiment X wherein R₂ is C₁-C₄ alkyl. Preferably, R₂ is selected from the group consisting of methyl, ethyl, propyl and butyl.

Embodiment AA comprises compounds according to Embodiment X wherein R₂ is C₂-C₃ alkenyl. Preferably, R₂ is propenyl.

Embodiment BB comprises compounds according to Embodiment X wherein R₃ is aryl optionally substituted with 1 or 2 C₁-C₃ alkoxy groups. Preferably, R₃ is phenyl.

Embodiment CC comprises compounds according to Embodiment X wherein R₆ is methyl, ethyl or propyl.

Embodiment DD comprises compounds according to formula I that is one of the following formulae: 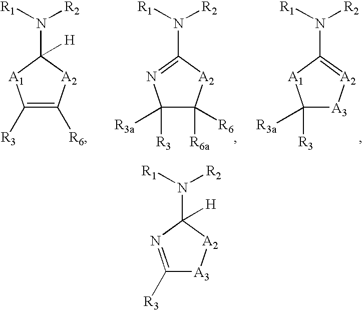
or pharmaceutically acceptable salts thereof, wherein
A₁, A₂ and A₃ are independently —C(R₆)(R₆ₐ)—, —C(R₆)═, —N═, —N(R₆)—, —O—, or —S—;
R₁ is —H, natural or non-natural amino acids, C₁-C₆ alkyl, C₃-C₆ cycloalkyl, —C₁-C₆ alkylaryl, —C₁-C₆ alkylheteroaryl, —C₁-C₆ alkylheterocyclyl, —C₀-C₆ alkylaryl-C(O)—N(R₅)(R₅ₐ), —C₀-C₆ alkylheteroaryl-C(O)—N(R₅)(R₅ₐ), —C₀-C₆ alkylaryl-NR₅—C(O)—R₅, —C₀-C₆ alkylheteroaryl-NR₅—C(O)—R₅, —C₀-C₆ alkylaryl-C(O)-heterocyclyl, —C₀-C₆ alkylheteroaryl-C(O)-heterocyclyl, —C₀-C₆ alkylaryl-C(O)-heteroaryl, —C₀-C₆ alkylheteroaryl-C(O)-heteroaryl, aryl, heteroaryl, heterocyclyl, wherein each of the alkyl, aryl, heterocyclyl, and heteroaryl is optionally substituted with 1 to 4 groups independently selected from R₇;
R₂ is —H, C₁-C₆ alkyl, C₂-C₆ alkenyl, C₂-C₆ alkynyl, —C₁-C₆ alkyl-N(R₄)(R₄ₐ), —C₁-C₆ alkylaryl, —C₁-C₆ alkylheteroaryl, —C₁-C₆ alkylheterocyclyl, —C₁-C₆ alkyl-O-aryl, —C₁-C₆ alkyl-O-heteroaryl, aryl, heteroaryl, heterocyclyl, wherein each of the alkyl, alkenyl, alkynyl, aryl, heterocyclyl, and heteroaryl is optionally substituted with 1 to 4 groups selected from oxo, —OH, —SH, C₁-C₆ alkyl, C₁-C₆ alkoxy, mono- to per-halogenated C₁-C₆ alkyl, mono- to per-halogenated C₁-C₆ alkoxy, —N(R₈)(R₈ₐ), —NO₂, halo, or —CN;
R₄ and R₄ₐ are independently —H, —OH, C₁-C₆ alkyl, —SH, C₂-C₆ alkenyl, aryl, C₂-C₆ alkynyl, —N(R₈)(R₈ₐ) or C₁-C₆ alkoxy;
R₅ and R₅ₐ are independently —H, —OH, C₁-C₆ alkyl, —C₀-C₆ alkyl-C(O)—OH, C₁-C₆ alkoxy, —C₁-C₆ alkyl-C₃-C₆ cycloalkyl, —C₀-C₆ alkylaryl, —C₀-C₆ alkylheteroaryl, wherein each of the alkyl, aryl, and heteroaryl is optionally substituted with 1 to 4 groups selected from oxo, —OH, —SH, —C(O)—OH, C₁-C₆ alkyl, C₁-C₆ alkoxy, —C(O)—OH, mono- to per-halogenated C₁-C₆ alkyl, mono- to per-halogenated C₁-C₆ alkoxy, —C₁-C₆ alkyl-N(R₄)(R₄ₐ), —N(R₈)(R₈ₐ), —NO₂, halo, or —CN;
the R₈ and R₈ₐ are independently —H or —C₁-C₆ alkyl;
R₆ and R₆ₐ are independently —H, —OH, C₁-C₆ alkyl, —SH, C₂-C₆ alkenyl, C₂-C₆ alkynyl, aryl, heteroaryl, heterocyclyl, —C(O)-aryl, —C(O)-heteroaryl, —C(O)-heterocyclyl, —C(O)—R₄, —N(R₈)(R₈ₐ) or C₁-C₆ alkoxy, wherein each of the alkyl, aryl, heterocyclyl, and heteroaryl is optionally substituted with 1 to 4 groups selected from oxo, —OH, —SH, C₁-C₆ alkyl, C₁-C₆ alkoxy, mono- to per-halogenated C₁-C₆ alkyl, mono- to per-halogenated C₁-C₆ alkoxy, aryl, heteroaryl, heterocyclyl, C₁-C₆ alkyl-N(R₄)(R₄ₐ), —N(R₈)(R₈ₐ), —NO₂, halo, or —CN;
R₇ is oxo, —OH, —SH, C₁-C₆ alkyl, C₁-C₆ alkoxy, —C(O)—OH, mono- to per-halogenated C₁-C₆ alkyl, mono- to per-halogenated C₁-C₆ alkoxy, —N(R₈)(R₈ₐ), —NO₂, halo, or —CN;
R₃ and R₃ₐ are independently —H, C₁-C₆ alkyl, C₁-C₆ alkoxy, mono- to per-halogenated C₁-C₆ alkyl, —C₁-C₆ alkylaryl, —C₁-C₆ alkylheteroaryl, —C₁-C₆ alkylheterocyclyl, aryl, heteroaryl, heterocyclyl, wherein each of the alkyl, aryl, heterocyclyl, and heteroaryl is optionally substituted with 1 to 4 groups selected from oxo, —OH, —SH, C₁-C₆ alkyl, C₁-C₆ alkoxy, mono- to per-halogenated C₁-C₆ alkyl, mono- to per-halogenated C₁-C₆ alkoxy, aryl, heteroaryl, heterocyclyl, —C₁-C₆ alkyl-N(R₄)(R₄ₐ), —N(R₈)(R₈ₐ), —NO₂, halo, or —CN; or
R₃ or R₃ₐ together with a substituent attached to A₃ form an aryl, heteroaryl, heterocyclyl or cycloalkyl group, wherein each of the aryl, heterocyclyl, cycloalkyl, and heteroaryl is optionally substituted with 1 to 4 groups selected from oxo, —OH, —SH, C₁-C₆ alkyl, C₁-C₆ alkoxy, mono- to per-halogenated C₁-C₆ alkyl, mono- to per-halogenated C₁-C₆ alkoxy, aryl, heteroaryl, heterocyclyl, —C₁-C₆ alkyl-N(R₄)(R₄ₐ), —N(R₈)(R₈ₐ), —NO₂, halo, or —CN; or
R₃ or R₃ₐ is aryl optionally substituted in the meta and para position with 1 to 4 groups selected from oxo, —OH, —SH, C₁-C₆ alkyl, C₁-C₆ alkoxy, mono- to per-halogenated C₁-C₆ alkyl, mono- to per-halogenated C₁-C₆ alkoxy, aryl, heteroaryl, heterocyclyl, —C₁-C₆ alkyl-N(R₄)(R₄ₐ), —N(R₈)(R₈ₐ), —NO₂, halo, or —CN; and
provided that when R₃ together with a substituent attached to A₃ form an aryl, heteroaryl, heterocyclyl or cycloalkyl group, R₁ is —C₀-C₆ alkylaryl-C(O)—N(R₅)(R₅ₐ) or —C₀-C₆ alkylaryl-C(O)-heterocyclyl, and R₂ is C₁-C₆ alkyl, C₂-C₆ alkenyl, —C₁-C₆ alkyl-N(R₄)(R₄ₐ), —C₁-C₆ alkylaryl, —C₁-C₆ alkylheteroaryl, —C₁-C₆ alkyl-O-aryl or aryl.

Embodiment EE comprises compounds according to Embodiment DD that is one of the following formulae: 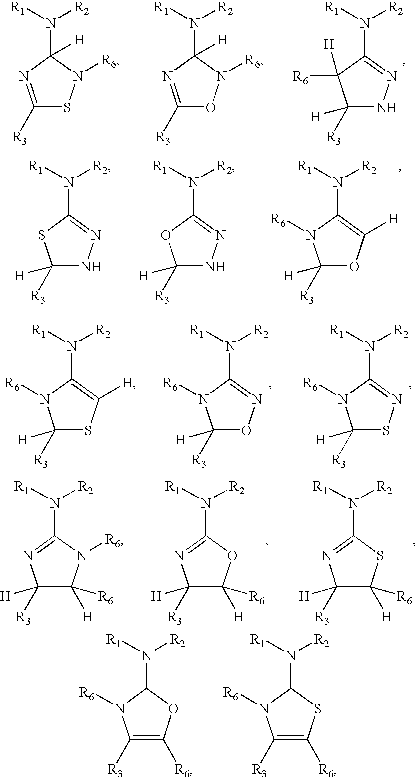
or pharmaceutically acceptable salts thereof.

In a second aspect, the invention comprises a pharmaceutical composition comprising an inhibitor of ubiquitination according to the first aspect and Embodiments A-EE of the invention and a pharmaceutically acceptable carrier, excipient, or diluent.

In the third aspect, the invention provides methods of inhibiting ubiquitination in a cell comprising contacting the cell in which inhibition of ubiquitination is desired with a compound according to the invention or a pharmaceutical composition according to the first and second aspect of the invention. The compounds and formulations of the invention can inhibit ubiquitination in cells derived from animals, particularly, mammalian cells.

In the fourth aspect, the invention provides for methods of treating cell proliferative diseases or conditions that involve ubiquitination comprising administering to a patient an effective amount of a compound or pharmaceutical composition according to the first and second aspect of the invention. Cell proliferative diseases or conditions include, but are not limited to, cancers, such as cancers of the breast, immune system, bone, nervous system, brain, blood, lymphatic system, and skin. Particularly, the compounds and pharmaceutical compositions of the invention are useful for treating cell proliferative diseases or conditions that involve MDM2. For example, MDM2 is overexpressed in several tumors. Overexpression of MDM2 is seen in human leukemias (Bueso-Ramos C. E., Manshouri. T., Haidar. M. A., Huh Y. O., Keating M. J., Multiple patterns of MDM-2 deregulation in human leukemias: implications in leukemogenesis and prognosis, USA Leukemia & Lymphoma (March 1995), 17(1-2):13-8), testicular cancer (Bak M., Geczi L., Institioris E., Eid H., Bodrogi I., The clinical value of mdm-2 (proto-oncogene) expression in testicular cancer, correlation with tumor progression, Orvosi Hetilap (Aug. 15, 1999), 140(33):1837-40), gastric cancer (Villaseca M., Araya J. C., Roa I., Roa J. C., Gastric cancer and tumor growth regulation. Study of cell proliferation markers and protein complex p53/p21WAF1/CIP1/mdm-2, Revista Medica de Chile (February 2000), 128(2):127-36), laryngeal carcinoma (Pruneri G., Pignataro L., Carboni N., Luminari S., Capaccio P., Neri A., Buffa R., MDM-2 oncoprotein overexpression in laryngeal squamous cell carcinoma: association with wild-type p53 accumulation, Modern Pathology (August 1997 August), 10(8):785-92).

In the fifth aspect, the invention provides for methods of inhibiting ubiquitin ligase comprising administering to a patient an effective amount of a compound or pharmaceutical composition according to the first and second aspect of the invention. The compounds and methods of the invention are useful to treat a patient who suffers from a condition or disease that involves a process selected from the group consisting of inflammation, adaptive immunity, innate immunity, bone metabolism, LPS-induced angiogenesis, osteoporosis, osteopinneal diseases, lymph node development, mammary gland development, skin development, and central nervous system development.

Particularly, the compounds and pharmaceutical compositions are useful for treating conditions or diseases that involve ubiquitin ligase such as those related to inflammation, adaptive immunity, innate immunity, bone metabolism, LPS-induced angiogenesis, osteoporosis, osteopinneal diseases, lymph node development, mammary gland development, skin development, and central nervous system development.

The compounds according to the first aspect of the invention are also useful as general ubiquitin ligase inhibitors. For example, the compounds of the invention can be used as inhibitors of E3 enzymes that contain HECT and RING finger domains and variants, U-box-containing proteins, and APC complex. Accordingly, the compounds of the invention are useful as protein modulators, immunologic agentsanti-inflammatory agents, anti-osteoporosis agents, anti-viral agents, for example, inhibitors of variola viruses such as smallpox, HIV and related conditions, human papillomavirus, HSV, adenovirus, coxsackie virus, HCMV, KSHV, EBV, paramyxovirus, myxomavirus, ebola, retrovirus, and rhabdovirus, anti-protozoan agents, for example, inhibitors of the malaria parasite. The compounds of the invention are also useful as oncologic and anti-proliferative agents that inhibit aberrant cell growth, cancers, restenosis, psoriasis, and neoplastic cell proliferation.

Inhibition of ubiquitination may also serve as a therapeutic target in diseases and conditions that involve non-degradative ubiquitination. For example, TRAF6 acts as an E3 ubiquitin ligase that mediates kinase activation by K-63 linked, non-degradative ubiquitination.

Some useful compounds according to one aspect of the invention are given in the following table and can be used in pharmaceutical compositions. (Terminal hydrogen atoms are not displayed in the structures of Table 1. Those skilled in the art can readily determine the position and number of hydrogens based on the standard number of valences for each atom.)

TABLE 1

| Cpd. | |
|---|---|
| # | Structure |
| 1 | 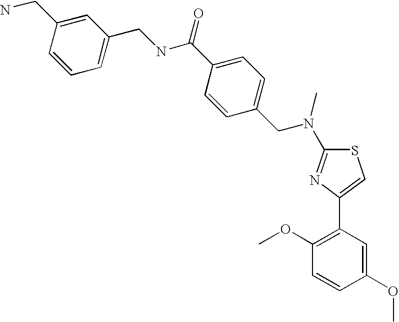 |
| 2 | 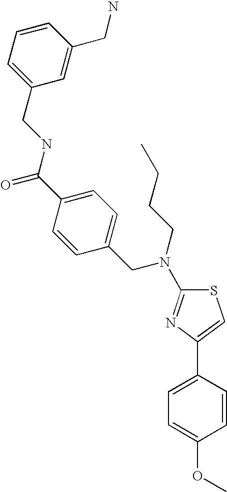 |
| 3 | 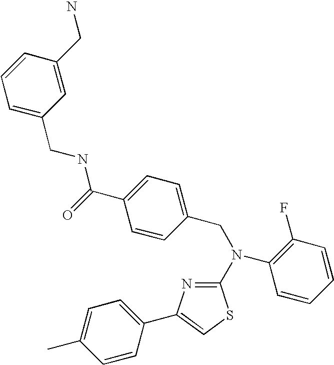 |
| 4 | 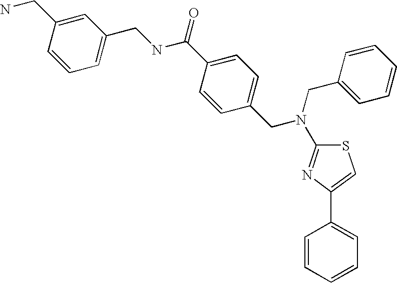 |
| 5 | 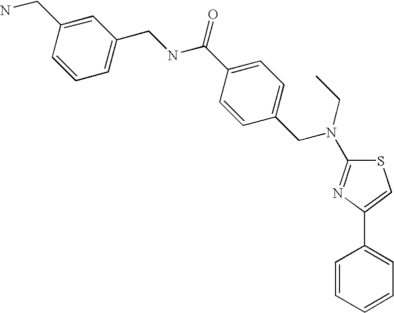 |
| 6 | 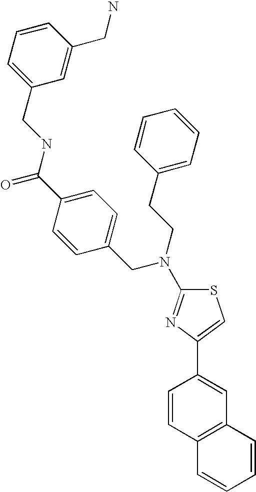 |
| 7 | 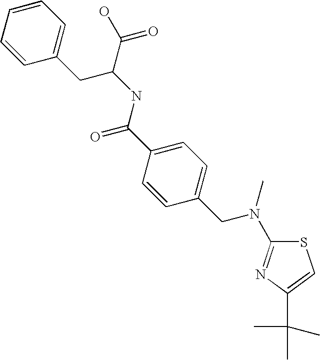 |
| 8 | 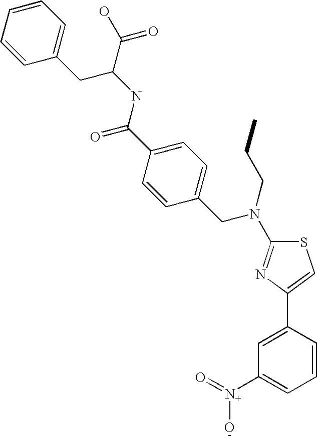 |
| 9 | 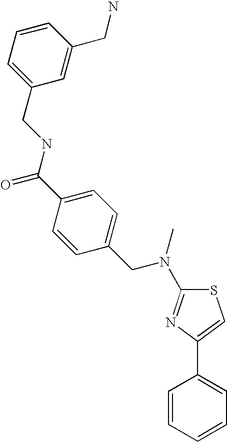 |
| 10 | 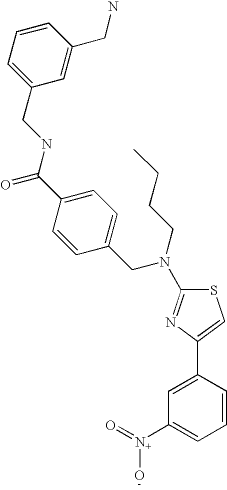 |
| 11 | 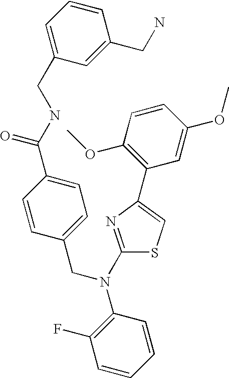 |
| 12 | 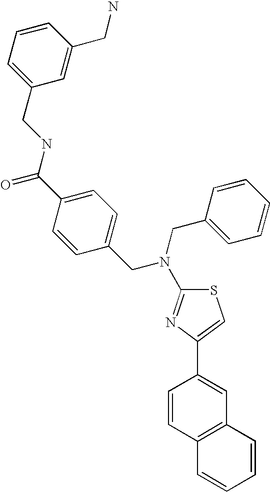 |
| 13 | 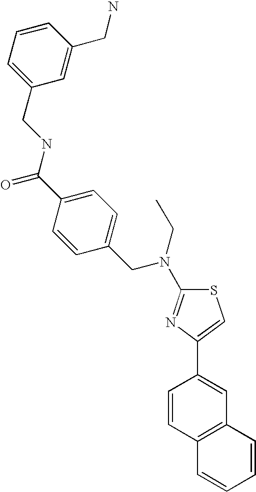 |
| 14 | 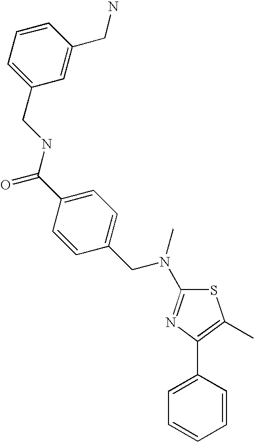 |
| 15 | 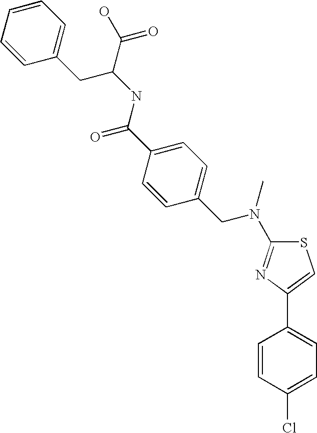 |
| 16 | 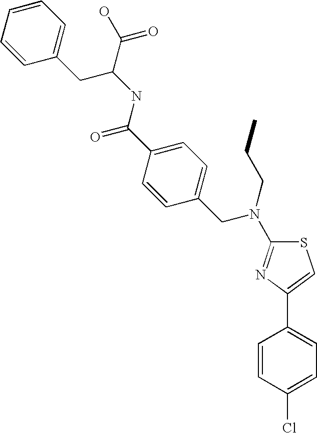 |
| 17 | 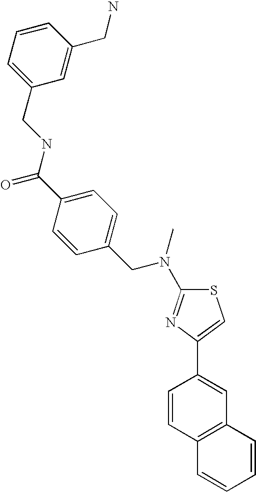 |
| 18 | 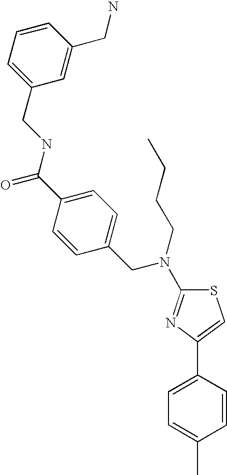 |
| 19 | 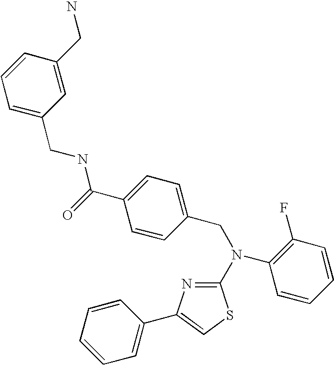 |
| 20 | 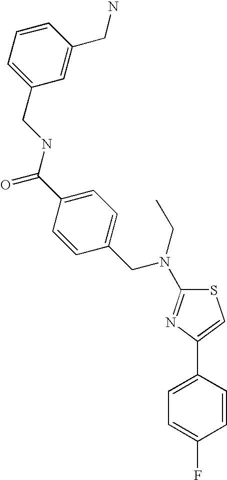 |
| 21 | 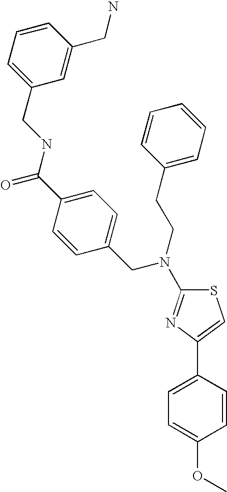 |
| 22 | 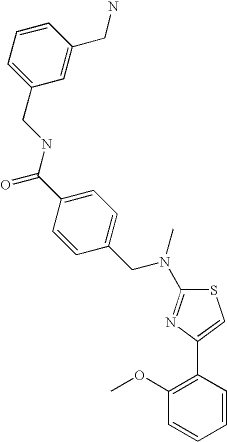 |
| 23 | 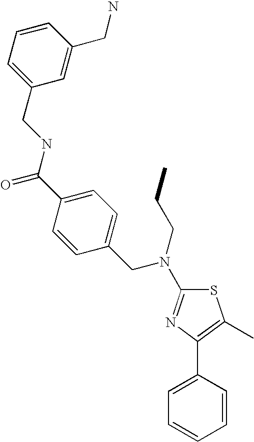 |
| 24 | 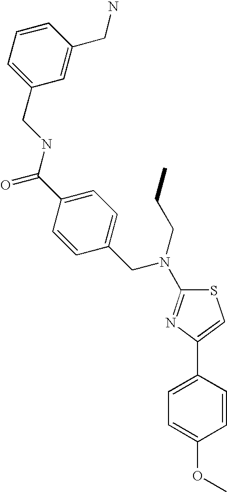 |
| 25 | 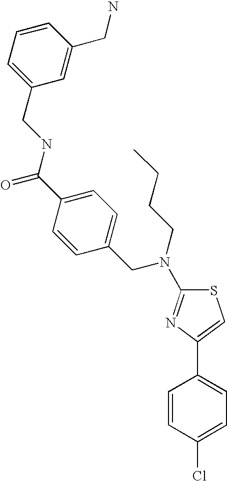 |
| 26 | 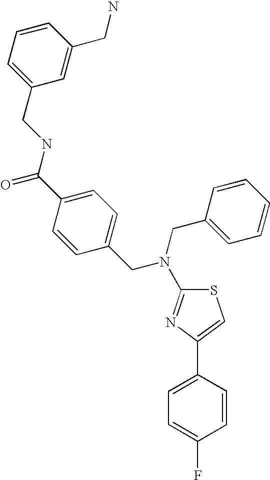 |
| 27 | 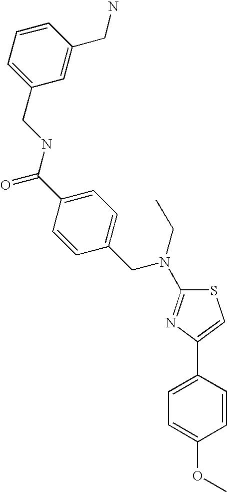 |
| 28 | 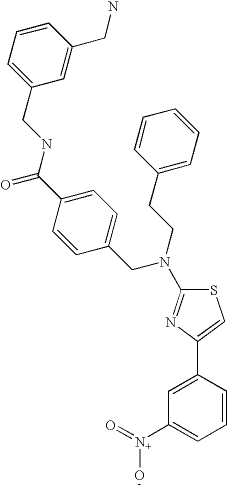 |
| 29 | 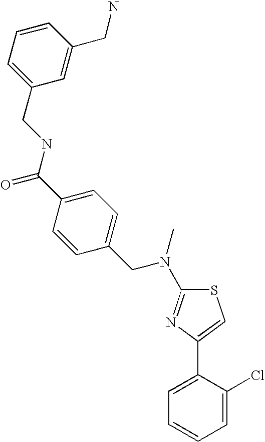 |
| 30 | 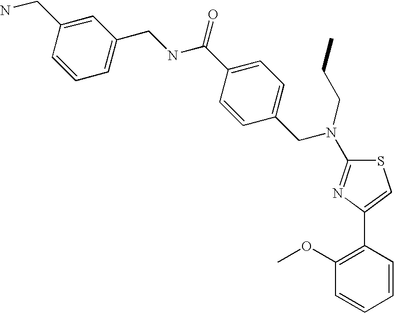 |
| 31 | 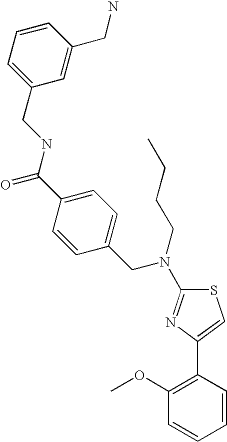 |
| 32 | 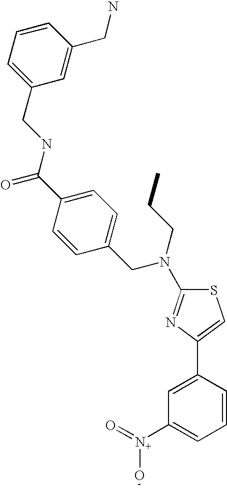 |
| 33 | 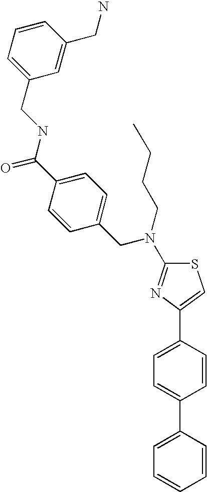 |
| 34 | 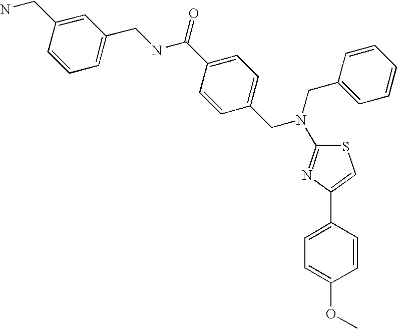 |
| 35 | 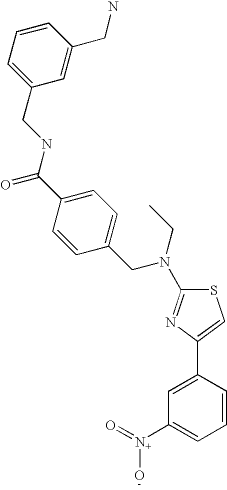 |
| 36 | 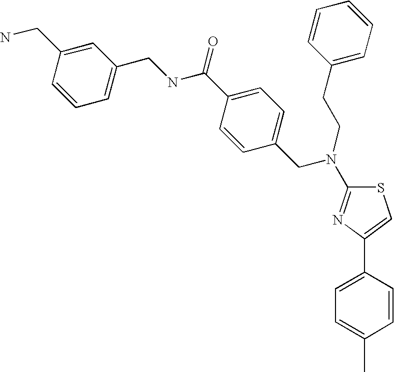 |
| 37 | 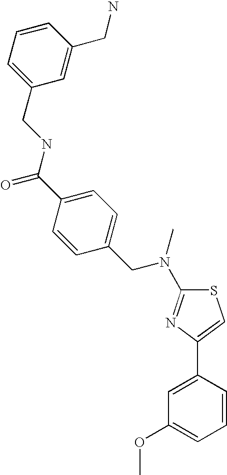 |
| 38 | 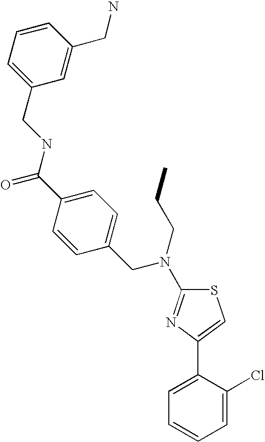 |
| 39 | 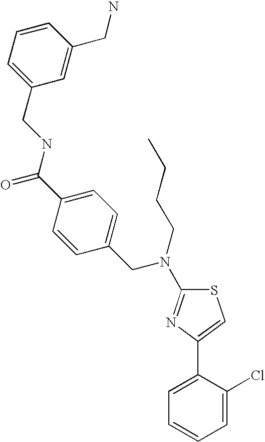 |
| 40 | 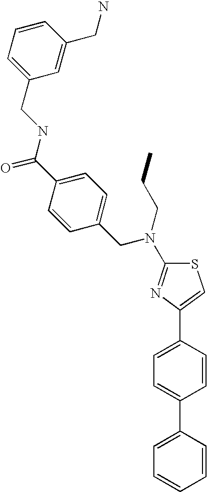 |
| 41 | 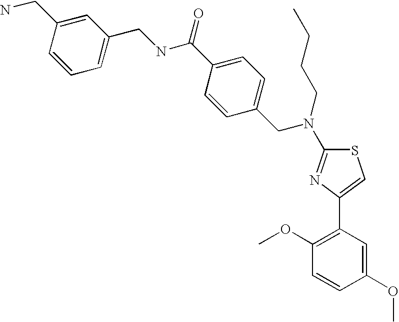 |
| 42 | 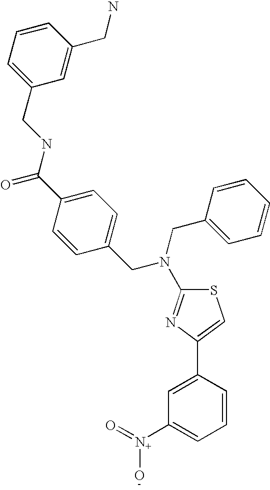 |
| 43 | 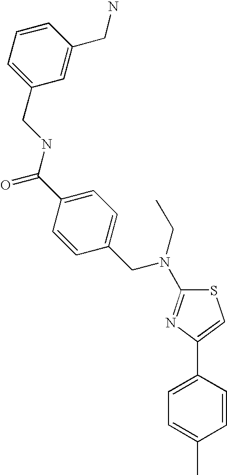 |
| 44 | 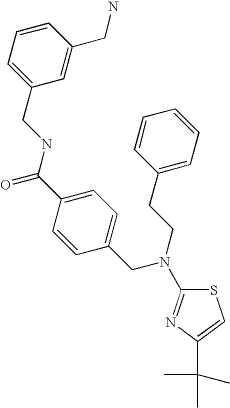 |
| 45 | 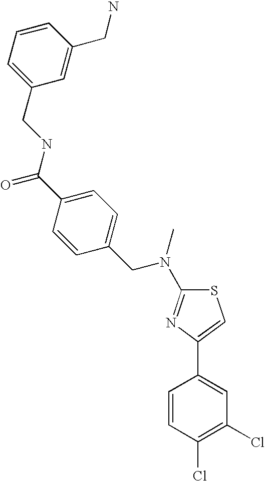 |
| 46 | 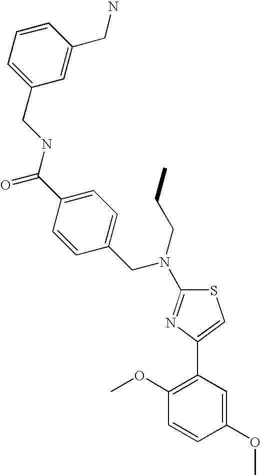 |
| 47 | 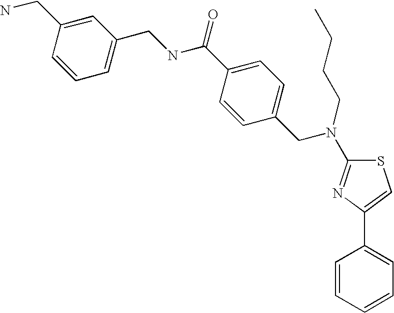 |
| 48 | 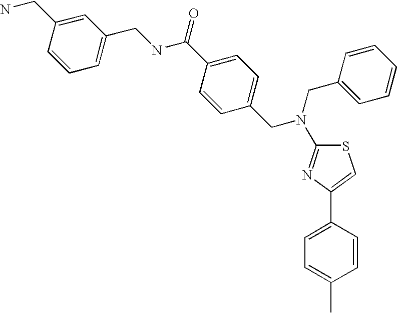 |
| 49 | 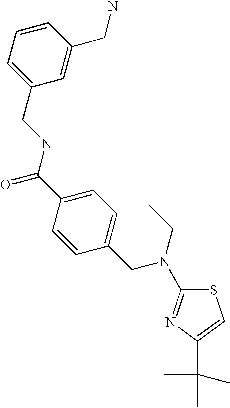 |
| 50 | 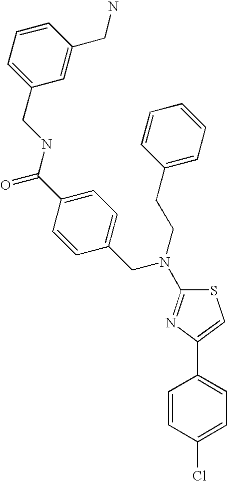 |
| 51 | 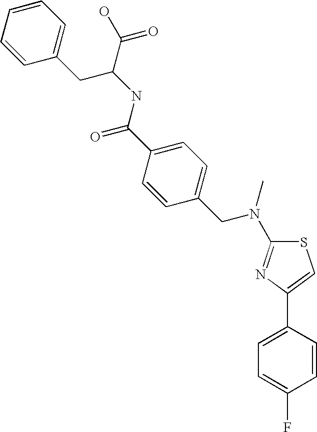 |
| 52 | 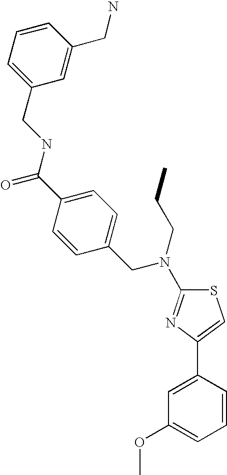 |
| 53 | 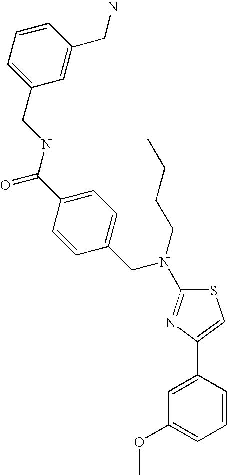 |
| 54 | 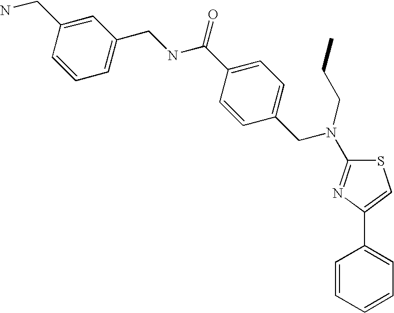 |
| 55 | 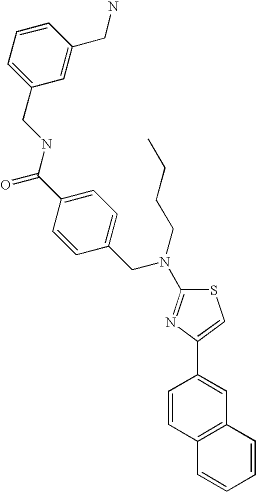 |
| 56 | 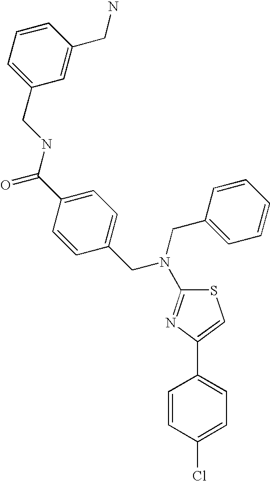 |
| 57 | 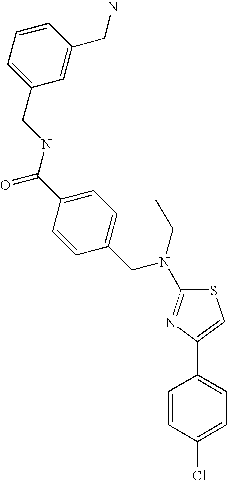 |
| 58 | 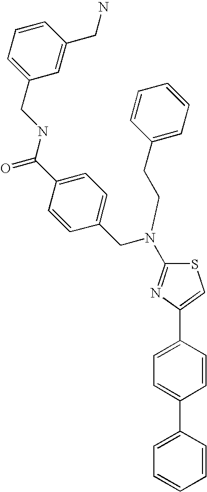 |
| 59 | 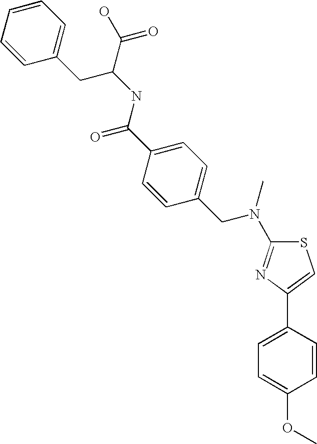 |
| 60 | 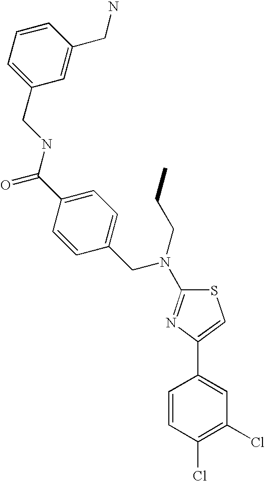 |
| 61 | 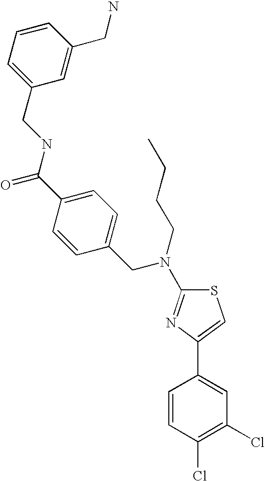 |
| 62 | 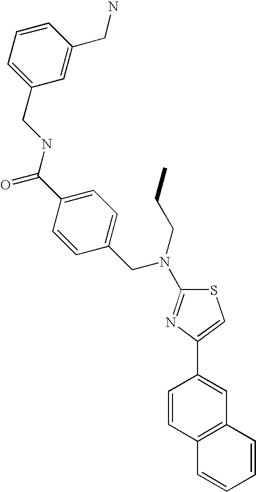 |
| 63 | 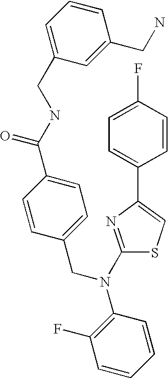 |
| 64 | 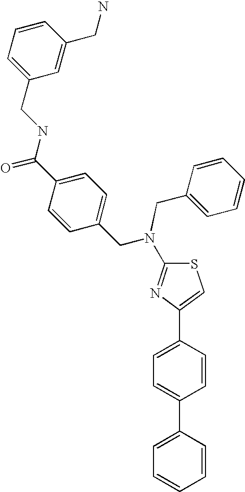 |
| 65 | 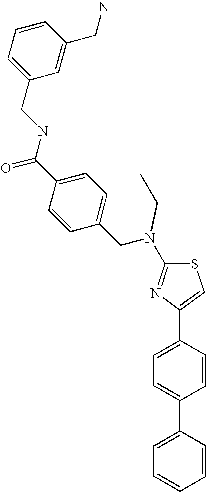 |
| 66 | 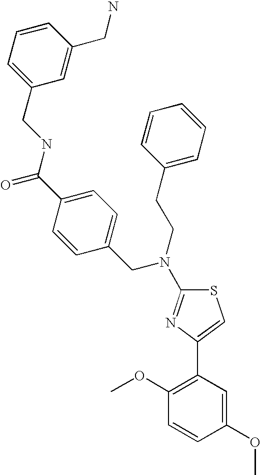 |
| 67 | 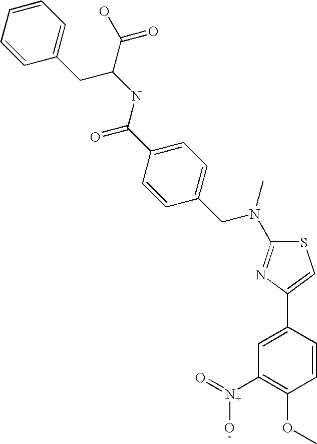 |
| 68 | 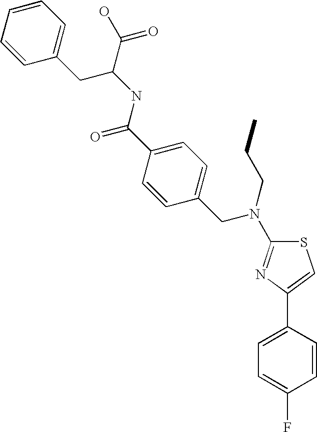 |
| 69 | 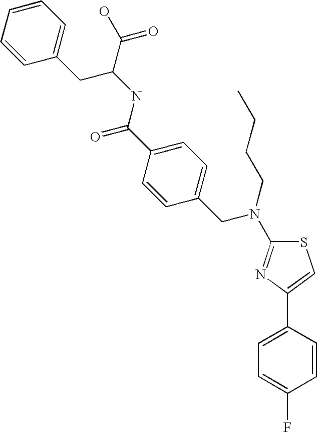 |
| 70 | 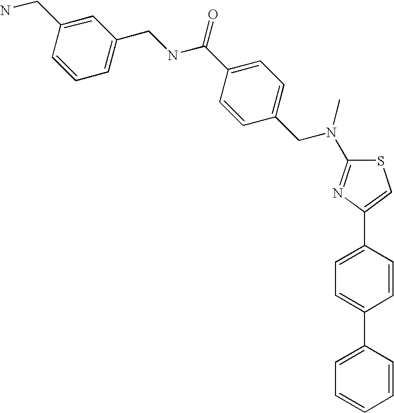 |
| 71 | 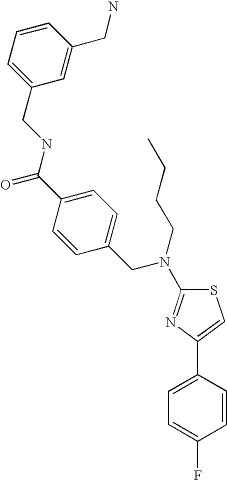 |
| 72 | 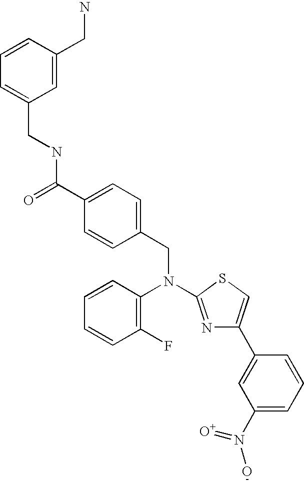 |
| 73 | 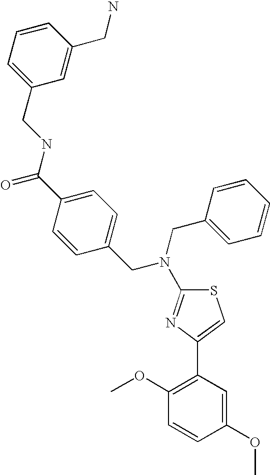 |
| 74 | 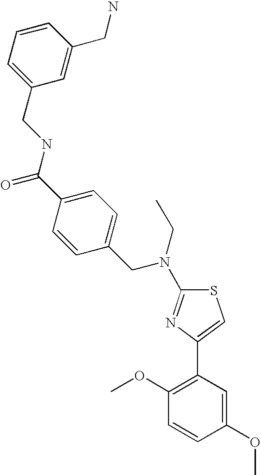 |
| 75 | 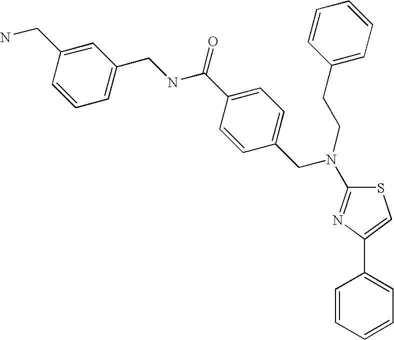 |
| 76 | 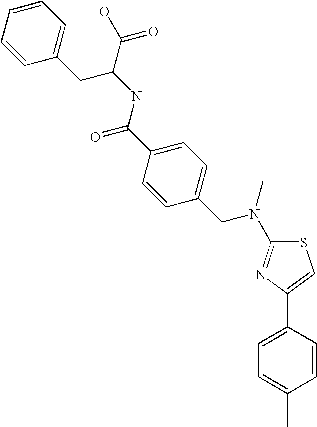 |
| 77 | 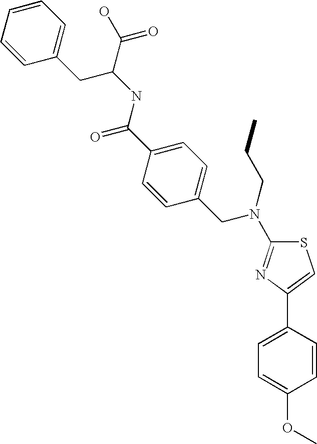 |
| 78 | 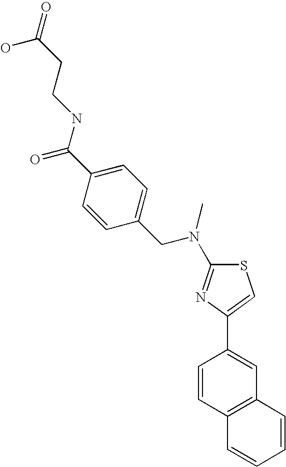 |
| 79 | 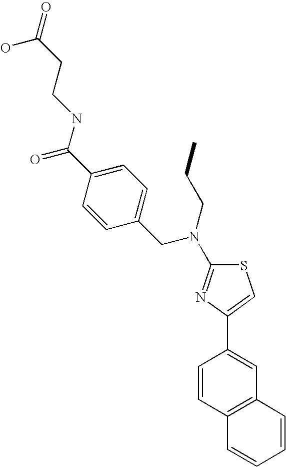 |
| 80 | 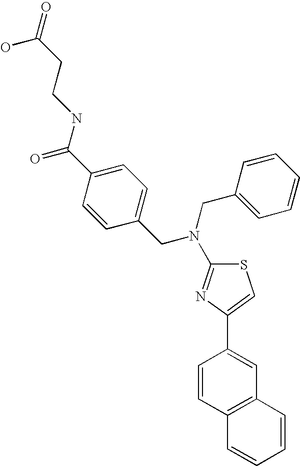 |
| 81 | 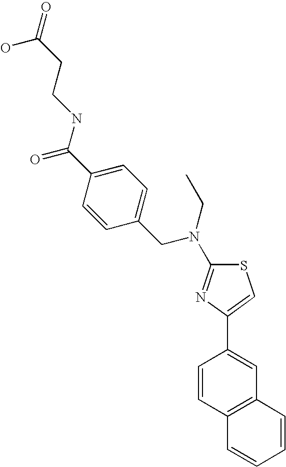 |
| 82 | 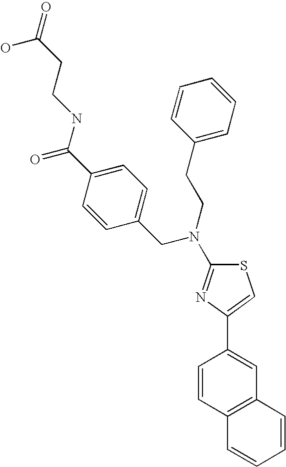 |
| 83 | 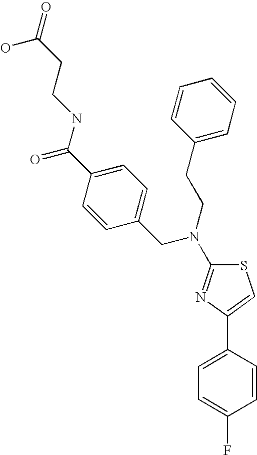 |
| 84 | 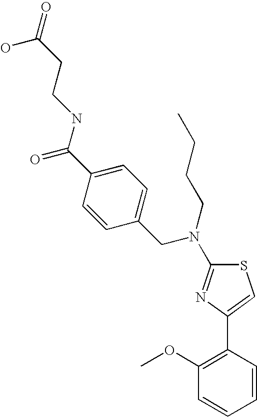 |
| 85 | 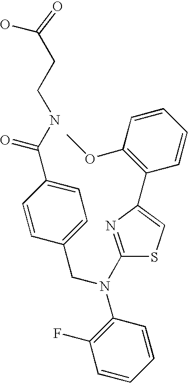 |
| 86 | 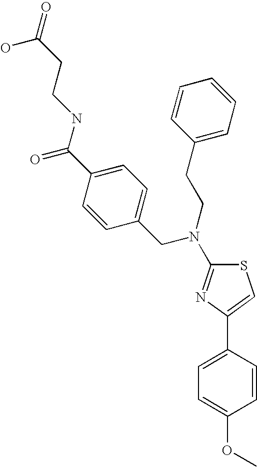 |
| 87 | 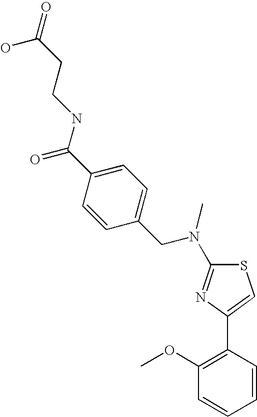 |
| 88 | 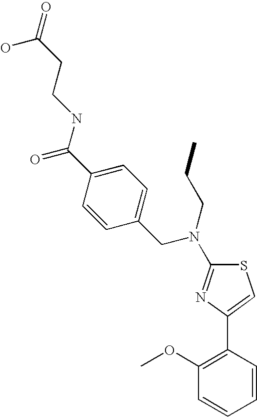 |
| 89 | 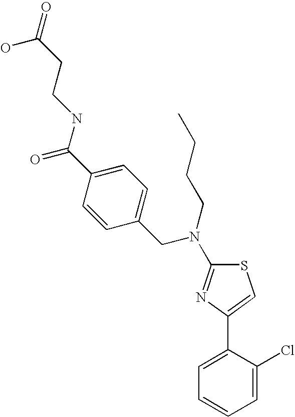 |
| 90 | 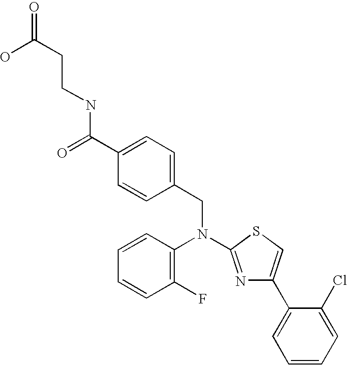 |
| 91 | 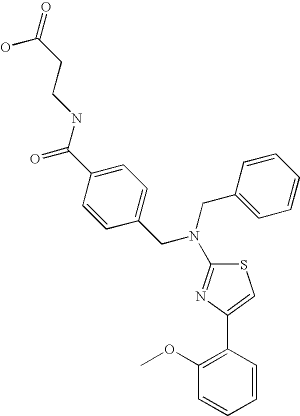 |
| 92 | 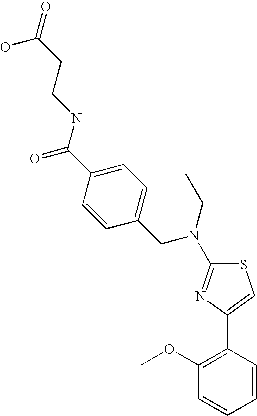 |
| 93 | 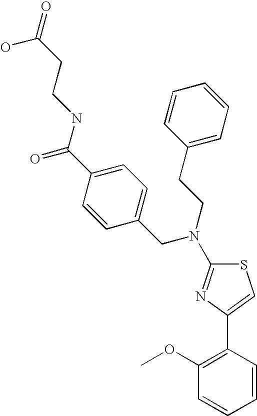 |
| 94 | 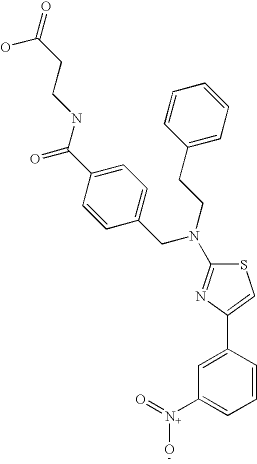 |
| 95 | 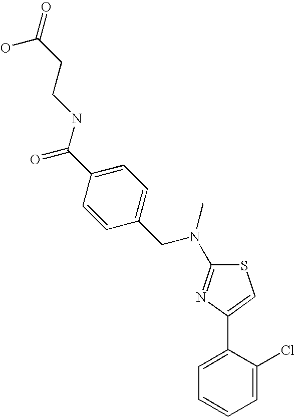 |
| 96 | 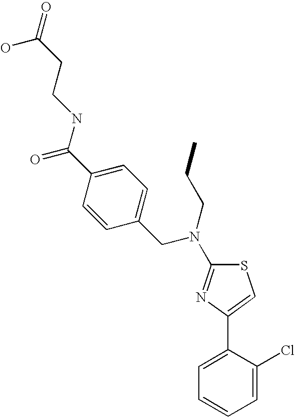 |
| 97 | 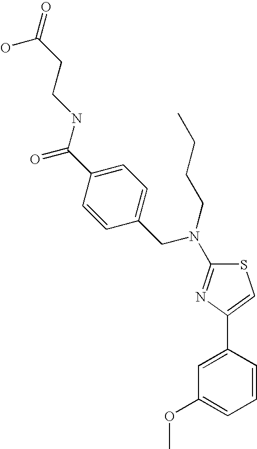 |
| 98 | 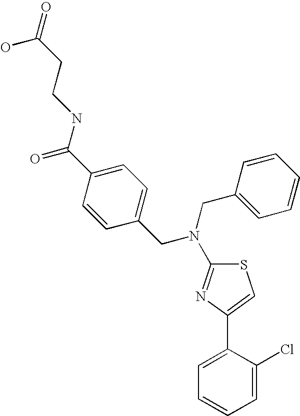 |
| 99 | 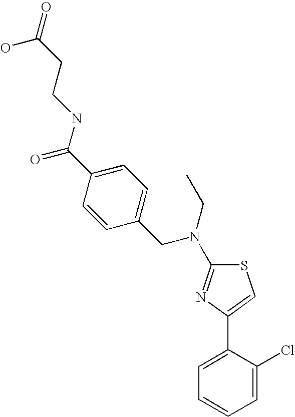 |
| 100 | 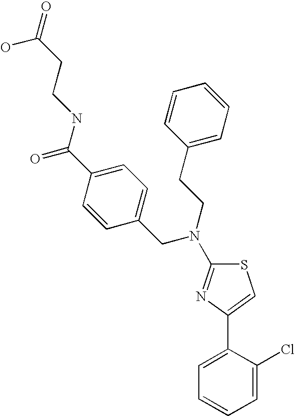 |
| 101 | 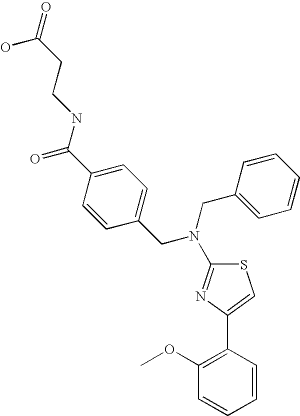 |
| 102 | 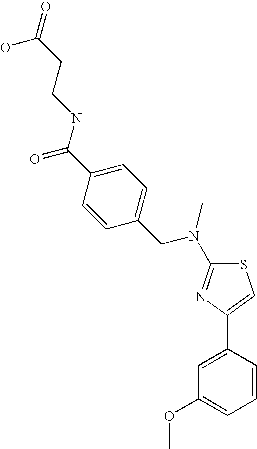 |
| 103 | 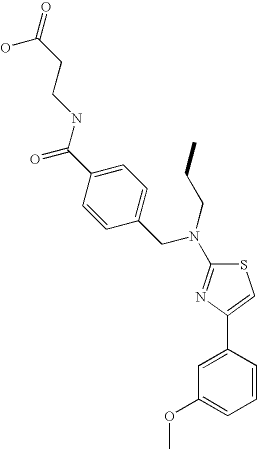 |
| 104 | 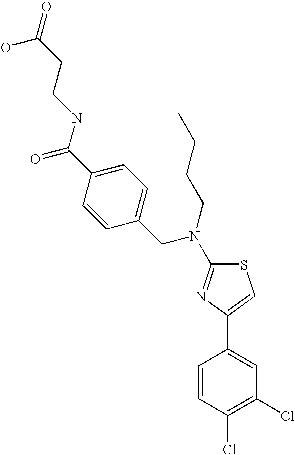 |
| 105 | 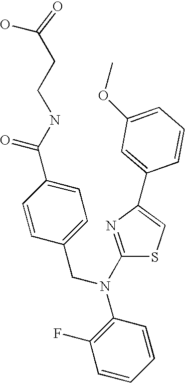 |
| 106 | 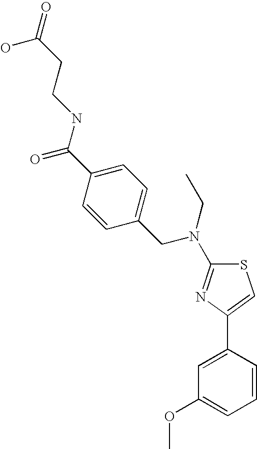 |
| 107 | 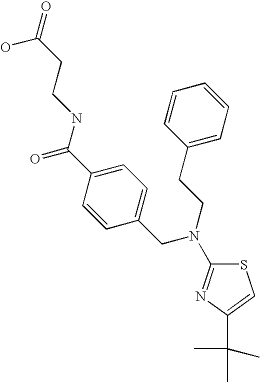 |
| 108 | 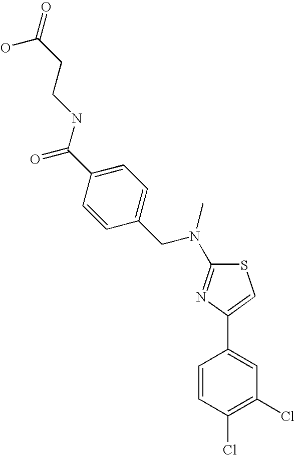 |
| 109 | 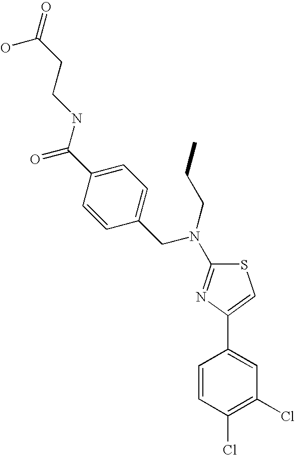 |
| 110 | 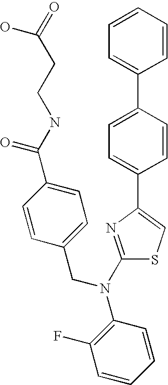 |
| 111 | 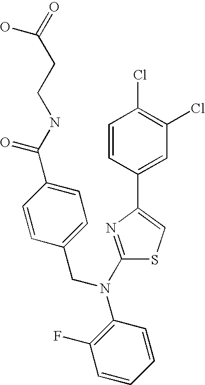 |
| 112 | 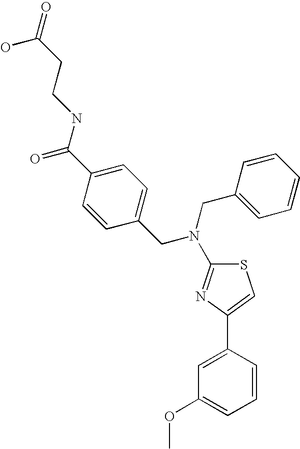 |
| 113 | 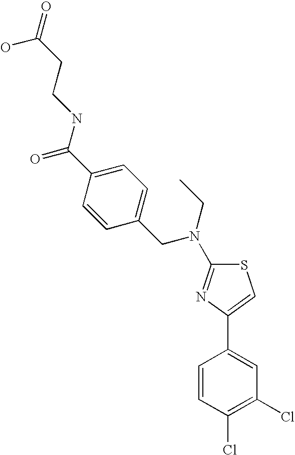 |
| 114 | 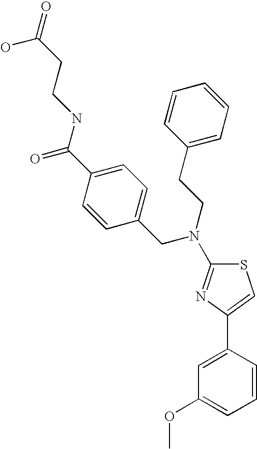 |
| 115 | 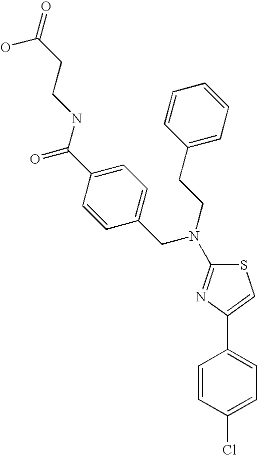 |
| 116 | 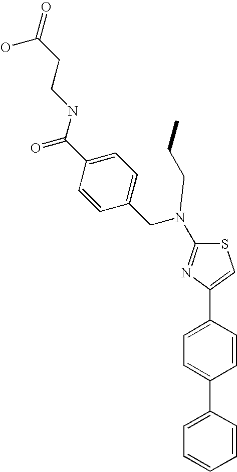 |
| 117 | 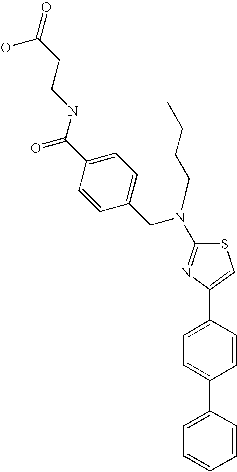 |
| 118 | 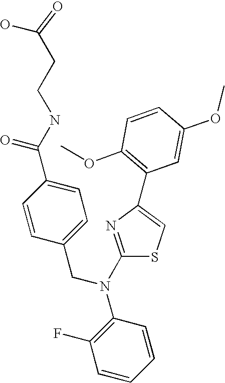 |
| 119 | 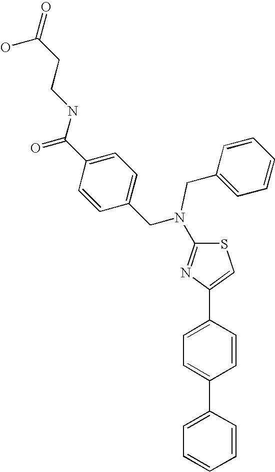 |
| 120 | 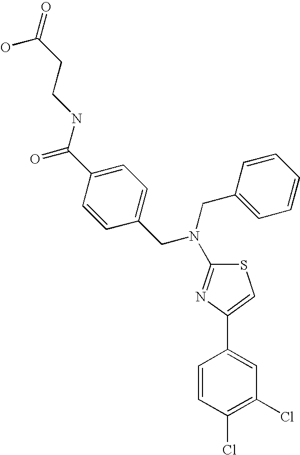 |
| 121 | 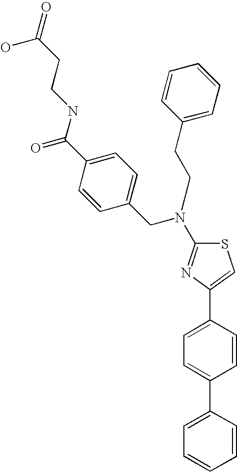 |
| 122 | 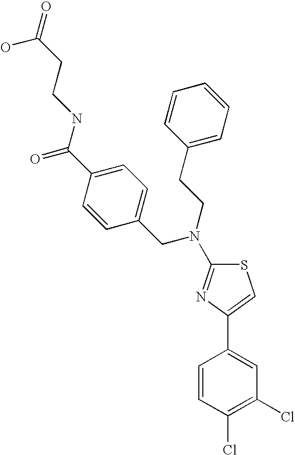 |
| 123 | 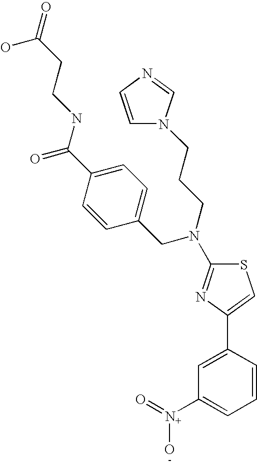 |
| 124 | 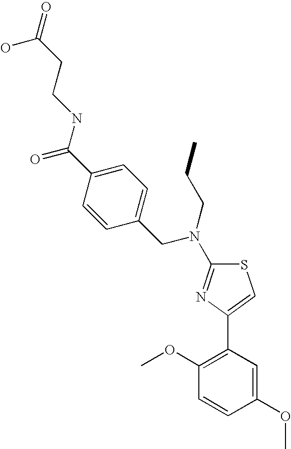 |
| 125 | 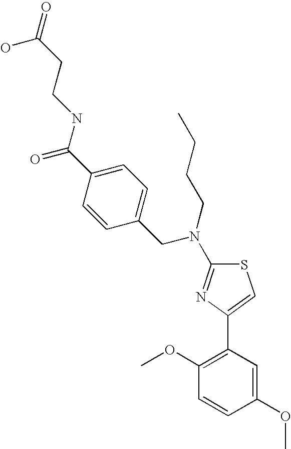 |
| 126 | 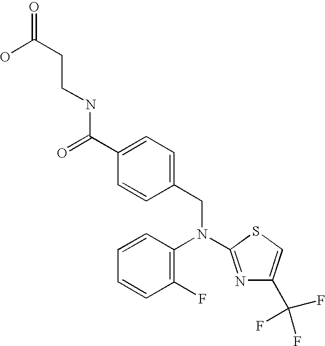 |
| 127 | 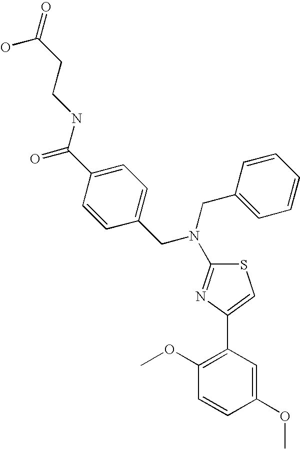 |
| 128 | 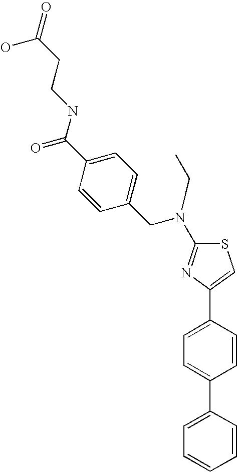 |
| 129 | 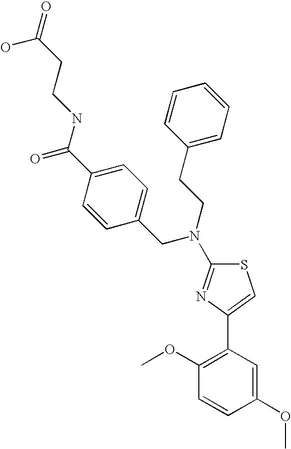 |
| 130 | 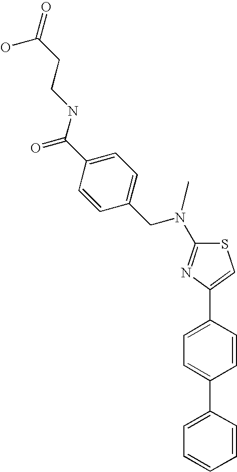 |
| 131 | 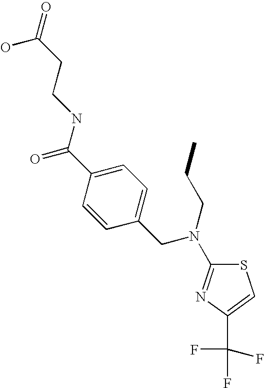 |
| 132 | 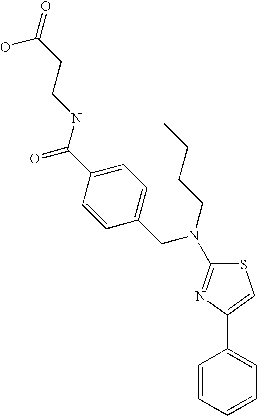 |
| 133 | 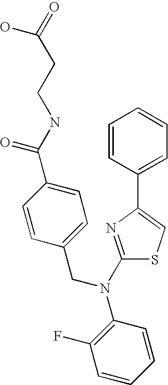 |
| 134 | 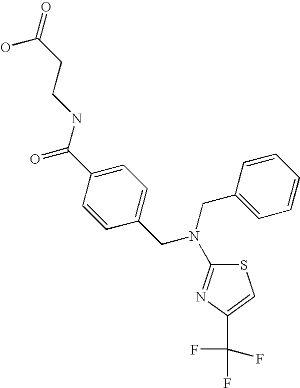 |
| 135 | 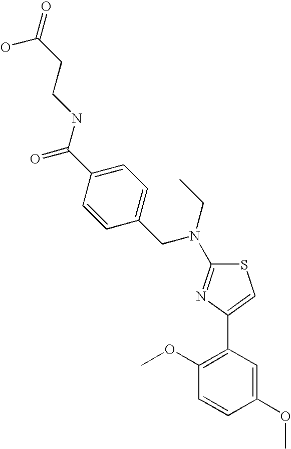 |
| 136 | 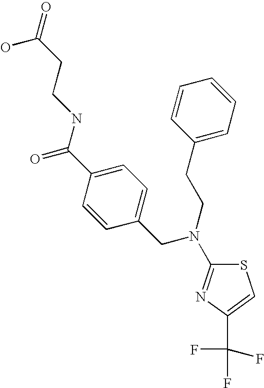 |
| 137 | 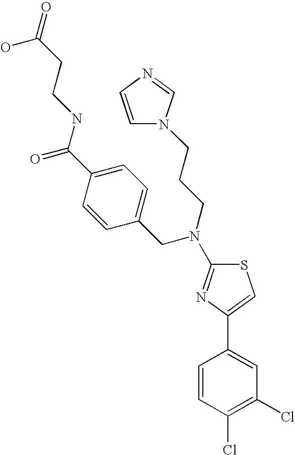 |
| 138 | 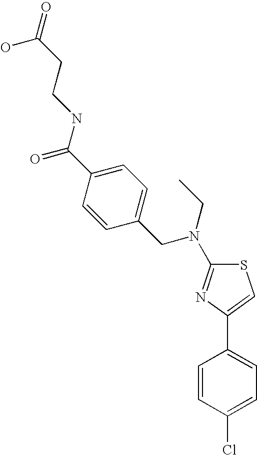 |
| 139 | 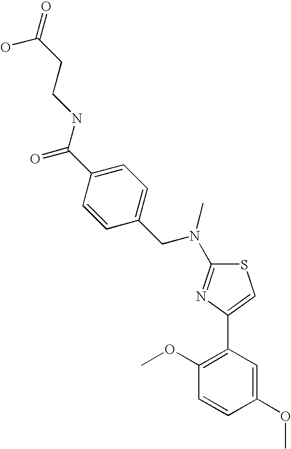 |
| 140 | 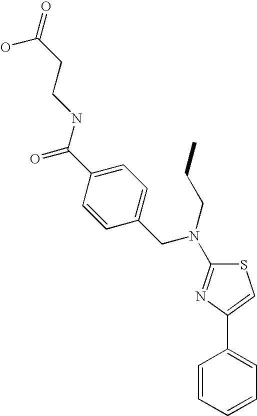 |
| 141 | 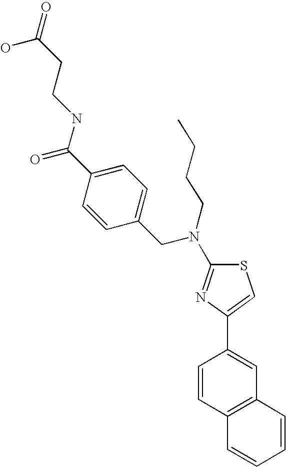 |
| 142 | 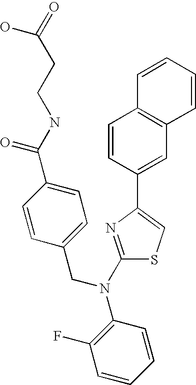 |
| 143 | 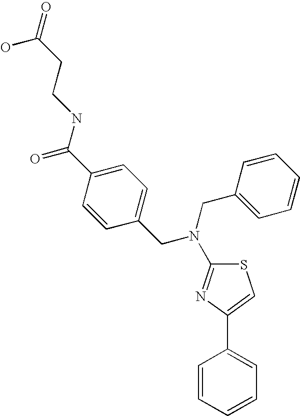 |
| 144 | 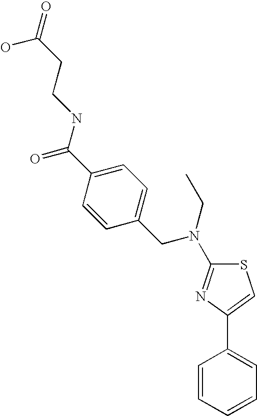 |
| 145 | 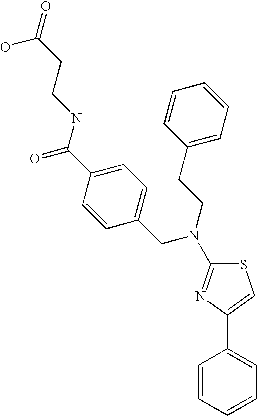 |
| 146 | 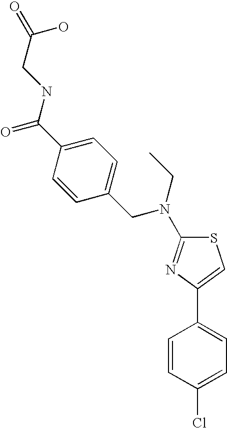 |
| 147 | 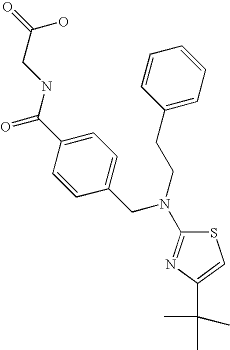 |
| 148 | 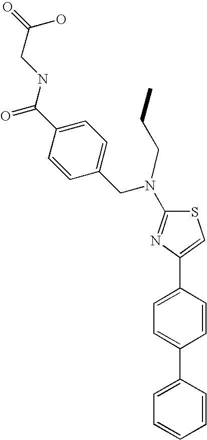 |
| 149 | 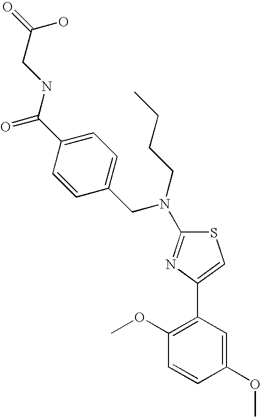 |
| 150 | 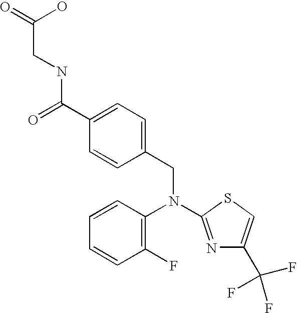 |
| 151 | 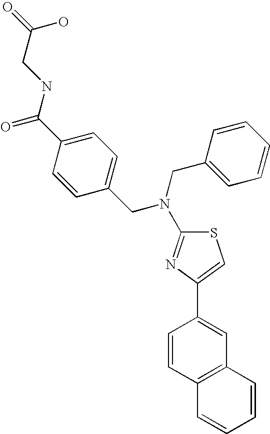 |
| 152 | 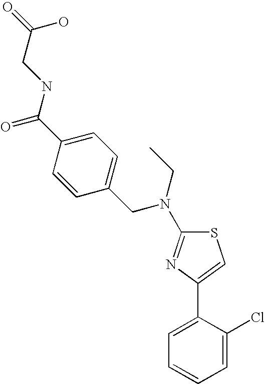 |
| 153 | 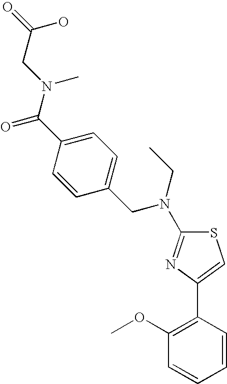 |
| 154 | 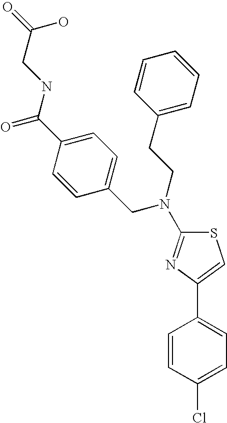 |
| 155 | 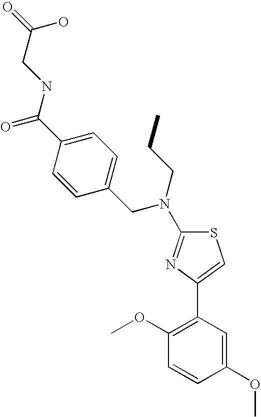 |
| 156 | 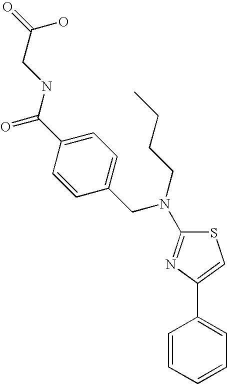 |
| 157 | 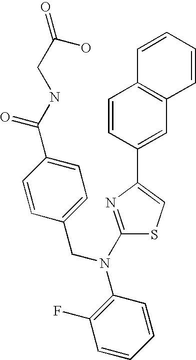 |
| 158 | 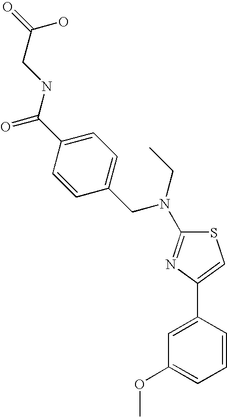 |
| 159 | 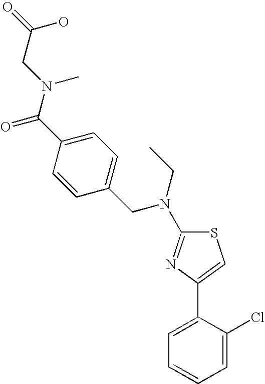 |
| 160 | 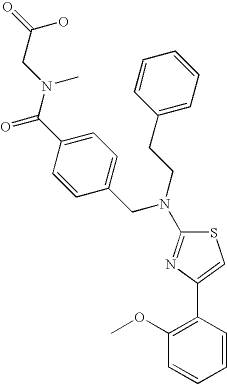 |
| 161 | 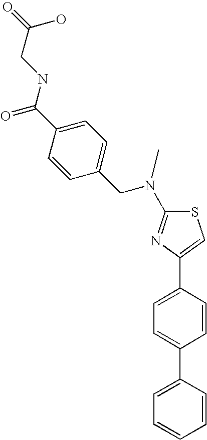 |
| 162 | 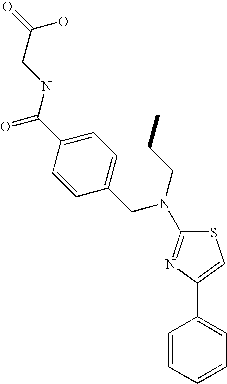 |
| 163 | 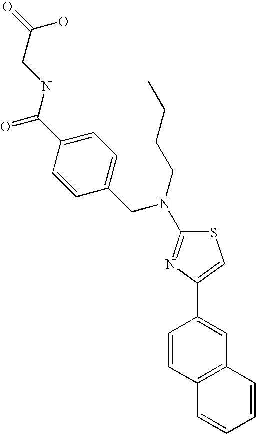 |
| 164 | 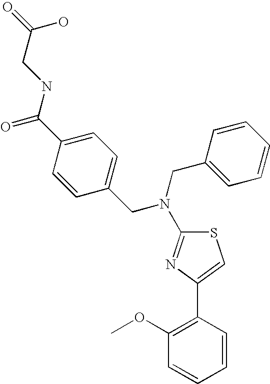 |
| 165 | 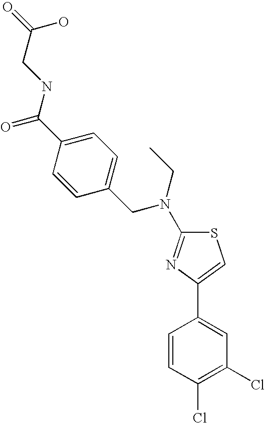 |
| 166 | 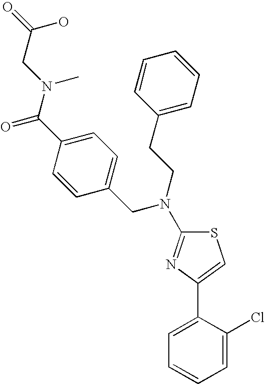 |
| 167 | 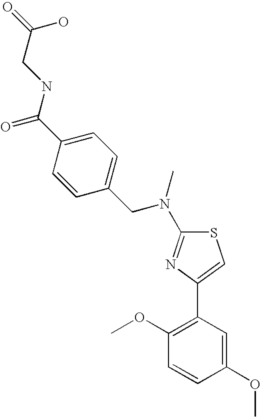 |
| 168 | 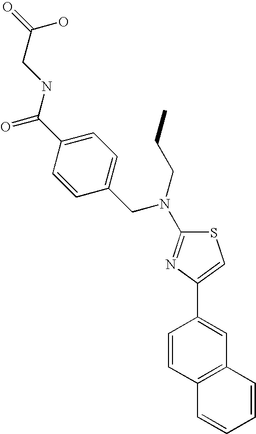 |
| 169 | 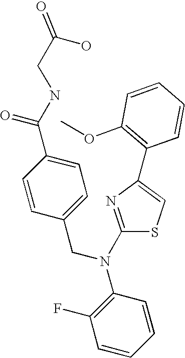 |
| 170 | 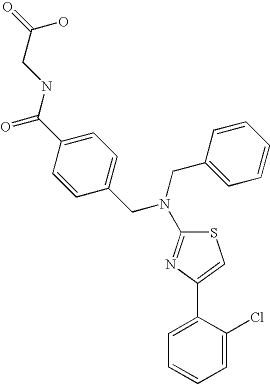 |
| 171 | 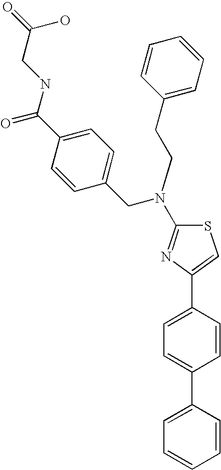 |
| 172 | 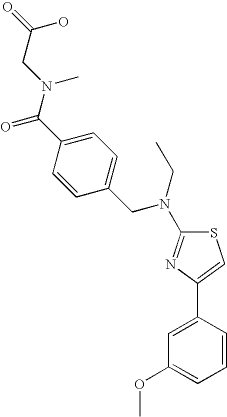 |
| 173 | 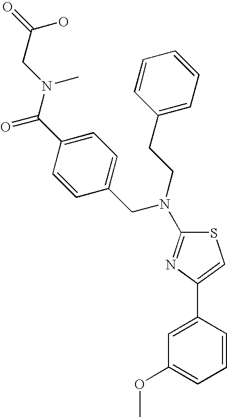 |
| 174 | 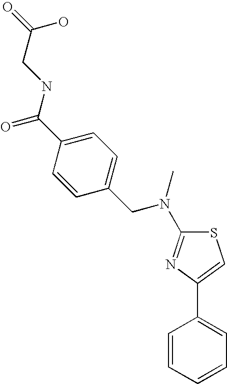 |
| 175 | 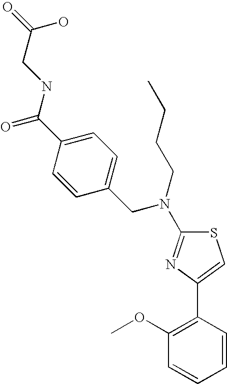 |
| 176 | 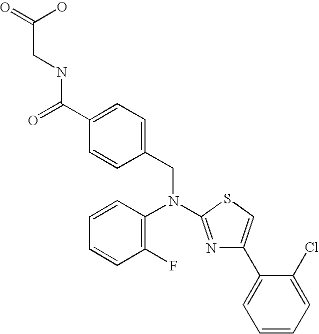 |
| 177 | 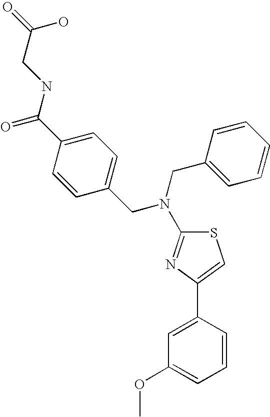 |
| 178 | 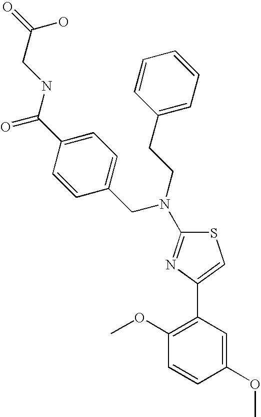 |
| 179 | 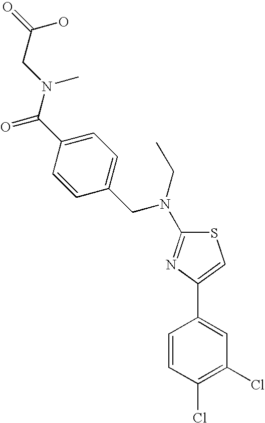 |
| 180 | 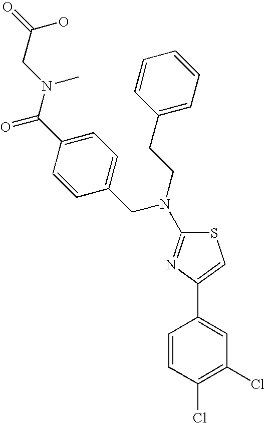 |
| 181 | 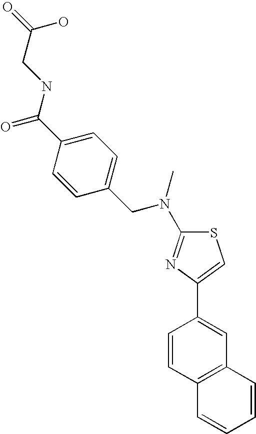 |
| 182 | 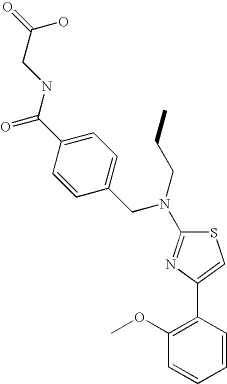 |
| 184 | 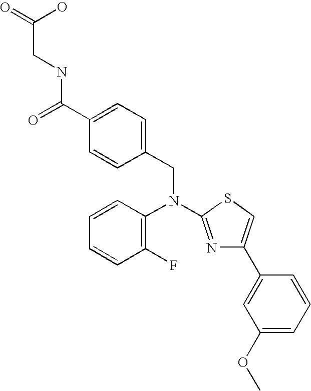 |
| 185 | 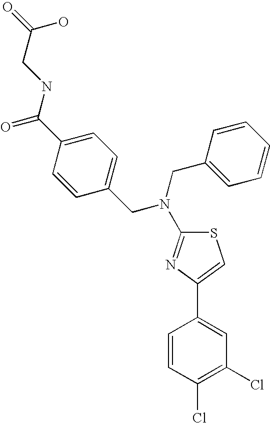 |
| 186 | 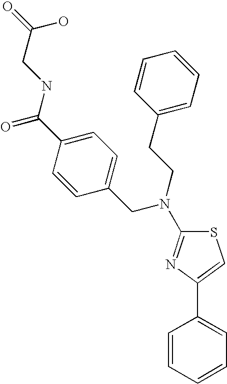 |
| 187 | 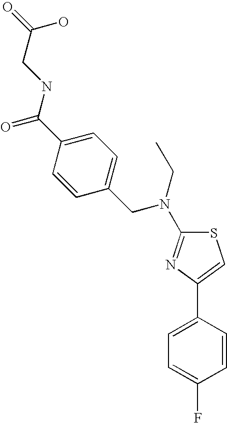 |
| 188 | 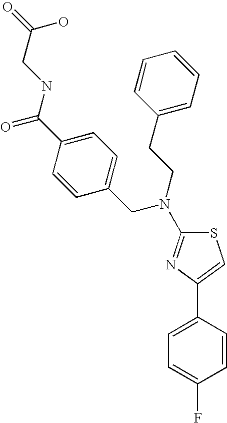 |
| 189 | 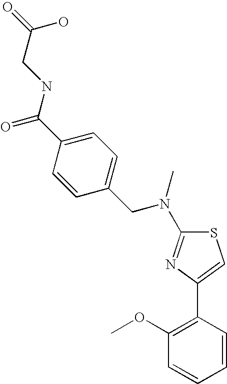 |
| 190 | 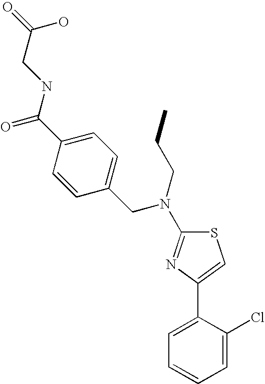 |
| 191 | 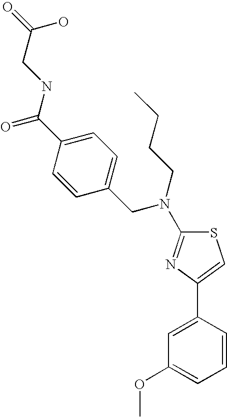 |
| 192 | 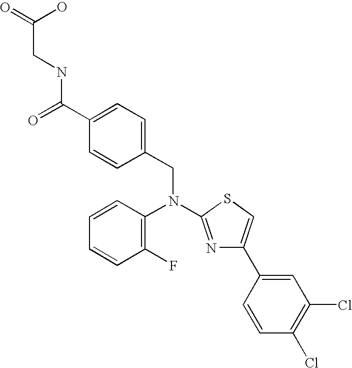 |
| 193 | 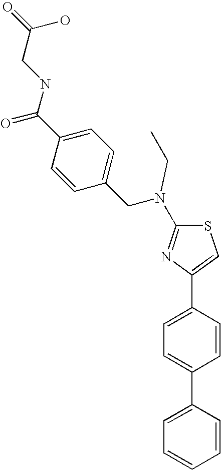 |
| 194 | 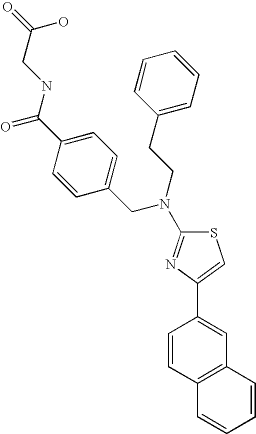 |
| 195 | 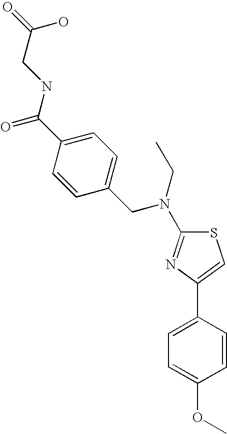 |
| 196 | 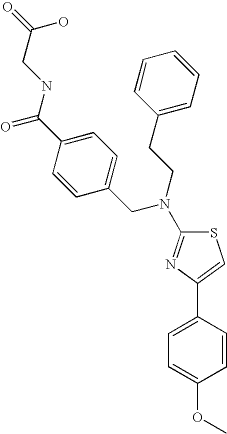 |
| 197 | 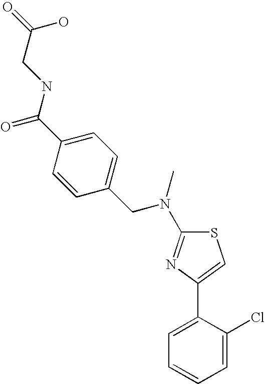 |
| 198 | 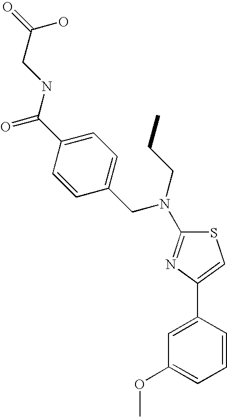 |
| 199 | 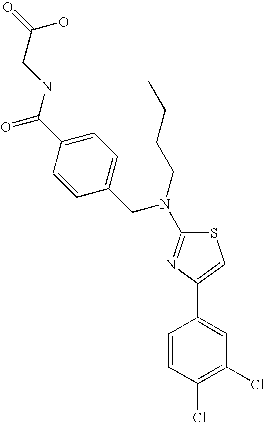 |
| 200 | 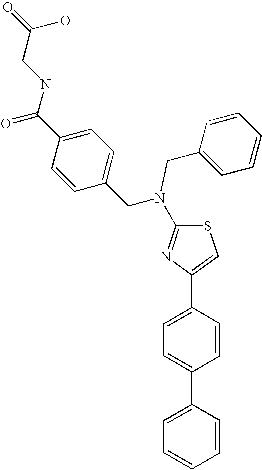 |
| 201 | 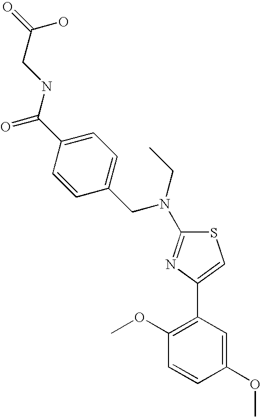 |
| 202 | 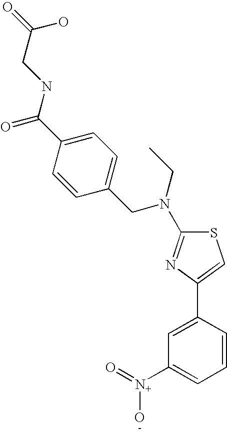 |
| 203 | 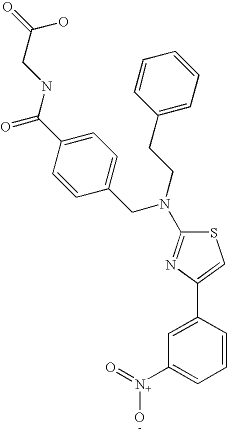 |
| 204 | 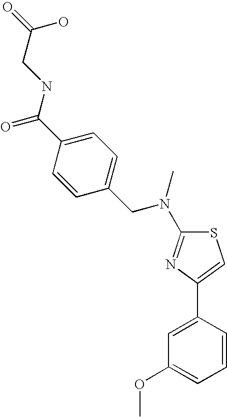 |
| 205 | 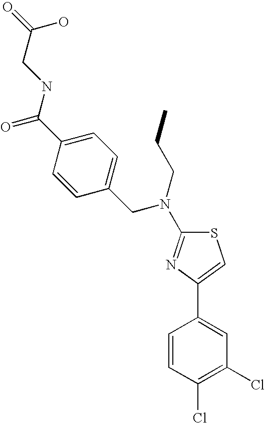 |
| 206 | 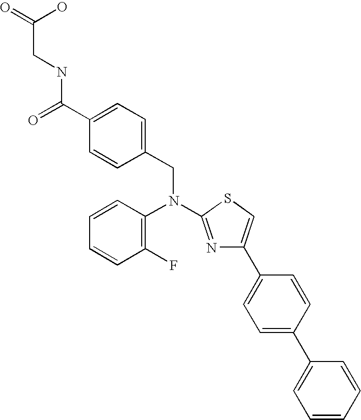 |
| 207 | 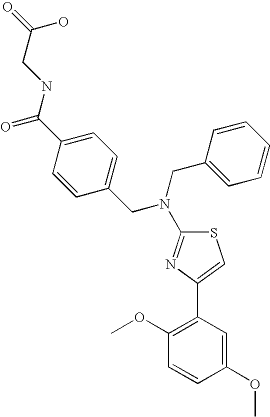 |
| 208 | 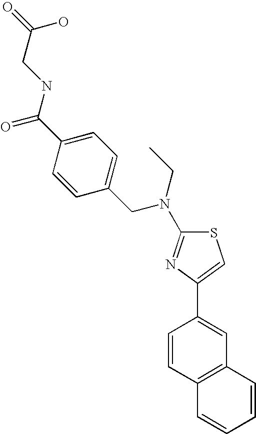 |
| 209 | 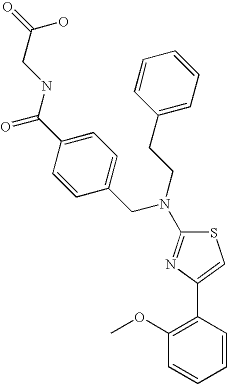 |
| 210 | 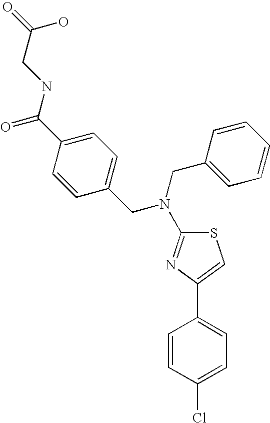 |
| 211 | 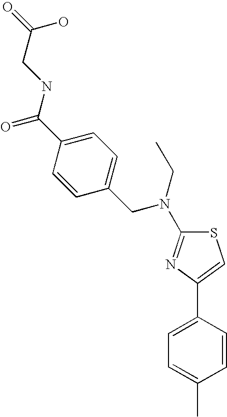 |
| 212 | 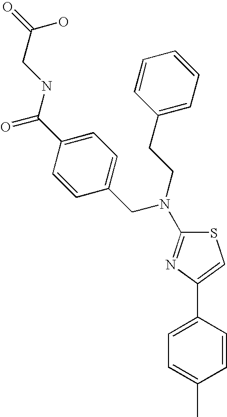 |
| 213 | 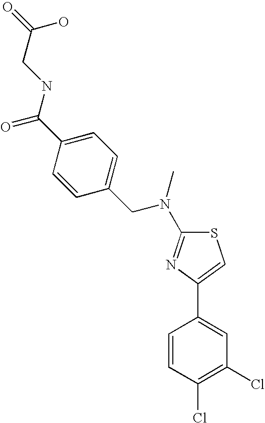 |
| 214 | 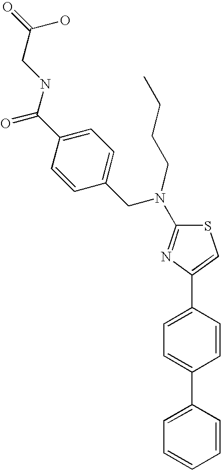 |
| 215 | 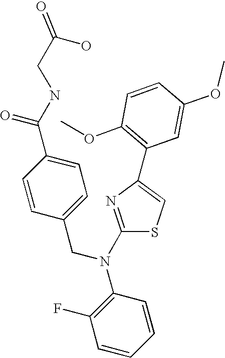 |
| 216 | 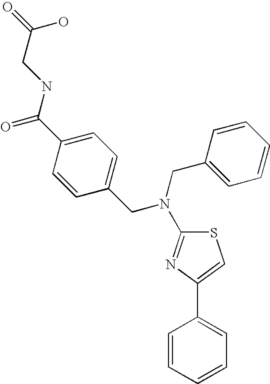 |
| 217 | 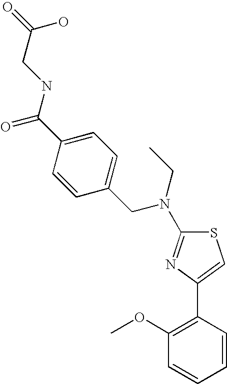 |
| 218 | 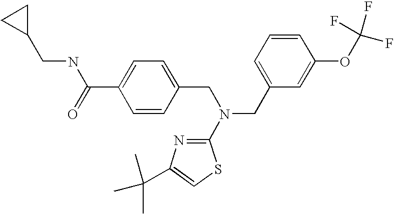 |
| 219 | 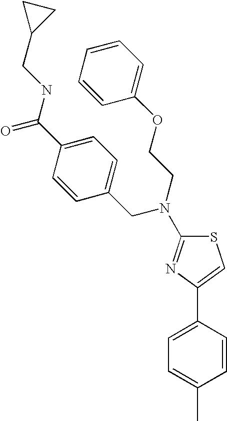 |
| 220 | 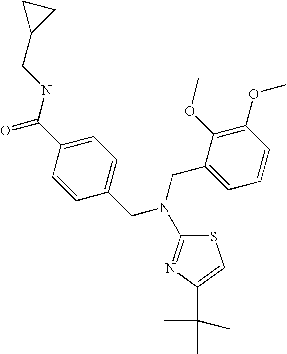 |
| 221 | 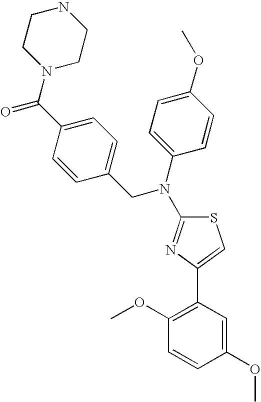 |
| 222 | 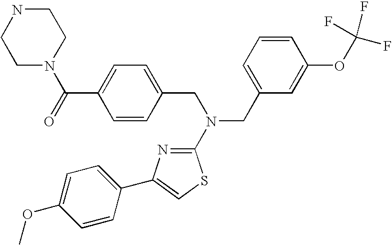 |
| 223 | 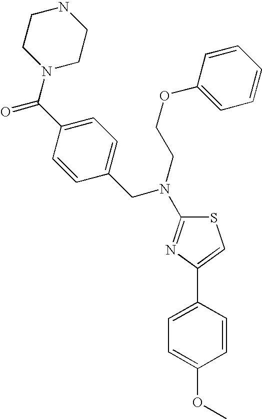 |
| 224 | 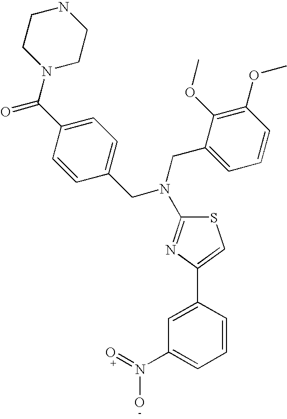 |
| 225 | 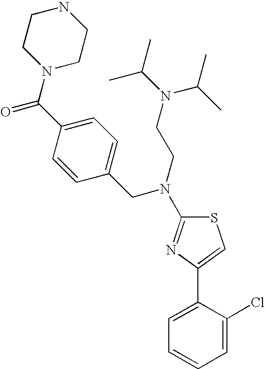 |
| 226 | 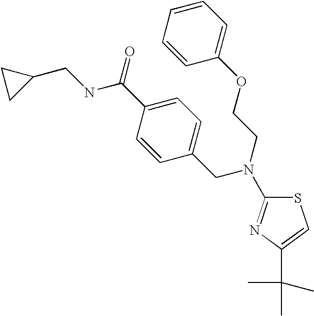 |
| 227 | 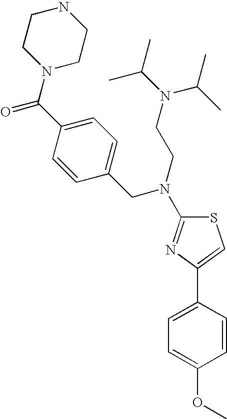 |
| 228 | 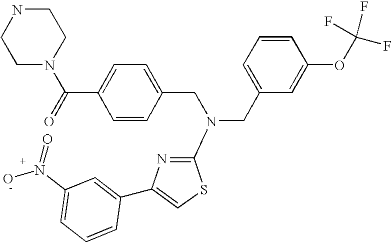 |
| 229 | 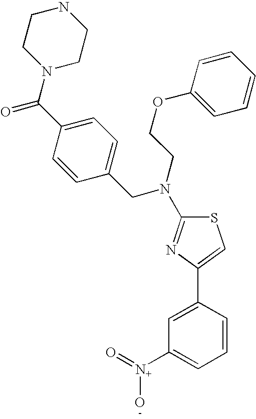 |
| 230 | 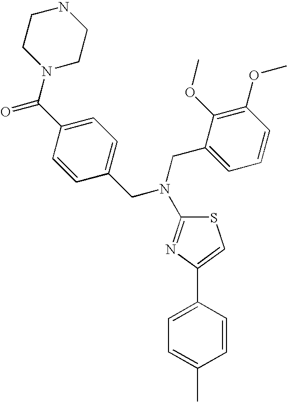 |
| 231 | 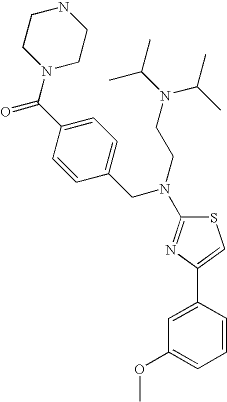 |
| 232 | 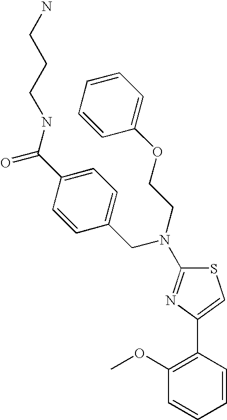 |
| 233 | 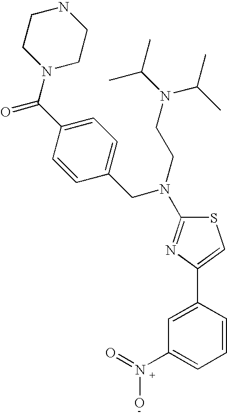 |
| 234 | 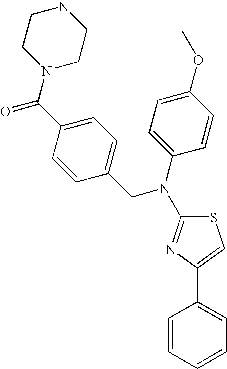 |
| 235 | 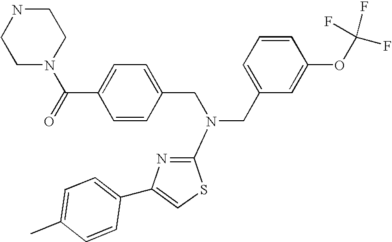 |
| 236 | 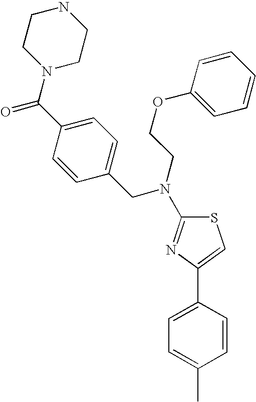 |
| 237 | 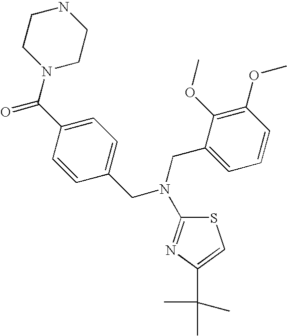 |
| 238 | 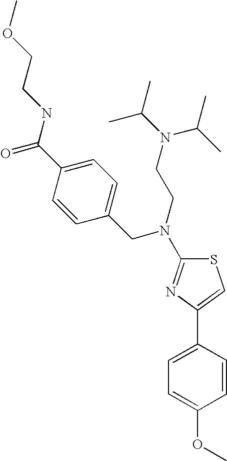 |
| 239 | 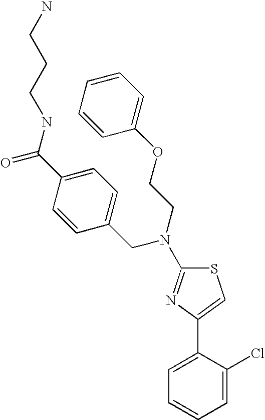 |
| 240 | 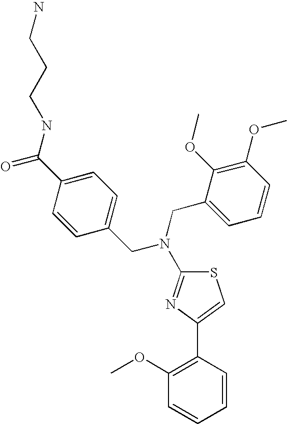 |
| 241 | 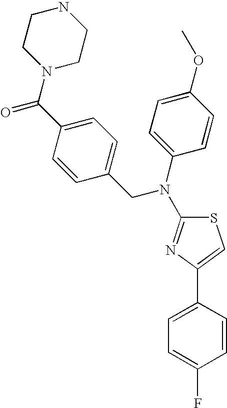 |
| 242 | 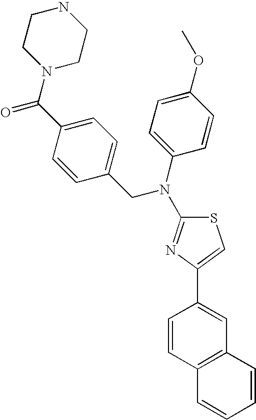 |
| 243 | 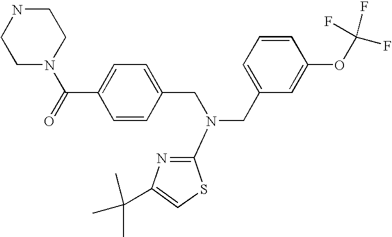 |
| 244 | 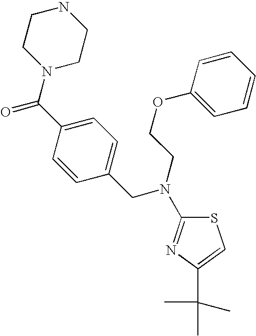 |
| 245 | 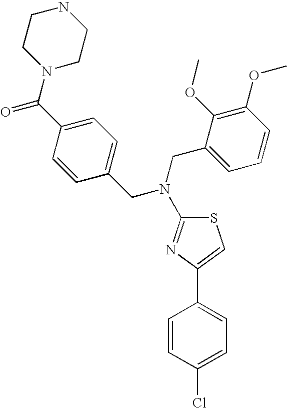 |
| 246 | 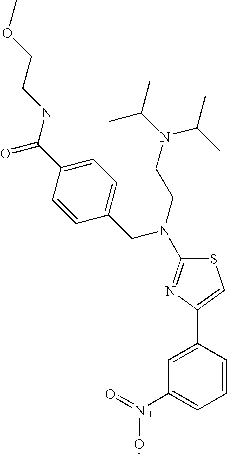 |
| 247 | 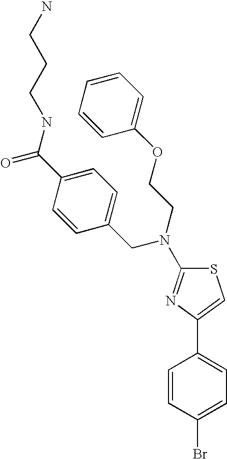 |
| 248 | 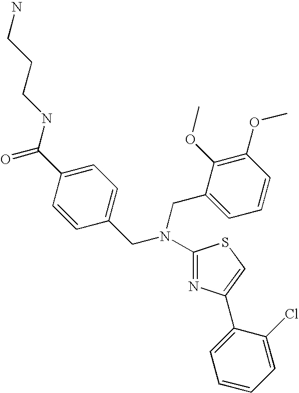 |
| 249 | 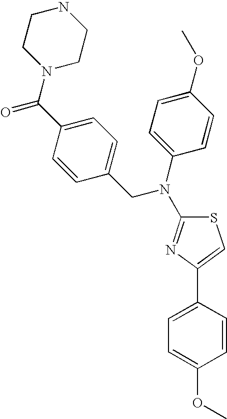 |
| 250 | 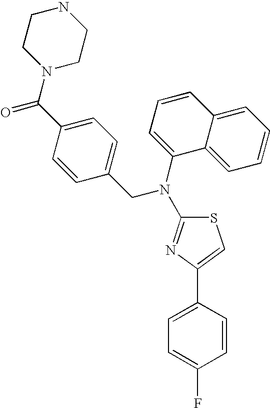 |
| 251 | 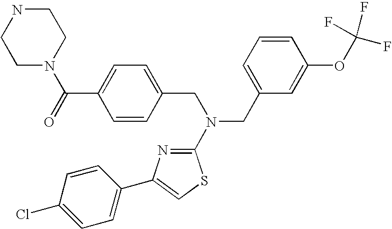 |
| 252 | 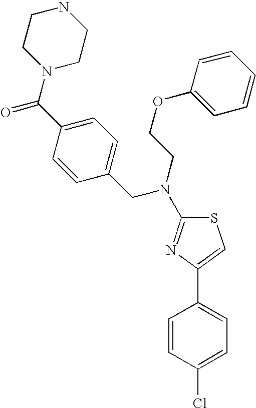 |
| 253 | 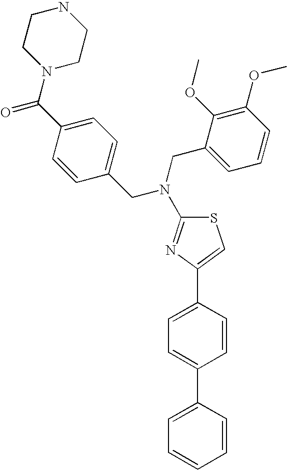 |
| 254 | 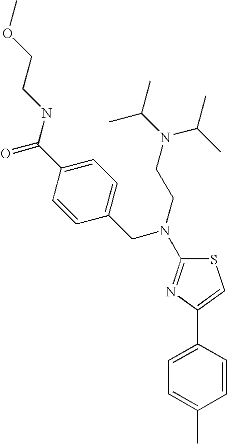 |
| 255 | 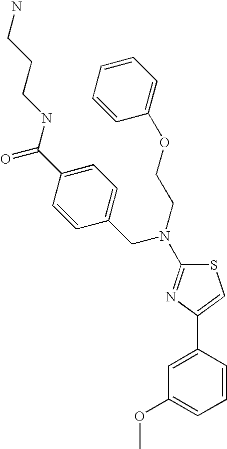 |
| 256 | 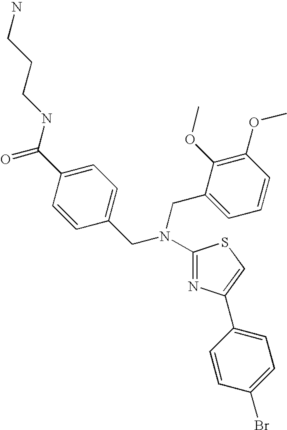 |
| 257 | 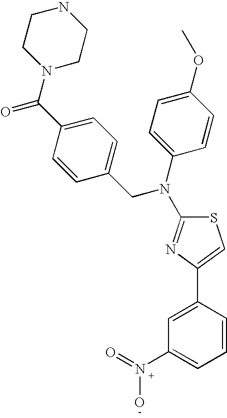 |
| 258 | 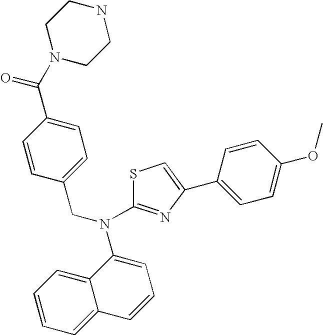 |
| 259 | 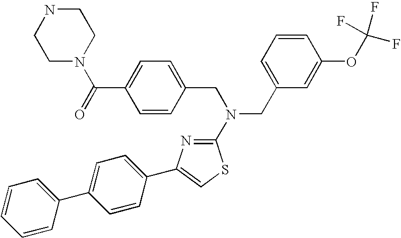 |
| 260 | 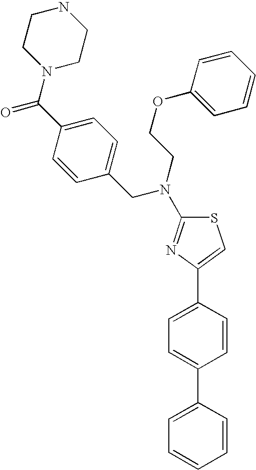 |
| 261 | 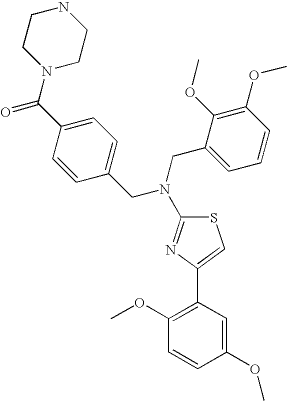 |
| 262 | 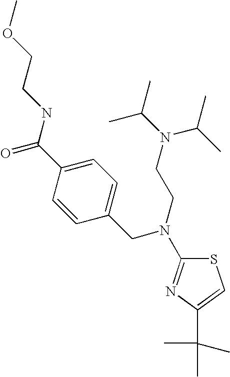 |
| 263 | 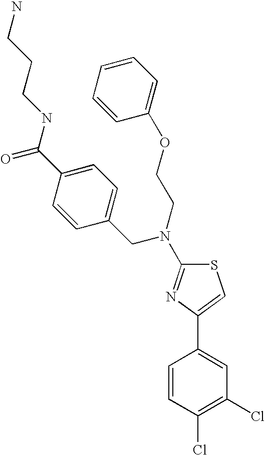 |
| 264 | 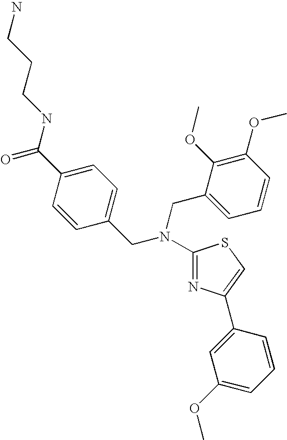 |
| 265 | 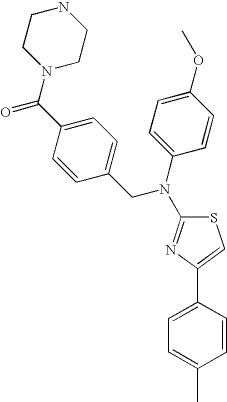 |
| 266 | 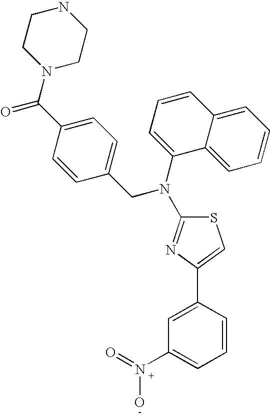 |
| 267 | 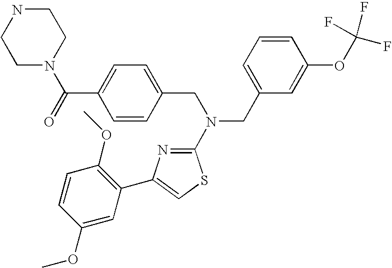 |
| 268 | 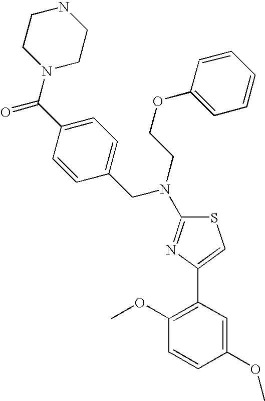 |
| 269 | 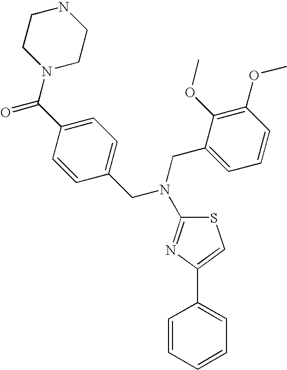 |
| 270 | 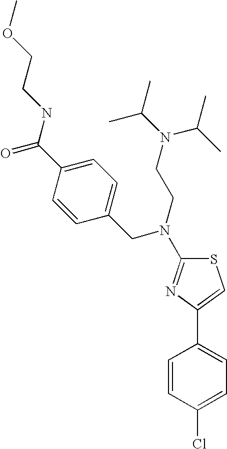 |
| 271 | 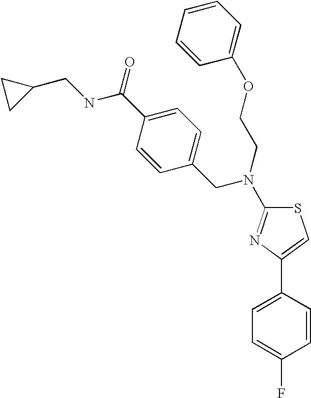 |
| 272 | 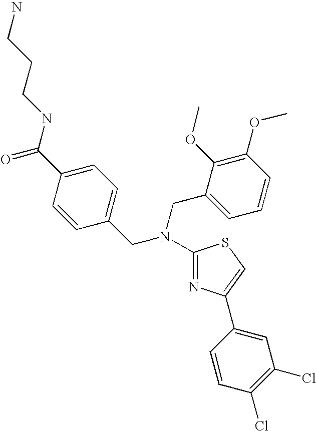 |
| 273 | 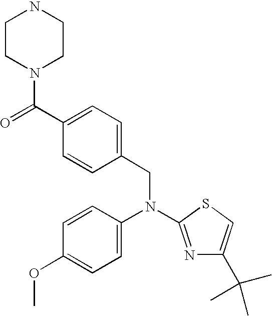 |
| 274 | 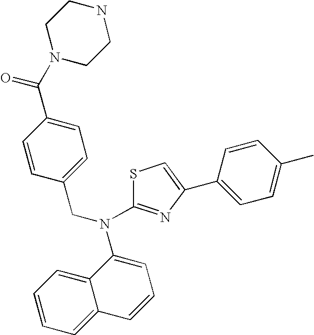 |
| 275 | 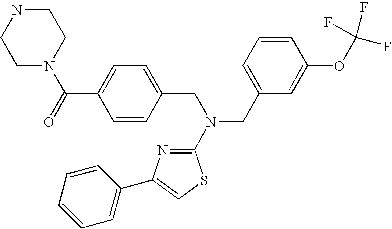 |
| 276 | 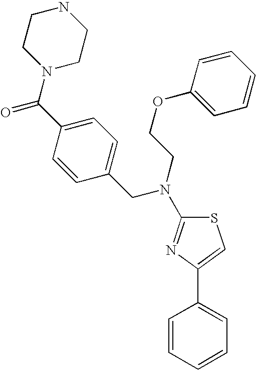 |
| 277 | 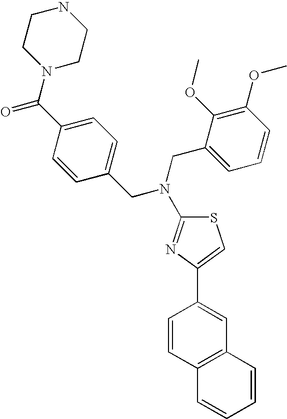 |
| 278 | 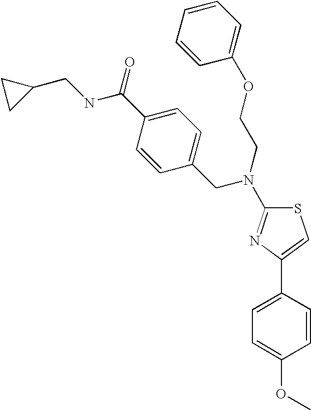 |
| 279 | 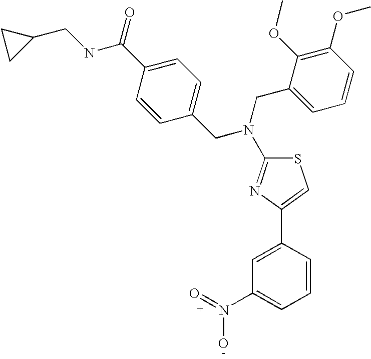 |
| 280 | 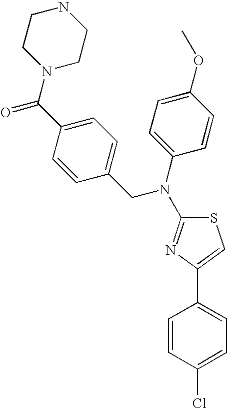 |
| 281 | 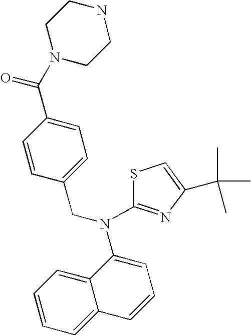 |
| 282 | 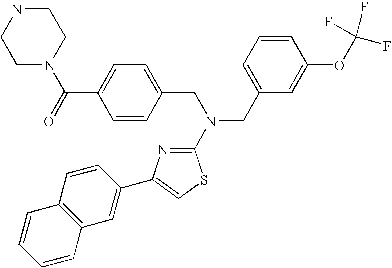 |
| 283 | 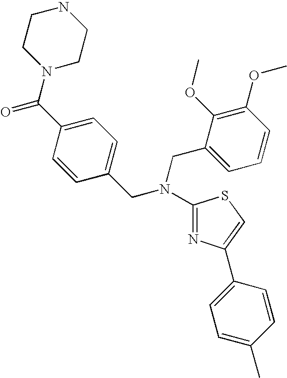 |
| 284 | 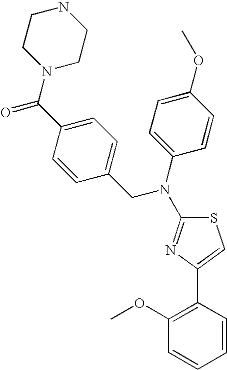 |
| 285 | 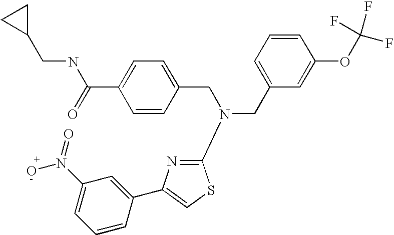 |
| 286 | 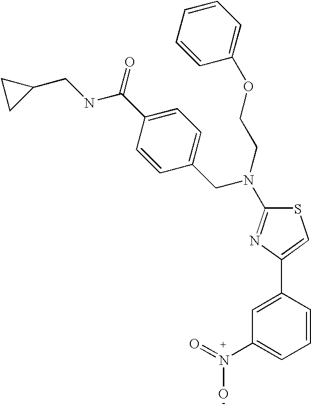 |
| 287 | 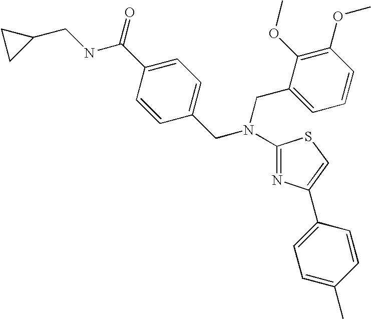 |
| 288 | 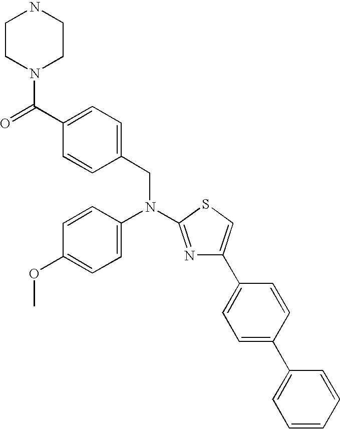 |
| 289 | 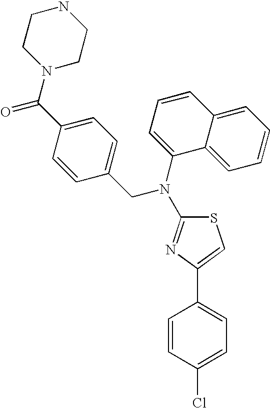 |
| 290 | 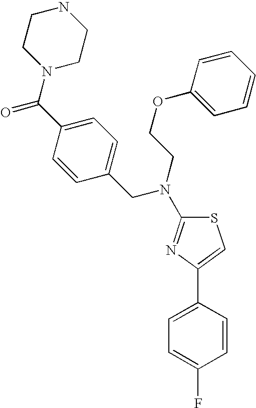 |
| 291 | 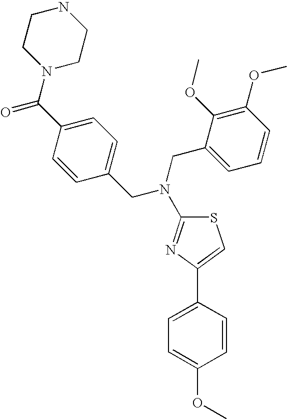 |
| 292 | 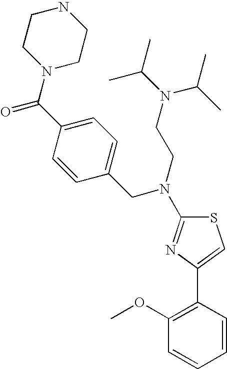 |
| 293 | 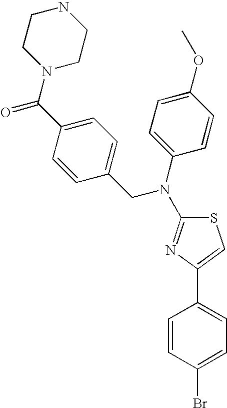 |
| 294 | 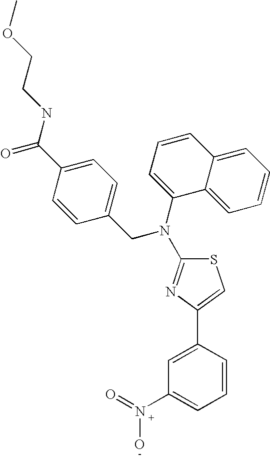 |
| 295 | 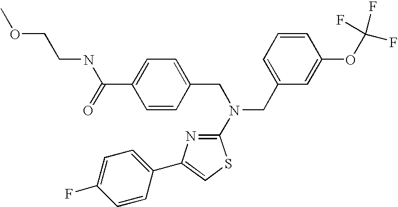 |
| 296 | 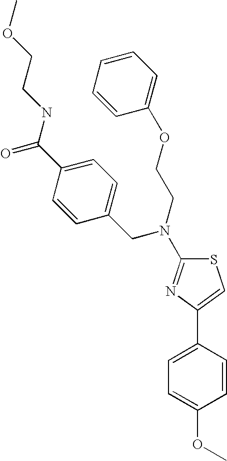 |
| 297 | 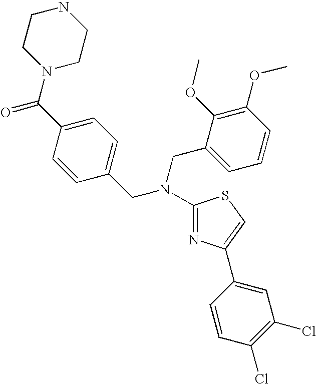 |
| 298 | 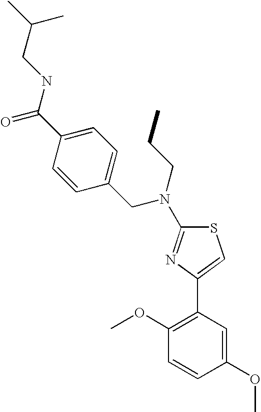 |
| 299 | 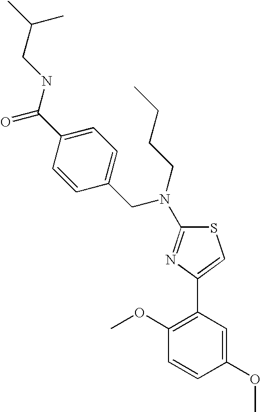 |
| 300 | 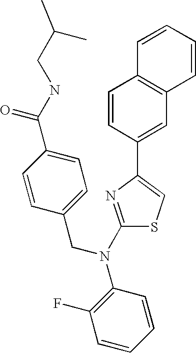 |
| 301 | 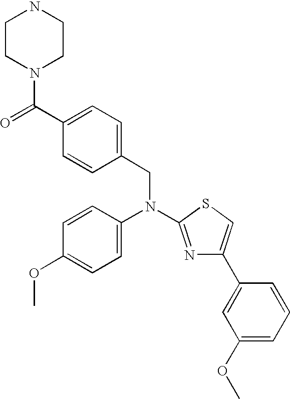 |
| 302 | 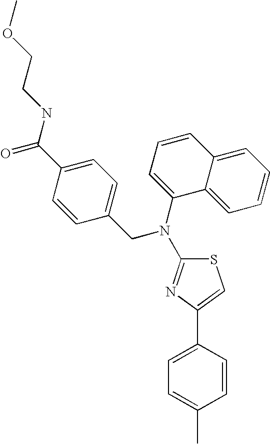 |
| 303 | 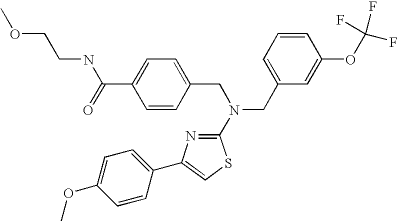 |
| 304 | 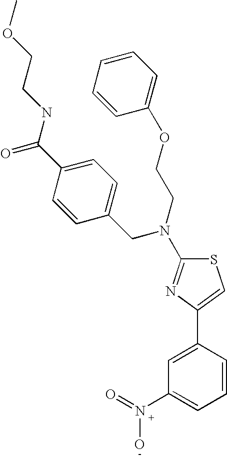 |
| 305 | 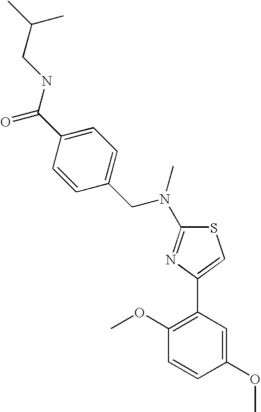 |
| 306 | 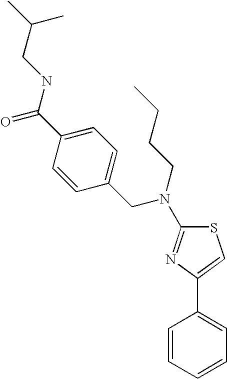 |
| 307 | 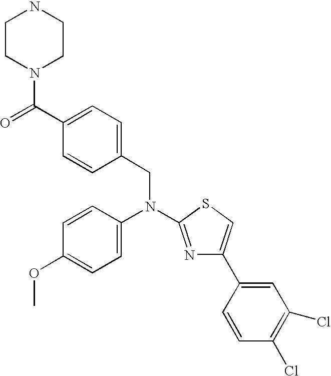 |
| 308 | 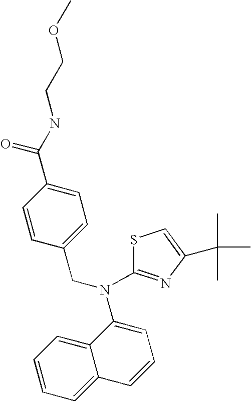 |
| 309 | 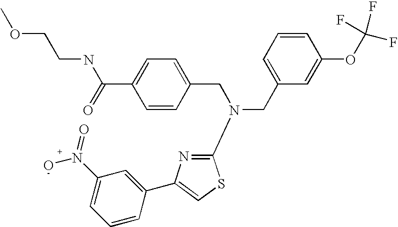 |
| 310 | 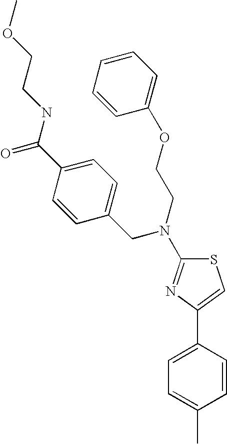 |
| 311 | 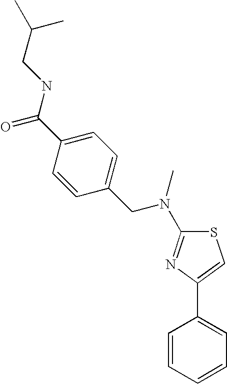 |
| 312 | 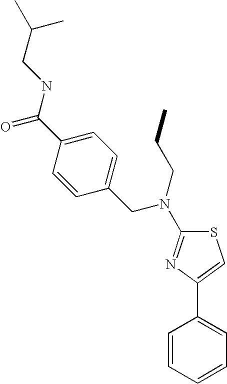 |
| 313 | 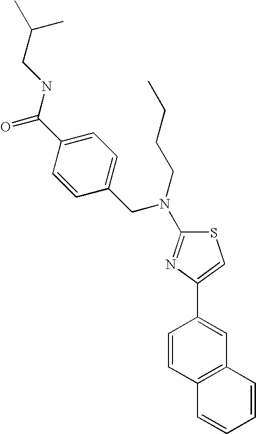 |
| 314 | 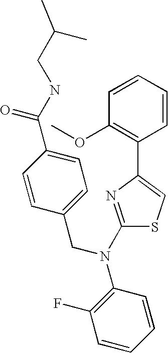 |
| 315 | 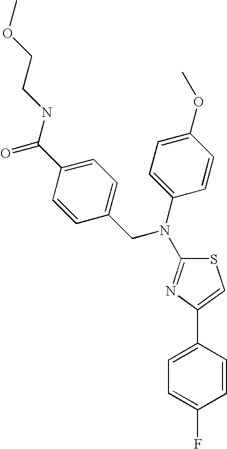 |
| 316 | 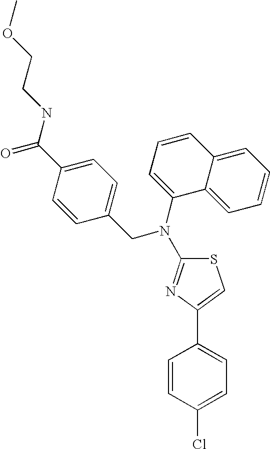 |
| 317 | 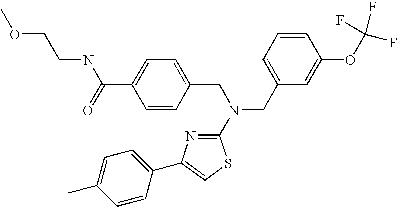 |
| 318 | 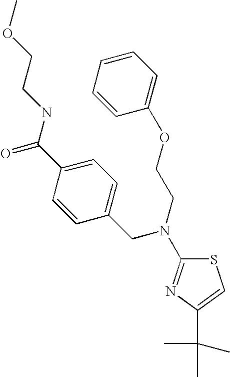 |
| 319 | 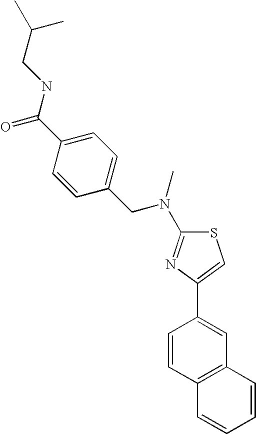 |
| 320 | 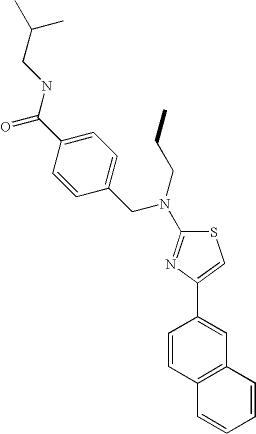 |
| 321 | 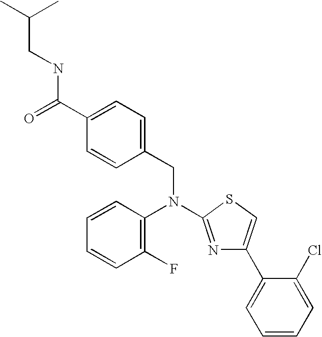 |
| 322 | 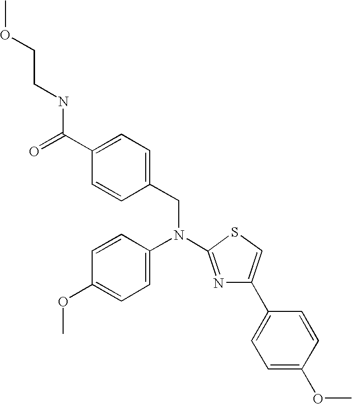 |
| 323 | 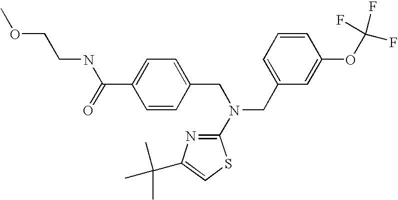 |
| 324 | 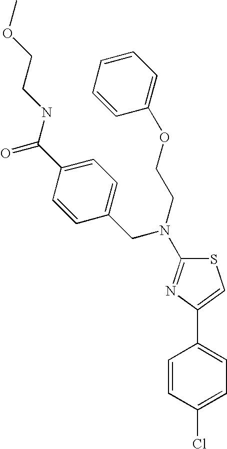 |
| 325 | 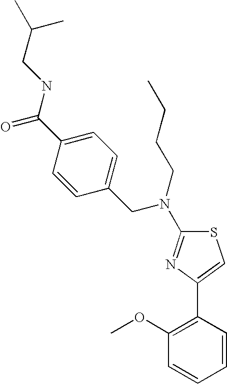 |
| 326 | 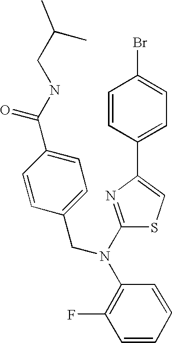 |
| 327 | 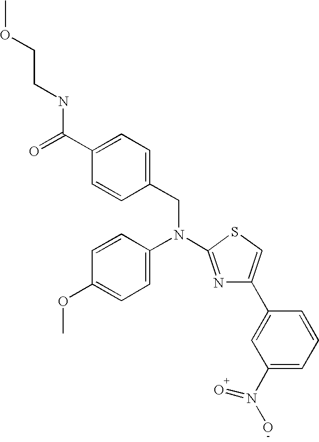 |
| 328 | 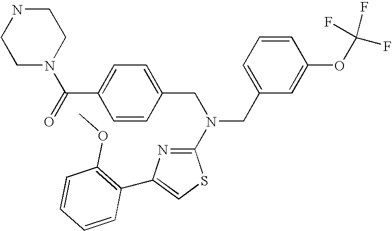 |
| 329 | 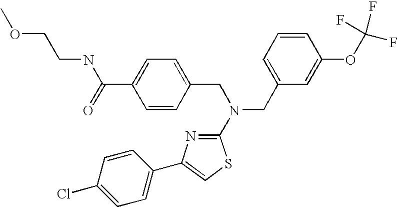 |
| 330 | 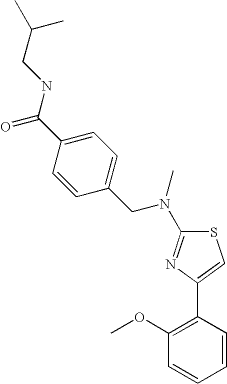 |
| 331 | 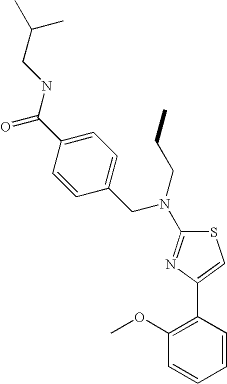 |
| 332 | 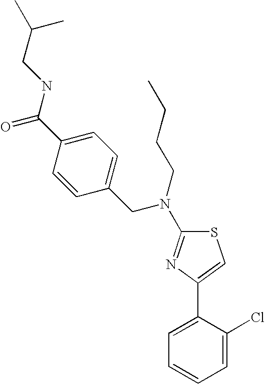 |
| 333 | 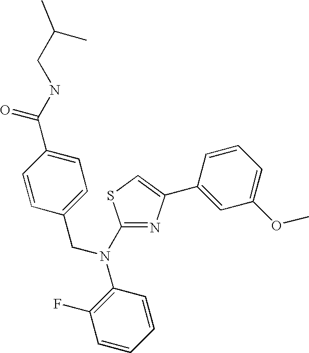 |
| 334 | 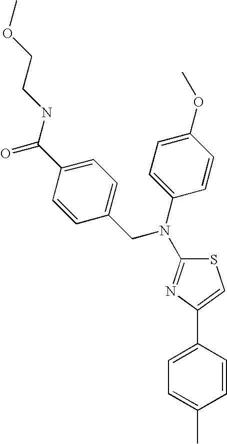 |
| 335 | 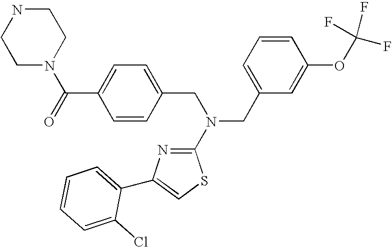 |
| 336 | 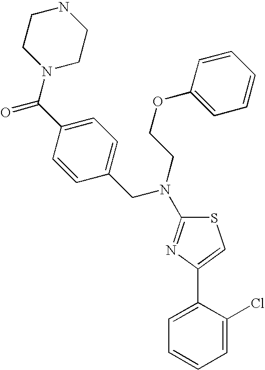 |
| 337 | 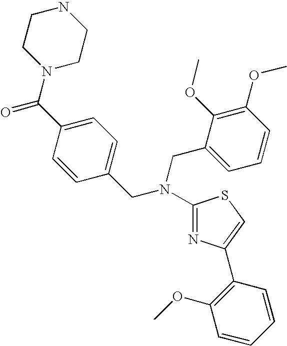 |
| 338 | 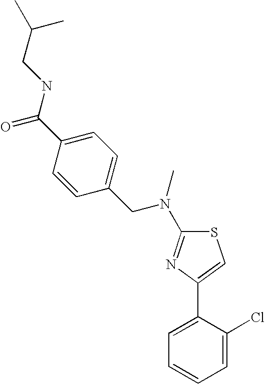 |
| 339 | 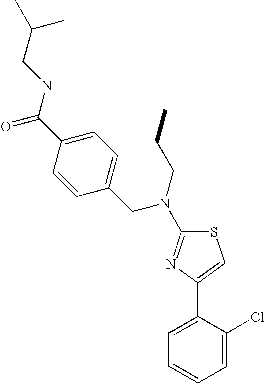 |
| 340 | 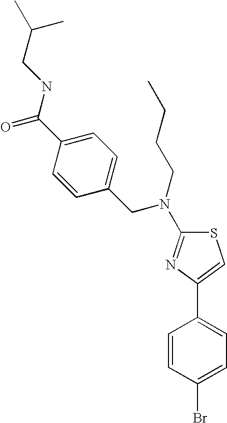 |
| 341 | 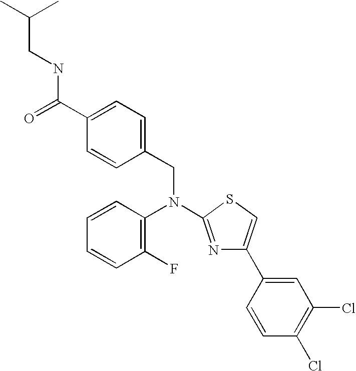 |
| 342 | 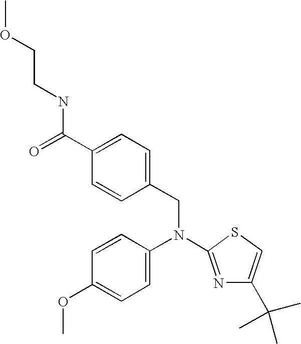 |
| 343 | 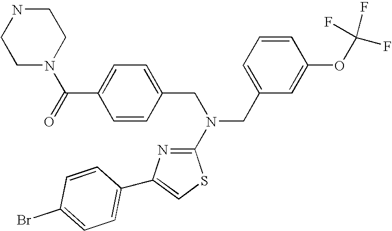 |
| 344 | 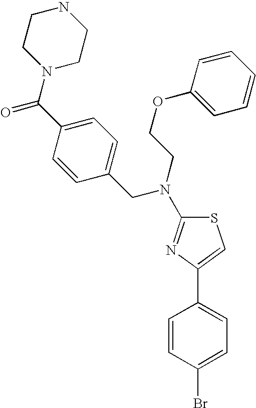 |
| 345 | 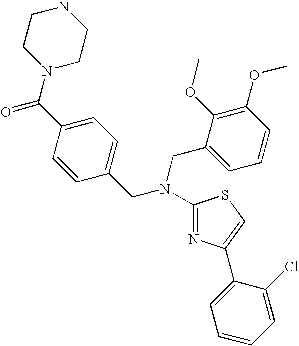 |
| 346 | 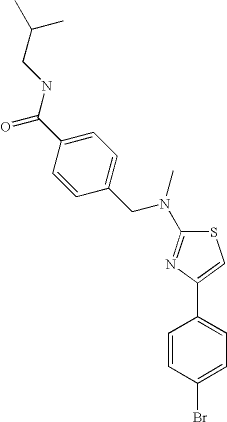 |
| 347 | 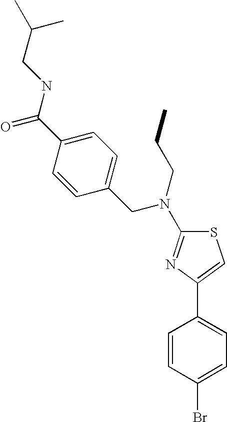 |
| 348 | 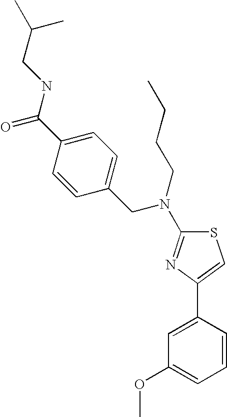 |
| 349 | 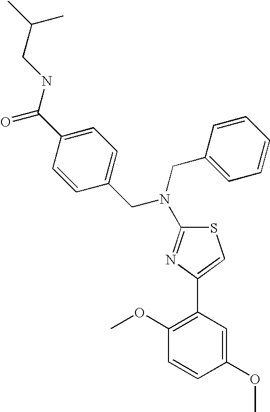 |
| 350 | 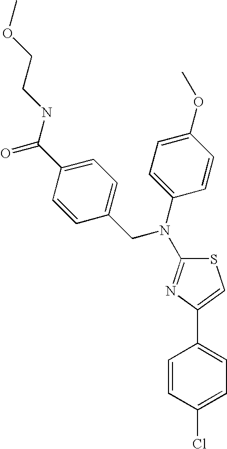 |
| 351 | 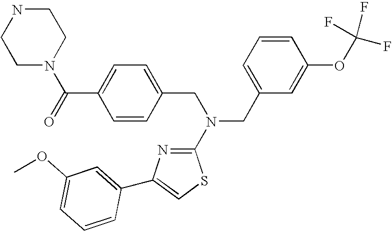 |
| 352 | 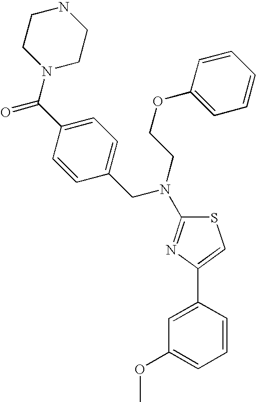 |
| 353 | 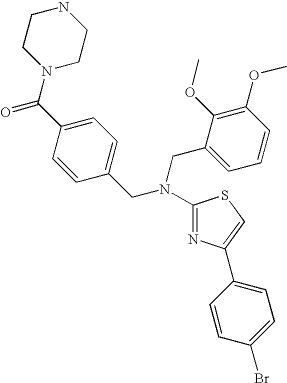 |
| 354 | 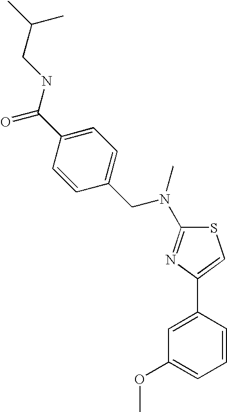 |
| 355 | 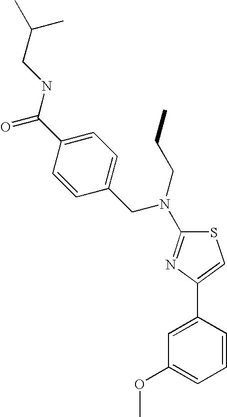 |
| 356 | 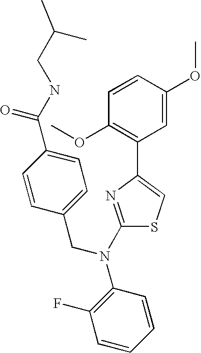 |
| 357 | 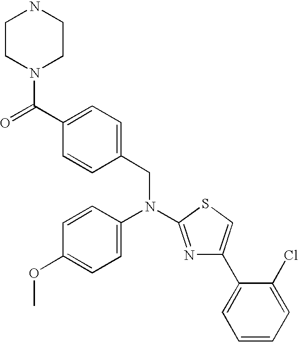 |
| 358 | 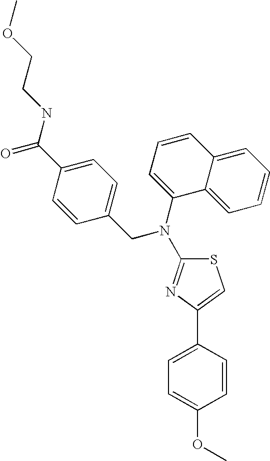 |
| 359 | 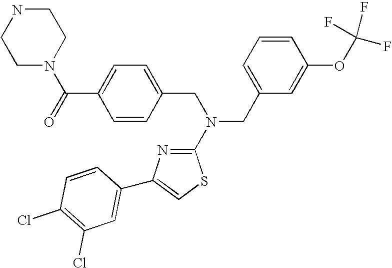 |
| 360 | 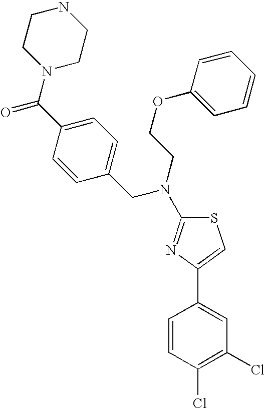 |
| 361 | 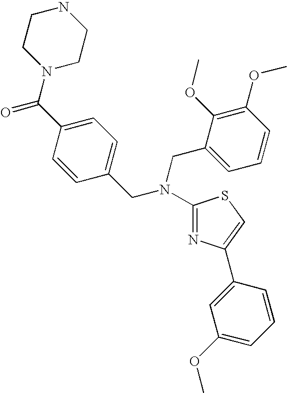 |
| 362 | 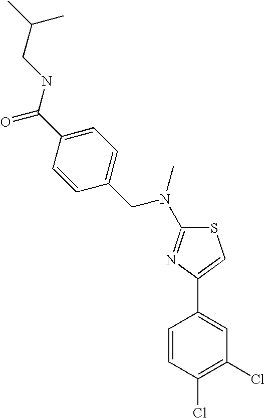 |
| 363 | 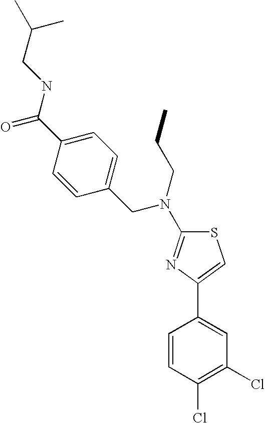 |
| 364 | 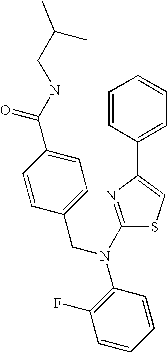 |
| 365 | 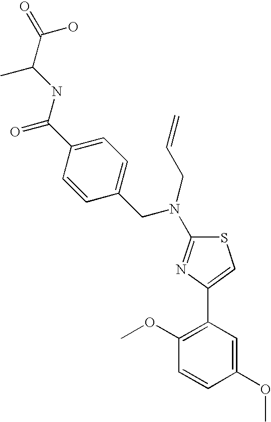 |
| 366 | 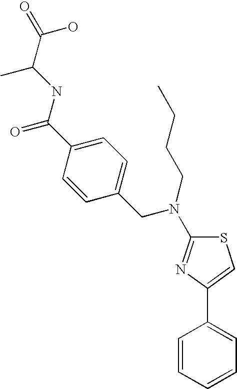 |
| 367 | 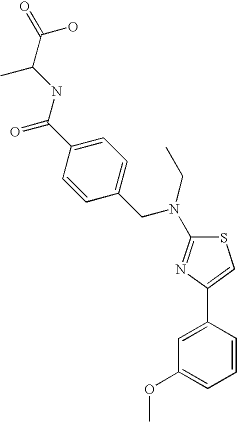 |
| 368 | 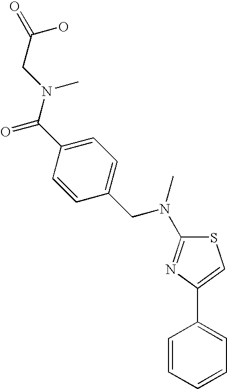 |
| 369 | 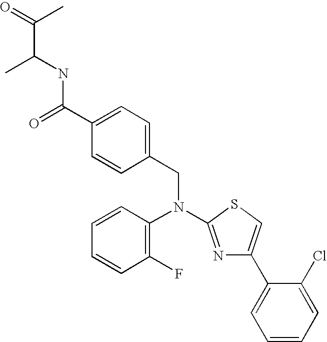 |
| 370 | 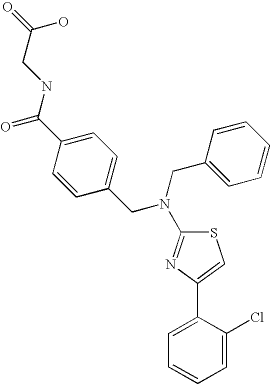 |
| 371 | 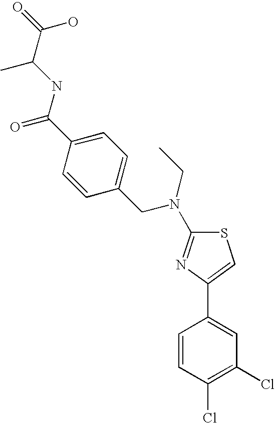 |
| 372 | 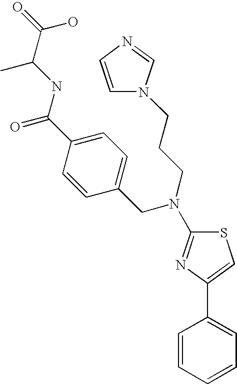 |
| 373 | 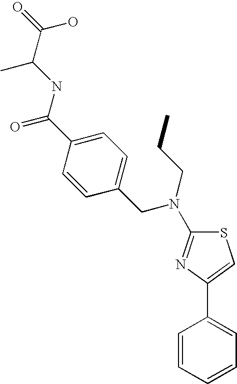 |
| 374 | 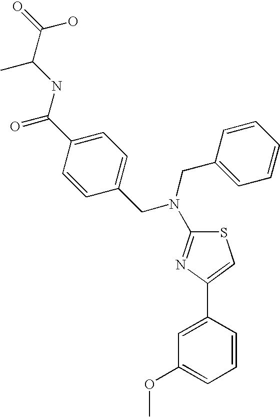 |
| 375 | 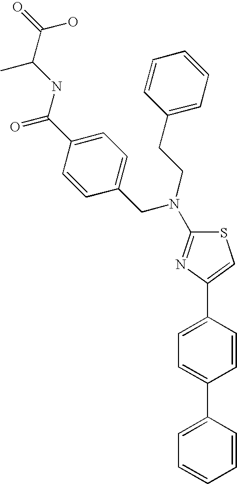 |
| 376 | 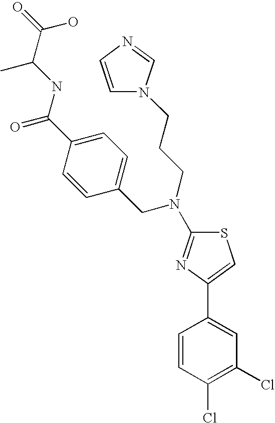 |
| 377 | 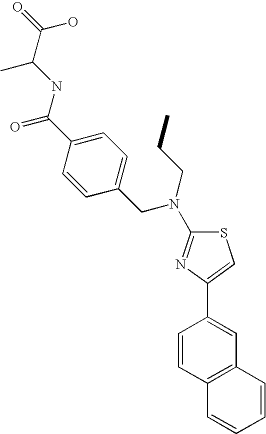 |
| 378 | 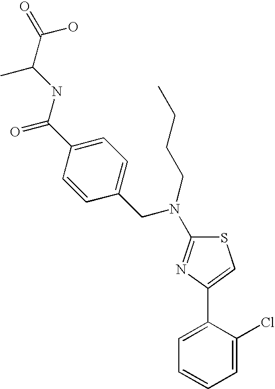 |
| 379 | 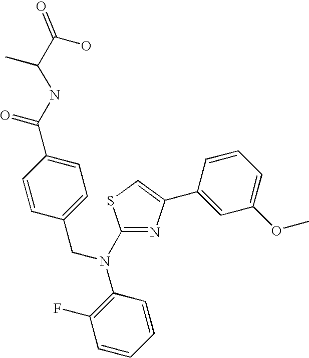 |
| 380 | 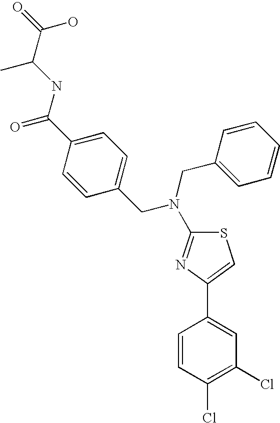 |
| 381 | 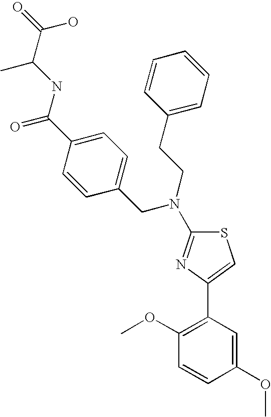 |
| 382 | 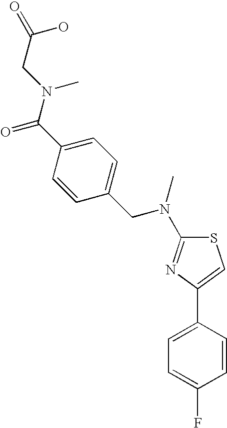 |
| 383 | 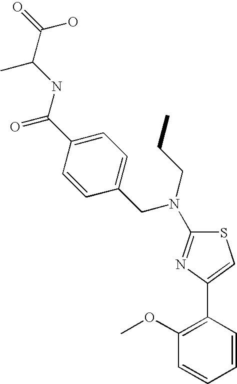 |
| 384 | 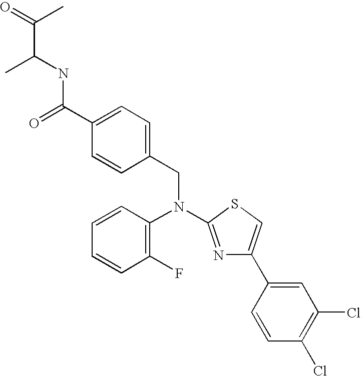 |
| 385 | 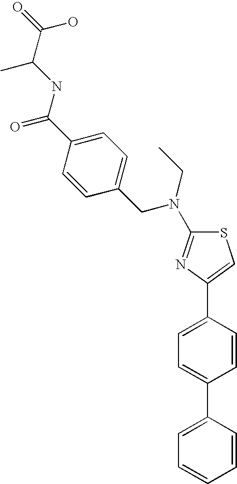 |
| 386 | 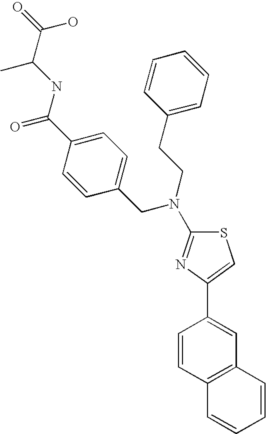 |
| 387 | 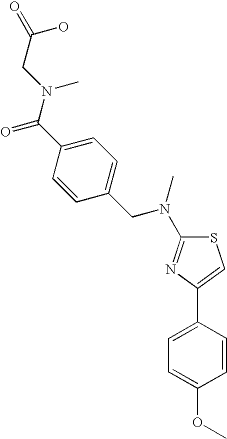 |
| 388 | 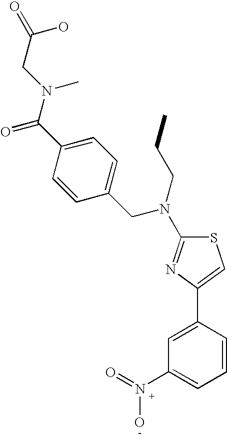 |
| 389 | 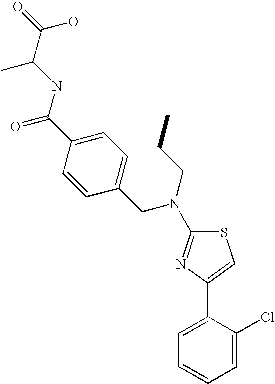 |
| 390 | 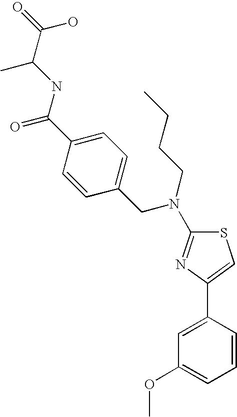 |
| 391 | 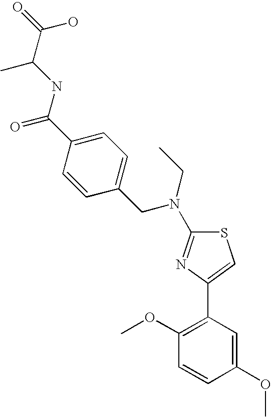 |
| 392 | 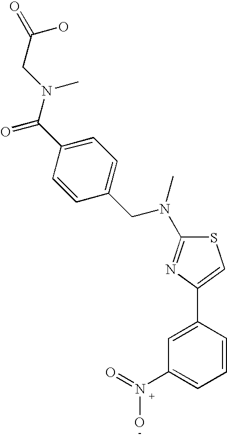 |
| 393 | 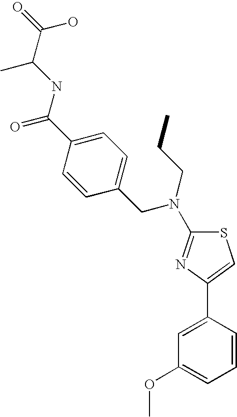 |
| 394 | 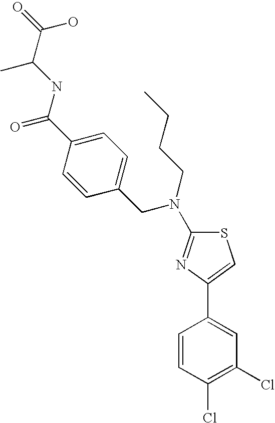 |
| 395 | 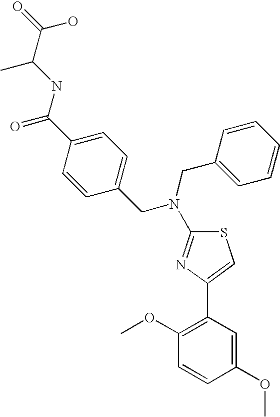 |
| 396 | 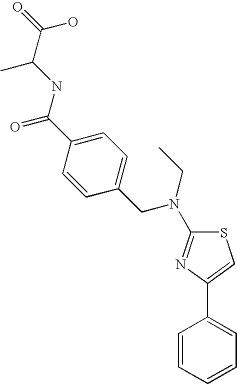 |
| 397 | 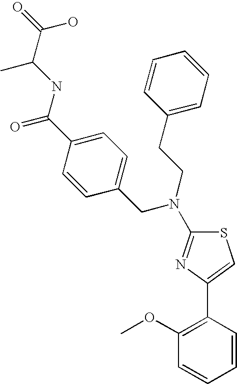 |
| 398 | 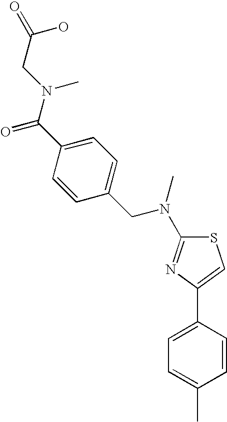 |
| 399 | 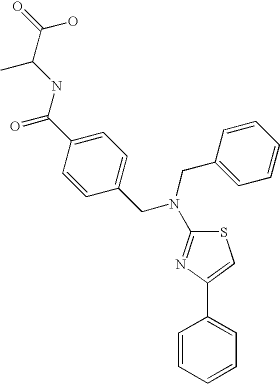 |
| 400 | 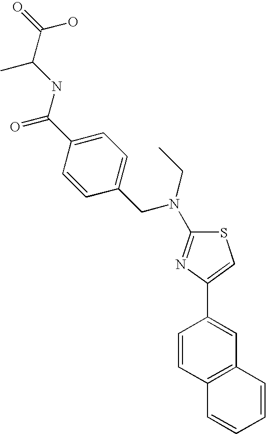 |
| 401 | 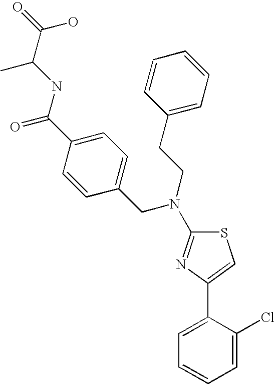 |
| 402 | 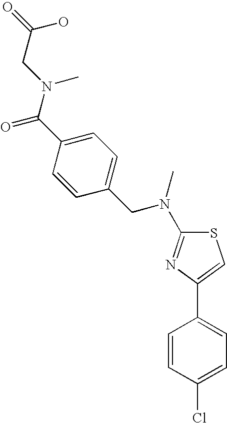 |
| 403 | 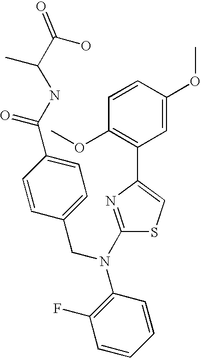 |
| 404 | 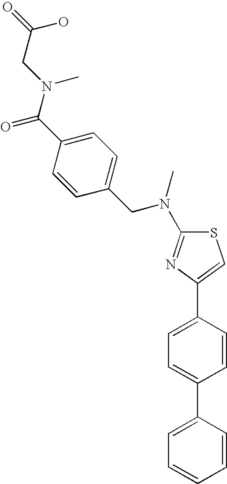 |
| 405 | 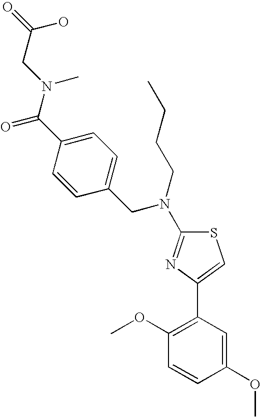 |
| 406 | 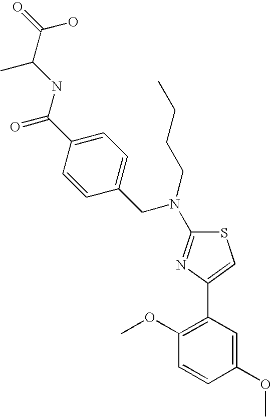 |
| 407 | 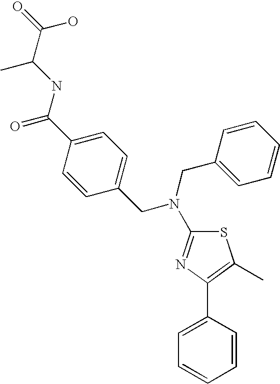 |
| 408 | 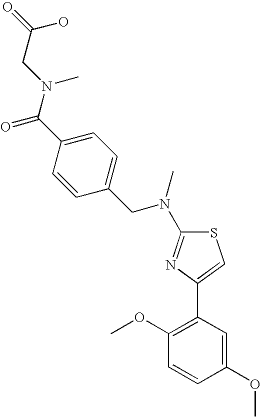 |
| 409 | 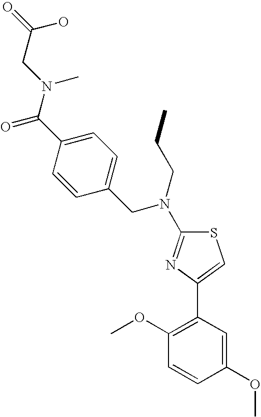 |
| 410 | 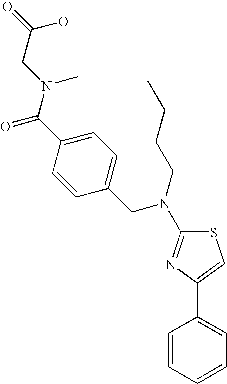 |
| 411 | 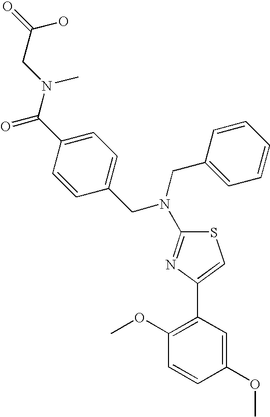 |
| 412 | 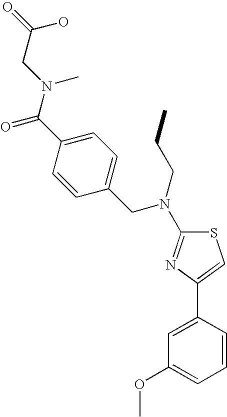 |
| 413 | 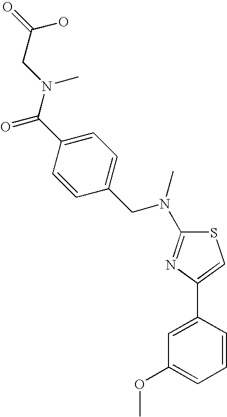 |
| 414 | 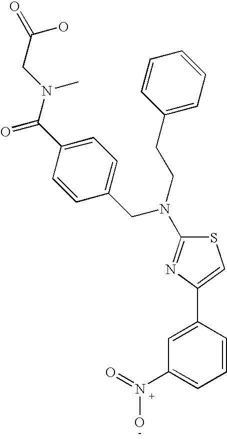 |
| 415 | 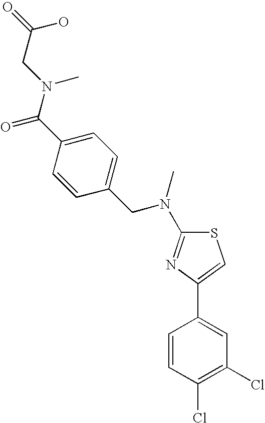 |
| 416 | 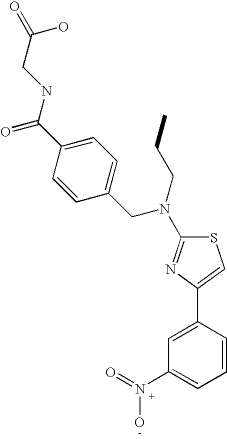 |
| 417 | 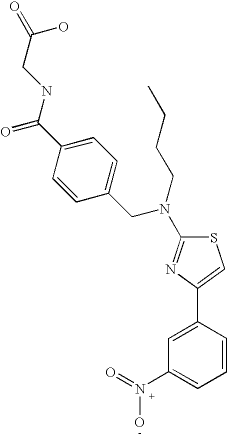 |
| 418 | 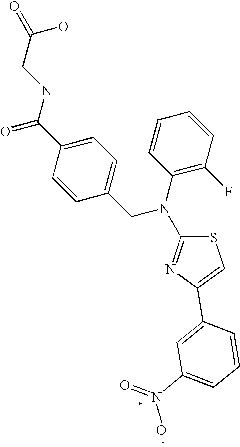 |
| 419 | 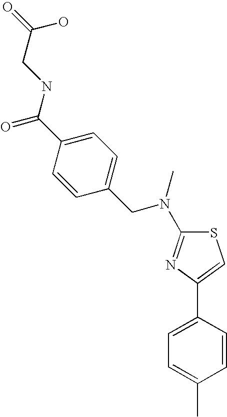 |
| 420 | 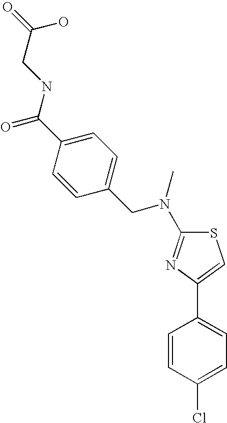 |
| 421 | 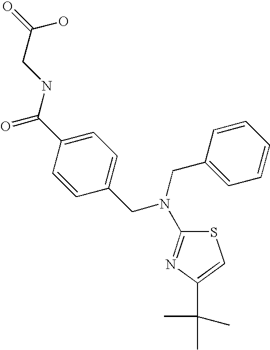 |

The compounds in the table above can be prepared using art recognized methods.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances-clear to those skilled in the art. For example, while an “alkyl” moiety generally refers to a monovalent group (e.g. CH₃—CH₂—), in certain circumstances a bivalent linking moiety can be “alkyl,” in which case those skilled in the art will understand the alkyl to be a divalent group (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene.) All atoms are understood to have their normal number of valences for bond formation (i.e., 4 for carbon, 3 for N, 2 for O, and 2, 4, or 6 for S, depending on the oxidation state of the S). On occasion a moiety may be defined, for example, as (A)ₐ-B-, wherein a is 0 or 1. In such instances, when a is 0 the moiety is B- and when a is 1 the moiety is A-B-.

When a substituent is referred to, for example, as “—C₁-C₆ alkylaryl”, it means that the substituent is attached by way of the “—C₁-C₆ alkyl.” For example, a group X substituted with “—C₁-C₆ alkylaryl” is X—C₁-C₆ alkylaryl.

For simplicity, reference to a “Cₙ-Cₘ” heterocyclyl or “Cₙ-Cₘ” heteroaryl means a heterocyclyl or heteroaryl having from “n” to “m” annular atoms, where “n” and “m” are integers. Thus, for example, a C₅-C₆-heterocyclyl is a 5- or 6-membered ring having at least one heteroatom, and includes pyrrolidinyl (C₅) and piperidinyl (C₆); C₆-hetoaryl includes, for example, pyridyl and pyrimidyl.

The term “hydrocarbyl” refers to a straight, branched, or cyclic alkyl, alkenyl, or alkynyl, each as defined herein. A “C₀” hydrocarbyl is used to refer to a covalent bond. Thus, “C₀-C₃-hydrocarbyl” includes a covalent bond, methyl, ethyl, ethenyl, ethynyl, propyl, propenyl, propynyl, and cyclopropyl.

The term “alkyl” as employed herein refers to straight and branched chain aliphatic groups having from 1 to 12 carbon atoms, preferably 1-8 carbon atoms, and more preferably 1-6 carbon atoms, which is optionally substituted with one, two or three substituents. Preferred alkyl groups include, without limitation, methyl, ethyl, propyl, isopropyl, butyl, isobutyl, sec-butyl, tertbutyl, pentyl, and hexyl. A “C₀” alkyl (as in “C₀-C₃-alkyl”) is a covalent bond (like “C₀” hydrocarbyl).

The term “alkenyl” as used herein means an unsaturated straight or branched chain aliphatic group with one or more carbon-carbon double bonds, having from 2 to 12 carbon atoms, preferably 2-8 carbon atoms, and more preferably 2-6 carbon atoms, which is optionally substituted with one, two or three substituents. Preferred alkenyl groups include, without limitation, ethenyl, propenyl, butenyl, pentenyl, and hexenyl.

The term “alkynyl” as used herein means an unsaturated straight or branched chain aliphatic group with one or more carbon-carbon triple bonds, having from 2 to 12 carbon atoms, preferably 2-8 carbon atoms, and more preferably 2-6 carbon atoms, which is optionally substituted with one, two or three substituents. Preferred alkynyl groups include, without limitation, ethynyl, propynyl, butynyl, pentynyl, and hexynyl.

An “alkylene,” “alkenylene,” or “alkynylene” group is an alkyl, alkenyl, or alkynyl group, as defined hereinabove, that is positioned between and serves to connect-two other chemical groups. Preferred alkylene groups include, without limitation, methylene, ethylene, propylene, and butylene. Preferred alkenylene groups include, without limitation, ethenylene, propenylene, and butenylene. Preferred alkynylene groups include, without limitation, ethynylene, propynylene, and butynylene.

The term “cycloalkyl” as employed herein includes saturated and partially unsaturated cyclic hydrocarbon groups having 3 to 12 carbons, preferably 3 to 8 carbons, and more preferably 3 to 6 carbons, wherein the cycloalkyl group additionally is optionally substituted. Preferred cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroalkyl” refers to an alkyl group, as defined hereinabove, wherein one or more carbon atoms in the chain are replaced by a heteroatom selected from the group consisting of O, S, and N.

An “aryl” group is a C₆-C₁₄ aromatic moiety comprising one to three aromatic rings, which is optionally substituted. Preferably, the aryl group is a C₆-C₁₀ aryl group. Preferred aryl groups include, without limitation, phenyl, naphthyl, anthracenyl, and fluorenyl. An “aralkyl” or “arylalkyl” group comprises an aryl group covalently linked to an alkyl group, either of which may independently be optionally substituted or unsubstituted. Preferably, the aralkyl group is (C₁-C₆)alk(C₆-C₁₀)aryl, including, without limitation, benzyl, phenethyl, and naphthylmethyl.

A “heterocyclic” group (or “heterocyclyl) is an optionally substituted non-aromatic mono-, bi-, or tricyclic structure having from about 3 to about 14 atoms, wherein one or more atoms are selected from the group consisting of N, O, and S. One ring of a bicyclic heterocycle or two rings of a tricyclic heterocycle may be aromatic, as in indan and 9,10-dihydro anthracene. The heterocyclic group is optionally substituted on carbon with oxo or with one of the substituents listed above. The heterocyclic group may also independently be substituted on nitrogen with alkyl, aryl, aralkyl, alkylcarbonyl, alkylsulfonyl, arylcarbonyl, arylsulfonyl, alkoxycarbonyl, aralkoxycarbonyl, or on sulfur with oxo or lower alkyl. Preferred heterocyclic groups include, without limitation, epoxy, aziridinyl, tetrahydrofuranyl, pyrrolidinyl, piperidinyl, piperazinyl, thiazolidinyl, oxazolidinyl, oxazolidinonyl, and morpholino. In certain preferred embodiments, the heterocyclic group is fused to an aryl, heteroaryl, or cycloalkyl group. Examples of such fused heterocycles include, without limitation, tetrahydroquinoline and dihydrobenzofuran. Specifically excluded from the scope of this term are compounds where an annular O or S atom is adjacent to another O or S atom.

In certain preferred embodiments, the heterocyclic group is a heteroaryl group. As used herein, the term “heteroaryl” refers to optionally substituted groups having 5 to 14 ring atoms, preferably 5, 6, 9, or 10 ring atoms; having 6, 10, or 14 pi electrons shared in a cyclic array; and having, in addition to carbon atoms, between one or more heteroatoms selected from the group consisting of N, O, and S. For example, a heteroaryl group may be pyrimidinyl, pyridinyl, benzimidazolyl, thienyl, benzothiazolyl, benzofuranyl and indolinyl. Preferred heteroaryl groups include, without limitation, thienyl, benzothienyl, furyl, benzofuryl, dibenzofuryl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, and isoxazolyl.

A “heteroaralkyl” or “heteroarylalkyl” group comprises a heteroaryl group covalently linked to an alkyl group, either of which is independently optionally substituted or unsubstituted. Preferred heteroalkyl groups comprise a C₁-C₆ alkyl group and a heteroaryl group having 5, 6, 9, or 10 ring atoms. Specifically excluded from the scope of this term are compounds having adjacent annular O and/or S atoms. Examples of preferred heteroaralkyl groups include pyridylmethyl, pyridylethyl, pyrrolylmethyl, pyrrolylethyl, imidazolylmethyl, imidazolylethyl, thiazolylmethyl, and thiazolylethyl.

An “arylene,” “heteroarylene,” or “heterocyclylene” group is an aryl, heteroaryl, or heterocyclyl group, as defined hereinabove, that is positioned between and serves to connect two other chemical groups.

Preferred heterocyclyls and heteroaryls include, but are not limited to, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

As employed herein, when a moiety (e.g., cycloalkyl, hydrocarbyl, aryl, heteroaryl, heterocyclic, urea, etc.) is described as “optionally substituted” it is meant that the group optionally has from one to four, preferably from one to three, more preferably one or two, non-hydrogen substituents. Suitable substituents include, without limitation, halo, hydroxy, oxo (e.g., an annular —CH-substituted with oxo is —C(O)—) nitro, halohydrocarbyl, hydrocarbyl, aryl, aralkyl, alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups. Preferred substituents, which are themselves not further substituted (unless expressly stated otherwise) are:

(a) halo, cyano, oxo, carboxy, formyl, nitro, amino, amidino, guanidino,

(b) C₁-C₅ alkyl or alkenyl or arylalkyl imino, carbamoyl, azido, carboxamido, mercapto, hydroxy, hydroxyalkyl, alkylaryl, arylalkyl, C₁-C₈ alkyl, C₁-C₈ alkenyl, C₁-C₈ alkoxy, C₁-C₈ alkoxycarbonyl, aryloxycarbonyl, C₂-C₈ acyl, C₂-C₈ acylamino, C₁-C₈ alkylthio, arylalkylthio, arylthio, C₁-C₈ alkylsulfinyl, arylalkylsulfinyl, arylsulfinyl, C₁-C₈ alkylsulfonyl, arylalkylsulfonyl, arylsulfonyl, C₀-C₆ N-alkyl carbamoyl, C₂-C₁₅ N,N-dialkylcarbamoyl, C₃-C₇ cycloalkyl, aroyl, aryloxy, arylalkyl ether, aryl, aryl fused to a cycloalkyl or heterocycle or another aryl ring, C₃-C₇ heterocycle, C₅-C₁₅ heteroaryl or any of these rings fused or spiro-fused to a cycloalkyl, heterocyclyl, or aryl, wherein each of the foregoing is further optionally substituted with one more moieties listed in (a), above; and

(c) —(CH₂), —NR³⁰R³¹, wherein s is from 0 (in which case the nitrogen is directly bonded to the moiety that is substituted) to 6, and R³⁰ and R³¹ are each independently hydrogen, cyano, oxo, carboxamido, amidino, C₁-C₈ hydroxyalkyl, C₁-C₃ alkylaryl, aryl-C₁-C₃ alkyl, C₁-C₈ alkyl, C₁-C₈ alkenyl, C₁-C₈ alkoxy, C₁-C₈ alkoxycarbonyl, aryloxycarbonyl, aryl-C₁-C₃ alkoxycarbonyl, C₂-C₈ acyl, C₁-C₈ alkylsulfonyl, arylalkylsulfonyl, arylsulfonyl, aroyl, aryl, cycloalkyl, heterocyclyl, or heteroaryl, wherein each of the foregoing is further optionally substituted with one more moieties listed in (a), above; or

R³⁰ and R³¹ taken together with the N to which they are attached form a heterocyclyl or heteroaryl, each of which is optionally substituted with from 1 to 3 substituents from (a), above.

In addition, substituents on cyclic moieties (i.e., cycloalkyl, heterocyclyl, aryl, heteroaryl) include 5-6 membered mono- and 9-14 membered bi-cyclic moieties fused to the parent cyclic moiety to form a bi- or tri-cyclic fused ring system. For example, an optionally substituted phenyl includes, but not limited to, the following:

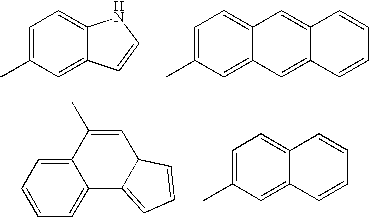

A “halohydrocarbyl” is a hydrocarbyl moiety in which from one to all hydrogens have been replaced with one or more halo.

The term “halogen” or “halo” as employed herein refers to chlorine, bromine, fluorine, or iodine. As herein employed, the term “acyl” refers to an alkylcarbonyl or arylcarbonyl substituent. The term “acylamino” refers to an amide group attached at the nitrogen atom (i.e., R—CO—NH—). The term “carbamoyl” refers to an amide group attached at the carbonyl carbon atom (i.e., NH₂—CO—). The nitrogen atom of an acylamino or carbamoyl substituent is additionally substituted. The term “sulfonamido” refers to a sulfonamide substituent attached by either the sulfur or the nitrogen atom. The term “amino” is meant to include NH₂, alkylamino, arylamino, and cyclic amino groups. The term “ureido” as employed herein refers to a substituted or unsubstituted urea moiety.

A moiety that is substituted is one in which one or more hydrogens have been independently replaced with another chemical substituent. As a non-limiting example, substituted phenyls include 2-flurophenyl, 3,4-dichlorophenyl, 3-chloro-4-fluoro-phenyl, 2-fluoro-3-propylphenyl. As another non-limiting example, substituted N-octyls include 2,4 dimethyl-5-ethyl-octyl and 3-cyclopentyl-octyl. Included within this definition are methylenes (—CH₂—) substituted with oxygen to form carbonyl —CO—).

An “unsubstituted” moiety as defined above (e.g., unsubstituted cycloalkyl, unsubstituted heteroaryl, etc.) means that moiety as defined above that does not have any of the optional substituents for which the definition of the moiety (above) otherwise provides. Thus, for example, while an “aryl” includes phenyl and phenyl substituted with a halo, “unsubstituted aryl” does not include phenyl substituted with a halo.

Some compounds of the invention may have chiral centers and/or geometric isomeric centers (E- and Z-isomers), and it is to be understood that the invention encompasses all such optical, diastereoisomers and geometric isomers. The invention also comprises all tautomeric forms of the compounds disclosed herein.

The compounds of the invention may be administered in the form of an in vivo hydrolyzable ester or in vivo hydrolyzable amide. An in vivo hydrolyzable ester of a compound of the invention containing carboxy or hydroxy group is, for example, a pharmaceutically acceptable ester which is hydrolyzed in the human or animal body to produce the parent acid or alcohol. Suitable pharmaceutically acceptable esters for carboxy include C₁₋₆-alkoxymethyl esters (e.g., methoxymethyl), C₁₋₆-alkanoyloxymethyl esters (e.g., for example pivaloyloxymethyl), phthalidyl esters, C₃₋₈cycloalkoxycarbonyloxyC₁₋₆-alkyl esters (e.g., 1-cyclohexylcarbonyloxyethyl); 1,3-dioxolen-2-onylmethyl esters (e.g., 5-methyl-1,3-dioxolen-2-onylmethyl; and C₁₋₆-alkoxycarbonyloxyethyl esters (e.g., 1-methoxycarbonyloxyethyl) and may be formed at any carboxy group in the compounds of this invention.

An in vivo hydrolyzable ester of a compound of the invention containing a hydroxy group includes inorganic esters such as phosphate esters and a-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolyzable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(N,N-dialkylaminoethyl)-N-alkylcarbamoyl (to give carbamates), N,N-dialkylaminoacetyl and carboxyacetyl. Examples of substituents on benzoyl include morpholino and piperazino linked from a ring nitrogen atom via a methylene group to the 3- or 4-position of the benzoyl ring. A suitable value for an in vivo hydrolyzable amide of a compound of the invention containing a carboxy group is, for example, a N—C₁₋₆-alkyl or N,N-di-C₁₋₆-alkyl amide such as N-methyl, N-ethyl, N-propyl, N,N-dimethyl, N-ethyl-N-methyl or N,N-diethyl amide.

### Pharmaceutical Compositions

In a second aspect, the invention provides pharmaceutical compositions comprising an inhibitor of histone deacetylase according to the invention and a pharmaceutically acceptable carrier, excipient, or diluent. Compounds of the invention may be formulated by any method well known in the art and may be prepared for administration by any route, including, without limitation, parenteral, oral, sublingual, transdermal, topical, intranasal, intratracheal, or intrarectal. In certain preferred embodiments, compounds of the invention are administered intravenously in a hospital setting. In certain other preferred embodiments, administration may preferably be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's The Science and Practice of Pharmacy, 20th Edition, 2000.

As used herein, the term pharmaceutically acceptable salts refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z-, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate). As used herein, the term “salt” is also meant to encompass complexes, such as with an alkaline metal or an alkaline earth metal.

The active compound is included in the pharmaceutically acceptable carrier or diluent in an amount sufficient to deliver to a patient a therapeutically effective amount without causing serious toxic effects in the patient treated. A preferred dose of the active compound for all of the above-mentioned conditions is in the range from about 0.01 to 300 mg/kg, preferably 0.1 to 100 mg/kg per day, more generally 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

### Synthetic Schemes and Experimental Procedures

The compounds of the invention may be synthesized according to the methods known in the art. For example, methods that may be used to make the compounds of the invention are described in Mallory, F. B. (Organic Syntheses, Coll. Vol. IV: pp 74-75 (John Wiley & Sons, 1993)); Smith, P. A. S. and Boyer, J. H. (Organic Syntheses, Coll. Vol. IV: pp 75-78 (John Wiley & Sons, 1963)), and in Can. J. Chem., pp 2482-2484 (1969). Methods of making saturated and partially saturated compounds (such as those in embodiment DD) are known to those skilled in the art and include reduction of double bonds (e.g., hydrogenation). Methods of catalytic hydrogenation are known in the art and discussed, for example, in March, “Advanced Organic Chemistry” (5th Ed., John Wiley & Sons, Inc. 2001). These references are incorporated by references in their entirety.

One skilled in the art would readily recognize that the compounds of the invention can be synthesized by procedures known in the art from a variety of starting materials and synthetic route. The starting materials are readily available from several commercial sources. For example, α-amino acids, β-amino acids, γ-amino acids, and unnatural amino acids can be used as the starting material to achieve variation in chain length and type of groups attached to the amine in Schemes 1a-1e. Further, because R₂ comes from aldehydes and both R₃ and R₄ come from α-halo ketones, a variety of products can be produced. In addition, it is possible to synthesize bicyclic systems, such as 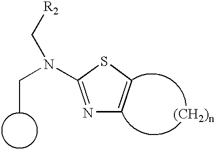
by using the appropriate synthetic routes.

Referring to Scheme 1a, protected amino acids 1 are coupled to for example a Wang resin 2 to give coupled products 3. The protecting group is removed to reveal a free amine of product 4. Polymer bound amino acid 4 can be used in a number of chemical transformations to make compounds of the invention. For example, 4 is subjected to reductive amination conditions in the presence of an aldehyde to produce 5. Next thiourea 6 is formed, followed by deprotection of the Fmoc group, and cycloaddition with α-haloketone 7 to form 2-amino-1,3-thiazole 8. Thiazole 8 is removed from the solid support via hydrolytic cleavage to give thiazoles of formula 9.

Referring to Scheme 1b, resin bound amino acids 4 are coupled with 10 via reductive Lamination with the aldehyde group of 10 to give 11. The acid group of 11 is reduced to the corresponding aldehyde which is subjected to reductive amination conditions to give 12. As described previously, 12 is converted to the corresponding thiourea 13, which in turn is cyclized with 7 to form resin bound thiazole 14. Hydrolysis gives thiazoles 15.

Referring to Scheme 1c, resin bound amino acids 4 are coupled with 10 via reductive amination with the aldehyde group of 10 to give 11. The Fmoc is removed and the compound is converted to the corresponding thiourea 16, which in turn is cyclized with 7 to form resin bound thiazole 17. In this example, the acid group of 17 is subjected to amide bond forming conditions with an amino acid, for example, to give products 18. Hydrolysis gives thiazoles 19.

Referring to Scheme id, resin bound amino acids 4 are coupled with 20 via reductive amination with the aldehyde group of 20 to give 21. As described previously, 21 is converted to the corresponding thiourea 22, which in turn is cyclized with 7 to form resin bound thiazole 23. Hydrolysis gives thiazoles 24.

Referring to Scheme 1e, resin bound amino acids 4 are coupled with 10 via amide bond formation with the acid group of 10 to give 25. Reductive amination with the aldehyde group of 25 and the appropriate amine gives 26. As described previously, 26 is converted to the corresponding thiourea 27, which in turn is cyclized with 7 to form resin bound thiazole 28. Hydrolysis gives thiazoles 29.

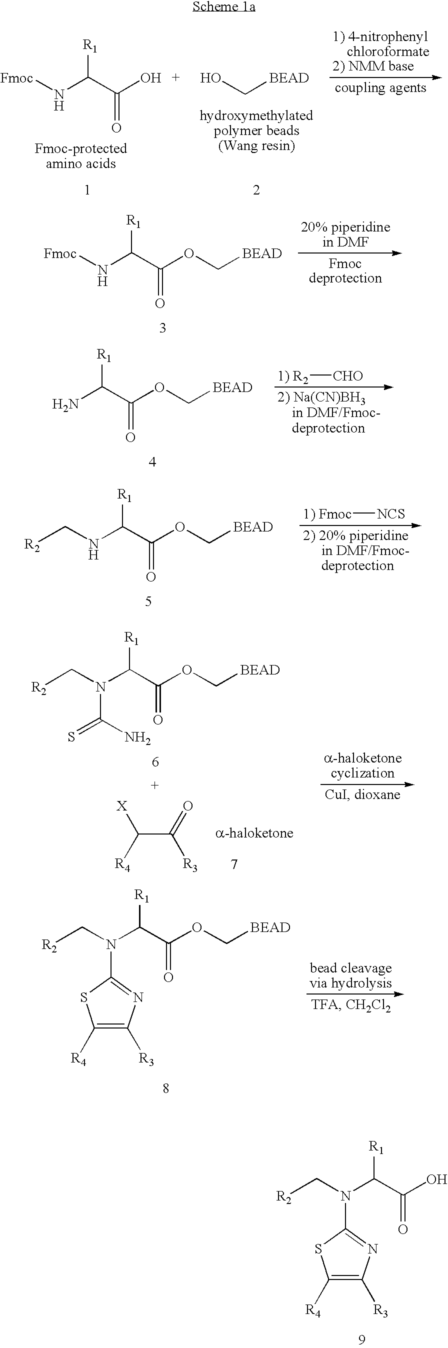

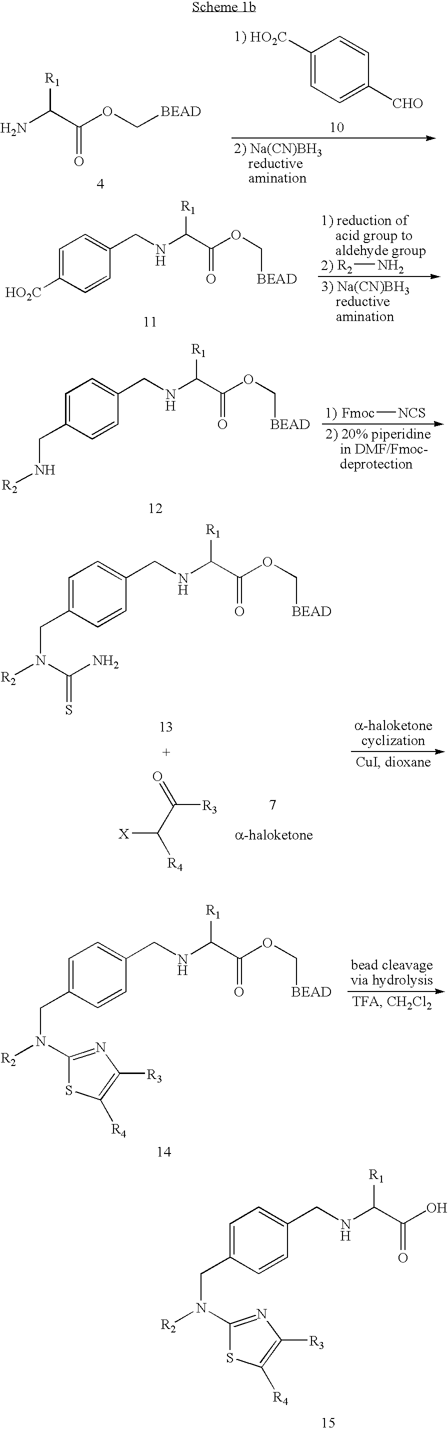

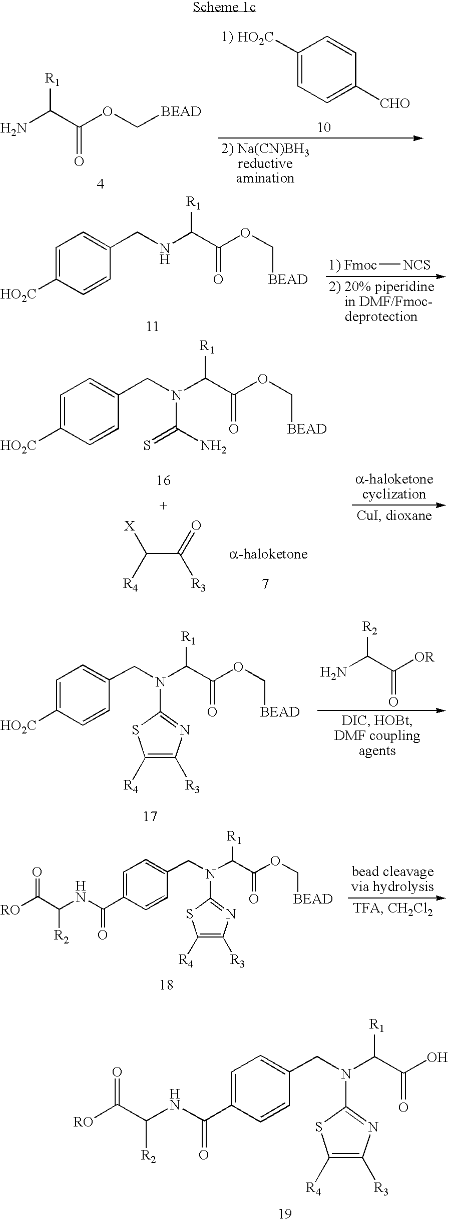

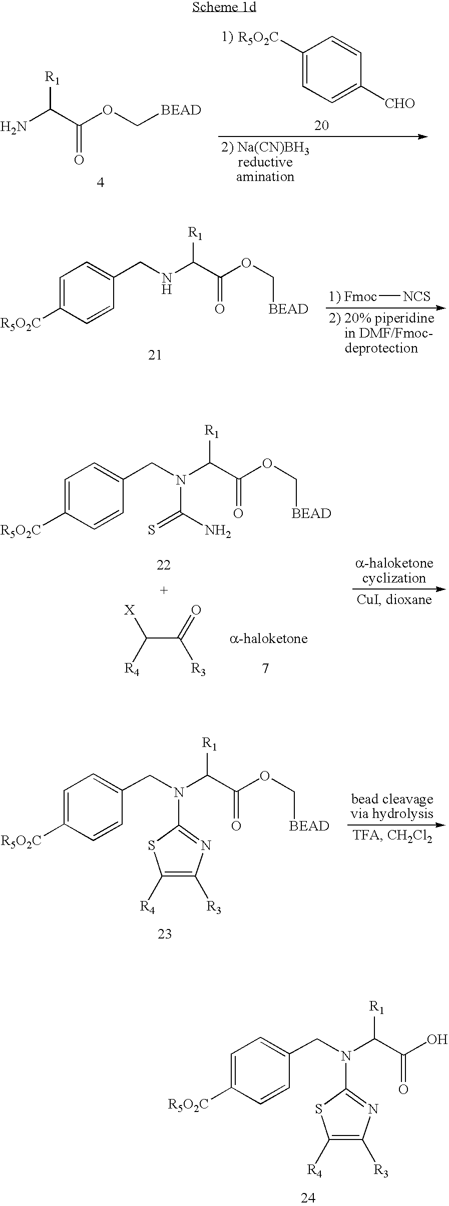

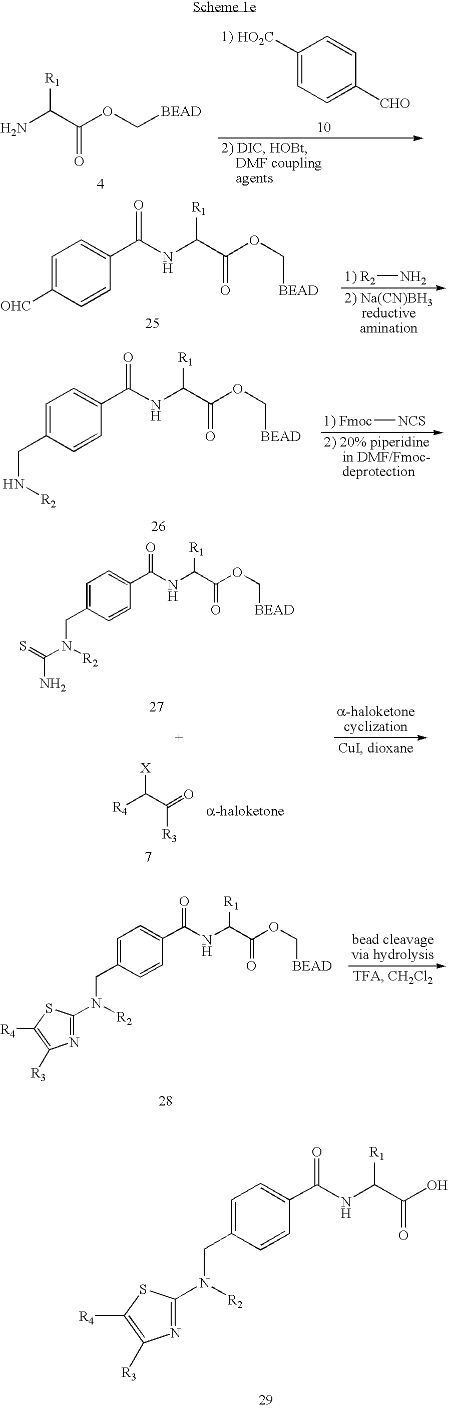

### EXAMPLE 1

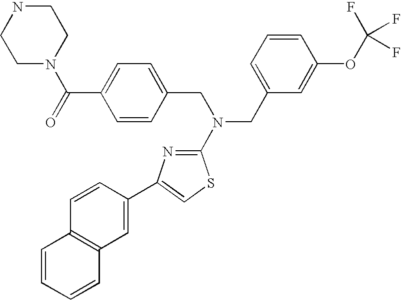

### 4-(2-naphthyl)-N-[4-(piperazin-1-ylcarbonyl)benzyl]-N-[3-(trifluoromethoxy)benzyl]-1,3-thiazol-2-amine (Cpd. No. 282)

Compounds of the invention that are analogs of structure 36 in Scheme 2, such as Cpd. No. 282 can be made by solid phase synthesis using Wang resin according to Scheme 2. Standard wash refers to the following washing sequence: DMF, DCM, DMF, DCM, MeOH, DCM, MeOH (X2), and ether (X2). Resin swelling in solvents will be based on a standard of 10 ml of solvent per gram of resin.

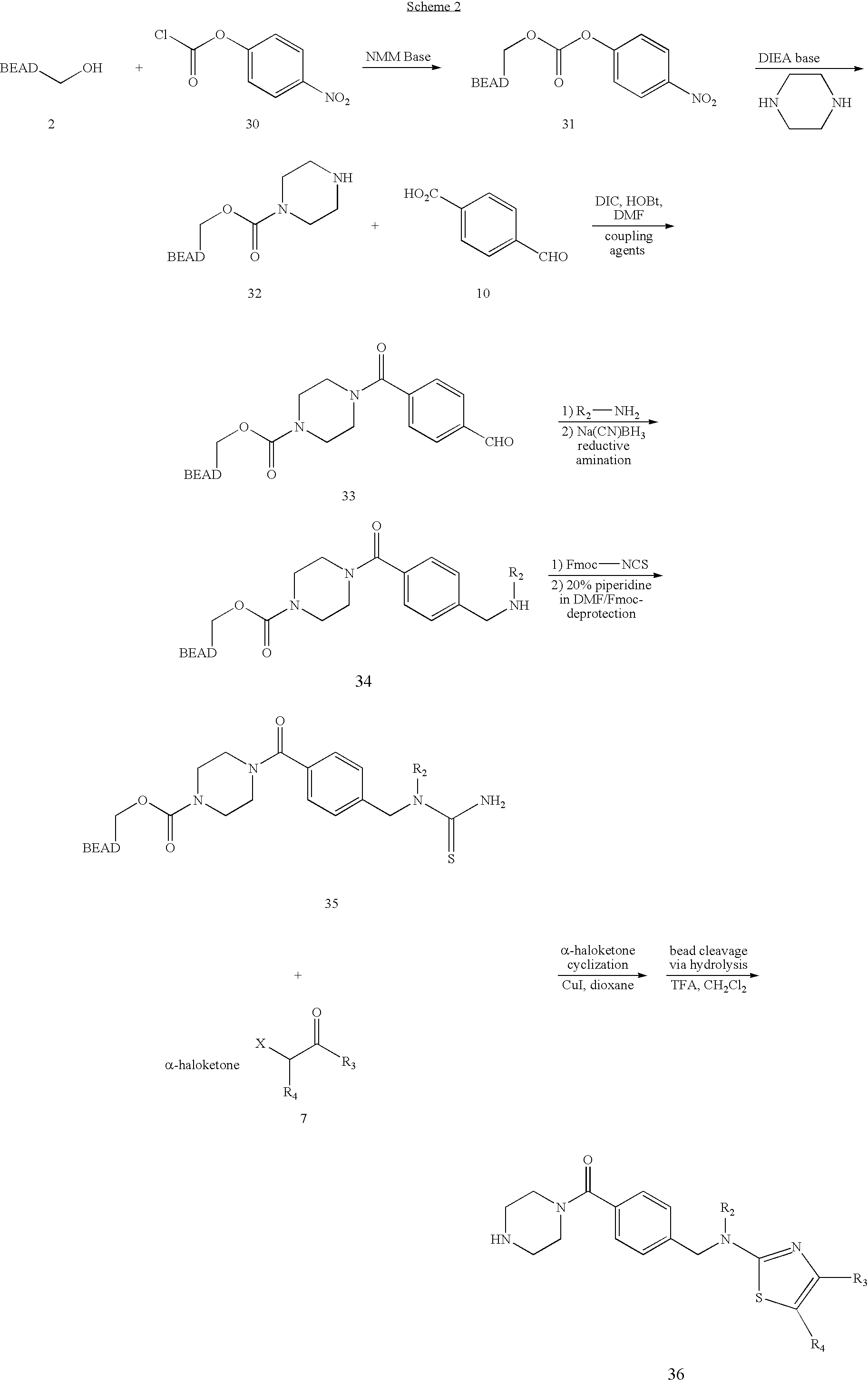

Referring to Scheme 2, a Wang resin 2 is coupled to chloroformate 30 to give resin-bound activated carbonate 31. Carbonate 31 is reacted with piperazine (or other appropriate amine) to give carbamate 32. Carbamate 32 is reacted with 10 under amide bond forming conditions to form 33. The aldehyde group of 33 is subjected to reductive amination conditions in the presence of an amine to produce 34. Next, thiourea 35 is formed, followed by deprotection of the Fmoc group, and cycloaddition with α-haloketone 7 to form the corresponding thiazole. Thiazole 36 is removed from the solid support via hydrolytic cleavage.

The following conditions and procedures apply to the steps in Scheme 2 and some of these conditions apply by analogy to Schemes 1a-e and 3.”.

Conversion of resin 2 to carbamate 32: 3 eq. 4-nitrophenol chloroformate, 3 eq. N-methylmorpholine, dry DCM, 0° C. to at room temperature and overnight. Then the following is added: 5 eq. diamine, 5 eq. iPr₂NEt, dry DMF at room temperature and overnight.

Conversion of 32 to 33: 4-Carboxybenzaldehyde loading on to amine resin using 3.5 eq. 4-carboxybenzaldehyde, 3.5 eq. DIC, 3.5 eq. HOBt at room temperature and overnight. The 4-carboxybenzaldehyde was dissolved in dry DMF. DIC (3.5 eq.) and HOBt (3.5 eq.) were added and the solution was shaken for 15 minutes. The resin was added and the reaction was shaken at room temperature overnight. The resin was then filtered and washed.

Conversion of 33 to 34: Reductive Amination using 10 eq. AcOH, 5 eq. R₂—NH₂, 5 eq. Na(CN)BH₃, MeOH/THF 1/1 at room temperature and overnight. The resin was swollen in a 1:1 mixture of MeOH/THF prior to the addition of acetic acid (10 eq.), the amine (5 eq.) and sodium cyanoborohydride (5 eq.). The slurry was shaken at room temperature overnight and then washed with MeOH and then the standard wash.

Conversion of 34 to 35: Thiourea Formation using 3 eq N-Fmoc thioisocyanate, dry DCM at room temperature and overnight. The resin was swollen in a solution of FmocNCS (3.0 eq.) in dry DCM. The reactions were shaken overnight at room temperature and then filtered and washed. Then the following was performed. De-Fmoculation using a solution of 20% piperidine in DMF at room temperature for 2 hours. The resin was suspended in a solution of 20% piperidine in DMF and shaken for 2 h. The reaction was then filtered and washed with the standard wash.

Conversion of resin 35 to carbamate 36: Cycloaddition using 5 eq. α-haloketone, dry dioxane, 5% CuI, room temperature for 3 days. The resin was suspended in a solution of the α-haloketone (5 eq.) in dry dioxane. A catalytic quantity of sodium iodide was added and the reactions were shaken at room temperature for 3 days, filtered and then washed with water and then the standard wash. Then the following was performed. TFA cleavage using 95% TFA in DCM. The resin (swollen in DCM) was cleaved by addition of 500 μl (X 2) and 400 μl aliquots of 95% TFA in DCM. The TFA and DCM were then removed under reduced pressure in a genevac.

### EXAMPLE 2

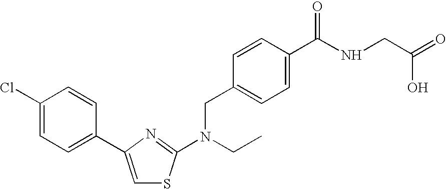

### N-(4-{[[4-(4-chlorophenyl)-1,3-thiazol-2-yl](ethyl)amino]methyl}benzoyl)glycine (Cpd. No. 146)

Compounds of the invention that are analogs of Cpd. No. 146 can be made by solid phase synthesis according to Scheme 3.

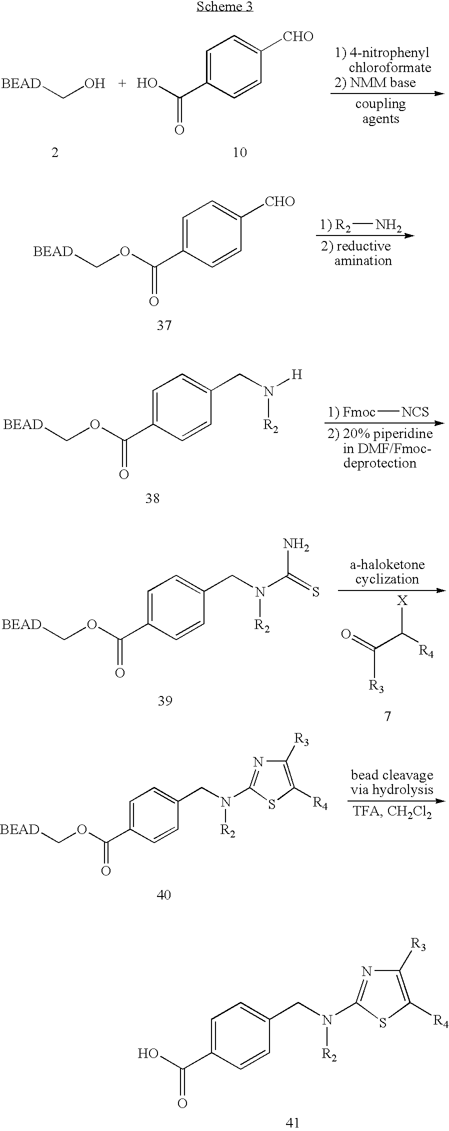

Referring to Scheme 3, a Wang resin 2 is coupled to 10 via it's acid group to give 37. The aldehyde group of 37 is subjected to reductive amination conditions in the presence of an amine to produce 38. Next, thiourea 39 is formed, and cycloaddition with α-haloketone 7 is affected to form the corresponding thiazole 40. Hydrolysis gives thiazoles 41.

### Biological Activity

The following examples illustrate the biological activity assays of the compounds of the invention. Various types of assays were used to show the inhibitory activity of the compounds of the invention towards ubiquitin ligases. The examples described below are not meant to limit in any way the use of the compounds of the invention as ubiquitin ligase inhibitors.

### EXAMPLE A

### Plate-Based E3 Ligase Assay (APC FLAG)

E3 (His-APC11/APC2—“APC”) auto-ubiquitination was measured as described in U.S. patent application Ser. No. 09/826,312 (Publication No. US-2002-0042083-A1), which is incorporated herein in its entirety. Details of the protocol are described below. Activity in the presence of the compound was determined relative to a parallel control in which only DMSO was added. Values of the IC₅₀ were typically determined using 6 or 8 different concentrations of the compound, although as few as 2 concentrations may be used to approximate the IC₅₀ value. Active compounds were those that exhibited an IC₅₀ values of 25 μM or lower.

Nickel-coated 96-well plates (Pierce 15242) were blocked for 1 hour with 100 μl of blocking buffer at room temperature. The plates were washed 4 times with 225 μl of 1×PBS and 80 μl of the reaction buffer were added that contained 100 ng/well of Flag-ubiquitin. To this, 10 μl of the test compound diluted in DMSO were added. After the test compound was added, 10 μl of E1 (human), E2 (Ubch5c), and APC in Protein Buffer was added to obtain a final concentration of 5 ng/well of E1, 20 ng/well: of E2 and 100 ng/well of APC. The plate were shaken for 10 minutes and incubated at room temperature for 1 hour. After incubation, the plates were washed 4 times with 225 μl of 1×PBS and 100 μl/well of Antibody Mix were added to each well. The plates were incubate at room temperature for another hour after which they were washed 4 times with 225 μl of 1×PBS and 100 μl/well of Lumino substrate were added to each well. The luminescence was measured by using a BMG luminescence microplate reader.

To prepare the Blocking Buffer (1 liter; 1% Casein in 1×PBS), 10 grams of Casein (Hammersten Grade Casein from Gallard-Schlesinger Inc. #440203) were placed into 1 liter of 1×PBS, stirred on a hot plate and kept between 50-60° C. for an hour. The buffer was allowed to cool to room temperature and then filtered using a Buchner Funnel (Buchner filter funnel 83 mm 30310-109) and Whatman filter paper (Whatman Grade No. 1 Filter paper 28450-070). It was stored at 4° C. until used.

The reaction buffer consisted of 62.5 mM Tris pH 7.6 (Trizma Base—Sigma T-8524), 3 mM MgCl₂ (Magnesium Chloride—Sigma M-2393), 1 mM DTT (Sigma D-9779), 2.5 mM ATP (Roche Boehringer Mann Corp. 635-316), 100 ng/well of Flag-ubiquitin, 0.1% BSA (Sigma A-7906), and 0.05% Tween-20 (Sigma P-7949).

The Protein Buffer consisted of 20 mM Tris pH 7.6, 10% glycerol (Sigma G-5516) and 1 mM DTT.

The antibody mix consisted of 0.25% BSA (Sigma A-7906) in 1×PBS, 1/50,000 anti-Flag (Sigma F-3165), 1/100,000 of anti-Mouse IgG-HRP (Jackson Immunoresearch #115-035-146).

The substrate mix consisted of SuperSignal Substrate from Pierce (catalog number 37070ZZ) and was prepared by mixing 100 ml of the peroxide solution, 100 ml of the enhancer solution and 100 ml of Milli-Q® water.

In similar manner, autoubiquitination of the E3 MDM2 was determined (MDM2-FLAG). Further instruction in such assays can be found in U.S. patent application Ser. No. 10/108,767 (Publication No. US-2003-0104474 A1), which is incorporated herein in its entirety.

### EXAMPLE B

### Plate-Based MDM2 Substrate Ligation Assay (MDM2-P53-FLAG)

This assay was performed essentially as described in WO 03/076899 (page 77, line 5 to page 80, line 13), hereby incorporated by reference in its entirety.

### EXAMPLE C

### Gel-Based E2 Ligase Assay (Cell Titer-Aqueous)

E2 (Ubch5c) auto-ubiquitination was measured as described in U.S. patent application Ser. No. 09/826,312 (Publication No. US-2002-0042083-A1), which is incorporated herein in its entirety. Details of the protocol are described below. Activity in the presence of the test compound was determined relative to a parallel control in which only DMSO was added. The IC₅₀ values were typically determined using 6 or 8 different concentrations of compound, although as few as 2 concentrations may be used to approximate IC₅₀ values. Active compounds were those having IC₅₀ values of 25 μM or lower.

Corning 96-well plates (Corning 3650) were blocked with 100 μl of Blocking Buffer for 1 hour at room temperature. The plates were washed for 4 times with 225 μl of 1×PBS and 80 μl of the reaction buffer were added that contained 100 ng/well of Flag-ubiquitin. To this, 10 μl of the test compound diluted in DMSO were added. After the test compound was added, 10 μl of E1 (human) and E2 (Ubch5c) in Protein Buffer were added to obtain a final concentration of 5 ng/well of E1 and 20 ng/well of E2. The plates were shaken for 10 minutes and incubated at room temperature for 1 hour. After incubation, the reaction was stopped by adding 25 μl of loading buffer (non-reducing) per well and the plates were heated at 95° C. for 5 minutes. An aliquot of each well was run on a 10% NugePage Gel and analyzed by Western Blot using the antibody mix and Lumino substrate described below. The luminescence was measured using a BMG luminescence microplate reader.

To prepare the Blocking Buffer (1 liter; 1% Casein in 1×PBS), 10 grams of Casein (Hammersten Grade Casein from Gallard-Schlesinger Inc. #440203) were placed into 1 liter of 1×PBS, stirred on a hot plate and kept between 50-60° C. for an hour. The buffer was allowed to cool to room temperature and then filtered using a Buchner Funnel (Buchner filter funnel 83 mm 30310-109) and Whatman filter paper (Whatman Grade No. 1 Filter paper 28450-070). It was stored at 4° C. until used.

The reaction buffer consisted of 62.5 mM Tris pH 7.6 (Trizma Base—Sigma T-8524), 3 mM MgCl₂ (Magnesium Chloride—Sigma M-2393), 1 mM DTT (Sigma D-9779), 2.5 mM ATP (Roche Boehringer Mann Corp. 635-316), 100 ng/well of Flag-ubiquitin, 0.1% BSA (Sigma A-7906), and 0.05% Tween-20 (Sigma P-7949).

The Protein Buffer consisted of 20 mM Tris pH 7.6, 10% glycerol (Sigma G-5516) and 1 mM DTT.

The antibody mix consisted of 0.25% BSA (Sigma A-7906) in 1×PBS, 1/50,000 anti-Flag (Sigma F-3165), 1/100,000 of anti-Mouse IgG-HRP (Jackson Immunoresearch #115-035-146).

The substrate mix consisted of SuperSignal® Substrate from Pierce (catalog number 37070ZZ) and was prepared by mixing 100 ml of the peroxide solution, 100 ml of the enhancer solution and 100 ml of Milli-Q® water.

### EXAMPLE D

### Gel-Based E3 Ligase Assay

E3 (His-APC11/APC2—“APC”) auto-ubiquitination was measured as described in U.S. patent application Ser. No. 09/826,312 (Publication No. US-2002-0042083-A1), which is incorporated herein in its entirety. Details of the protocol are described below. Activity in the presence of compound was determined relative to a parallel control in which only DMSO is added. The IC₅₀ values were typically determined using 6 or 8 different concentrations of compound, although as few as 2 concentrations may be used to approximate the IC₅₀ values. Active compounds were those having IC₅₀ values of 25 μM or lower.

Corning 96-well plates (Corning 3650) were blocked with 100 μl of Blocking Buffer for 1 hour at room temperature. The plates were washed 4 times with 225 μl of 1×PBS and 80 μl of the reaction buffer were added that contained 100 ng/well of Flag-ubiquitin. To this, 10 μl of the test compound diluted in DMSO were added. After the test compound was added, 10 μl of E1 (human), E2 (Ubch5c) and APC in Protein Buffer were added to obtain a final concentration of 5 ng/well of E1, 20 ng/well of E2 and 100 ng/well APC. The plates were shaken for 10 minutes and incubated at room temperature for 1 hour. After incubation, the reaction was stopped by adding 25 μl of loading buffer (non-reducing) per well and the plates were heated at 95° C. for 5 minutes. An aliquot of each well was run on a 10% NugePage Gel and analyzed by Western Blot using the antibody mix and Lumino substrate described below. The luminescence was measured by using a BMG luminescence microplate reader.

To prepare the Blocking Buffer (1 liter; 1% Casein in 1×PBS), 10 grams of Casein (Hammersten Grade Casein from Gallard-Schlesinger Inc. #440203) were placed into 1 liter of 1×PBS, stirred on a hot plate and kept between 50-60° C. for an hour. The buffer was allowed to cool to room temperature and then filtered using a Buchner Funnel (Buchner filter funnel 83 mm 30310-109) and Whatman filter paper (Whatman Grade No. 1 Filter paper 28450-070). It was stored at 4° C. until used.

The reaction buffer consisted of 62.5 mM Tris pH 7.6 (Trizma Base—Sigma T-8524), 3 mM MgCl₂ (Magnesium Chloride—Sigma M-2393), 1 mM DTT (Sigma D-9779), 2.5 mM ATP (Roche Boehringer Mann Corp. 635-316), 100 ng/well of Flag-ubiquitin, 0.1% BSA (Sigma A-7906), and 0.05% Tween-20 (Sigma P-7949).

The Protein Buffer consisted of 20 mM Tris pH 7.6, 10% glycerol (Sigma G-5516) and 1 mM DTT.

The antibody mix consisted of 0.25% BSA (Sigma A-7906) in 1×PBS, 1/50,000 anti-Flag (Sigma F-3165), 1/100,000 of anti-Mouse IgG-HRP (Jackson Immunoresearch #115-035-146).

### EXAMPLE E

### Cell Proliferation Assays

Cell Culture Preparation

A549 (ATCC# CCL 185), HeLa (ATCC# CCL 2), HCT116 (ATCC# CCL-247), and H 1299 (ATCC# CRL-5803) cells were maintained in T175 flasks following the ATCC recommended media and handling procedures. Flasks reaching approximately 70% confluency were trypsinized and resuspended in RPMI media (Cell Gro catalog number 10 040 CM) modified to contain 5% FBS, 100 ug/mlPen/Strep (Cell Gro catalog number 30 002 CL), and 0.3 mg/ml L Glutamine (Cell Gro catalog number 25 003 CL). A 20,000 cells/ml solution was made for plating. Cells were plated in black Packard 96 well plates by placing 100 μl per well (2,000 cells per well).

Cell Treatment with Compounds

Compounds and additional media were added 24′ hours after cell plating. A compound master plate was created with concentrations 500 times greater than the final concentration added to the cells. All compound testing was done in duplicate using 3 fold dilutions starting with 10 mM. All outside wells (and 4 internal wells) were DMSO controls. Taxol and at least one additional control were run on all plates. Three microliters of the compound master plate were added to deep well blocks containing 750 μl of RPMI media. One hundred microliters were transferred from the compound/media deep well blocks to the plated cells resulting in a 500 fold dilution of the compounds. Cells were grown at 37° C., 5% CO₂ for 48 hours.

Photographic Image Analysis of Proliferation, Apoptosis and Death (PAD Assay)

Cells to be analyzed by photography were fixed and stained. One hundred microliters of media were removed and 100 μl of 2% paraformaldehyde was added to each well. Plates were left on the benchtop for 45 minutes. A staining solution containing 1.55 μl of 1 mg/ml DAPI added to 18.75 ml PBS was warmed for 15 minutes at 37° C. The cells were aspirated prior to washing with 100 μl of PBS. Seventy microliters of PBS were aspirated and 170 μl of the DAPI solution were added to each well of fixed cells. Plates were left at room temperature for one hour then aspirated and washed twice with 100 μl of PBS. The stained cells were left at 4° C. for a minimum of 16 hours before photographic analysis with Array Scan II (Cellomics). Analysis of the photographic images to determine numbers of live cells (proliferation), apoptotic cells and dead cells, were according to the methods described in U.S. Provisional Patent Application Ser. No. 60/406,714, which incorporated herein in its entirety.

Non Photographic Proliferation Analysis

Some cell plates were treated with Promega Cell titer Aqueous 1 kit (Promega—VWR catalog number G3580). In this case, 48 hours after the test compound were added, 100 μl of media were removed and 20 μl of cell titer reagent were added to all wells. Plates were incubated at 37° C. for 45 minutes prior to absorbance reads on the Wallac plate reader at 490 nm for 0.1 sec/well. Similar experiments can be carried out using cell lines U2OS and DLD-1.

### EXAMPLE F

### p53-Dependent Transcription Assay Cell Line

Stable lines of U2OS (osteosarcoma; Wild-type p53 positive), HCT116 (colorectal carcinoma; Wild-type p53 positive) and H1299 (lung non-small cell carcinoma; p53 null) carrying p53-dependent transcriptional regulatory element linked to a Luciferase reporter gene and H1299 carrying a 5′-regulatory region of GADD45 linked to Luciferase were used.

Positive Control Drug

Etoposide (DNA-damaging reagent) and MG132 (Proteasome inhibitor) were used as positive control drugs.

Procedure

Day 1: Cells were re-plated into 96-well plates at suitable densities to make 50-60% confluence on Day 2. The plating densities are as follows: 7.5×10³ cells/well (or 2.5×10⁴ cells/cm²) for U2OS; 1.5×10⁴ cells/well (or 5.0×10⁴ cells/cm²) for HCT116; and 1.0×10⁴ cells/well (or 3.3×10⁴ cells/cm²) for H1299.

Day 2: The compounds or control drugs were diluted in dimethyl sulfoxide (DMSO) and added to the wells to create 6-points/2-fold serial dilution (20, 10, 5, 2.5, 1.25, 0.625 and 0 μM) for each compound. The DMSO concentration was adjusted to 0.2% in all wells.

Day 3: Twenty to 24 hours after compound addition, cells were twice washed with PBS and lysed with Glo-lysis buffer (100 μl/well, Promega; Cat#E2661). Plates were subjected to gentle agitation on a table-top shaker for 5 minutes at room temperature. Fifty μl of lysate was transferred to an assay plate to test for luciferase activity and mixed with 2× assay buffer (Promega; Cat# E1483). The reaction mixture was left at room temperature for 2 minutes and then luminescence was measure by a luminometer (Victor™, 1420 Multilabel Counter, Wallac).

Analysis:

Data analysis was done on Excel software (Microsoft). Values in Table 4 represent maximal fold induction over negative control (DMSO only) and are the mean of at least duplicate experiments. Compounds that elicited greater than 2-fold induction in wild-type-p53-expressing lines (U2OS and HCT116) are defined as positive while those that showed greater than 1.5-fold induction in H1299 lines are off-target compounds.

The compounds in the table immediately below illustrate the inhibitory activity of the compounds of the invention.

TABLE 2

| Cpd. No. | MDM2 IC₅₀ | APC IC₅₀ | E2 IC₅₀ |
|---|---|---|---|
| 14 | + | + | |
| 15 | ++ | ++ | |
| 16 | ++ | | |
| 23 | + | + | |
| 65 | ++ | ++ | |
| 146 | + | | |
| 177 | +++ | | |
| 186 | ++ | | |
| 193 | ++ | | |
| 207 | ++ | | |
| 215 | +++ | ++ | |
| 216 | ++ | | |
| 222 | + | + | |
| 223 | + | + | |
| 224 | + | + | |
| 225 | + | + | |
| 227 | + | + | |
| 228 | + | ++ | |
| 230 | + | ++ | |
| 231 | + | + | |
| 233 | + | + | |
| 235 | ++ | ++ | |
| 245 | + | ++ | |
| 251 | + | + | |
| 252 | + | ++ | |
| 267 | + | ++ | |
| 268 | + | + | |
| 275 | ++ | ++ | |
| 282 | ++ | ++ | |
| 283 | + | + | |
| 289 | + | + | |
| 290 | + | + | |
| 291 | + | ++ | |
| 292 | + | + | |
| 297 | ++ | ++ | + |
| 328 | ++ | ++ | + |
| 343 | ++ | ++ | |
| 344 | ++ | ++ | |
| 345 | + | ++ | + |
| 351 | ++ | ++ | + |
| 352 | ++ | ++ | + |
| 353 | ++ | ++ | + |
| 359 | ++ | ++ | |
| 360 | ++ | ++ | + |

+++ means less than 1 μM
++ means between 1 and 20 μM
+ means 20 μM or greater

The compounds in the tables immediately below further illustrate the inhibitory activity of the compounds of the invention using a number of assays.

TABLE 3

| | MDM2 Plate | MDM2 Gel | APC Plate | E1/E2 plate |
|---|---|---|---|---|
| Cpd. No. | IC50 (μM) | 20 μM | IC50 (μM) | μM |
| 360 | +++ | Potent | ++ | + |
| 353 | ++ | Active | ++ | |
| 352 | ++ | Weak | + | |
| 351 | ++ | Active | ++ | |
| 282 | ++ | Potent | ++ | |
| 275 | ++ | Weak | ++ | |

+++ means less than 1 μM
++ means between 1 and 20 μM
+ means 20 μM or greater

TABLE 4
Cell-based Reporter Assay Data Fold Induction of p53 activity

| | p53 +ve | p53 +ve | p53 −ve | p53 −ve |
|---|---|---|---|---|
| | cell type | Cell type | Cell type | cell type |
| Cpd. No. | U20SRE | A549RE | H1299RE | H1299GADD45 |
| 360 | b | a | c | c |
| 353 | a | a | c | c |
| 352 | b | a | c | c |
| 351 | b | a | c | c |
| 282 | b | a | | |
| 275 | b | a | | |
| 233 | a | a | | |

a means 2 or less
b means greater than 2
c means 1.5 or less

TABLE 5
Cell-based PAD (Proliferation-Apoptosis-DNA content)
Assay Anti-Proliferation Activity Assay

| | PAD/U20S | PAD/HCT | PAD/H1299 | PAD/DLD-1 |
|---|---|---|---|---|
| Cpd. No. | IC50 (μM) | IC50 (μM) | IC50 (μM) | IC50 (μM) |
| 360 | ++ | | ++ | |
| 360 | ++ | ++ | ++ | ++ |
| 353 | ++ | ++ | ++ | |
| 352 | ++ | | ++ | |
| 351 | ++ | ++ | ++ | |
| 282 | ++ | ++ | ++ | ++ |
| 275 | ++ | ++ | ++ | ++ |

++ means between 1 and 20 μM

The following abbreviations apply for Tables 2-5.

hMDM2 Plate/Gel is Mouse double minute 2, human homolog. This protein belongs to the E3 RING finger ligase group.

APC Plate/gel is Anaphase Promoting Complex. This protein also belongs to E3 RING finger ligase group.

E1/E2 is E1 and E2 (UBCH5a) enzyme counter assay.

A549RE is a p53 wild-type A549 cell line containing p53 reporter system. If the compound acts against MDM2, p53 activity increases.

U20SRE is a p53 wild-type U2OS cell line containing p53 reporter system. If the compound acts against MDM2, p53 activity increases.

H1299RE is p53 null U2OS cell line containing p53 reporter system. Numbers indicate the selectivity of the compound with smaller number being higher selectivity.

H1299 GADD45 Reporter System is a system in which GADD45 is induced in p53-null cells in response to DNA damage. This cell line expresses the 5′-regulatory region of the GADD45 gene. This assay provides a way to rule out the possibility of DNA damage due to the compounds.

What is claimed is:

1. A compound of the formula 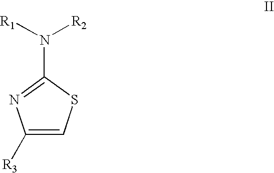
or pharmaceutically acceptable salts thereof, wherein
R₁ is —C₁-C₆ alkylaryl, —C₁-C₆ alkylheteroaryl, —C₁-C₆ alkylheterocyclyl, —C₀-C₆ alkylaryl-C(O)—N(R₅)(R₅ₐ), —C₀-C₆ alkylheteroaryl-C(O)—N(R₅)(R₅ₐ), —C₀-C₆ alkylaryl-NR₅—C(O)—R₅, —C₀-C₆ alkylheteroaryl-NR₅—C(O)—R₅, —C₀-C₆ alkylaryl-C(O)-heterocyclyl, —C₀-C₆ alkylheteroaryl-C(O)-heterocyclyl, —C₀-C₆ alkylaryl-C(O)-heteroaryl, —C₀-C₆ alkylheteroaryl-C(O)-heteroaryl, aryl, heteroaryl, heterocyclyl, wherein each of the alkyl, aryl, heterocyclyl, and heteroaryl is optionally substituted with 1 to 4 groups independently selected from R₇;
R₂ is —H, C₁-C₆ alkyl, C₂-C₆ alkenyl, C₂-C₆ alkynyl, —C₁-C₆ alkyl-N(R₄)(R₄ₐ), —C₁-C₆ alkylaryl, —C₁-C₆ alkylheteroaryl, —C₁-C₆ alkylheterocyclyl, —C₁-C₆ alkyl-aryl, aryl, heteroaryl, heterocyclyl, wherein each of the alkyl, alkenyl, alkynyl, aryl, heterocyclyl, and heteroaryl is optionally substituted with 1 to 4 groups selected from oxo, —OH, —SH, C₁-C₆ alkyl, C₁-C₆ alkoxy, mono- to per-halogenated C₁-C₆ alkyl, mono- to per-halogenated C₁-C₆ alkoxy, —N(R₈)(R₈ₐ), —NO₂, halo, or —CN;
R₄ and R₄ₐ are independently —H, —OH, C₁-C₆ alkyl, —SH, C₂-C₆ alkenyl, C₂-C₆ alkynyl, —N(R₈)(R₈ₐ) or C₁-C₆ alkoxy;
R₅ and R₅ₐ are independently —H, —OH, C₁-C₆ alkyl, —C₀-C₆ alkyl-C(O)—OH, C₁-C₆ alkoxy, —C₁-C₆ alkyl-C₃-C₆ cycloalkyl, —C₀-C₆ alkylaryl, —C₀-C₆ alkylheteroaryl, wherein each of the alkyl, aryl, and heteroaryl are optionally substituted with 1 to 4 groups selected from oxo, —C(O)—OH, —OH, —SH, C₁-C₆ alkyl, C₁-C₆ alkoxy, mono- to per-halogenated C₁-C₆ alkyl, mono- to per-halogenated C₁-C₆ alkoxy, —C₁-C₆ alkyl-N(R₄)₂, —N(R₈)(R₈ₐ), —NO₂, halo, or —CN;
R₈ and R₈ₐ are independently —H or —C₁-C₆ alkyl; and
R₇ is oxo, —OH, —SH, C₁-C₆ alkyl, C₁-C₆ alkoxy, —C(O)—OH, mono- to per-halogenated C₁-C₆ alkyl, mono- to per-halogenated C₁-C₆ alkoxy, —N(R₈)(R₈ₐ), —NO₂, halo, or —CN;
R₃ is C₁-C₆ alkyl, mono- to per-halogenated C₁-C₆ alkyl, —C₁-C₆ alkylaryl, —C₁-C₆ alkylheteroaryl, —C₁-C₆ alkylheterocyclyl, aryl, heteroaryl, heterocyclyl, wherein each of the alkyl, aryl, heterocyclyl, and heteroaryl is optionally substituted with 1 to 4 groups selected from oxo, —OH, —SH, C₁-C₆ alkyl, C₁-C₆ alkoxy, mono- to per-halogenated C₁-C₆ alkyl, mono- to per-halogenated C₁-C₆ alkoxy, aryl, heteroaryl, heterocyclyl, —C₁-C₆ alkyl-N(R₄)(R₄ₐ), —N(R₈)(R₈ₐ), —NO₂, halo, or —CN; or
R₃ is aryl optionally substituted in the meta and para position with 1 to 4 groups selected from oxo, —OH, —SH, C₁-C₆ alkyl, C₁-C₆ alkoxy, mono- to per-halogenated C₁-C₆ alkyl, mono- to per-halogenated C₁-C₆ alkoxy, aryl, heteroaryl, heterocyclyl, —C₁-C₆ alkyl-N(R₄)(R₄ₐ), —N(R₈)(R₈ₐ), —NO₂, halo, or —CN.

2. The compound according to claim 1, wherein
R₁ is —C₀-C₆ alkylaryl-C(O)—N(R₅)(R₅ₐ) or —C₀-C₆ alkylaryl-C(O)-heterocyclyl;
R₂ is C₁-C₆ alkyl, C₂-C₆ alkenyl, —C₁-C₆ alkyl-N(R₄)(R₄ₐ), —C₁-C₆ alkylaryl, —C₁-C₆ alkylheteroaryl, —C₁-C₆ alkyl-O-aryl, or aryl, wherein each of the alkyl, alkenyl, aryl, and heteroaryl is optionally substituted with 1 to 4 groups selected from mono- to per-halogenated C₁-C₆ alkoxy, C₁-C₆ alkoxy or halo;
R₃ is C₁-C₆ alkyl, mono- to per-halogenated C₁-C₆ alkyl, or aryl wherein each of the alkyl or aryl is optionally substituted with 1 to 4 groups selected from C₁-C₆ alkyl, C₁-C₆ alkoxy, aryl, —NO₂, or halo; or
R₃ is aryl optionally substituted in the meta and para position with 1 to 4 groups selected from C₁-C₆ alkyl, C₁-C₆ alkoxy, aryl, —NO₂, or halo;
R₄ and R₄ₐ are independently —H or C₁-C₆ alkyl; and
R₅ and R₅ₐ are independently —H, C₁-C₆ alkyl, —C₀-C₆ alkyl-C(O)—OH, —C₁-C₆ alkyl-C₃-C₆ cycloalkyl, —C₀-C₆ alkylaryl, wherein each of the alkyl and aryl is optionally substituted with 1 to 4 groups selected from C₁-C₆ alkoxy, —C(O)—OH, —NH₂, or —C₁-C₆ alkyl-N(R₄)(R₄ₐ).

3. The compound according to claim 2, wherein
R₁ is —C₀-C₆ alkylaryl-C(O)—N(R₅)(R₅ₐ);
R₂ is C₁-C₆ alkyl, C₂-C₆ alkenyl, C₁-C₆ alkylaryl, or aryl, wherein the aryl is optionally substituted with 1 to 4 groups selected from halo;
R₃ is aryl optionally substituted with 1 to 4 groups selected from C₁-C₆ alkoxy, —NO₂, aryl, or halo; or
R₃ is aryl optionally substituted in the meta and para position with 1 to 4 groups selected from C₁-C₆ alkoxy, —NO₂, aryl, or halo;
R₄ and R₄ₐ are —H; and
R₅ and R₅ₐ are independently —H, C₁-C₆ alkyl, —C₀-C₆ alkyl-C(O)—OH or —C₀-C₆ alkylaryl, wherein each of the alkyl and aryl is optionally substituted with 1 to 4 groups selected from —C(O)—OH or —C₁-C₆ alkyl-N(R₄)(R₄ₐ).

4. The compound according to claim 3, wherein R₁ is —C₁-C₃ alkylaryl-C(O)—N(R₅)(R₅ₐ).

5. The compound according to claim 4, wherein R₁ is —CH₂-aryl-C(O)—N(R₅)(R₅ₐ).

6. The compound according to claim 5, wherein the aryl is phenyl.

7. The compound according to claim 5, wherein one R₅ is —H and R₅ₐ is —C₁-C₃ alkyl-C(O)—OH.

8. The compound according to claim 7, wherein R₅ₐ is —CH₂—C(O)—OH.

9. The compound according to claim 5, wherein one R₅ is —H and R₅ₐ is C₁-C₃ alkyl substituted with —C(O)—OH.

10. The compound according to claim 9, wherein R₅ₐ is —CH(C(O)—OH)-CH₃.

11. The compound according to claim 5, wherein R₅ is —H and R₅ₐ is —C₁-C₃ alkylaryl, wherein each of the alkyl and aryl is optionally substituted with a group selected from —C(O)—OH or —C₁-C₃ alkyl-NH₂.

12. The compound according to claim 11, wherein R₅ₐ is —CH₃-aryl, wherein the aryl is substituted with —CH₂—NH₂.

13. The compound according to claim 12, wherein the aryl is phenyl.

14. The compound according to claim 11, wherein R₅ₐ is —CH₂-aryl, wherein the —CH₂— is substituted with —C(O)—OH.

15. The compound according to claim 14, wherein the aryl is phenyl.

16. The compound according to claim 3, wherein R₂ is C₁-C₄ alkyl.

17. The compound according to claim 16, wherein R₂ is selected from the group consisting of methyl, ethyl, propyl and butyl.

18. The compound according to claim 3, wherein R₂ is C₂-C₃ alkenyl.

19. The compound according to claim 18, wherein R₂ is propenyl.

20. The compound according to claim 3, wherein R₂ is C₁-C₃ alkylaryl.

21. The compound according to claim 20, wherein R₂ is —CH₂-aryl or —C₂H₄-aryl.

22. The compound according to claim 21, wherein the aryl is phenyl.

23. The compound according to claim 3, wherein R₂ is aryl optionally substituted with 1 to 2 groups selected from halo.

24. The compound according to claim 23, wherein the halo is fluoro, chloro or bromo.

25. The compound according to claim 23, wherein the aryl is phenyl.

26. The compound according to claim 3, wherein R₃ is aryl optionally substituted with 1 to 2 groups selected from C₁-C₃ alkoxy, phenyl, —NO₂, or halo.

27. The compound according to claim 26, wherein R₃ is phenyl substituted with 1 to 2 groups selected from methoxy, phenyl, choro, fluoro or bromo.

28. The compound according to claim 26, wherein R₃ is phenyl or naphthyl.

29. The compound according to claim 26, wherein R₃ is phenyl optionally substituted in the meta and para position with 1 or 2 groups selected from halo, C₁-C₆ alkyl, C₁-C₆ alkoxy, aryl, or —NO₂.

30. The compound according to claim 29, wherein R₃ is phenyl substituted in the meta and para position with 1 or 2 groups selected from —NO₂, methoxy, chloro, fluoro, bromo, methyl, or phenyl.

31. The compound according to claim 2, wherein
R₁ is —C₀-C₆ alkylaryl-C(O)-heterocyclyl;
R₂ is —C₁-C₆ alkyl-N(R₄)(R₄ₐ), —C₁-C₆ alkylaryl, —C₁-C₆ alkyl-O-aryl, or aryl, wherein each of the alkyl and aryl is optionally substituted with 1 to 4 groups selected from mono- to per-halogenated C₁-C₆ alkoxy, or C₁-C₆ alkoxy;
R₃ is aryl optionally substituted with 1 to 4 groups selected from C₁-C₆ alkoxy, aryl, —NO₂, or halo; or
R₃ is aryl optionally substituted in the meta and para position with 1 to 4 groups selected from C₁-C₆ alkoxy, aryl, —NO₂, or halo; and
R₄ is C₁-C₆ alkyl.

32. The compound according to claim 31, wherein R₁ is —C₁-C₃ alkylaryl-C(O)-heterocyclyl.

33. The compound according to claim 32, wherein the aryl is phenyl.

34. The compound according to claim 33, wherein R₁ is —CH₂-phenyl-C(O)-heterocyclyl.

35. The compound according to claim 34, wherein the heterocyclyl is selected from the group consisting of piperazinyl and morpholinyl.

36. The compound according to claim 31, wherein R₂ is —C₁-C₆ alkylaryl wherein the aryl is optionally substituted with 1 to 2 groups selected from mono- to per-halogenated C₁-C₆ alkoxy, or C₁-C₆ alkoxy.

37. The compound according to claim 48, wherein R₂ is —C₁-C₃ alkylaryl substituted with 1 to 2 groups selected from mono- to per-halogenated C₁-C₆ alkoxy or C₁-C₆ alkoxy.

38. The compound according to claim 37, wherein R₂ is —CH₂-phenyl substituted with 1 to 2 groups selected from trifluoromethoxy or methoxy.

39. The compound according to claim 31, wherein R₂ is —C₁-C₃ alkyl-O-aryl.

40. The compound according to claim 39, wherein R₂ is —C₂H₄—O-phenyl.

41. The compound according to claim 31, wherein R₂ is —C₁-C₃ alkyl-N(R₄)(R₄ₐ) and R₄ is C₁-C₃ alkyl.

42. The compound according to claim 41, wherein R₂ is —C₁-C₃ alkyl-N(isopropyl)₂.

43. The compound according to claim 42, wherein R₂ is —C₂H₄—N(isopropyl)₂.

44. The compound according to claim 31, wherein R₂ is aryl.

45. The compound according to claim 44, wherein aryl is naphthyl.

46. The compound according to claim 31, wherein R₃ is aryl optionally substituted with 1 to 2 groups selected from C₁-C₃ alkoxy, phenyl, —NO₂, or halo.

47. The compound according to claim 46, wherein R₃ is phenyl.

48. The compound according to claim 46, wherein R₃ is phenyl substituted with 1 to 2 groups selected from methoxy, phenyl, —NO₂, or halo.

49. The compound according to claim 48, wherein halo is selected from the group consisting of chloro, fluoro, and bromo.

50. The compound according to claim 46, wherein R₃ is phenyl optionally substituted in the meta and para position with 1 or 2 groups selected from halo, C₁-C₆ alkyl, C₁-C₆ alkoxy, aryl, or —NO₂.

51. The compound according to claim 50, wherein R₃ is phenyl substituted in the meta and para position with 1 or 2 groups selected from —NO₂, methoxy, chloro, fluoro, bromo, methyl, or phenyl.

52. The compound according to claim 1 that is selected from the group consisting of: 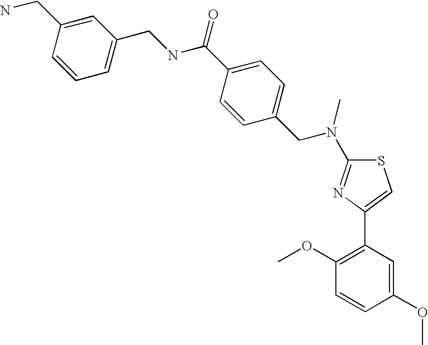 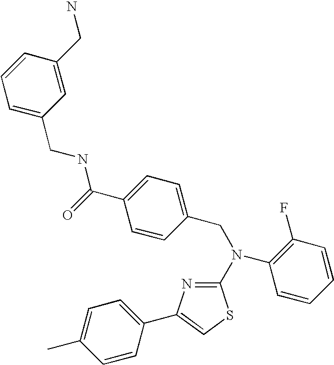 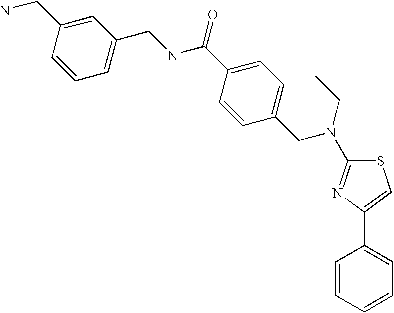 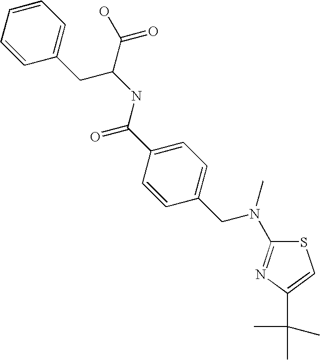 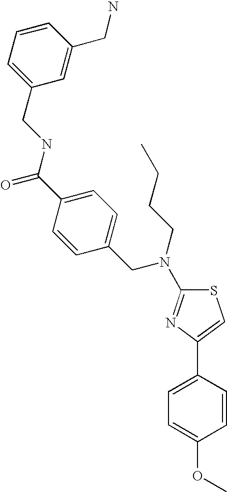 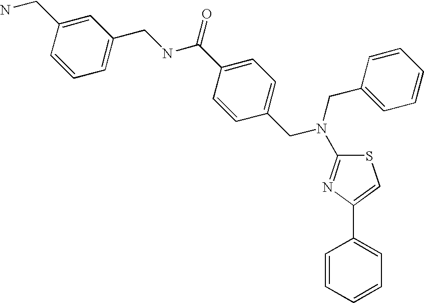 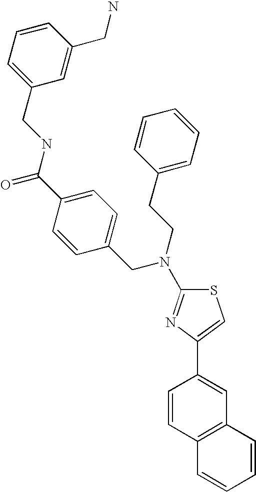 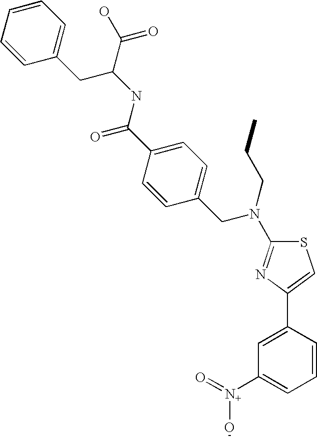 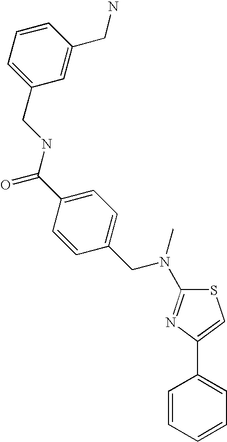 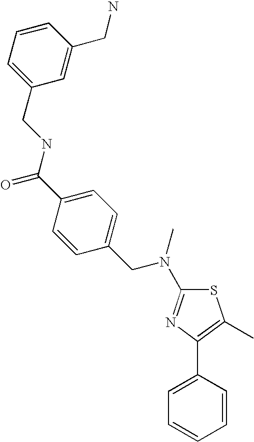 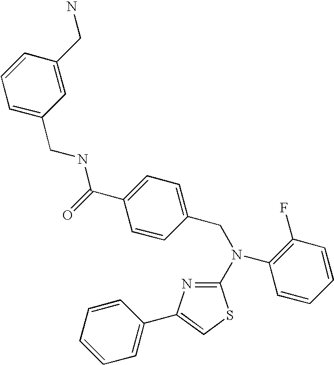 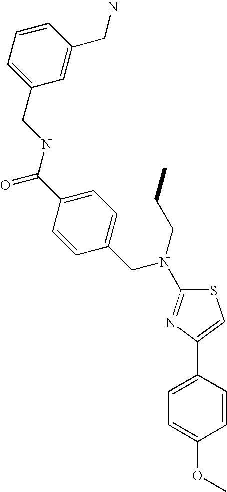 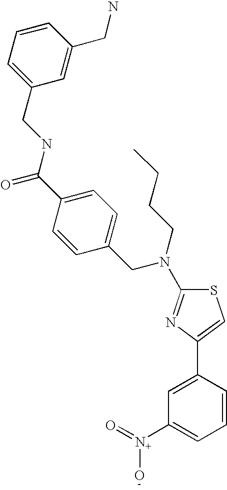 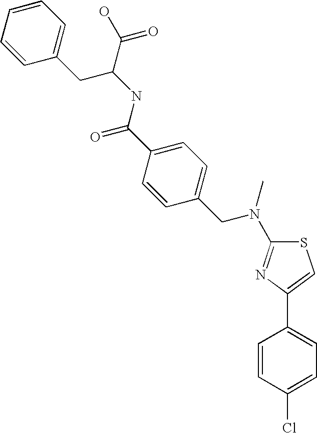 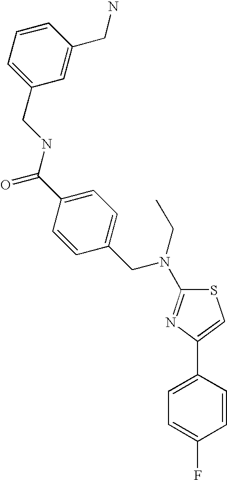 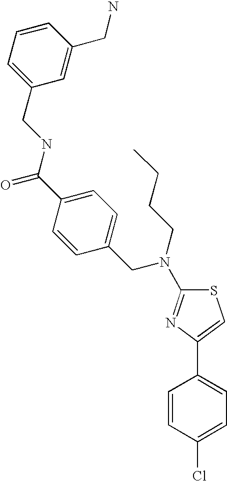 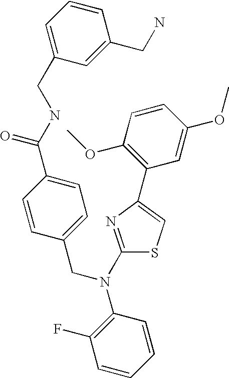 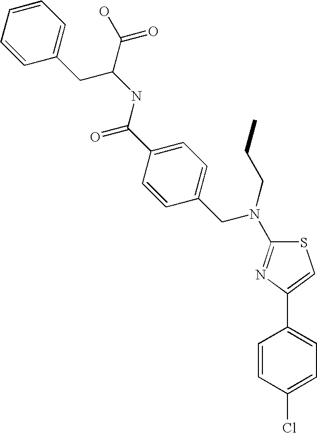 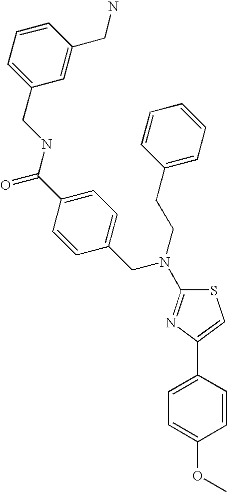 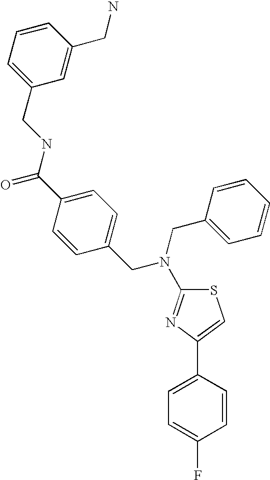 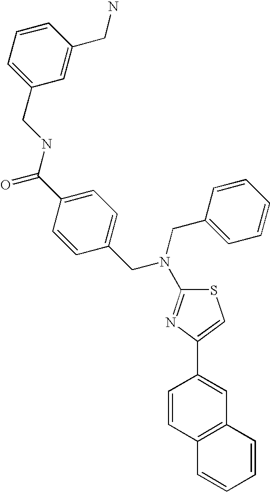 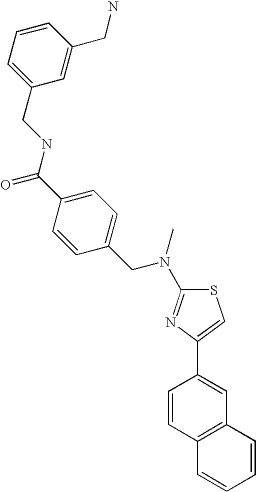 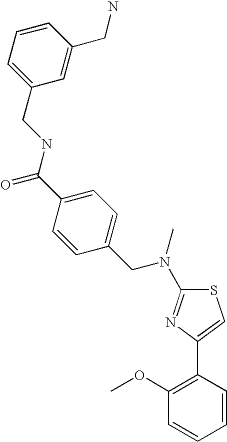 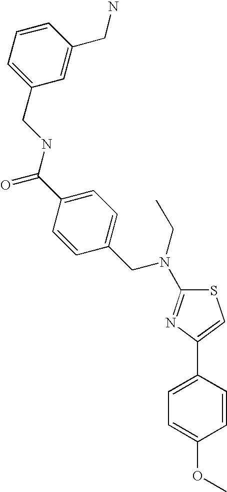 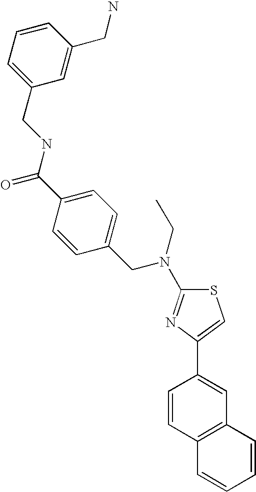 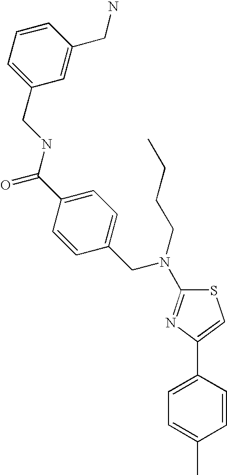 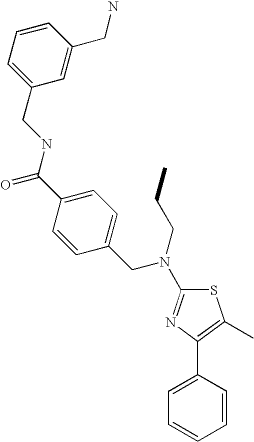 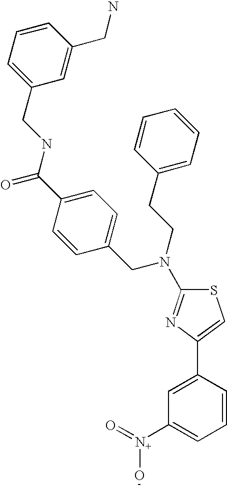 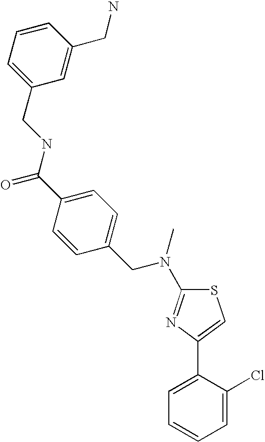 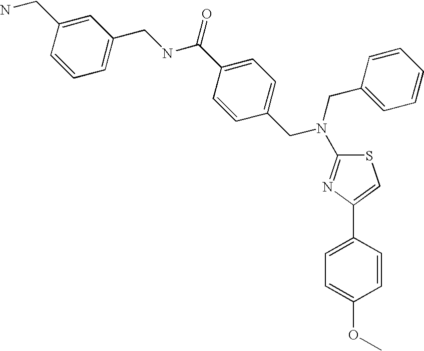 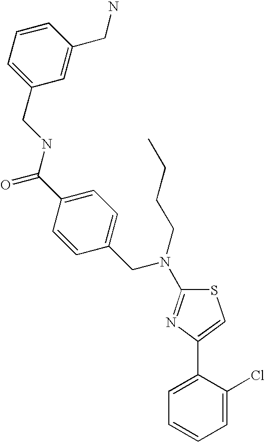 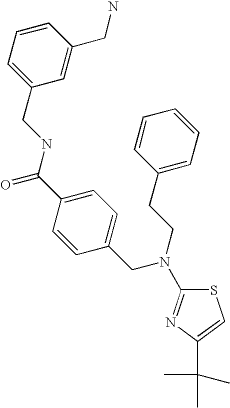 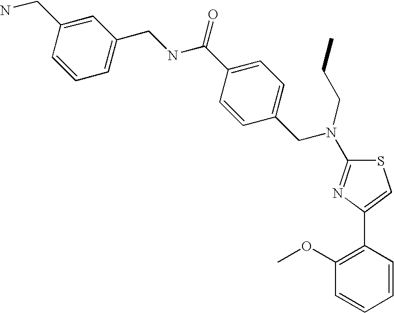 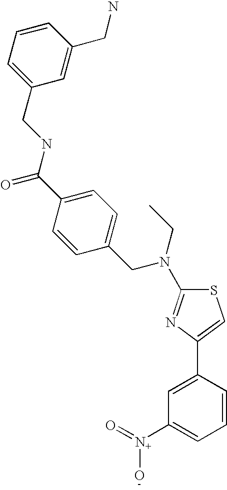 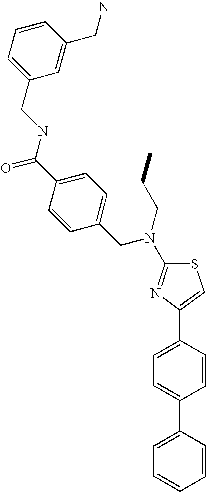 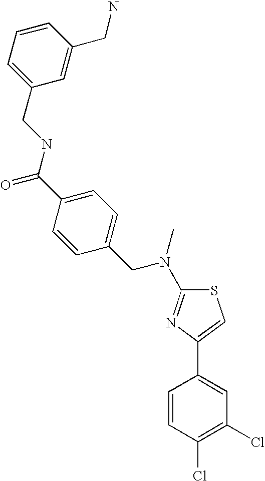 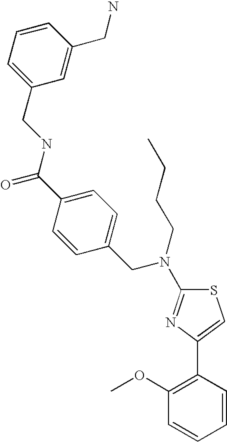 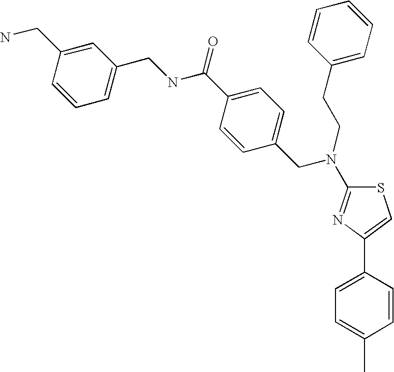 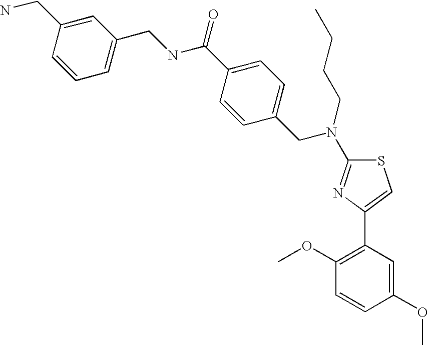 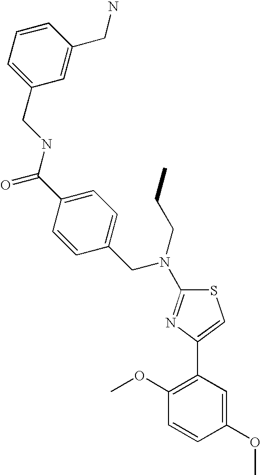 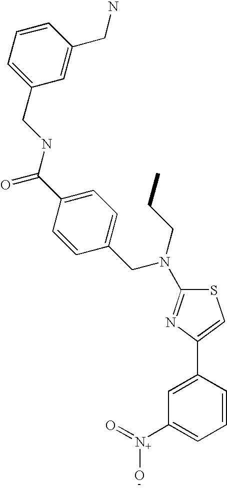 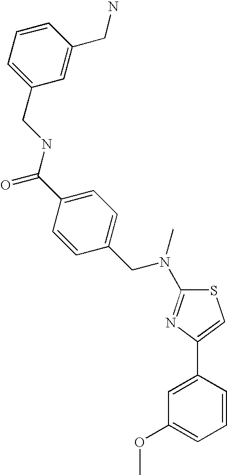 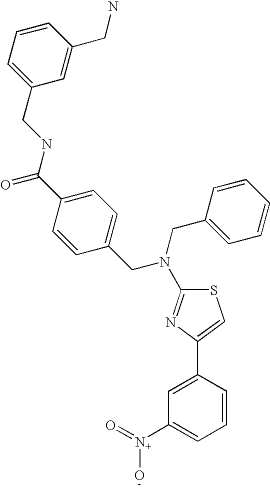 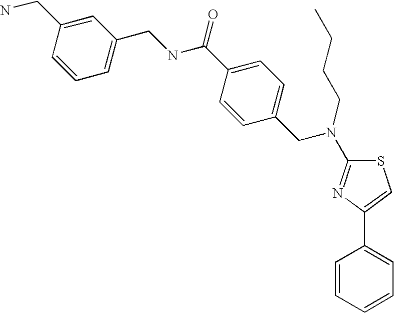 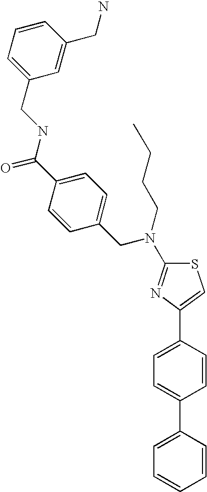 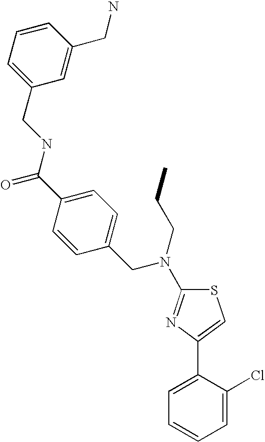 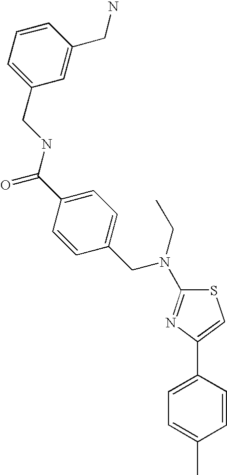 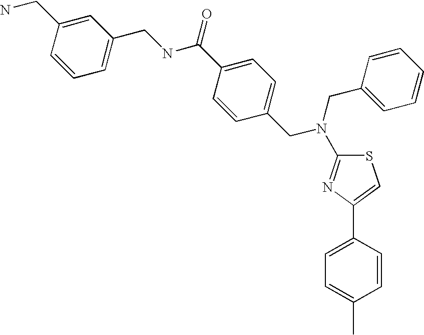 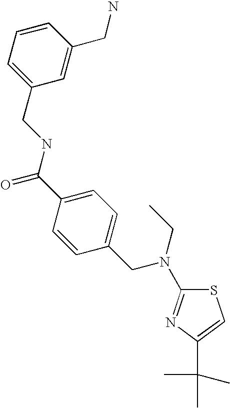 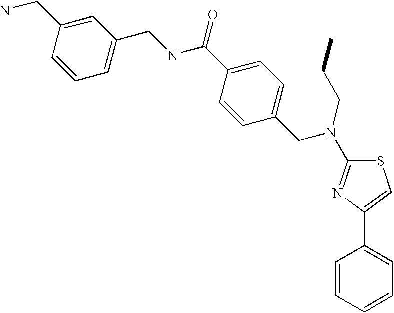 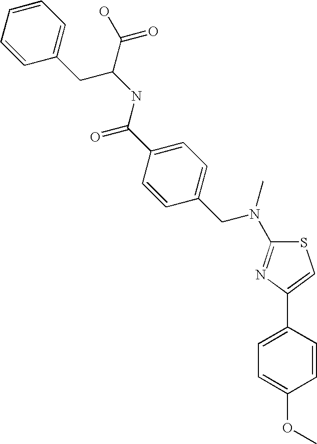 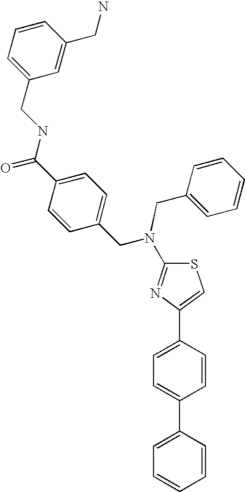 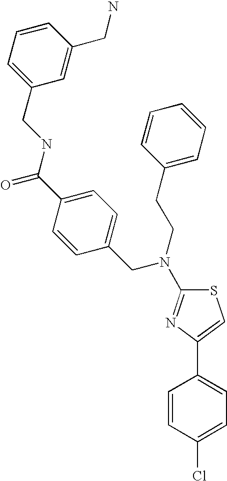 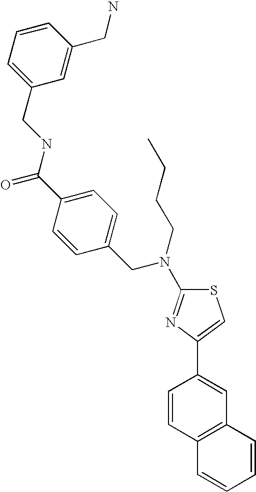 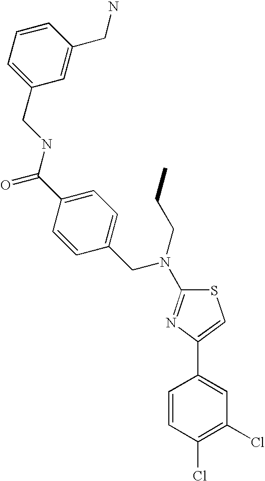 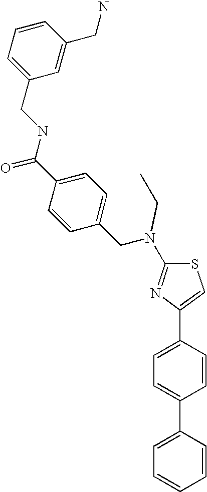 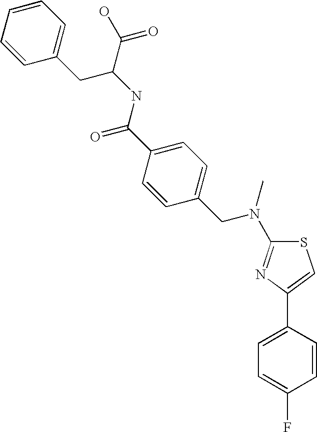 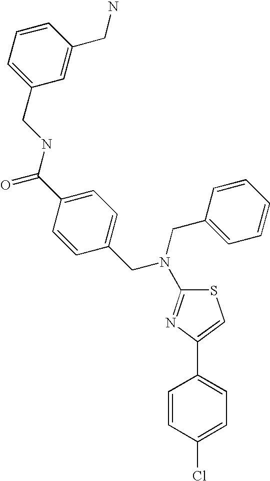 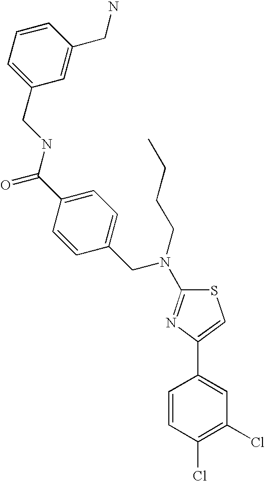 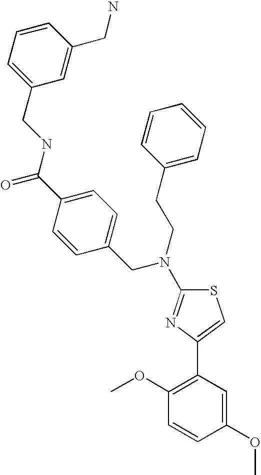 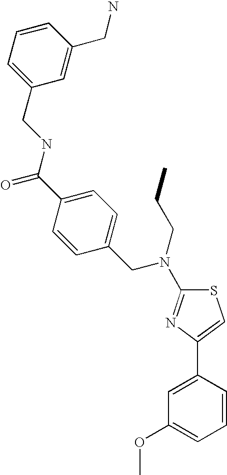 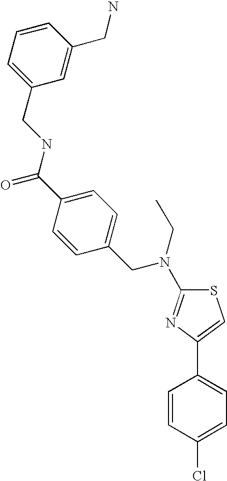 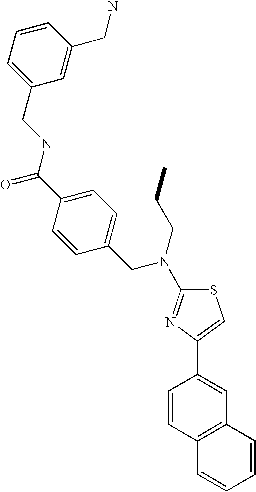 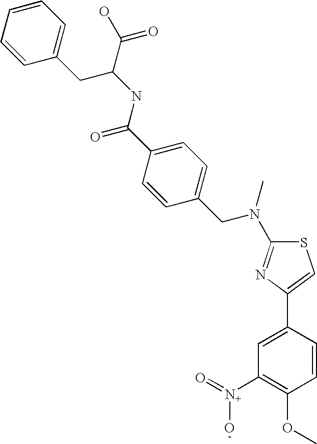 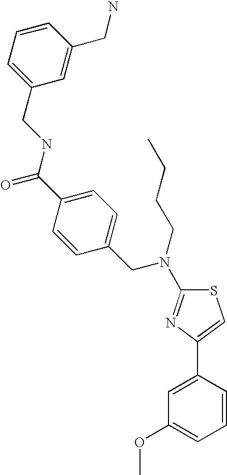 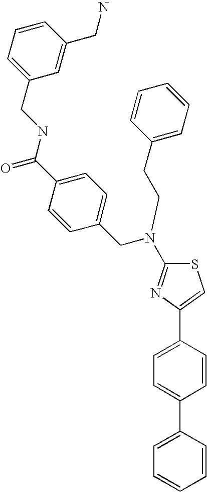 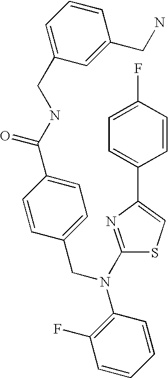 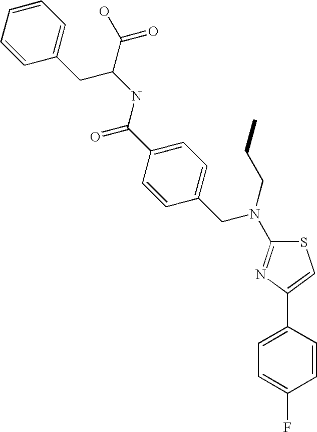 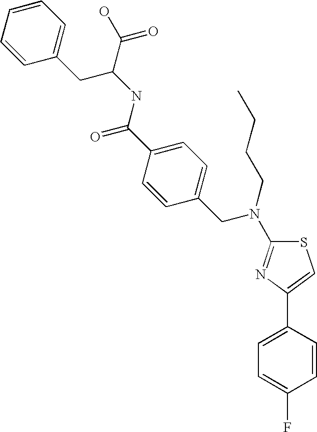 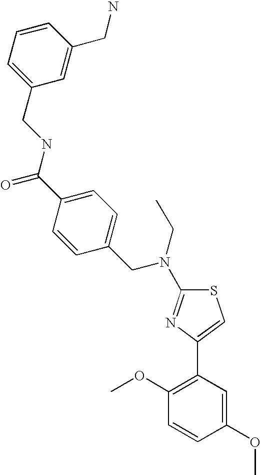 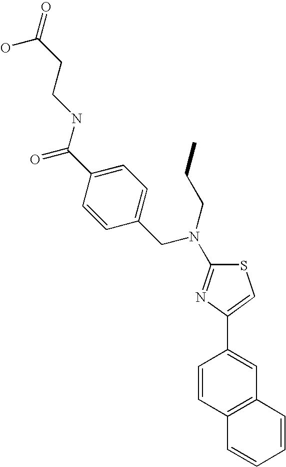 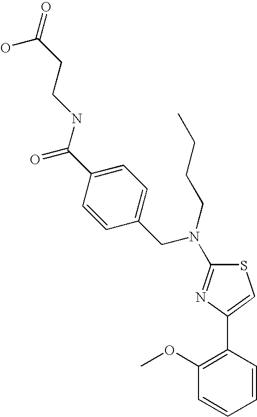 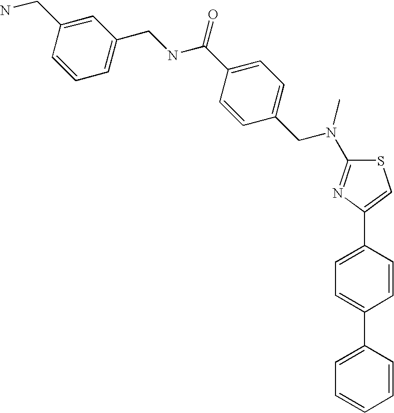 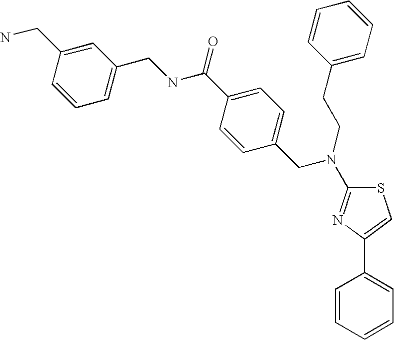 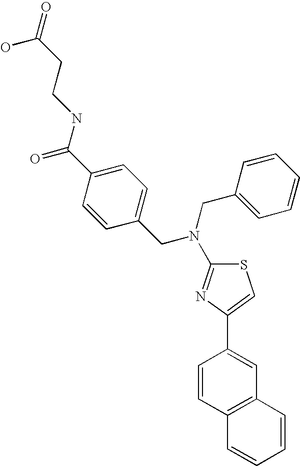 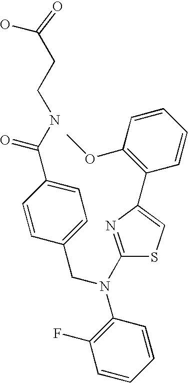 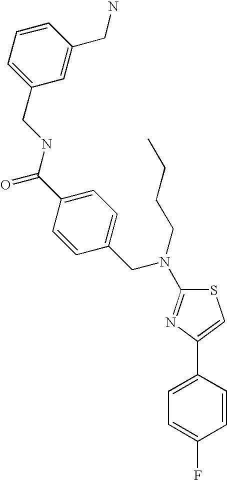 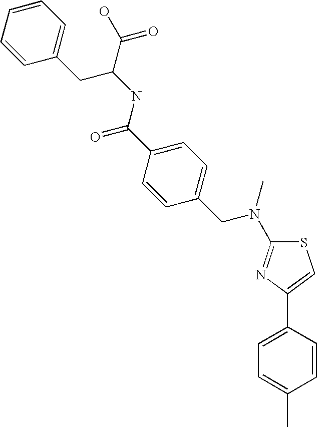 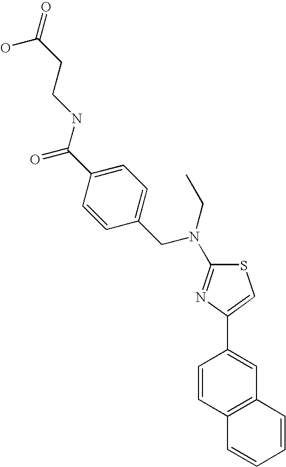 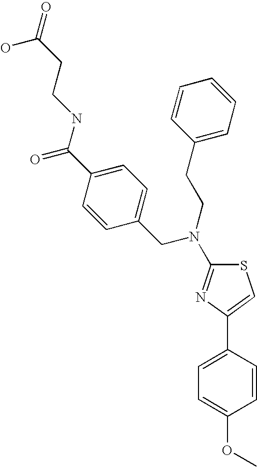 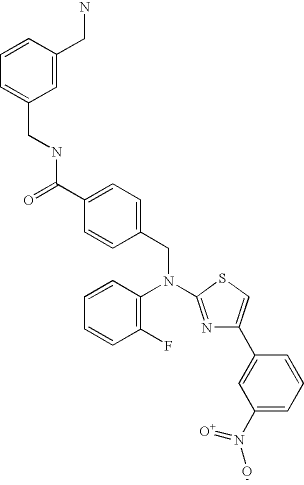 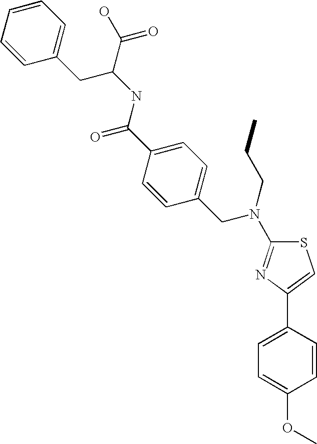 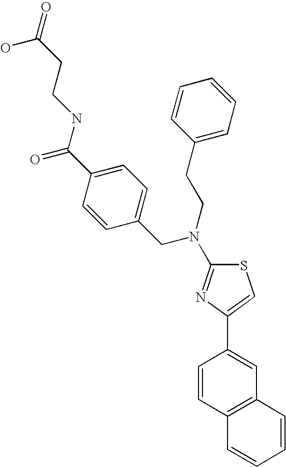 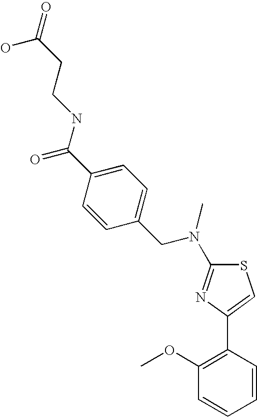 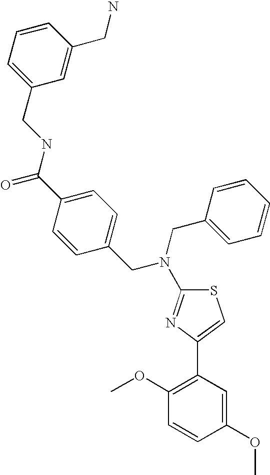 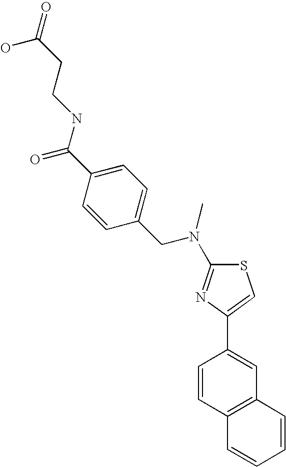 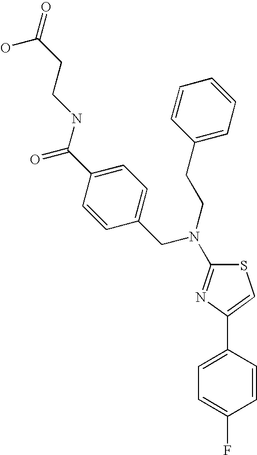 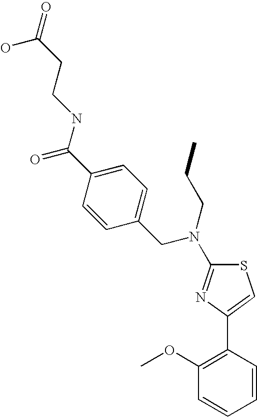 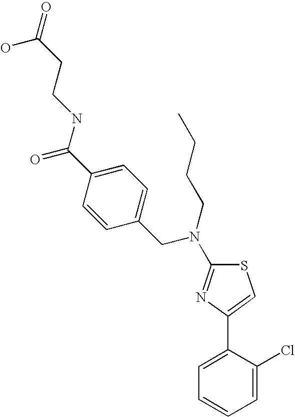 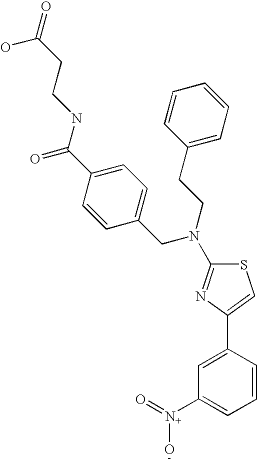 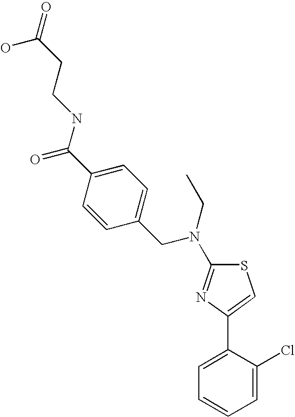 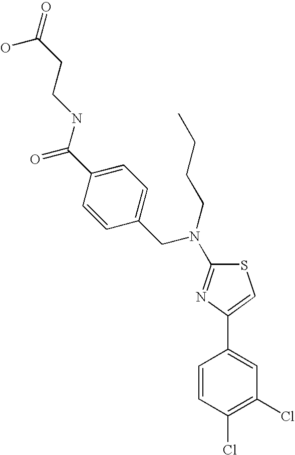 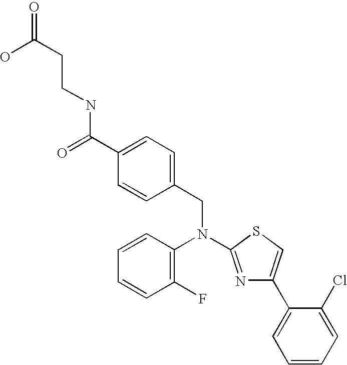 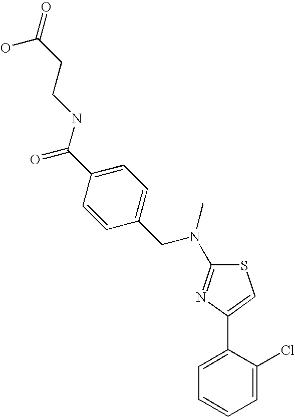 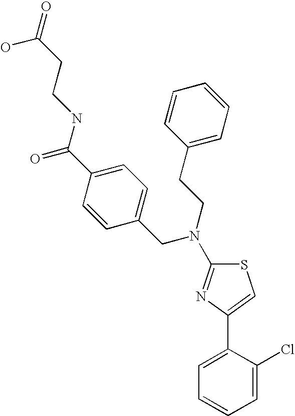 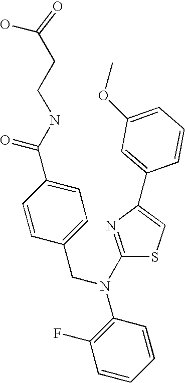 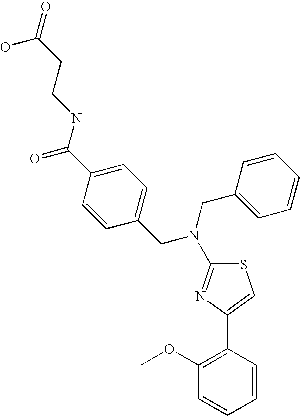 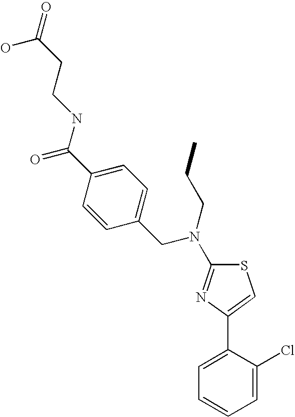 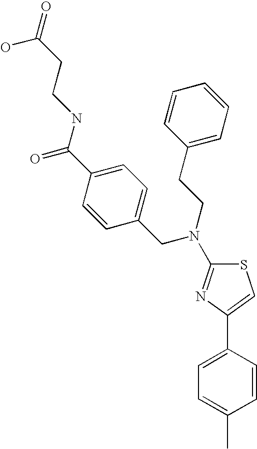 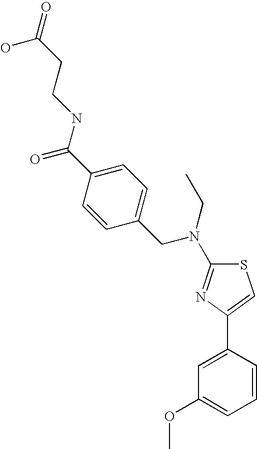 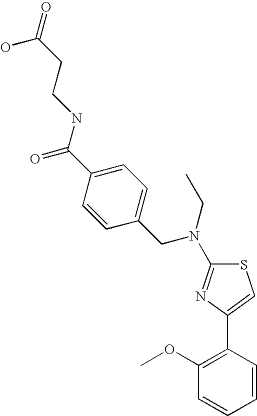 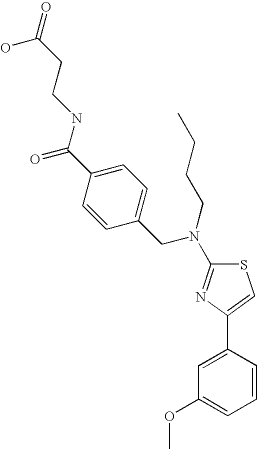 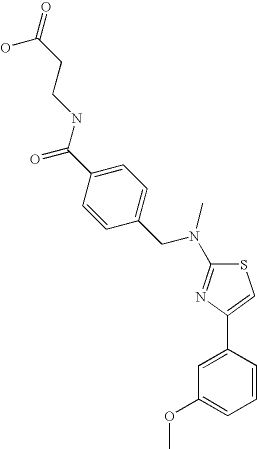 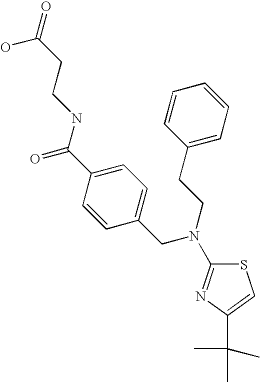 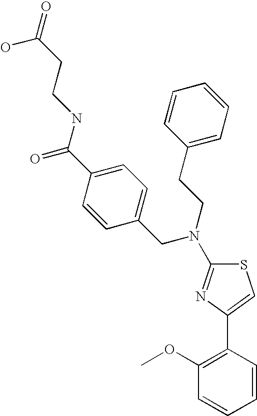 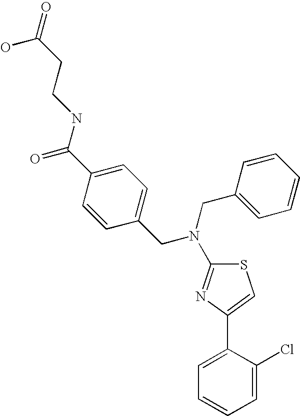 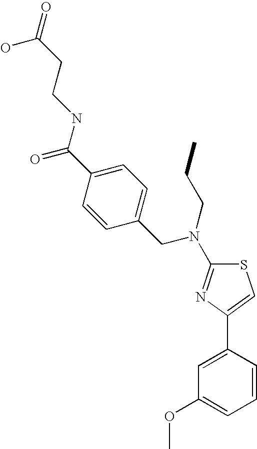 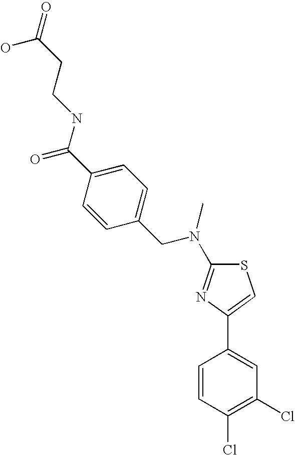 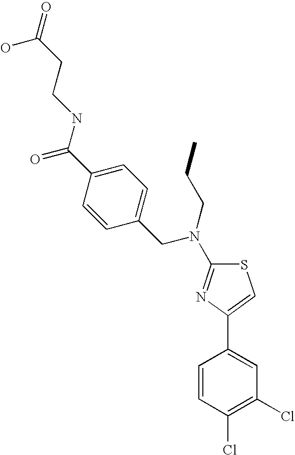 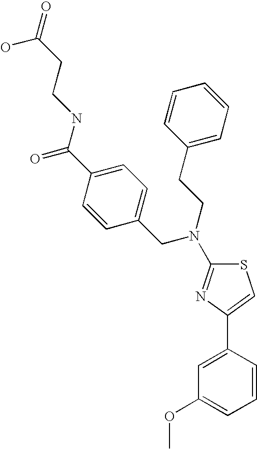 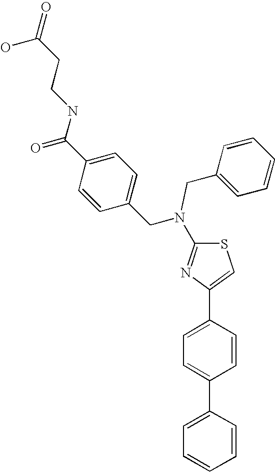 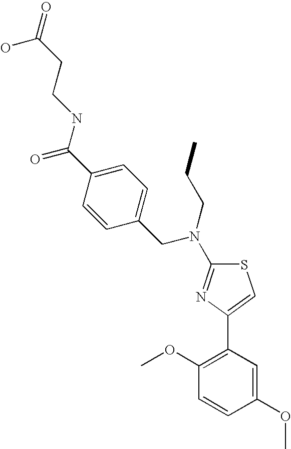 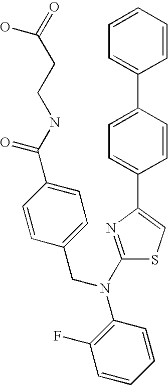 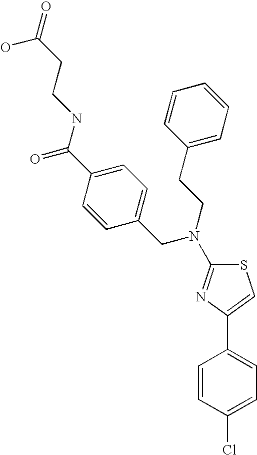 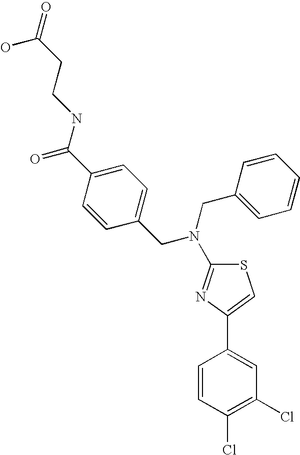 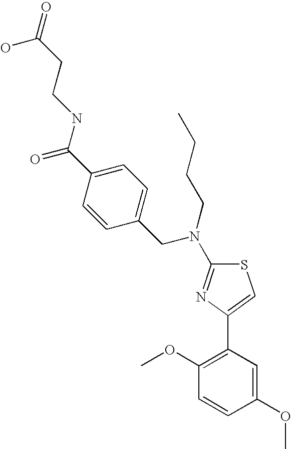 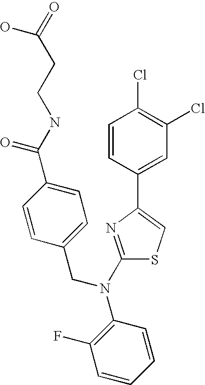 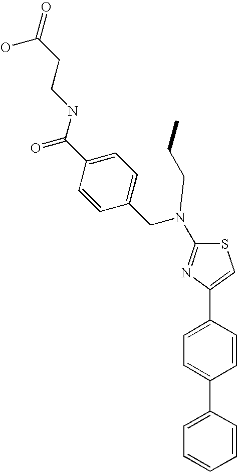 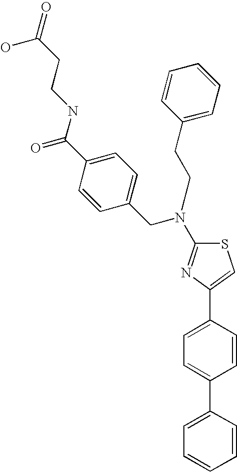 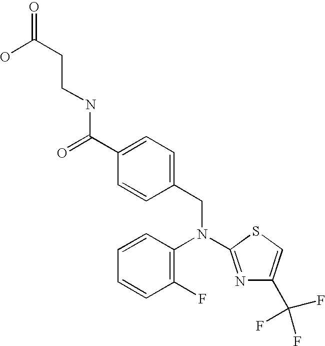 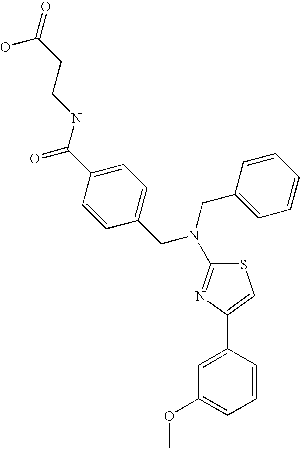 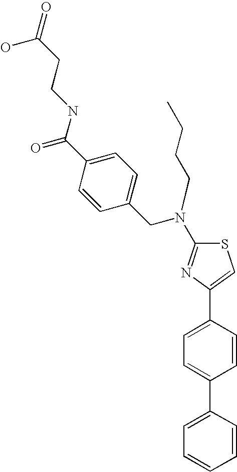 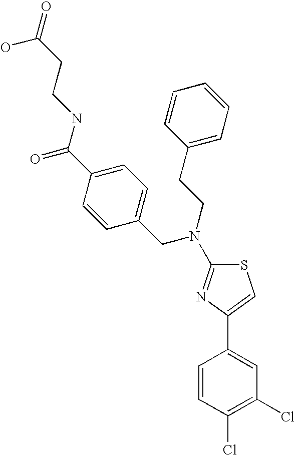 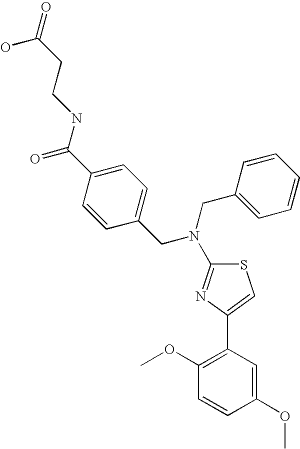 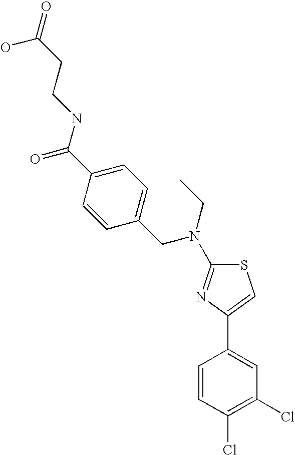 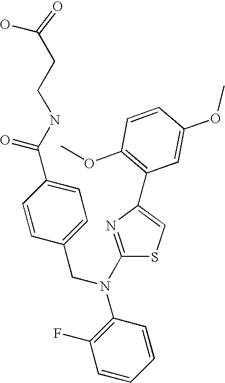 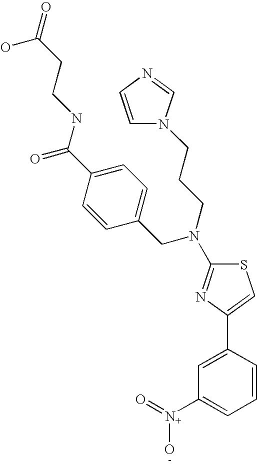 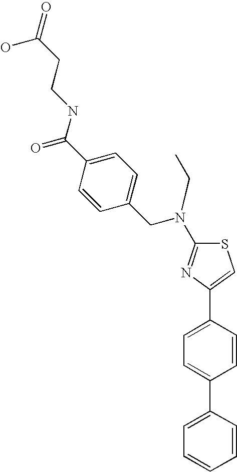 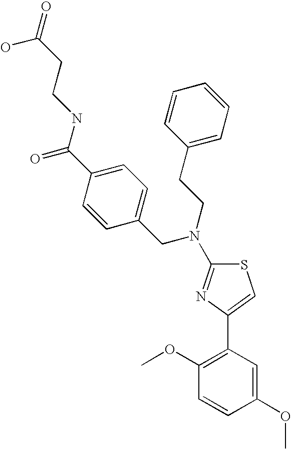 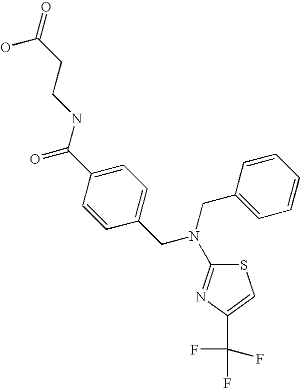 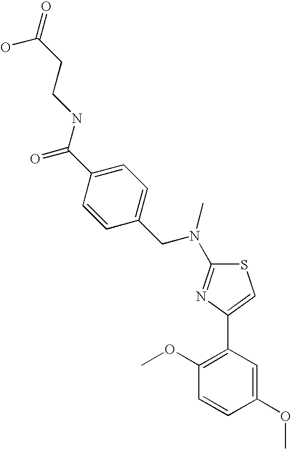 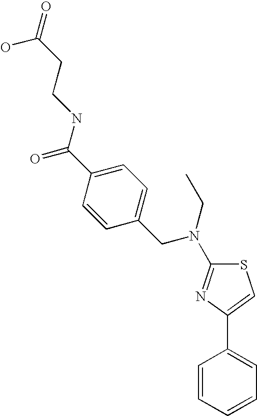 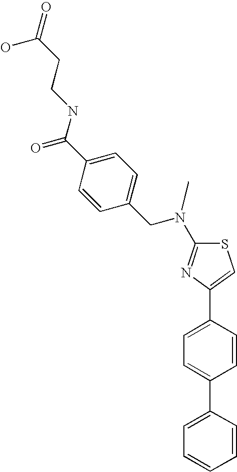 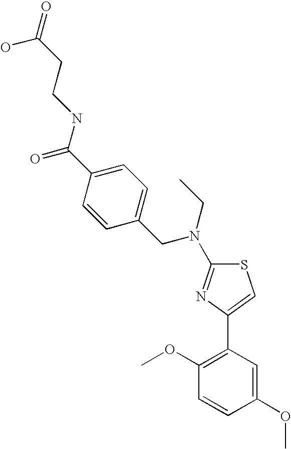 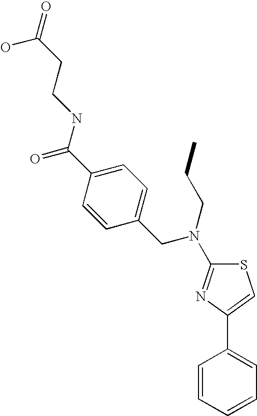 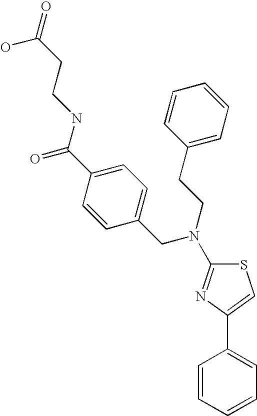 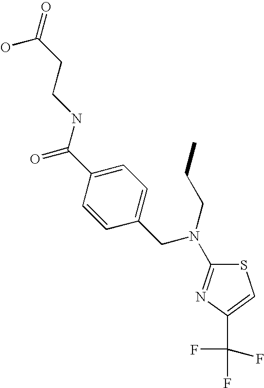 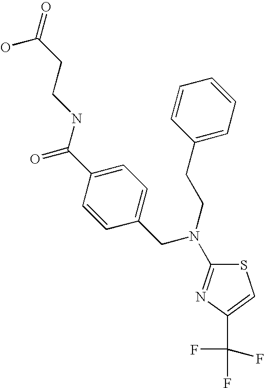 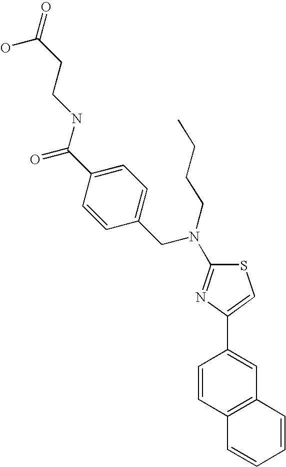 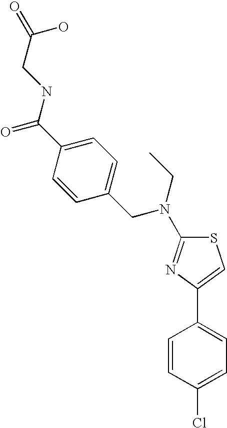 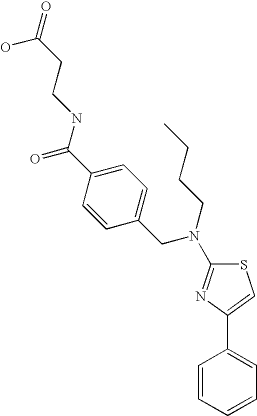 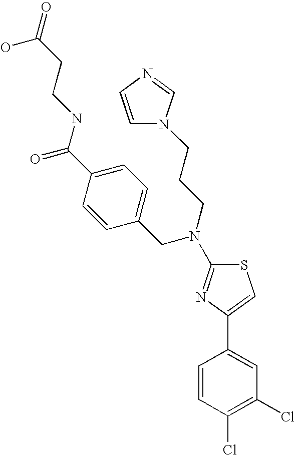 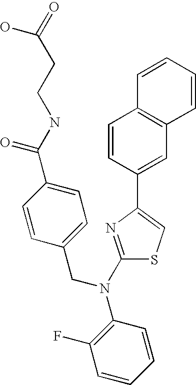 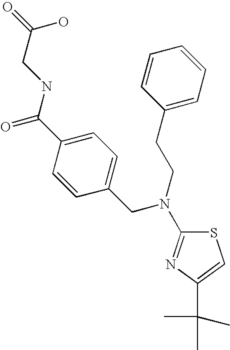 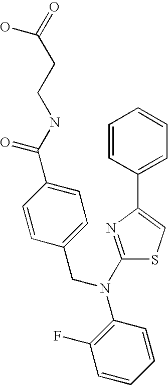 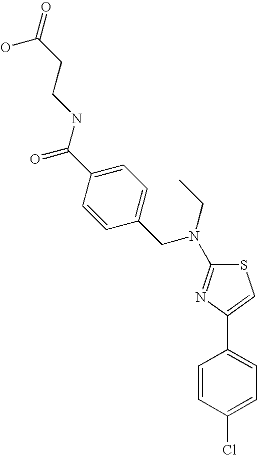 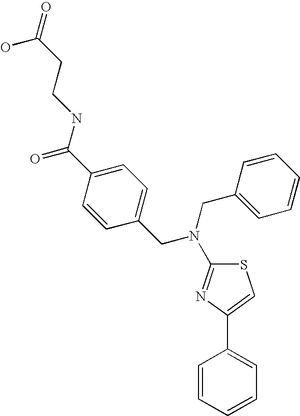 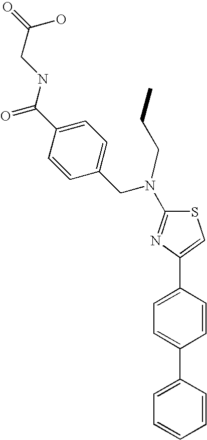 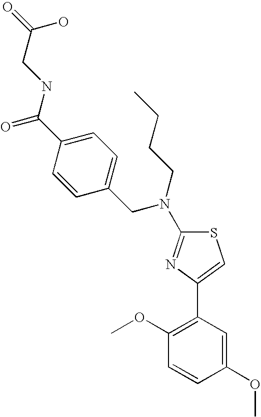 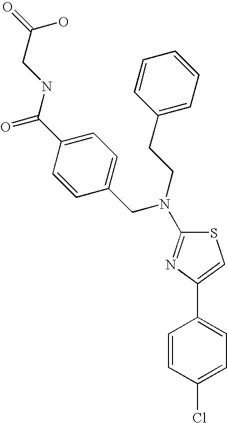 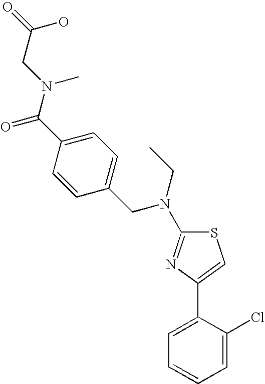 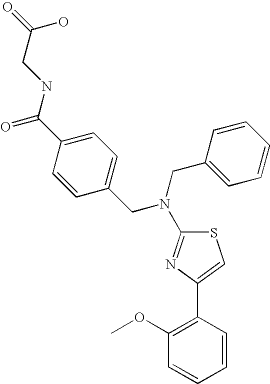 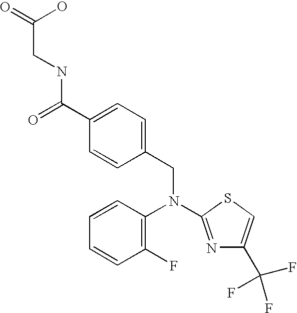 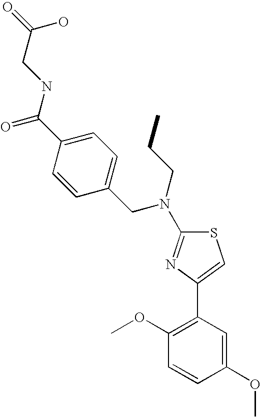 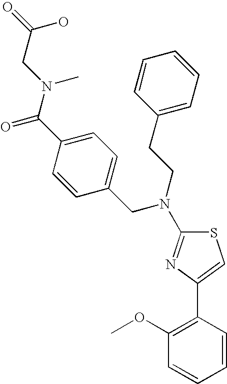 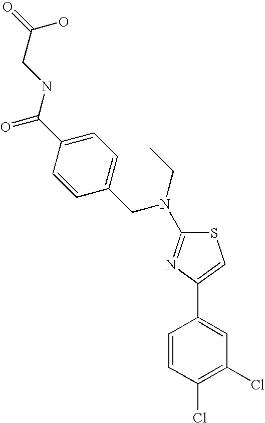 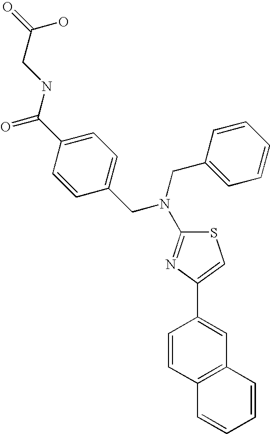 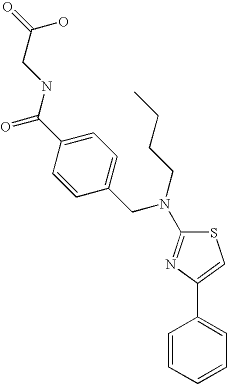 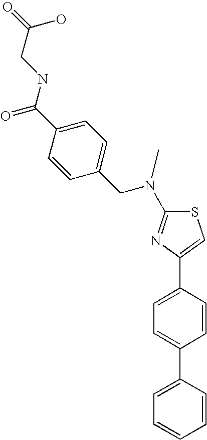 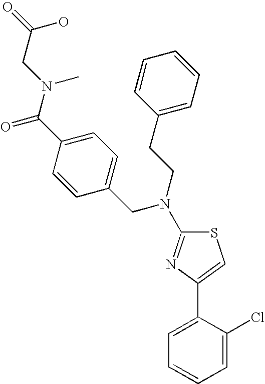 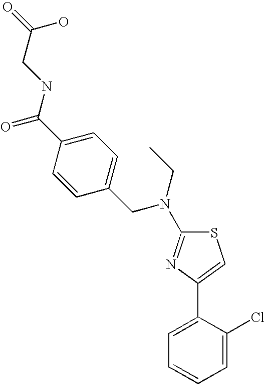 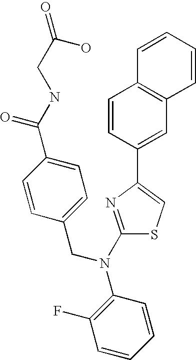 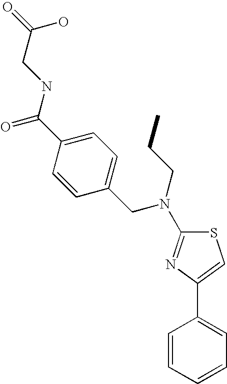 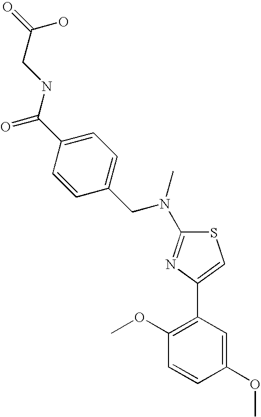 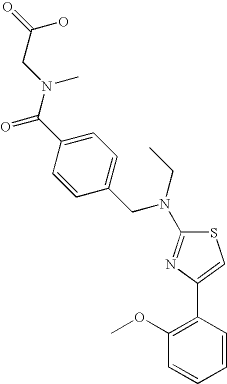 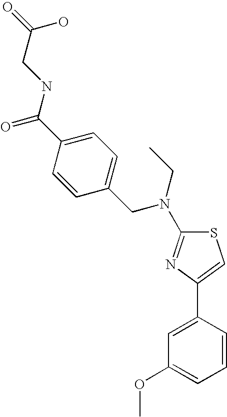 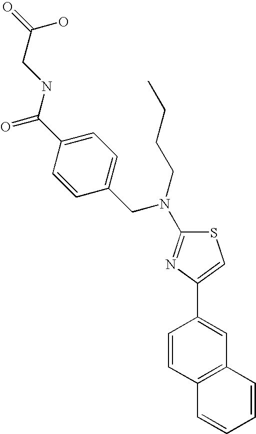 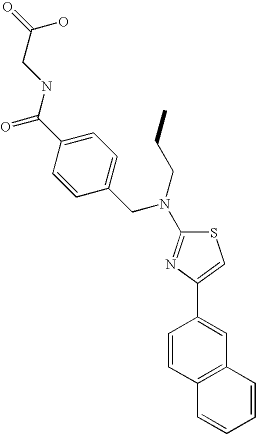 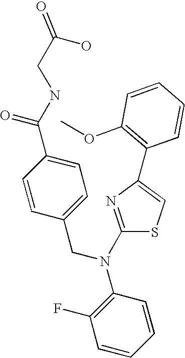 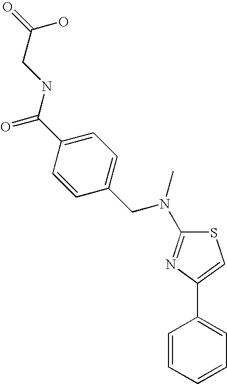 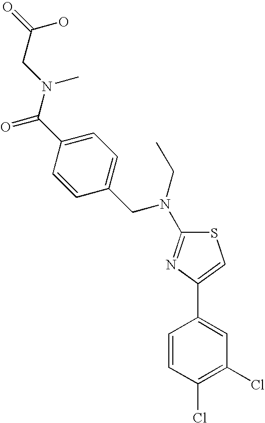 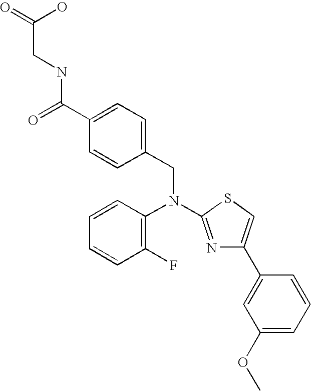 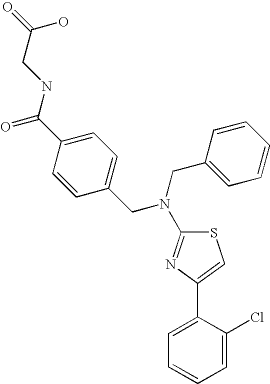 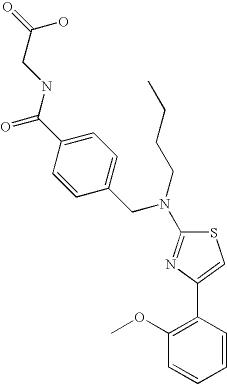 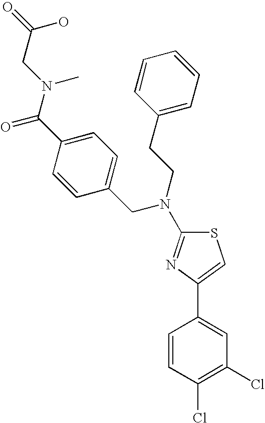 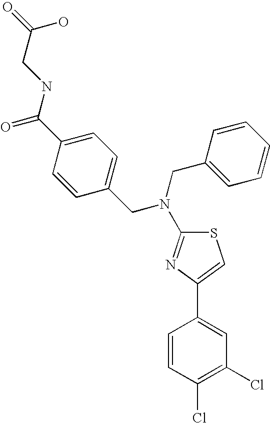 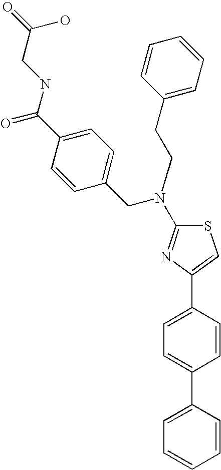 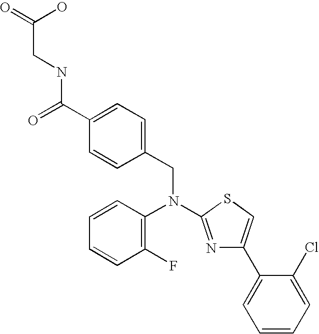 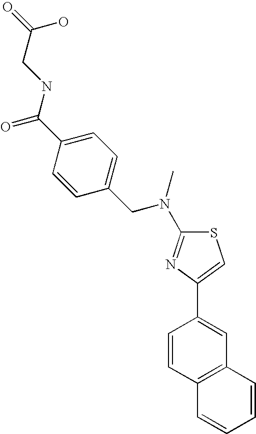 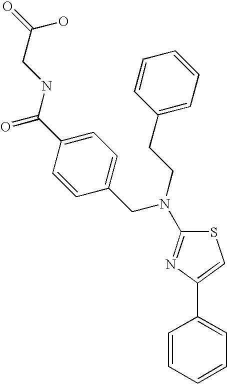 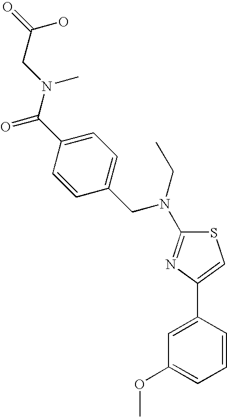 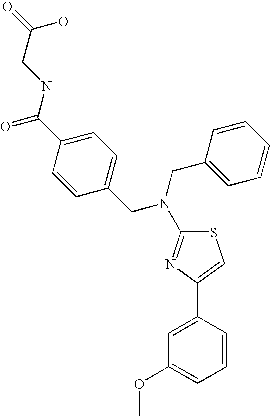 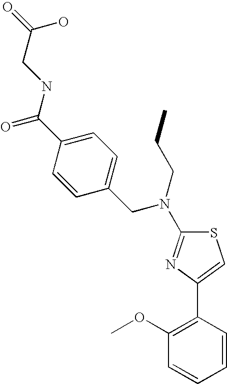 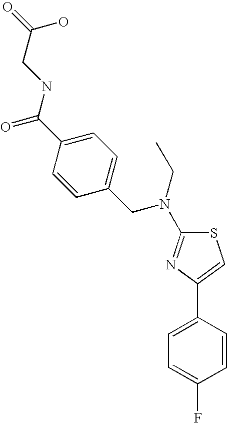 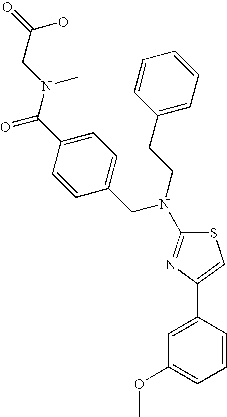 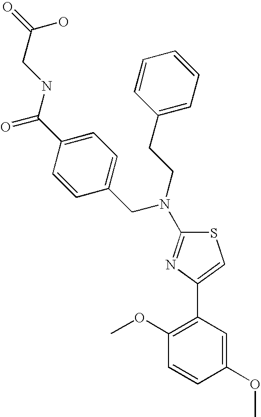 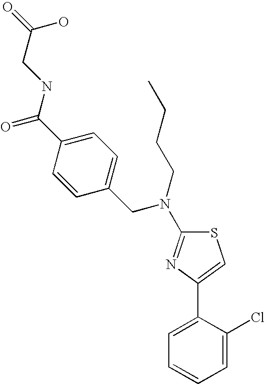 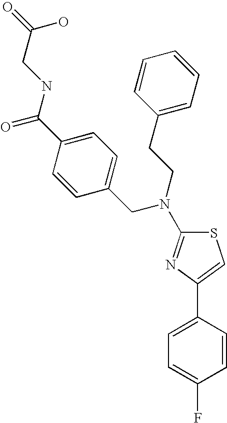 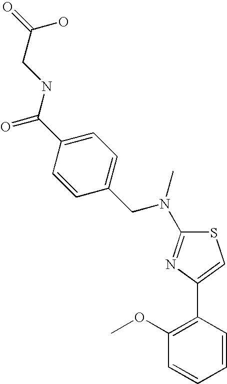 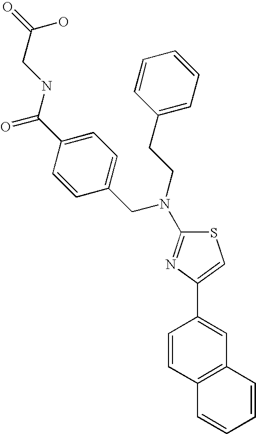 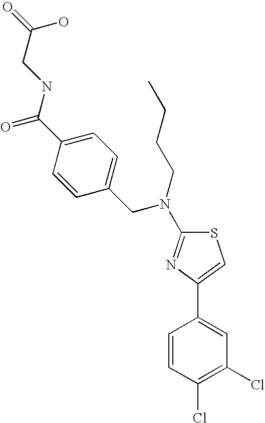 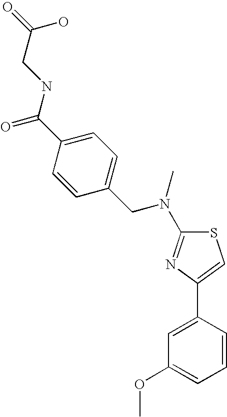 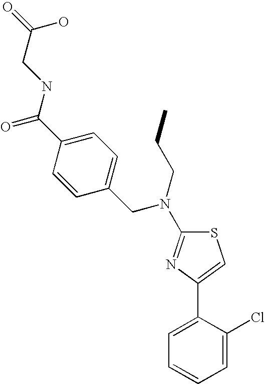 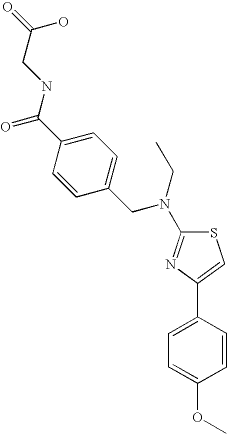 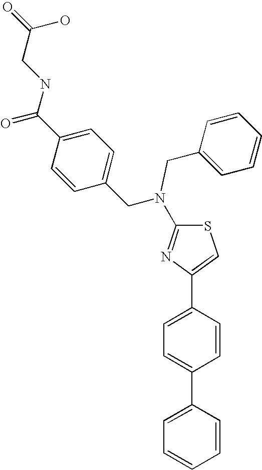 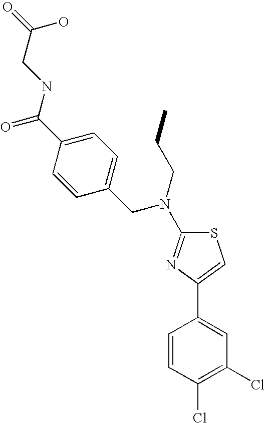 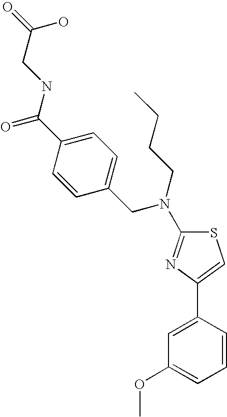 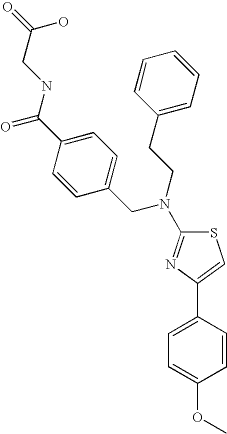 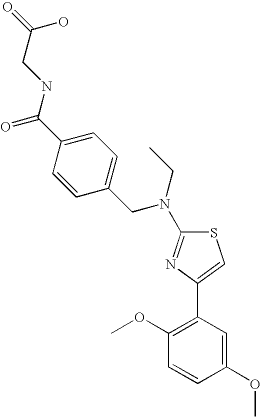 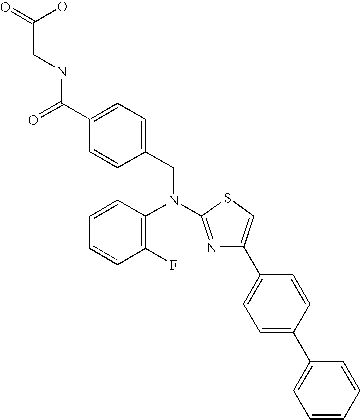 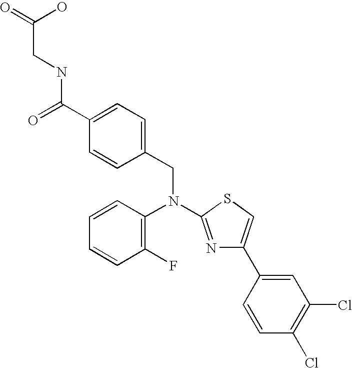 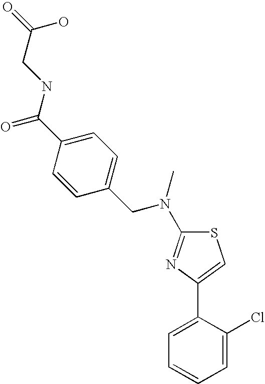 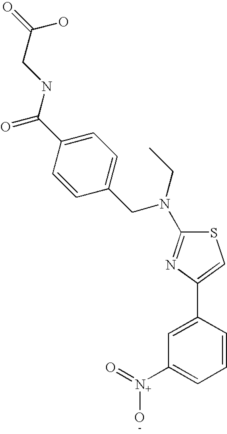 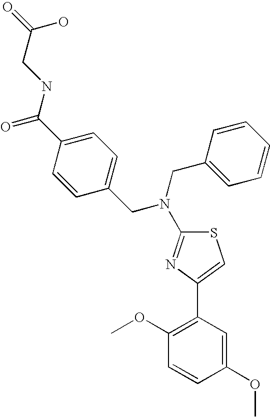 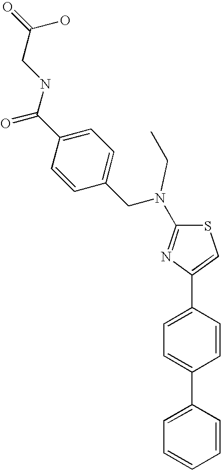 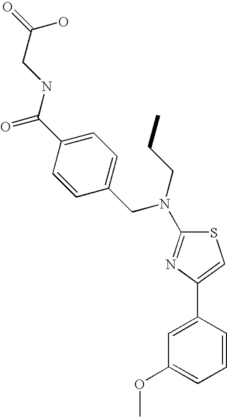 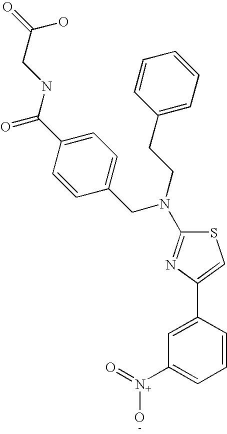 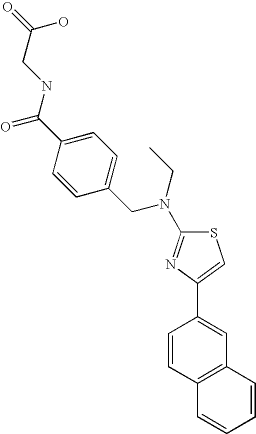 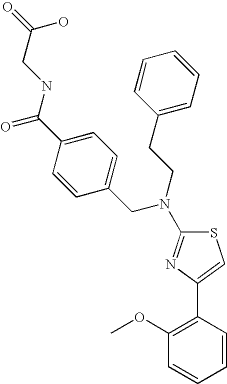 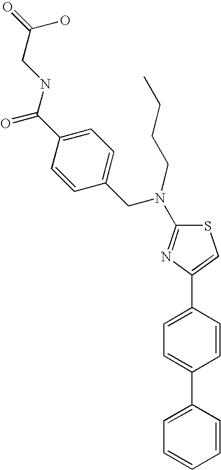 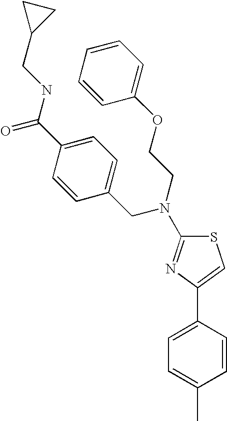 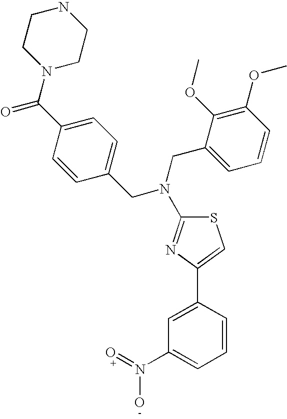 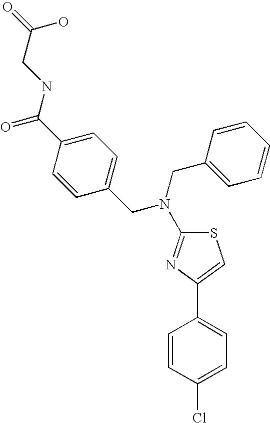 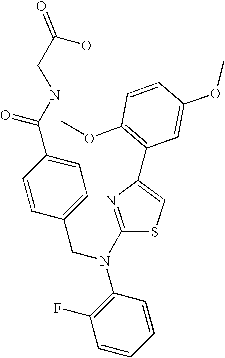 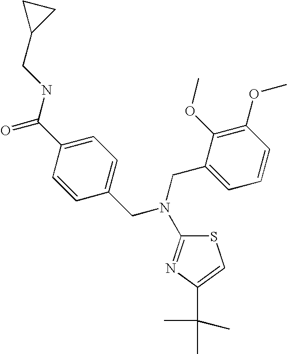 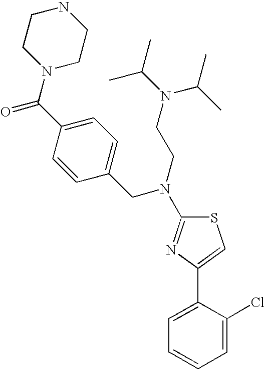 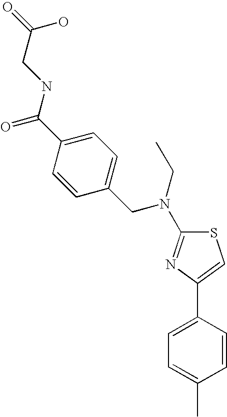 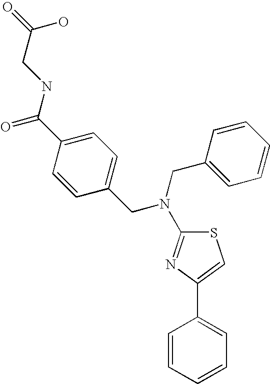 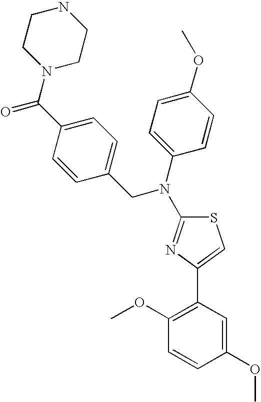 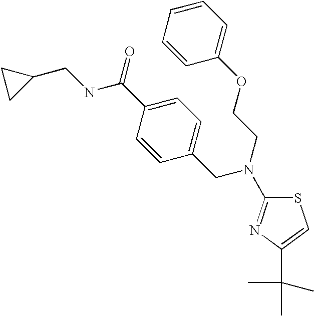 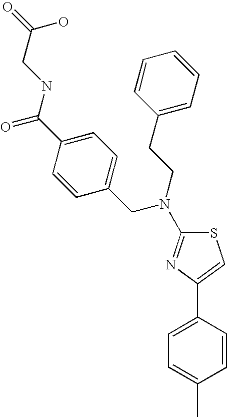 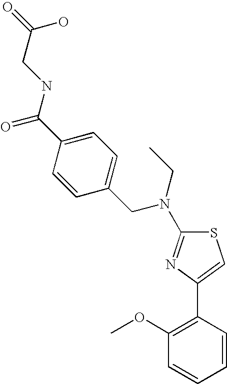 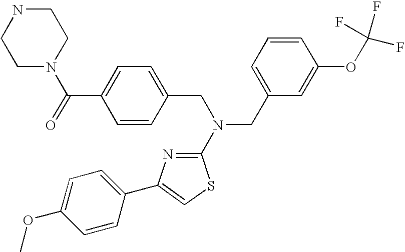 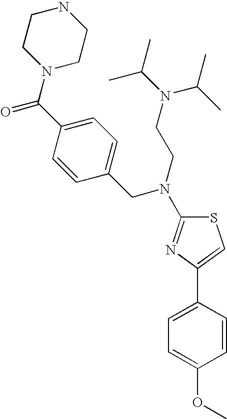 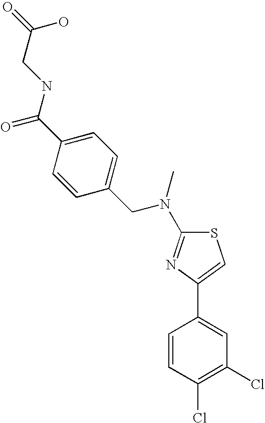 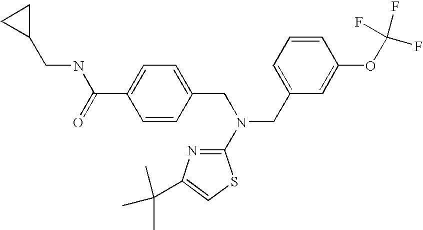 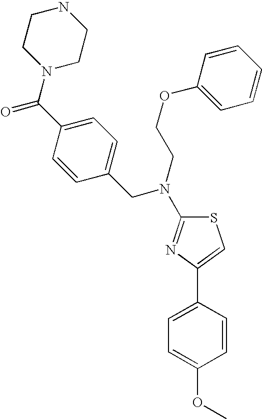 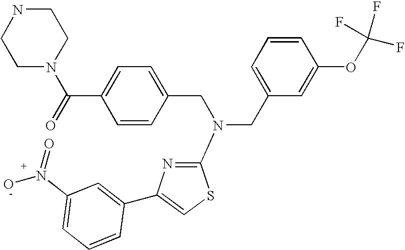 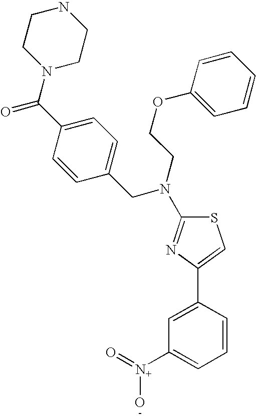 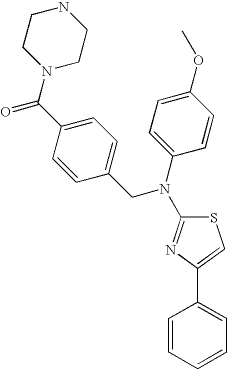 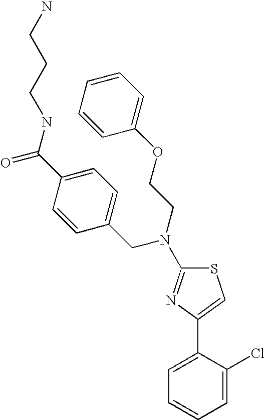 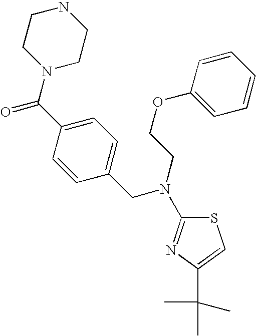 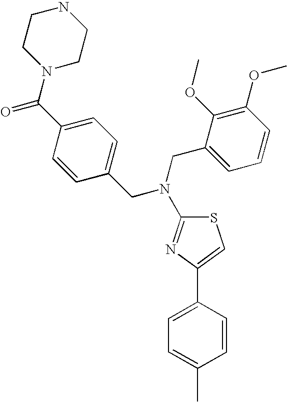 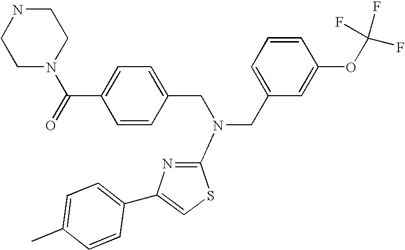 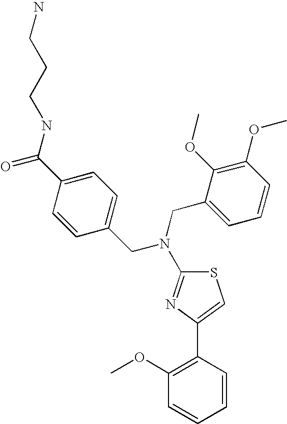 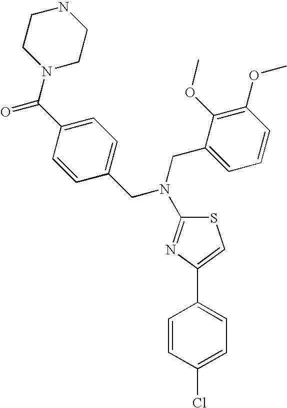 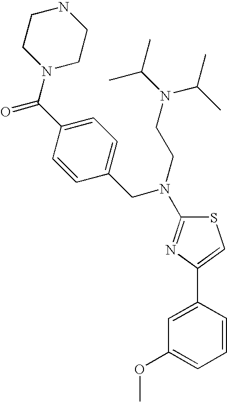 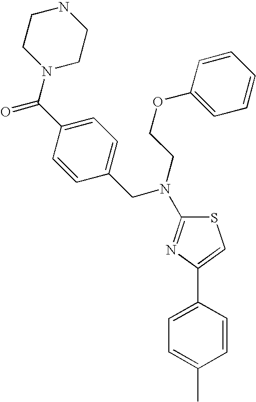 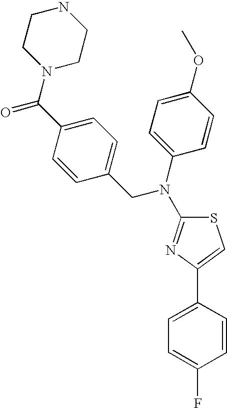 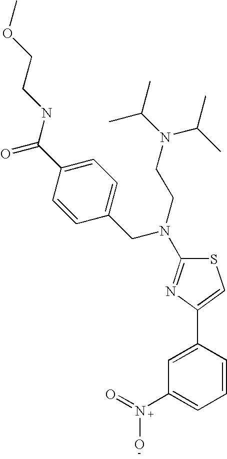 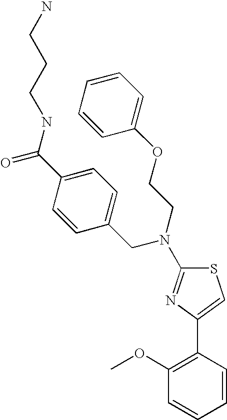 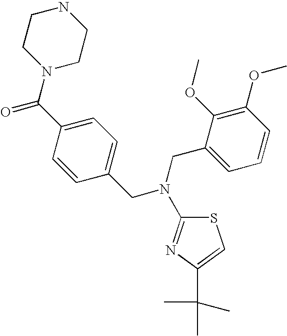 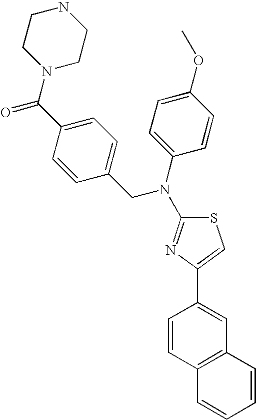 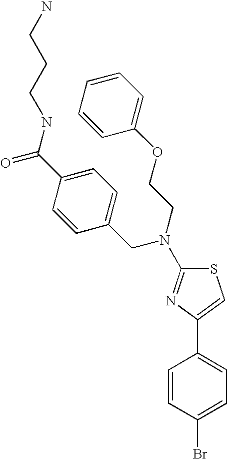 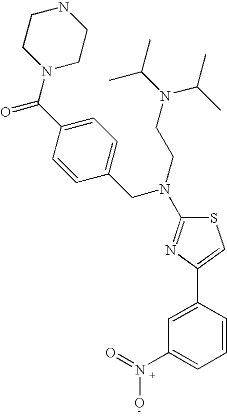 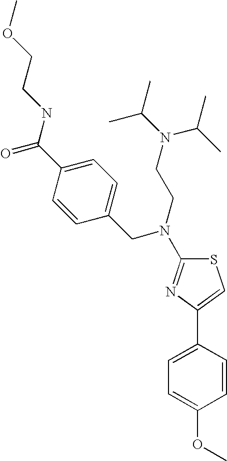 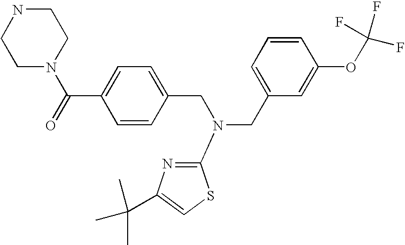 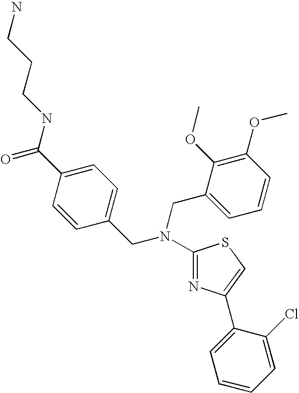 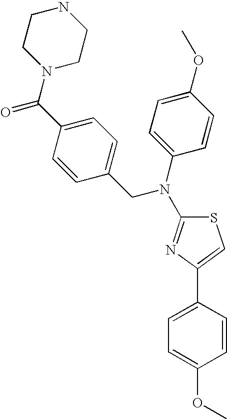 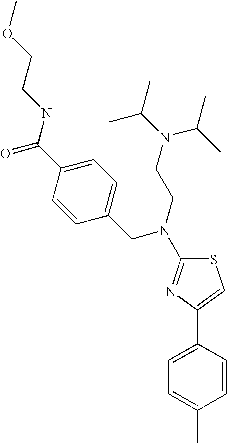 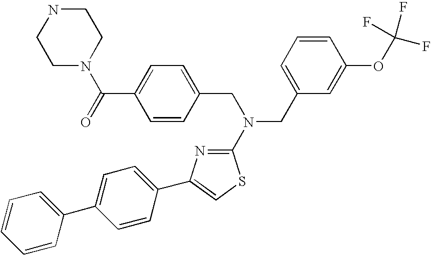 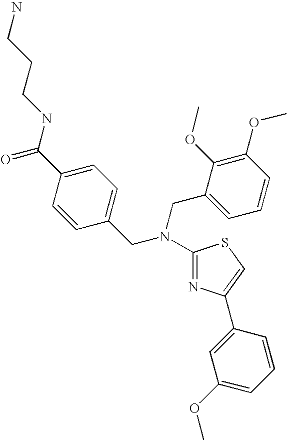 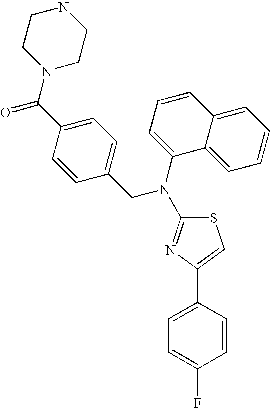 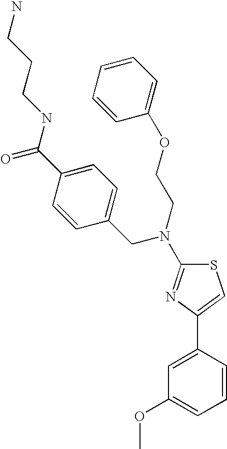 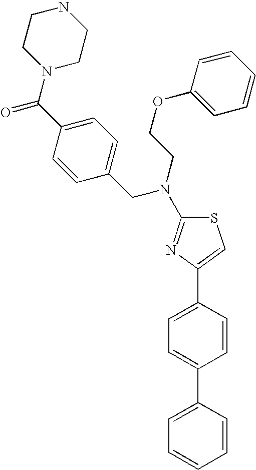 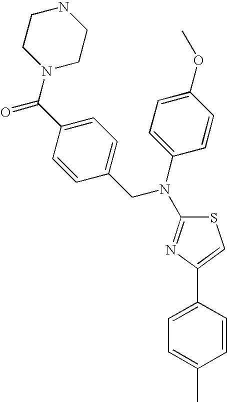 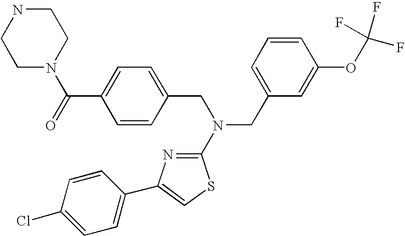 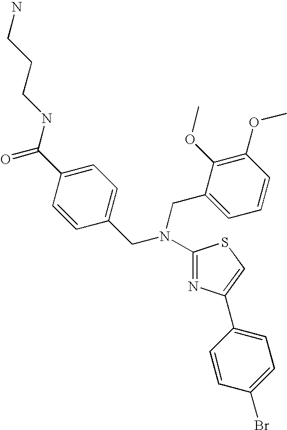 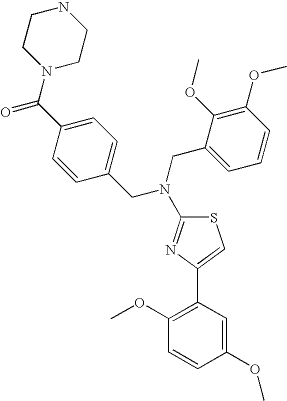 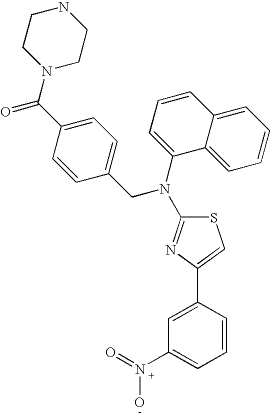 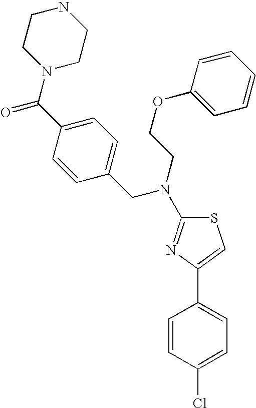 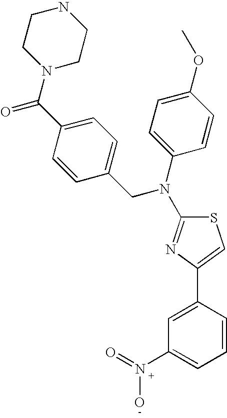 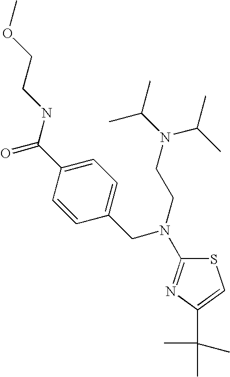 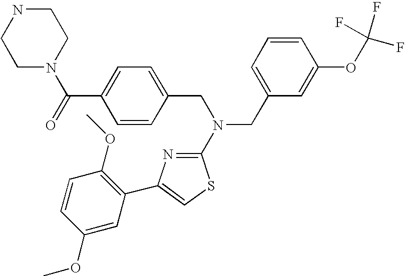 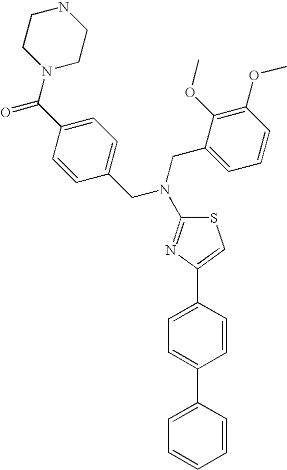 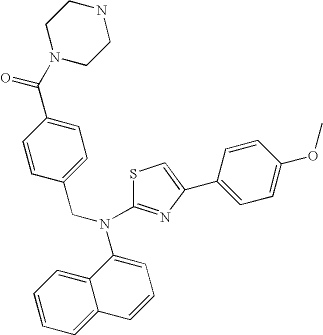 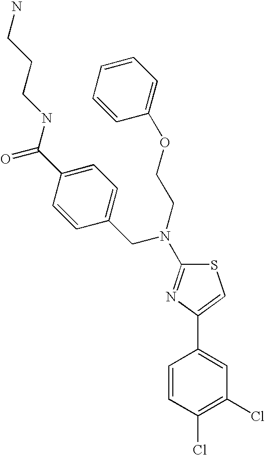 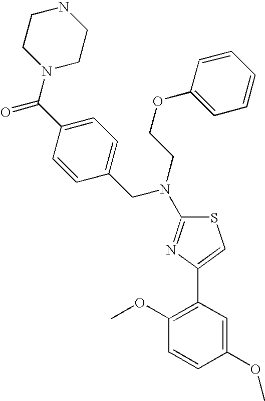 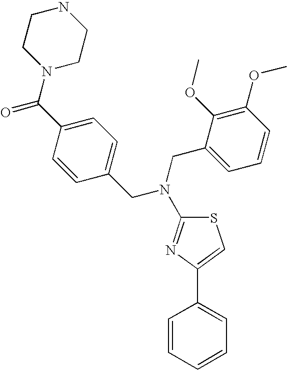 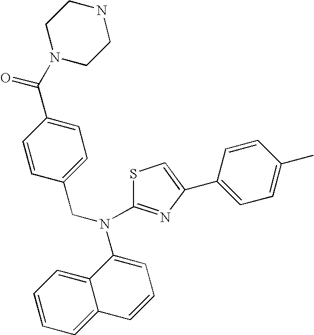 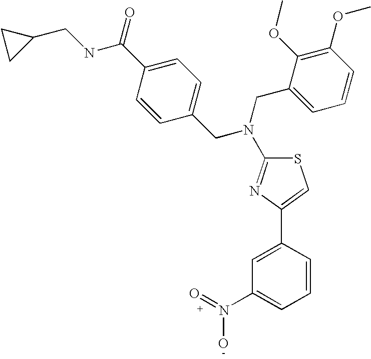 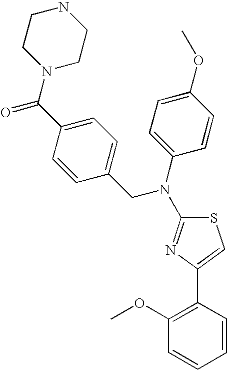 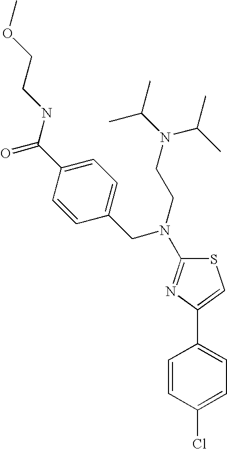 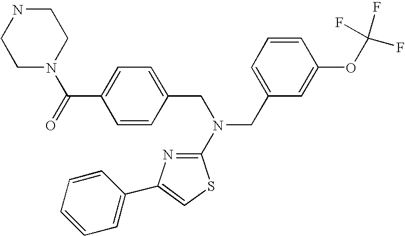 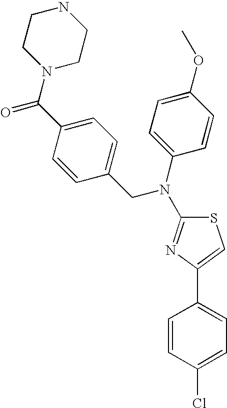 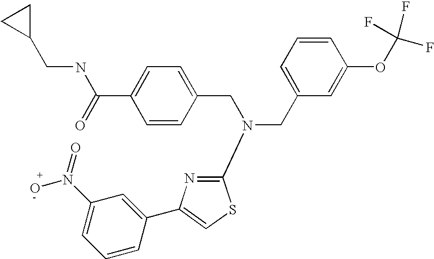 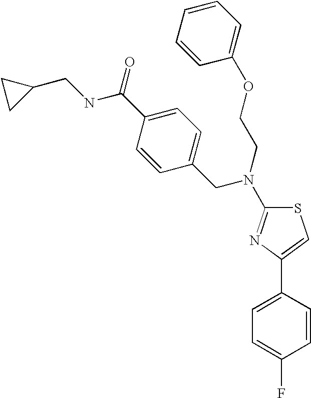 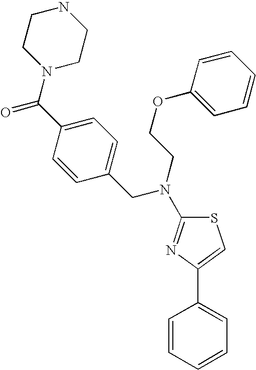 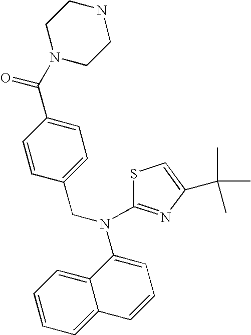 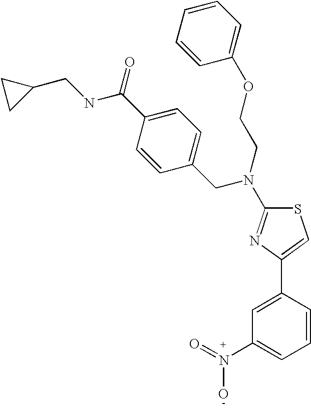 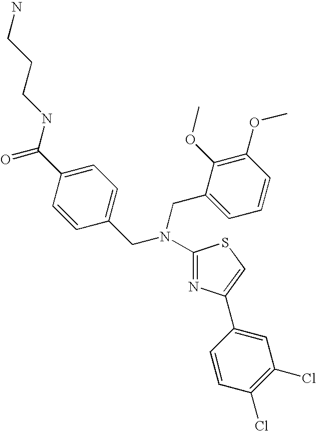 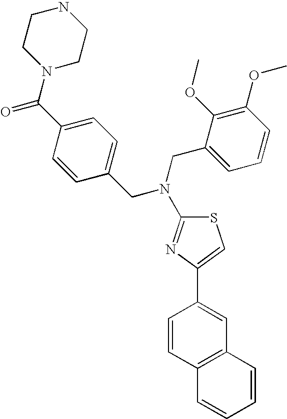 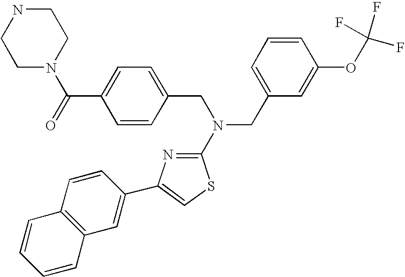 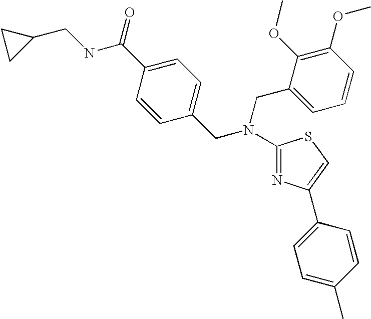 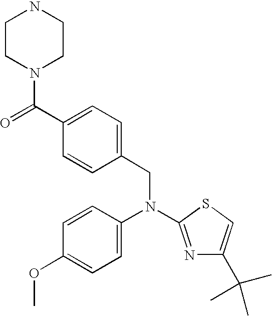 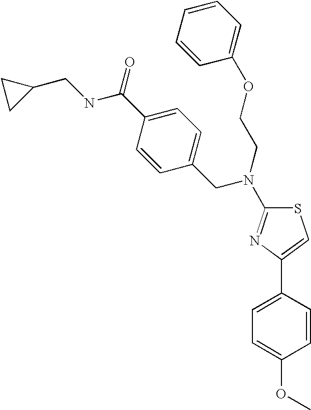 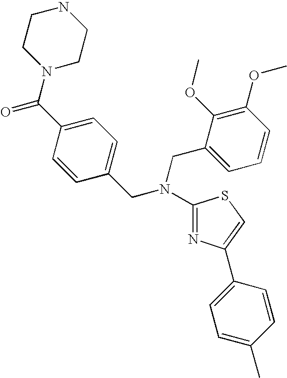 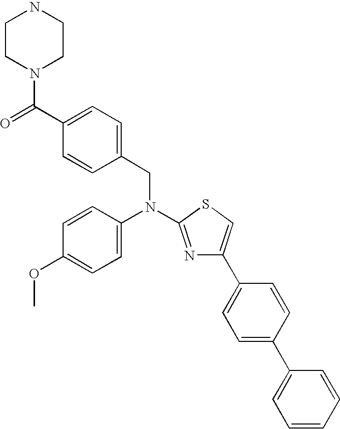 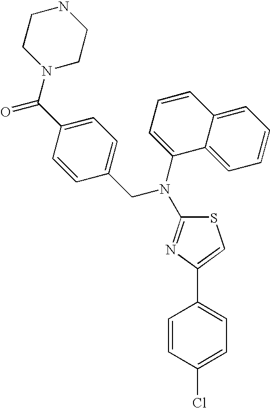 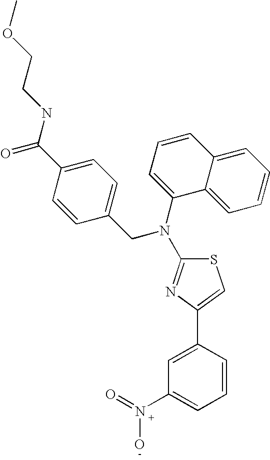 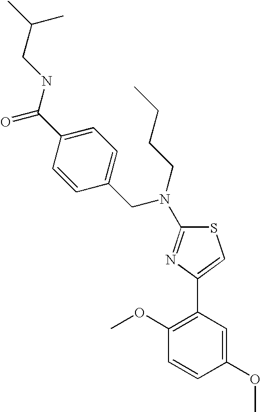 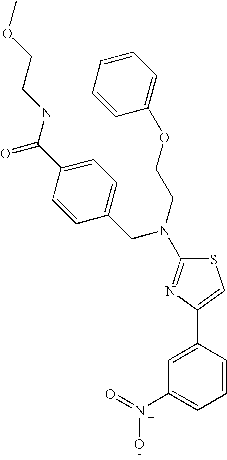 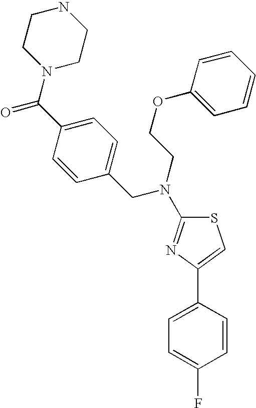 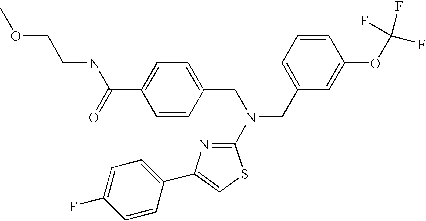 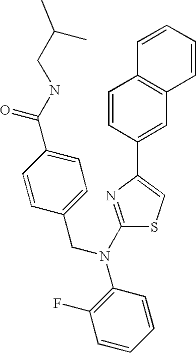 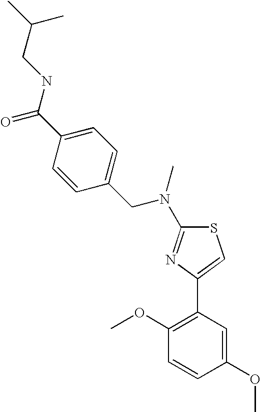 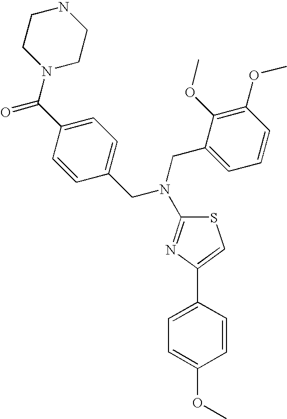 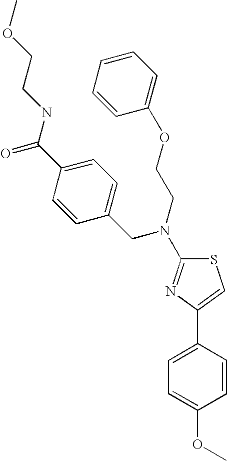 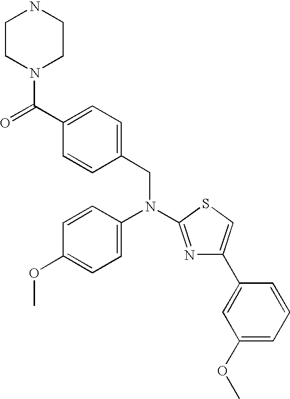 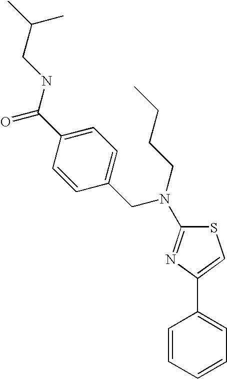 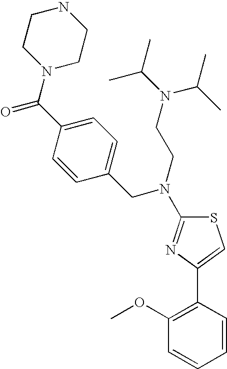 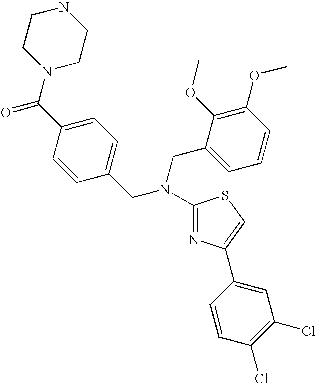 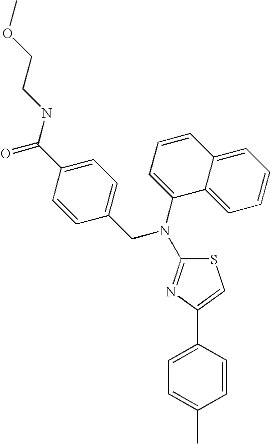 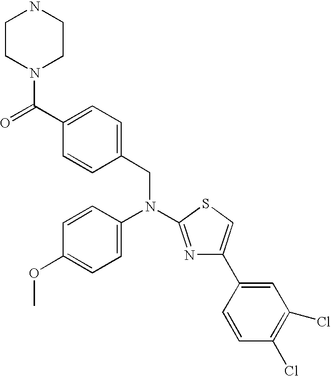 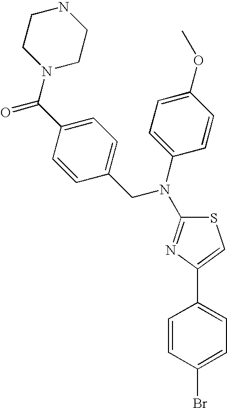 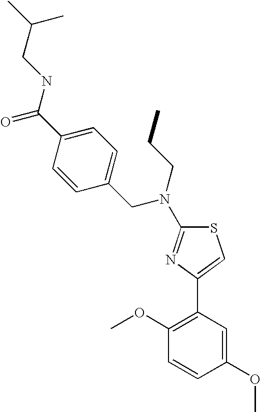 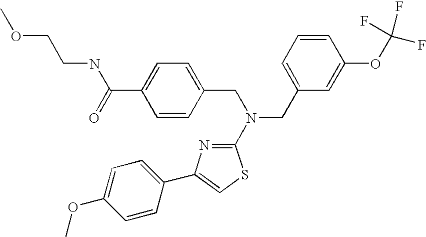 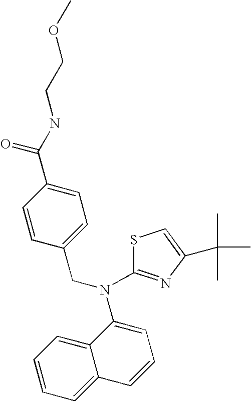 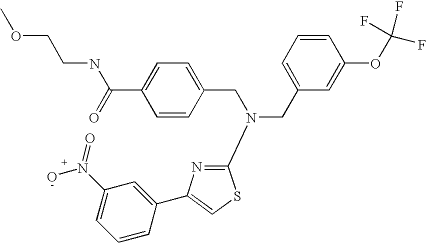 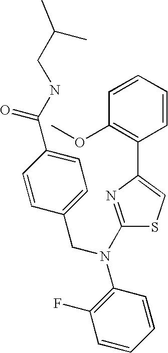 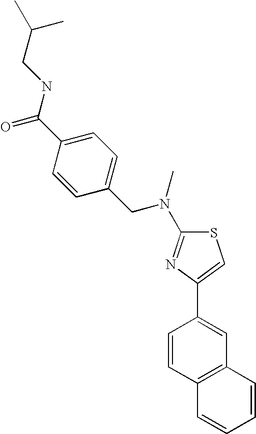 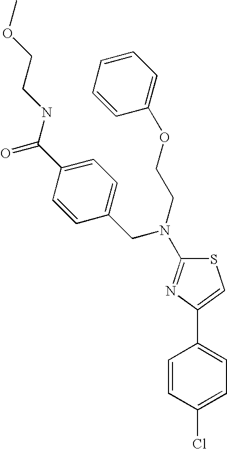 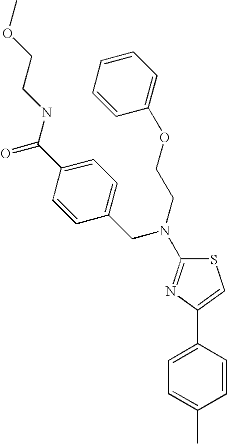 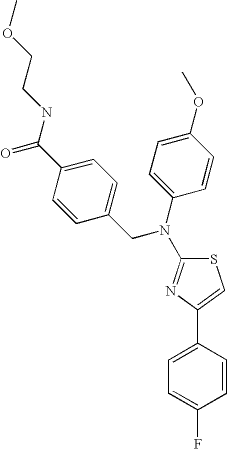 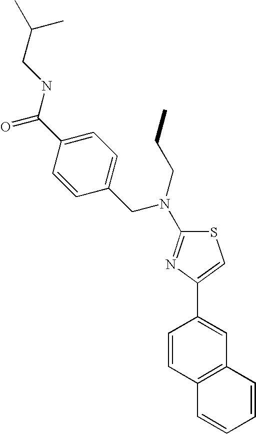 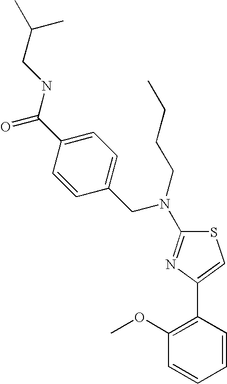 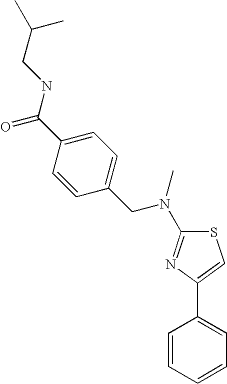 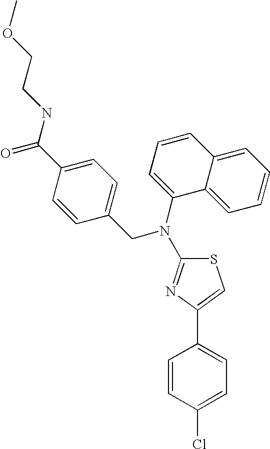 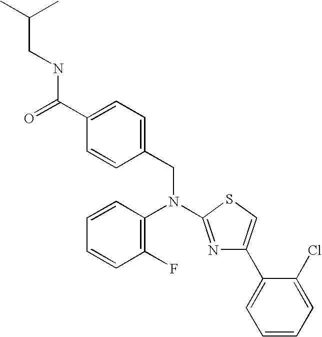 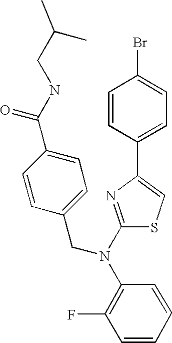 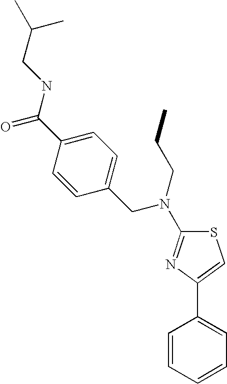 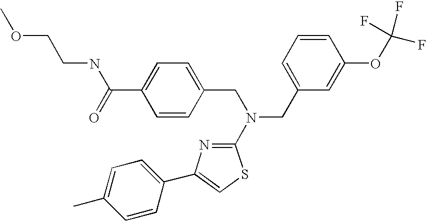 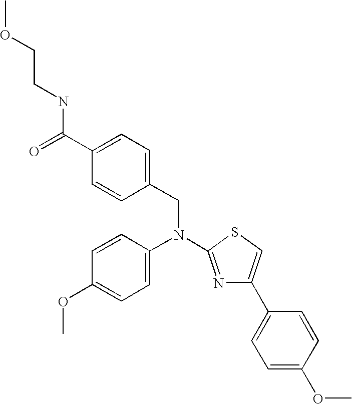 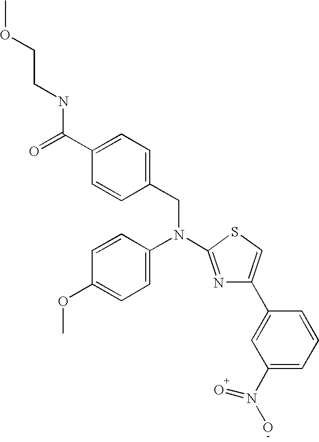 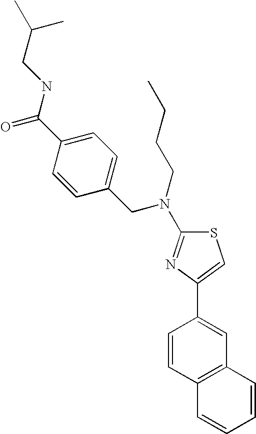 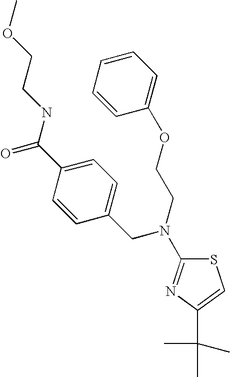 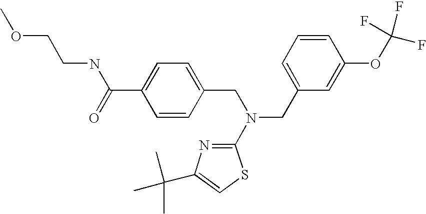 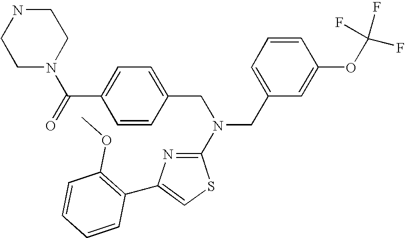 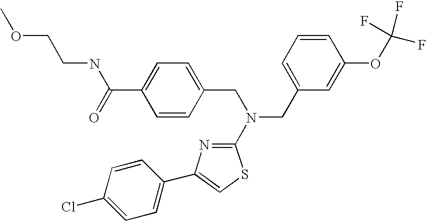 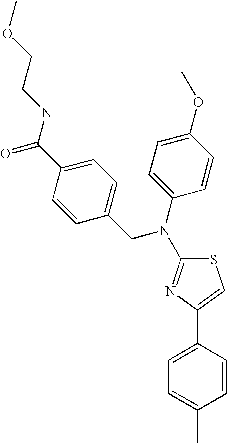 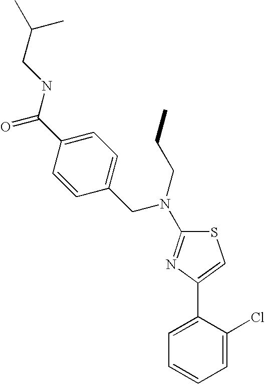 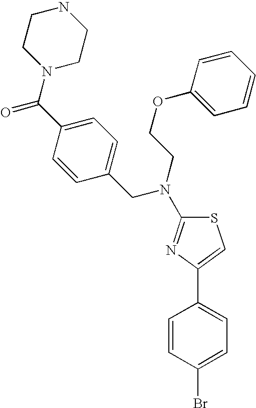 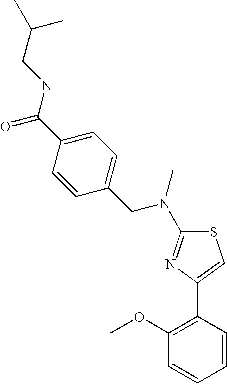 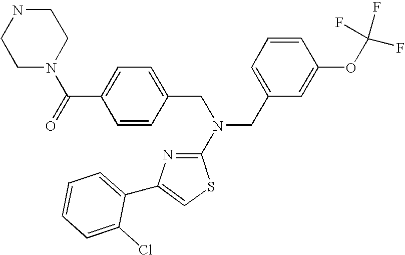 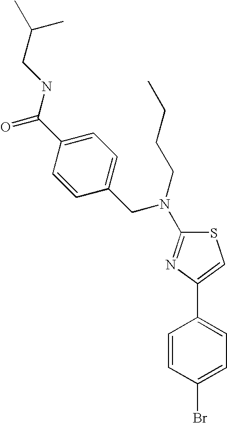 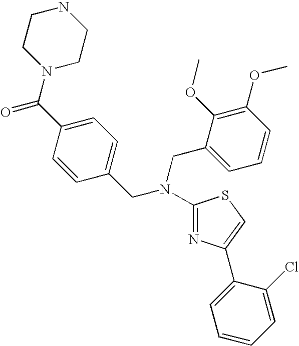 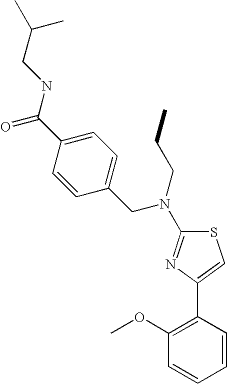 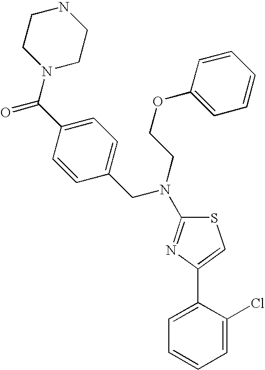 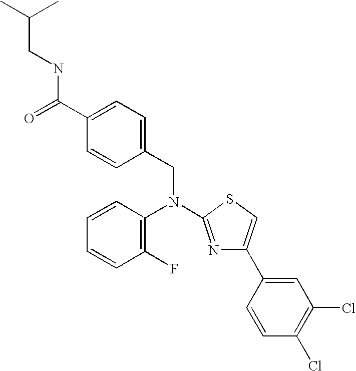 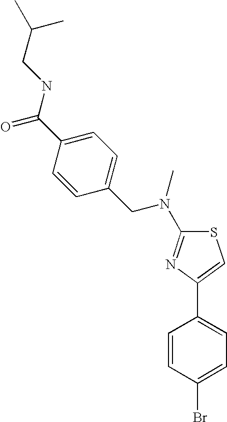 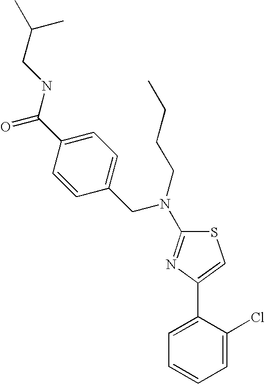 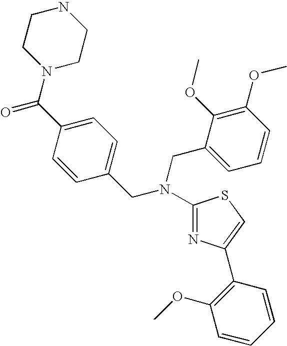 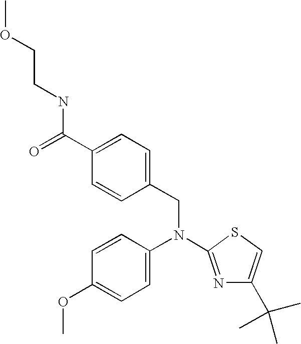 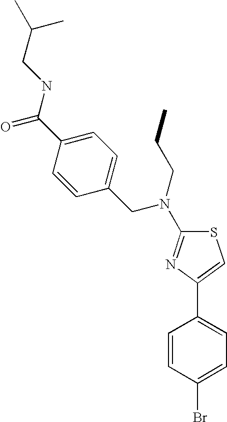 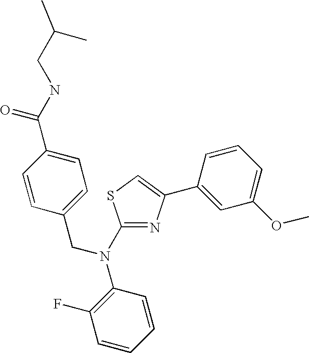 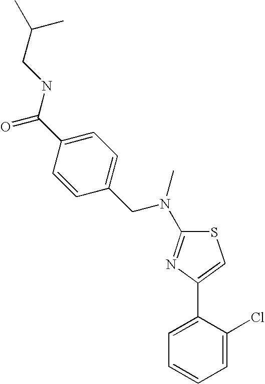 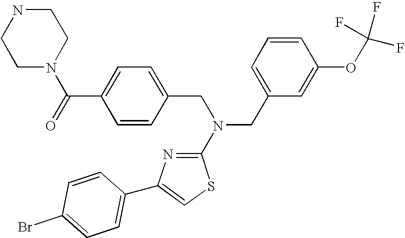 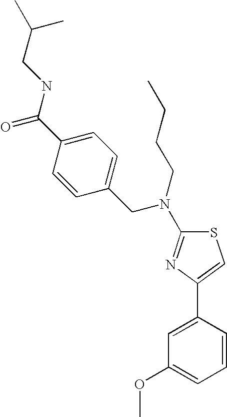 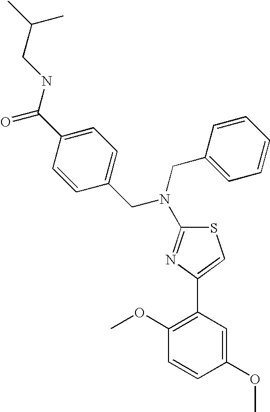 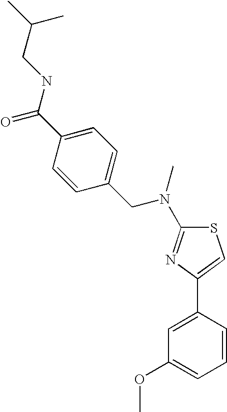 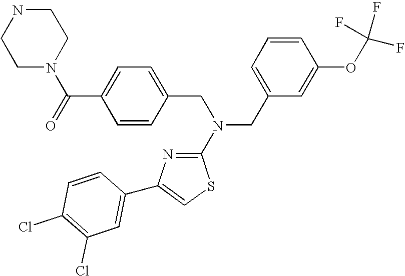 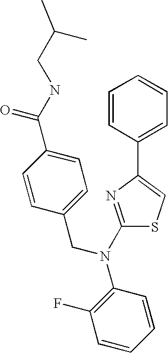 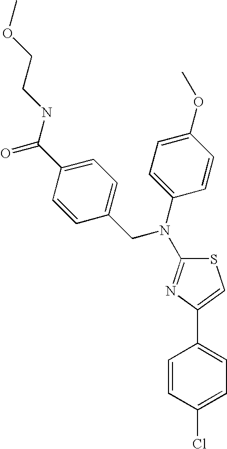 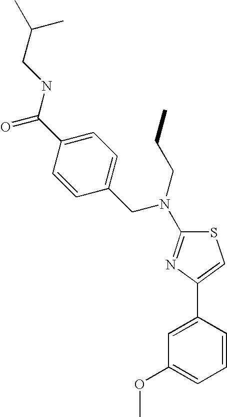 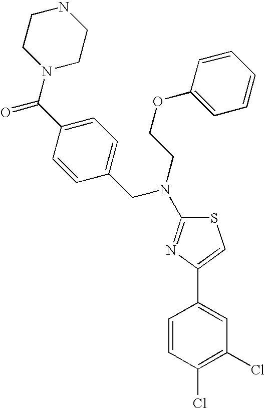 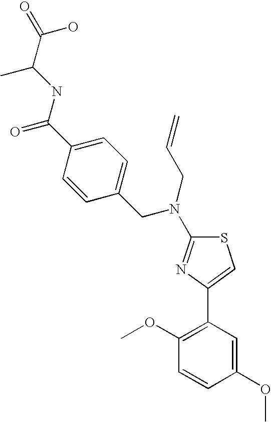 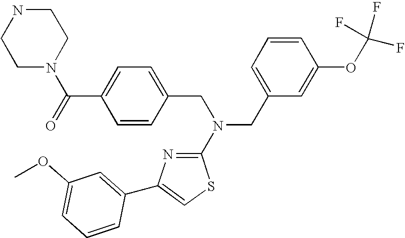 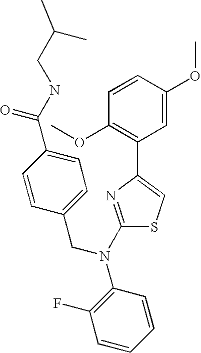 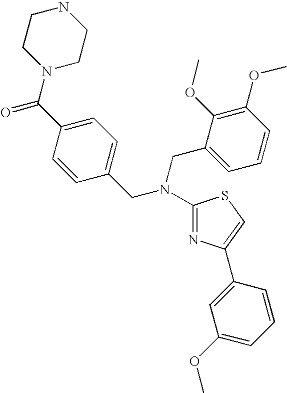 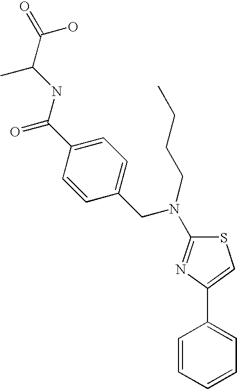 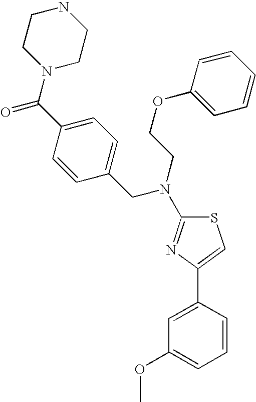 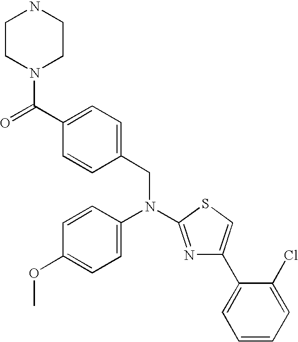 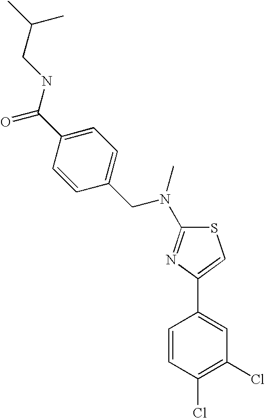 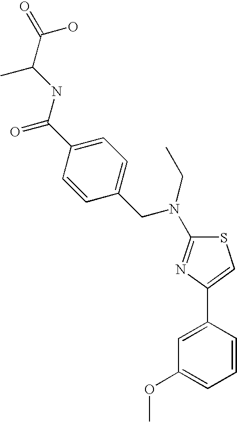 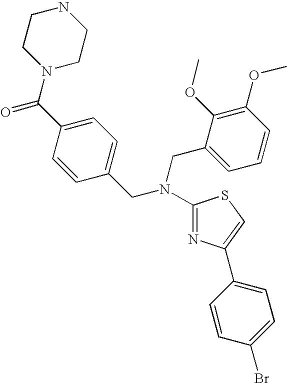 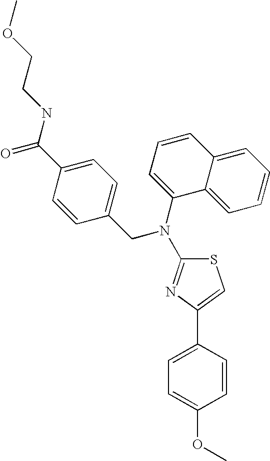 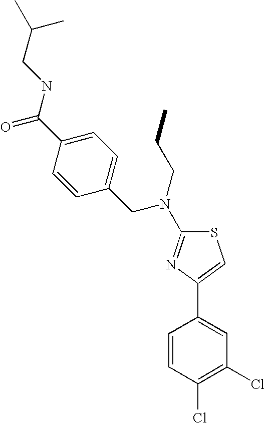 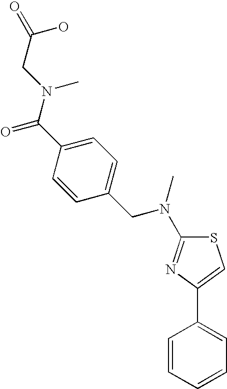 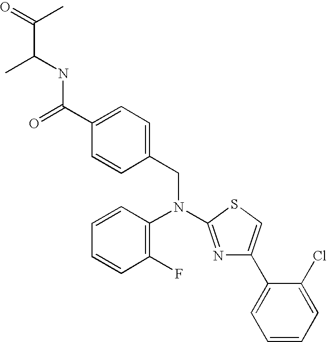 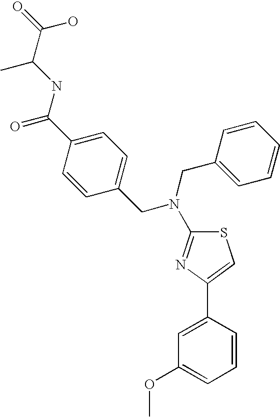 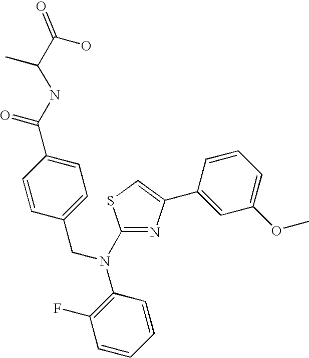 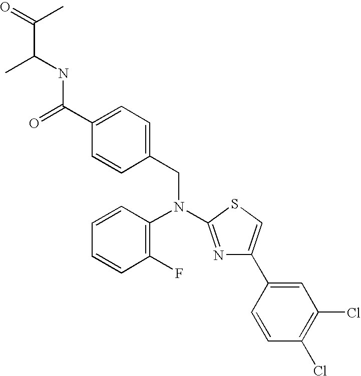 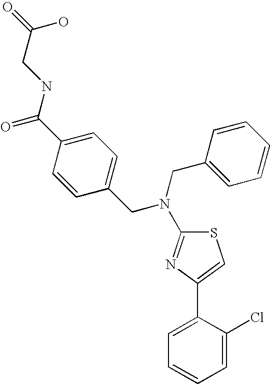 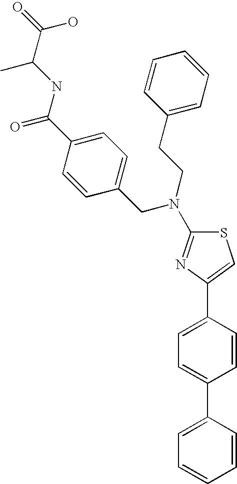 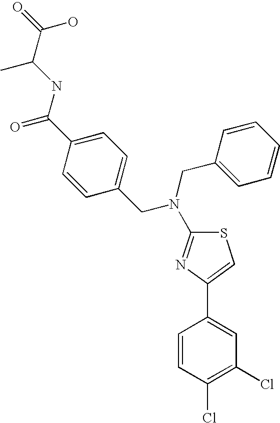 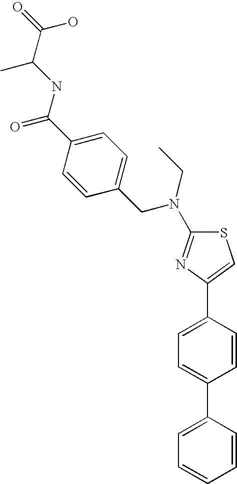 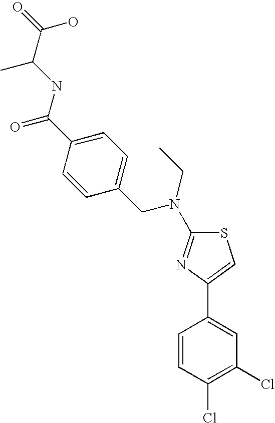 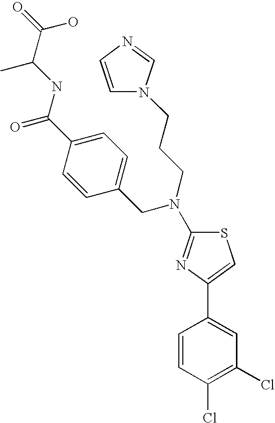 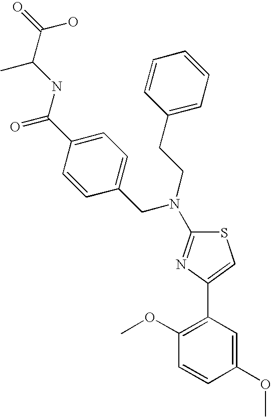 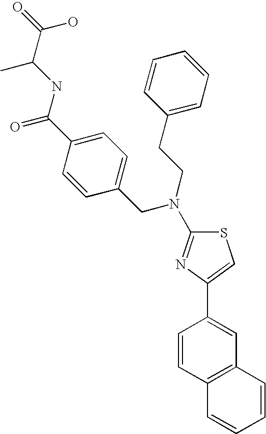 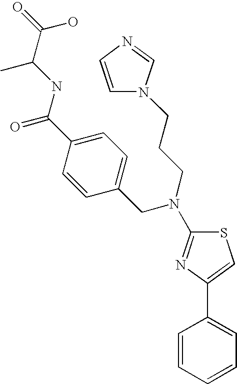 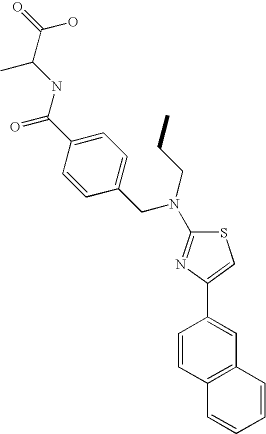 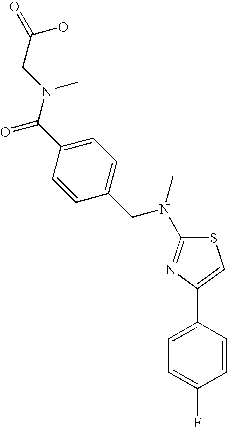 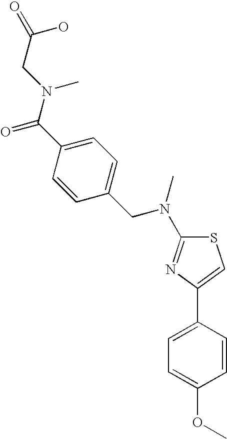 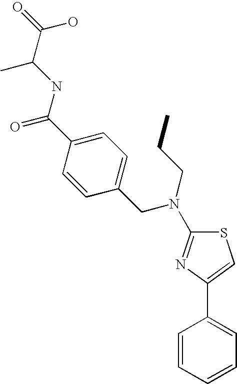 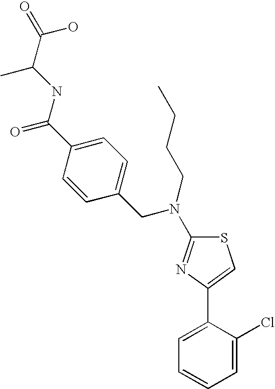 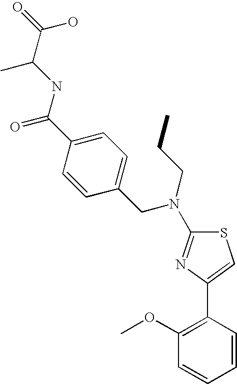 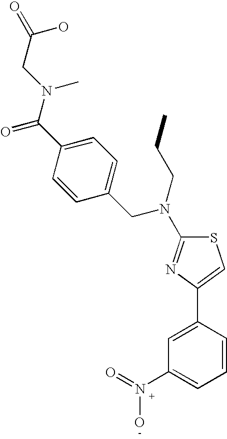 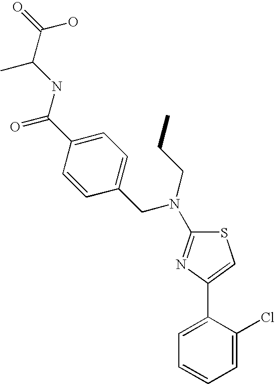 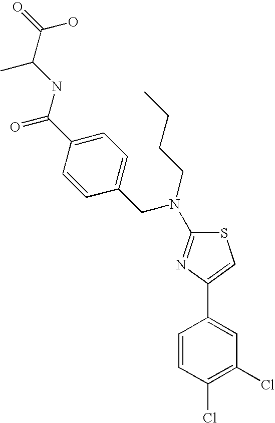 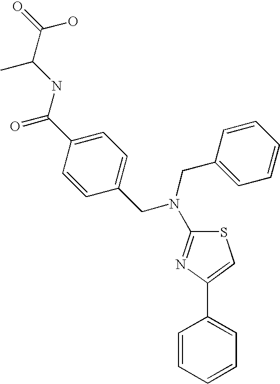 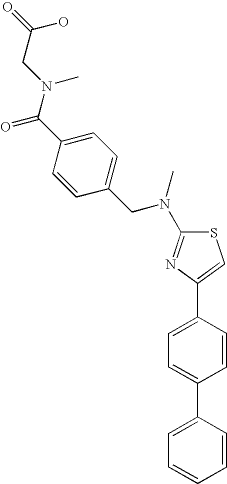 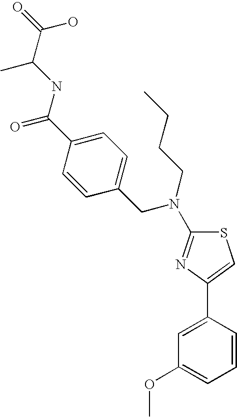 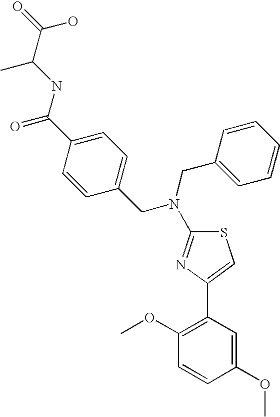 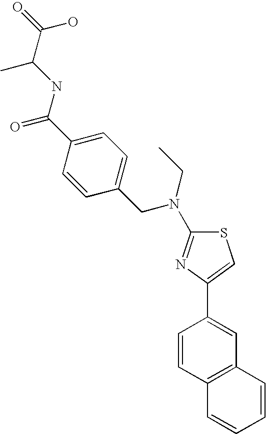 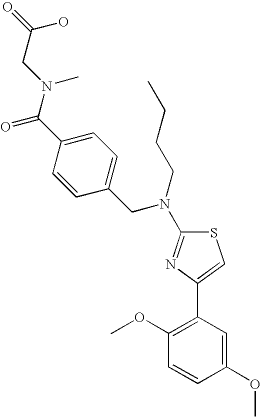 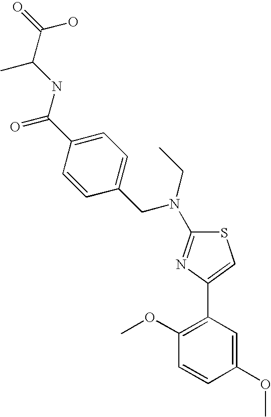 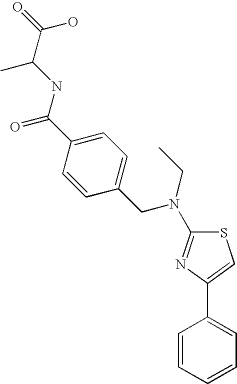 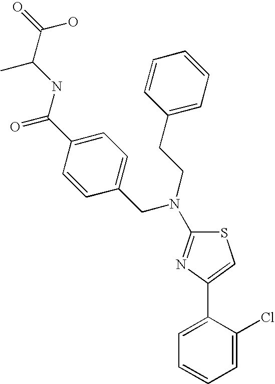 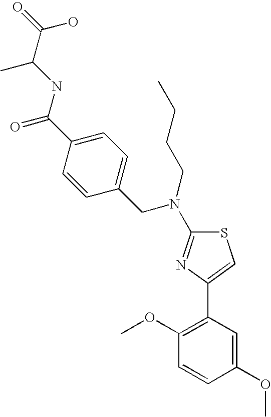 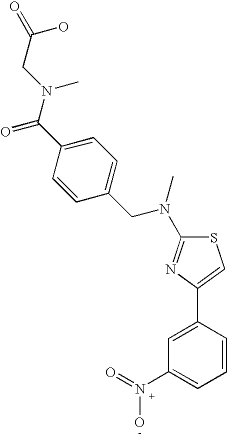 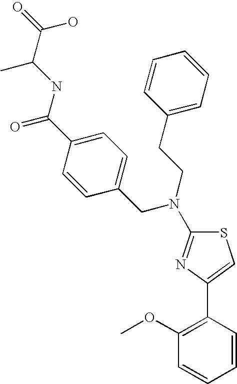 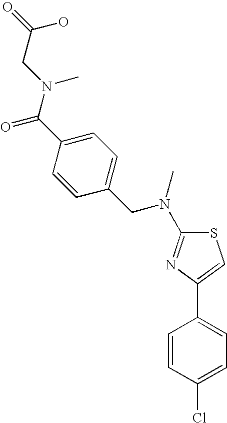 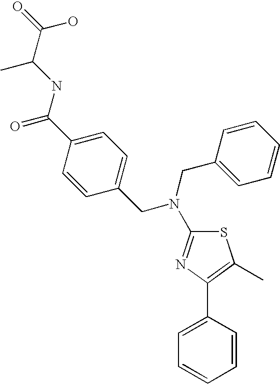 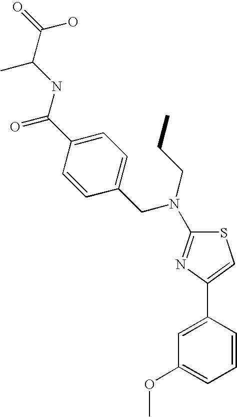 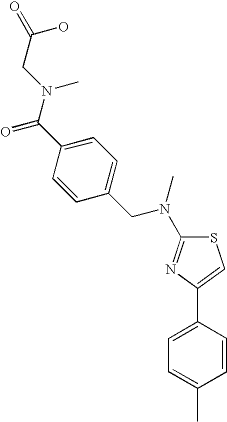 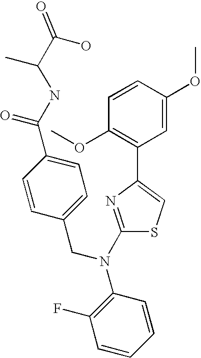 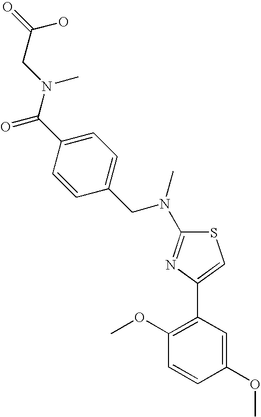 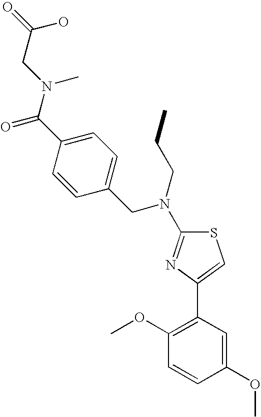 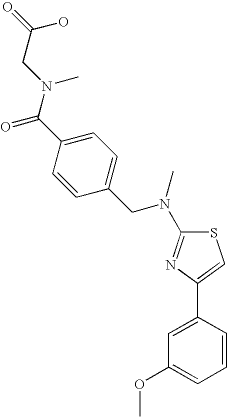 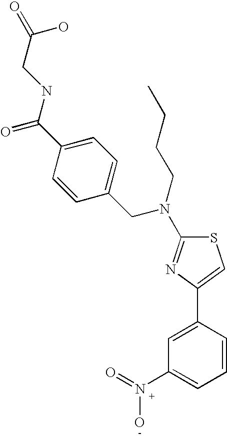 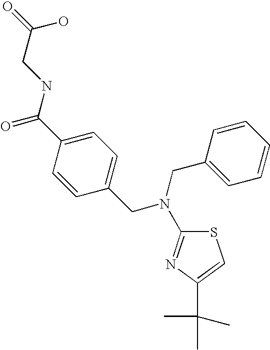 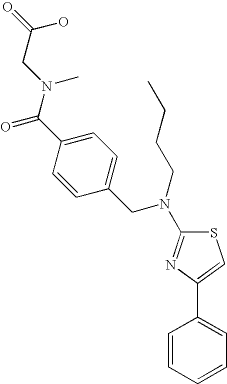 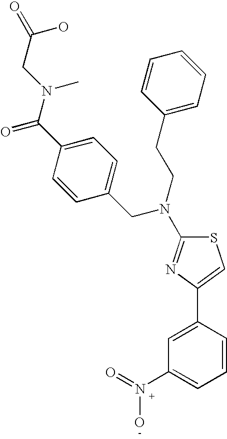 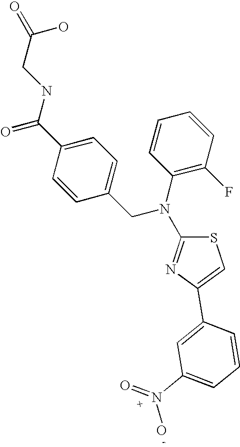 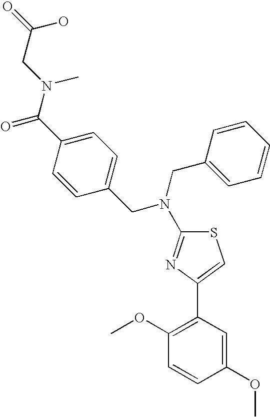 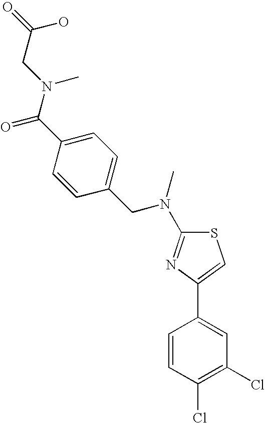 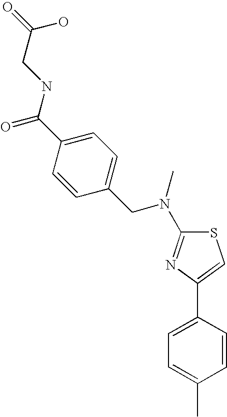 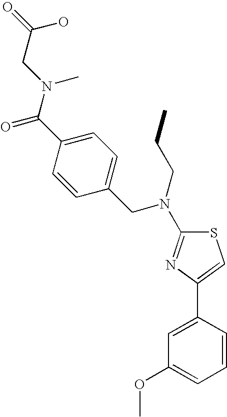 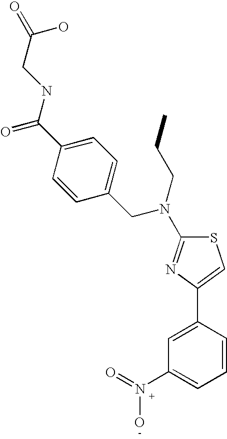 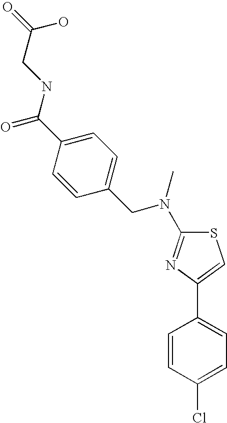
and pharmaceutically acceptable salts thereof.

53. A pharmaceutical composition comprising, together with a pharmaceutically acceptable carrier, diluent, or excipient, a compound (or a pharmaceutically acceptable salt thereof) according to claim 1.

54. A method of inhibiting ubiquitination in a cell comprising contacting the cell in which inhibition of ubiquitination is desired with a compound according to claim 1.

55. A method of inhibiting ubiquitination in a cell comprising contacting the cell in which inhibition of ubiquitination is desired with a composition according to claim 53.

56. The method according to claim 55, wherein the cell is an animal cell.

57. The method according to claim 56, wherein the animal cell is derived from a mammal.

58. A method of treating cell proliferative diseases or conditions comprising administering to a patient an effective amount of a composition according to claim 53.

59. The method according to claim 58, wherein the cell proliferative diseases are cancers.

60. A method of inhibiting MDM2, comprising administering to a patient an effective amount of a composition according to claim 53.

61. The method according to claim 60, wherein the patient suffers from a condition or disease that involves a process selected from the group consisting of inflammation, adaptive immunity, innate immunity, bone metabolism, LPS-induced angiogenesis, osteoporosis, osteopinneal diseases, lymph node development, mammary gland development, skin development, and central nervous system development.
